SEQUENCE LISTING (11 sequences)

SEQ ID NO: 1 - protein, 796 aa, Homo sapiens
MLRGGRRGQLGWHSWAAGPGSLLAWLILASAGAAPCPDACCPHGSSGLRCTRDGALDSLHHLPGAENLTELYIENQQHLQHLELRDLRGLGELRNLTIVKSGLRFVAPDAFHFTPRLSRLNLSFNALESLSWKTVQGLSLQELVLSGNPLHCSCALRWLQRWEEEGLGGVPEQKLQCHGQGPLAHMPNASCGVPTLKVQVPNASVDVGDDVLLRCQVEGRGLEQAGWILTELEQSATVMKSGGLPSLGLTLANVTSDLNRKNVTCWAENDVGRAEVSVQVNVSFPASVQLHTAVEMHHWCIPFSVDGQPAPSLRWLFNGSVLNETSFIFTEFLEPAANETVRHGCLRLNQPTHVNNGNYTLLAANPFGQASASIMAAFMDNPFEFNPEDPIPVSFSPVDTNSTSGDPVEKKDETPFGVSVAVGLAVFACLFLSTLLLVLNKCGRRNKFGINRPAVLAPEDGLAMSLHFMTLGGSSLSPTEGKGSGLQGHIIENPQYFSDACVHHIKRRDIVLKWELGEGAFGKVFLAECHNLLPEQDKMLVAVKALKEASESARQDFQREAELLTMLQHQHIVRFFGVCTEGRPLLMVFEYMRHGDLNRFLRSHGPDAKLLAGGEDVAPGPLGLGQLLAVASQVAAGMVYLAGLHFVHRDLATRNCLVGQGLVVKIGDFGMSRDIYSTDYYRVGGRTMLPIRWMPPESILYRKFTTESDVWSFGVVLWEIFTYGKQPWYQLSNTEAIDCITQGRELERPRACPPEVYAIMRGCWQREPQQRHSIKDVHARLQALAQAPPVYLDVLG

SEQ ID NO: 2 - DNA, 2663 bp, Homo sapiens
tgcagctgggagcgcacagacggctgccccgcctgagcgaggcgggcgccgccgcgatgctgcgaggcggacggcgcgggcagcttggctggcacagctgggctgcggggccgggcagcctgctggcttggctgatactggcatctgcgggcgccgcaccctgccccgatgcctgctgcccccacggctcctcgggactgcgatgcacccgggatggggccctggatagcctccaccacctgcccggcgcagagaacctgactgagctctacatcgagaaccagcagcatctgcagcatctggagctccgtgatctgaggggcctgggggagctgagaaacctcaccatcgtgaagagtggtctccgtttcgtggcgccagatgccttccatttcactcctcggctcagtcgcctgaatctctccttcaacgctctggagtctctctcctggaaaactgtgcagggcctctccttacaggaactggtcctgtcggggaaccctctgcactgttcttgtgccctgcgctggctacagcgctgggaggaggagggactgggcggagtgcctgaacagaagctgcagtgtcatgggcaagggcccctggcccacatgcccaatgccagctgtggtgtgcccacgctgaaggtccaggtgcccaatgcctcggtggatgtgggggacgacgtgctgctgcggtgccaggtggaggggcggggcctggagcaggccggctggatcctcacagagctggagcagtcagccacggtgatgaaatctgggggtctgccatccctggggctgaccctggccaatgtcaccagtgacctcaacaggaagaacgtgacgtgctgggcagagaacgatgtgggccgggcagaggtctctgttcaggtcaacgtctccttcccggccagtgtgcagctgcacacggcggtggagatgcaccactggtgcatccccttctctgtggatgggcagccggcaccgtctctgcgctggctcttcaatggctccgtgctcaatgagaccagcttcatcttcactgagttcctggagccggcagccaatgagaccgtgcggcacgggtgtctgcgcctcaaccagcccacccacgtcaacaacggcaactacacgctgctggctgccaaccccttcggccaggcctccgcctccatcatggctgccttcatggacaaccctttcgagttcaaccccgaggaccccatccctgtctccttctcgccggtggacactaacagcacatctggagacccggtggagaagaaggacgaaacaccttttggggtctcggtggctgtgggcctggccgtctttgcctgcctcttcctttctacgctgctccttgtgctcaacaaatgtggacggagaaacaagtttgggatcaaccgcccggctgtgctggctccagaggatgggctggccatgtccctgcatttcatgacattgggtggcagctccctgtcccccaccgagggcaaaggctctgggctccaaggccacatcatcgagaacccacaatacttcagtgatgcctgtgttcaccacatcaagcgccgggacatcgtgctcaagtgggagctgggggagggcgcctttgggaaggtcttccttgctgagtgccacaacctcctgcctgagcaggacaagatgctggtggctgtcaaggcactgaaggaggcgtccgagagtgctcggcaggacttccagcgtgaggctgagctgctcaccatgctgcagcaccagcacatcgtgcgcttcttcggcgtctgcaccgagggccgccccctgctcatggtctttgagtatatgcggcacggggacctcaaccgcttcctccgatcccatggacctgatgccaagctgctggctggtggggaggatgtggctccaggccccctgggtctggggcagctgctggccgtggctagccaggtcgctgcggggatggtgtacctggcgggtctgcattttgtgcaccgggacctggccacacgcaactgtctagtgggccagggactggtggtcaagattggtgattttggcatgagcagggatatctacagcaccgactattaccgtgtgggaggccgcaccatgctgcccattcgctggatgccgcccgagagcatcctgtaccgtaagttcaccaccgagagcgacgtgtggagcttcggcgtggtgctctgggagatcttcacctacggcaagcagccctggtaccagctctccaacacggaggcaatcgactgcatcacgcagggacgtgagttggagcggccacgtgcctgcccaccagaggtctacgccatcatgcggggctgctggcagcgggagccccagcaacgccacagcatcaaggatgtgcacgcccggctgcaagccctggcccaggcacctcctgtctacctggatgtcctgggctagggggccggcccaggggctgggagtggttagccggaatactggggcctgccctcagcatcccccatagctcccagcagccccagggtgatctcaaagtatctaattcaccctcagcatgtgggaagggacaggtgggggctgggagtagaggatgttcctgcttctctaggcaaggtcccgtcatagcaattatatttattatcccttgaaaaaaaa

SEQ ID NO: 3 - protein, 760 aa, Homo sapiens
MKEAALICLAPSVPPILTVKSWDTMQLRAARSRCTNLLAASYIENQQHLQHLELRDLRGLGELRNLTIVKSGLRFVAPDAFHFTPRLSRLNLSFNALESLSWKTVQGLSLQELVLSGNPLHCSCALRWLQRWEEEGLGGVPEQKLQCHGQGPLAHMPNASCGVPTLKVQVPNASVDVGDDVLLRCQVEGRGLEQAGWILTELEQSATVMKSGGLPSLGLTLANVTSDLNRKNVTCWAENDVGRAEVSVQVNVSFPASVQLHTAVEMHHWCIPFSVDGQPAPSLRWLFNGSVLNETSFIFTEFLEPAANETVRHGCLRLNQPTHVNNGNYTLLAANPFGQASASIMAAFMDNPFEFNPEDPIPDTNSTSGDPVEKKDETPFGVSVAVGLAVFACLFLSTLLLVLNKCGRRNKFGINRPAVLAPEDGLAMSLHFMTLGGSSLSPTEGKGSGLQGHIIENPQYFSDACVHHIKRRDIVLKWELGEGAFGKVFLAECHNLLPEQDKMLVAVKALKEASESARQDFQREAELLTMLQHQHIVRFFGVCTEGRPLLMVFEYMRHGDLNRFLRSHGPDAKLLAGGEDVAPGPLGLGQLLAVASQVAAGMVYLAGLHFVHRDLATRNCLVGQGLVVKIGDFGMSRDIYSTDYYRVGGRTMLPIRWMPPESILYRKFTTESDVWSFGVVLWEIFTYGKQPWYQLSNTEAIDCITQGRELERPRACPPEVYAIMRGCWQREPQQRHSIKDVHARLQALAQAPPVYLDVLG

SEQ ID NO: 4 - DNA, 2581 bp, Homo sapiens
gcaccctggtcatctgcggactcagcctgagcttccagagggcctaggagcagtaagggagtgagtgggcaactcggcgcatgaaggaggccgccctcatctgcctggcaccctctgtacccccgatcttgacggtgaagtcctgggacaccatgcagttgcgggctgctagatctcggtgcacaaacttgttggcagcaagctacatcgagaaccagcagcatctgcagcatctggagctccgtgatctgaggggcctgggggagctgagaaacctcaccatcgtgaagagtggtctccgtttcgtggcgccagatgccttccatttcactcctcggctcagtcgcctgaatctctccttcaacgctctggagtctctctcctggaaaactgtgcagggcctctccttacaggaactggtcctgtcggggaaccctctgcactgttcttgtgccctgcgctggctacagcgctgggaggaggagggactgggcggagtgcctgaacagaagctgcagtgtcatgggcaagggcccctggcccacatgcccaatgccagctgtggtgtgcccacgctgaaggtccaggtgcccaatgcctcggtggatgtgggggacgacgtgctgctgcggtgccaggtggaggggcggggcctggagcaggccggctggatcctcacagagctggagcagtcagccacggtgatgaaatctgggggtctgccatccctggggctgaccctggccaatgtcaccagtgacctcaacaggaagaacgtgacgtgctgggcagagaacgatgtgggccgggcagaggtctctgttcaggtcaacgtctccttcccggccagtgtgcagctgcacacggcggtggagatgcaccactggtgcatccccttctctgtggatgggcagccggcaccgtctctgcgctggctcttcaatggctccgtgctcaatgagaccagcttcatcttcactgagttcctggagccggcagccaatgagaccgtgcggcacgggtgtctgcgcctcaaccagcccacccacgtcaacaacggcaactacacgctgctggctgccaaccccttcggccaggcctccgcctccatcatggctgccttcatggacaaccctttcgagttcaaccccgaggaccccatccctgacactaacagcacatctggagacccggtggagaagaaggacgaaacaccttttggggtctcggtggctgtgggcctggccgtctttgcctgcctcttcctttctacgctgctccttgtgctcaacaaatgtggacggagaaacaagtttgggatcaaccgcccggctgtgctggctccagaggatgggctggccatgtccctgcatttcatgacattgggtggcagctccctgtcccccaccgagggcaaaggctctgggctccaaggccacatcatcgagaacccacaatacttcagtgatgcctgtgttcaccacatcaagcgccgggacatcgtgctcaagtgggagctgggggagggcgcctttgggaaggtcttccttgctgagtgccacaacctcctgcctgagcaggacaagatgctggtggctgtcaaggcactgaaggaggcgtccgagagtgctcggcaggacttccagcgtgaggctgagctgctcaccatgctgcagcaccagcacatcgtgcgcttcttcggcgtctgcaccgagggccgccccctgctcatggtctttgagtatatgcggcacggggacctcaaccgcttcctccgatcccatggacctgatgccaagctgctggctggtggggaggatgtggctccaggccccctgggtctggggcagctgctggccgtggctagccaggtcgctgcggggatggtgtacctggcgggtctgcattttgtgcaccgggacctggccacacgcaactgtctagtgggccagggactggtggtcaagattggtgattttggcatgagcagggatatctacagcaccgactattaccgtgtgggaggccgcaccatgctgcccattcgctggatgccgcccgagagcatcctgtaccgtaagttcaccaccgagagcgacgtgtggagcttcggcgtggtgctctgggagatcttcacctacggcaagcagccctggtaccagctctccaacacggaggcaatcgactgcatcacgcagggacgtgagttggagcggccacgtgcctgcccaccagaggtctacgccatcatgcggggctgctggcagcgggagccccagcaacgccacagcatcaaggatgtgcacgcccggctgcaagccctggcccaggcacctcctgtctacctggatgtcctgggctagggggccggcccaggggctgggagtggttagccggaatactggggcctgccctcagcatcccccatagctcccagcagccccagggtgatctcaaagtatctaattcaccctcagcatgtgggaagggacaggtgggggctgggagtagaggatgttcctgcttctctaggcaaggtcccgtcatagcaattatatttattatcccttgaaaaaaaaaa

SEQ ID NO: 5 - protein, 822 aa, Homo sapiens
MSSWIRWHGPAMARLWGFCWLVVGFWRAAFACPTSCKCSASRIWCSDPSPGIVAFPRLEPNSVDPENITEIFIANQKRLEIINEDDVEAYVGLRNLTIVDSGLKFVAHKAFLKNSNLQHINFTRNKLTSLSRKHFRHLDLSELILVGNPFTCSCDIMWIKTLQEAKSSPDTQDLYCLNESSKNIPLANLQIPNCGLPSANLAAPNLTVEEGKSITLSCSVAGDPVPNMYWDVGNLVSKHMNETSHTQGSLRITNISSDDSGKQISCVAENLVGEDQDSVNLTVHFAPTITFLESPTSDHHWCIPFTVKGNPKPALQWFYNGAILNESKYICTKIHVTNHTEYHGCLQLDNPTHMNNGDYTLIAKNEYGKDEKQISAHFMGWPGIDDGANPNYPDVIYEDYGTAANDIGDTTNRSNEIPSTDVTDKTGREHLSVYAVVVIASVVGFCLLVMLFLLKLARHSKFGMKGPASVISNDDDSASPLHHISNGSNTPSSSEGGPDAVIIGMTKIPVIENPQYFGITNSQLKPDTFVQHIKRHNIVLKRELGEGAFGKVFLAECYNLCPEQDKILVAVKTLKDASDNARKDFHREAELLTNLQHEHIVKFYGVCVEGDPLIMVFEYMKHGDLNKFLRAHGPDAVLMAEGNPPTELTQSQMLHIAQQIAAGMVYLASQHFVHRDLATRNCLVGENLLVKIGDFGMSRDVYSTDYYRVGGHTMLPIRWMPPESIMYRKFTTESDVWSLGVVLWEIFTYGKQPWYQLSNNEVIECITQGRVLQRPRTCPQEVYELMLGCWQREPHMRKNIKGIHTLLQNLAKASPVYLDILG

SEQ ID NO: 6 - DNA, 3194 bp, Homo sapiens
ggaaggtttaaagaagaagccgcaaagcgcagggaaggcctcccggcacgggtgggggaaagcggccggtgcagcgcggggacaggcactcgggctggcactggctgctagggatgtcgtcctggataaggtggcatggacccgccatggcgcggctctggggcttctgctggctggttgtgggcttctggagggccgctttcgcctgtcccacgtcctgcaaatgcagtgcctctcggatctggtgcagcgacccttctcctggcatcgtggcatttccgagattggagcctaacagtgtagatcctgagaacatcaccgaaattttcatcgcaaaccagaaaaggttagaaatcatcaacgaagatgatgttgaagcttatgtgggactgagaaatctgacaattgtggattctggattaaaatttgtggctcataaagcatttctgaaaaacagcaacctgcagcacatcaattttacccgaaacaaactgacgagtttgtctaggaaacatttccgtcaccttgacttgtctgaactgatcctggtgggcaatccatttacatgctcctgtgacattatgtggatcaagactctccaagaggctaaatccagtccagacactcaggatttgtactgcctgaatgaaagcagcaagaatattcccctggcaaacctgcagatacccaattgtggtttgccatctgcaaatctggccgcacctaacctcactgtggaggaaggaaagtctatcacattatcctgtagtgtggcaggtgatccggttcctaatatgtattgggatgttggtaacctggtttccaaacatatgaatgaaacaagccacacacagggctccttaaggataactaacatttcatccgatgacagtgggaagcagatctcttgtgtggcggaaaatcttgtaggagaagatcaagattctgtcaacctcactgtgcattttgcaccaactatcacatttctcgaatctccaacctcagaccaccactggtgcattccattcactgtgaaaggcaacccaaaaccagcgcttcagtggttctataacggggcaatattgaatgagtccaaatacatctgtactaaaatacatgttaccaatcacacggagtaccacggctgcctccagctggataatcccactcacatgaacaatggggactacactctaatagccaagaatgagtatgggaaggatgagaaacagatttctgctcacttcatgggctggcctggaattgacgatggtgcaaacccaaattatcctgatgtaatttatgaagattatggaactgcagcgaatgacatcggggacaccacgaacagaagtaatgaaatcccttccacagacgtcactgataaaaccggtcgggaacatctctcggtctatgctgtggtggtgattgcgtctgtggtgggattttgccttttggtaatgctgtttctgcttaagttggcaagacactccaagtttggcatgaaaggcccagcctccgttatcagcaatgatgatgactctgccagcccactccatcacatctccaatgggagtaacactccatcttcttcggaaggtggcccagatgctgtcattattggaatgaccaagatccctgtcattgaaaatccccagtactttggcatcaccaacagtcagctcaagccagacacatttgttcagcacatcaagcgacataacattgttctgaaaagggagctaggcgaaggagcctttggaaaagtgttcctagctgaatgctataacctctgtcctgagcaggacaagatcttggtggcagtgaagaccctgaaggatgccagtgacaatgcacgcaaggacttccaccgtgaggccgagctcctgaccaacctccagcatgagcacatcgtcaagttctatggcgtctgcgtggagggcgaccccctcatcatggtctttgagtacatgaagcatggggacctcaacaagttcctcagggcacacggccctgatgccgtgctgatggctgagggcaacccgcccacggaactgacgcagtcgcagatgctgcatatagcccagcagatcgccgcgggcatggtctacctggcgtcccagcacttcgtgcaccgcgatttggccaccaggaactgcctggtcggggagaacttgctggtgaaaatcggggactttgggatgtcccgggacgtgtacagcactgactactacagggtcggtggccacacaatgctgcccattcgctggatgcctccagagagcatcatgtacaggaaattcacgacggaaagcgacgtctggagcctgggggtcgtgttgtgggagattttcacctatggcaaacagccctggtaccagctgtcaaacaatgaggtgatagagtgtatcactcagggccgagtcctgcagcgaccccgcacgtgcccccaggaggtgtatgagctgatgctggggtgctggcagcgagagccccacatgaggaagaacatcaagggcatccataccctccttcagaacttggccaaggcatctccggtctacctggacattctaggctagggcccttttccccagaccgatccttcccaacgtactcctcagacgggctgagaggatgaacatcttttaactgccgctggaggccaccaagctgctctccttcactctgacagtattaacatcaaagactccgagaagctctcgagggaagcagtgtgtacttcttcatccatagacacagtattgacttctttttggcattatctctttctctctttccatctcccttggttgttcctttttctttttttaaattttctttttcttcttttttttcgtcttccctgcttcacgattcttaccctttcttttgaatcaatctggcttctgcattactattaactctgcatagacaaaggccttaacaaacgtaatttgttatatcagcagacactccagtttgcccaccacaactaacaatgccttgttgtattcctgcctttgatgtggatgaaaaaaagggaaaacaaatatttcacttaaactttgtcacttctgctgtacagatatcgagagtttctatggattcacttctatttatttattattattactgttcttattgtttttggatggcttaagcctgtgtataaaaaaaaaaaaaaatctaga

SEQ ID NO: 7 - protein, 839 aa, Homo sapiens [sequence omitted]

SEQ ID NO: 8 - DNA, 2715 bp, Homo sapiens
ggatccgcgtcggagatggatgtctctctttgcccagccaagtgtagtttctggcggattttcttgctgggaagcgtctggctggactatgtgggctccgtgctggcttgccctgcaaattgtgtctgcagcaagactgagatcaattgccggcggccggacgatgggaacctcttccccctcctggaagggcaggattcagggaacagcaatgggaacgccaatatcaacatcacggacatctcaaggaatatcacttccatacacatagagaactggcgcagtcttcacacgctcaacgccgtggacatggagctctacaccggacttcaaaagctgaccatcaagaactcaggacttcggagcattcagcccagagcctttgccaagaacccccatttgcgttatataaacctgtcaagtaaccggctcaccacactctcgtggcagctcttccagacgctgagtcttcgggaattgcagttggagcagaactttttcaactgcagctgtgacatccgctggatgcagctctggcaggagcagggggaggccaagctcaacagccagaacctctactgcatcaatgctgatggctcccagcttcctctcttccgcatgaacatcagtcagtgtgaccttcctgagatcagcgtgagccacgtcaacctgaccgtacgagagggtgacaatgctgttatcacttgcaatggctctggatcaccccttcctgatgtggactggatagtcactgggctgcagtccatcaacactcaccagaccaatctgaactggaccaatgttcatgccatcaacttgacgctggtgaatgtgacgagtgaggacaatggcttcaccctgacgtgcattgcagagaacgtggtgggcatgagcaatgccagtgttgccctcactgtctactatcccccacgtgtggtgagcctggaggagcctgagctgcgcctggagcactgcatcgagtttgtggtgcgtggcaaccccccaccaacgctgcactggctgcacaatgggcagcctctgcgggagtccaagatcatccatgtggaatactaccaagagggagagatttccgagggctgcctgctcttcaacaagcccacccactacaacaatggcaactataccctcattgccaaaaacccactgggcacagccaaccagaccatcaatggccacttcctcaaggagccctttccagagagcacggataactttatcttgtttgacgaagtgagtcccacacctcctatcactgtgacccacaaaccagaagaagacacttttggggtatccatagcagttggacttgctgcttttgcctgtgtcctgttggtggttctcttcgtcatgatcaacaaatatggtcgacggtccaaatttggaatgaagggtcccgtggctgtcatcagtggtgaggaggactcagccagcccactgcaccacatcaaccacggcatcaccacgccctcgtcactggatgccgggcccgacactgtggtcattggcatgactcgcatccctgtcattgagaacccccagtacttccgtcagggacacaactgccacaagccggacacgtatgtgcagcacattaagaggagagacatcgtgctgaagcgagaactgggtgagggagcctttggaaaggtcttcctggccgagtgctacaacctcagcccgaccaaggacaagatgcttgtggctgtgaaggccctgaaggatcccaccctggctgcccggaaggatttccagagggaggccgagctgctcaccaacctgcagcatgagcacattgtcaagttctatggagtgtgcggcgatggggaccccctcatcatggtctttgaatacatgaagcatggagacctgaataagttcctcagggcccatgggccagatgcaatgatccttgtggatggacagccacgccaggccaagggtgagctggggctctcccaaatgctccacattgccagtcagatcgcctcgggtatggtgtacctggcctcccagcactttgtgcaccgagacctggccaccaggaactgcctggttggagcgaatctgctagtgaagattggggacttcggcatgtccagagatgtctacagcacggattattacaggctctttaatccatctggaaatgatttttgtatatggtgtgaggtgggaggacacaccatgctccccattcgctggatgcctcctgaaagcatcatgtaccggaagttcactacagagagtgatgtatggagcttcggggtgatcctctgggagatcttcacctatggaaagcagccatggttccaactctcaaacacggaggtcattgagtgcattacccaaggtcgtgttttggagcggccccgagtctgccccaaagaggtgtacgatgtcatgctggggtgctggcagagggaaccacagcagcggttgaacatcaaggagatctacaaaatcctccatgctttggggaaggccaccccaatctacctggacattcttggctagtggtggctggtggtcatgaattcatactctgttgcctcctctctccctgcctcacatctcccttccacctcacaactccttccatccttgactgaagcgaacatcttcatataaactcaagtgcctgctacacatacaacactgaaaaaaggaaaaaaaaagaaaaaaaaaaaaaaccgc

SEQ ID NO: 9 - protein, 796 aa, Homo sapiens; identical to SEQ ID NO: 1

SEQ ID NO: 10 - protein, 822 aa, Homo sapiens; identical to SEQ ID NO: 5

SEQ ID NO: 11 - protein, 839 aa, Homo sapiens [sequence omitted]

(19) United States
(12) Patent Application Publication
(10) Pub. No.: US 2019/0247398 A1
(43) Pub. Date: Aug. 15, 2019

(54) FORMULATIONS OF A MACROCYCLIC TRK KINASE INHIBITOR

(71) Applicants: Array BioPharma Inc., Boulder, CO (US); Loxo Oncology, Inc., Stamford, CT (US)

(72) Inventors: Qian Zhao, Boulder, CO (US); Stacey Spencer, Boulder, CO (US); Yutong Jiang, Boulder, CO (US); Julia Haas, Boulder, CO (US); Charles Todd Eary, Longmont, CO (US)

(21) Appl. No.: 16/170,976

(22) Filed: Oct. 25, 2018

Related U.S. Application Data
(60) Provisional application No. 62/577,449, filed on Oct. 26, 2017.
(60) Provisional application No. 62/736,102, filed on Sep. 25, 2018.

(51) Int. Cl. A61K 31/519; A61P 35/00

(52) U.S. Cl. CPC A61K 31/519; A61P 35/00

(57) ABSTRACT

The present application in some embodiments provides a pharmaceutical composition comprising:

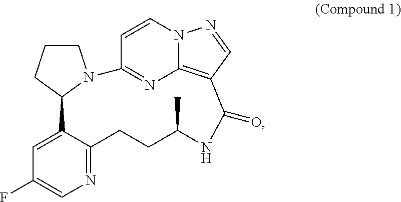 [SMILES: C[C@@H]1CCc2ncc(F)cc2[C@H]2CCCN2c2ccn3ncc(c3n2)C(=O)N1]

and a compounding agent. Further provided herein are methods of making and methods of using the pharmaceutical compositions, for example, in the treatment of cancer.

### CROSS-REFERENCE TO RELATED APPLICATIONS

This application claims priority to U.S. Provisional Application Ser. No. 62/736,102, filed on Sep. 25, 2018 and 62/577,449, filed on Oct. 26, 2017, the disclosures of which are hereby incorporated by reference in their entireties.

### TECHNICAL FIELD

The present application relates to oral formulations of the Trk kinase inhibitor (6R,15R)-9-fluoro-15-methyl-2,11,16,20,21,24-hexaazapentacyclo[16.5.2.0^{2,6}.0^{7,12}.0^{21,25}]pentacosa-1 (24),7,9,11,18(25), 19,22-heptaen-17-one, crystalline forms of the Trk kinase inhibitor, salt forms of the Trk kinase inhibitor, and crystalline forms of these salts, including methods of preparation thereof, where the formulations are useful in the treatment of the Trk-associated disorders such as cancer, pain, inflammation, neurodegenerative diseases and certain infectious diseases.

### BACKGROUND

Trks are high affinity receptor tyrosine kinases activated by a group of soluble growth factors called neurotrophins (NT). The Trk receptor family has three members: TrkA, TrkB and TrkC. Among the neurotrophins are (i) nerve growth factor (NGF) which activates TrkA, (ii) brain-derived neurotrophic factor (BDNF) and neurotrophin-4/5 which activate TrkB and (iii) neurotrophin-3 which activates TrkC. Inhibitors of the Trk/neurotrophin pathway have been demonstrated to be effective in numerous pre-clinical animal models of pain. Overexpression, activation, amplification and/or mutation of Trk kinases are associated with many cancers including neuroblastoma, ovarian and colorectal cancer, melanoma, head and neck cancer, gastric carcinoma, lung carcinoma, breast cancer, glioblastoma, medulloblastoma, secretory breast cancer, salivary gland cancer, papillary thyroid carcinoma, and adult myeloid leukemia. The neurotrophin/Trk pathway has been implicated in inflammatory diseases including asthma, interstitial cystitis, inflammatory bowel diseases including ulcerative colitis and Crohn's disease, and inflammatory skin diseases such as atopic dermatitis, eczema and psoriasis. The neurotrophin/Trk pathway has also been implicated in the etiology of neurodegenerative diseases including multiple sclerosis, Parkinson's disease and Alzheimer's disease. The TrkA receptor is also involved the disease process in the parasitic infection of Trypanosoma cruzi (Chagas disease) in human hosts. As such, inhibition of Trk kinases will be useful to provide therapeutic benefit to patients suffering from the aforementioned conditions.

New formulations of macrocyclic pyrazolo[1,5-a]pyrimidines can be useful in the treatment of these conditions.

### SUMMARY

The present disclosure in some embodiments is directed to a pharmaceutical composition comprising (6R,15R)-9-fluoro-15-methyl-2,11,16,20,21,24-hexaazapentacyclo[16.5.2.0^{2,6}.0^{7,12}.0^{21,25}]pentacosa-1 (24),7,9,11,18(25), 19,22-heptaen-17-one having the following structural formula:

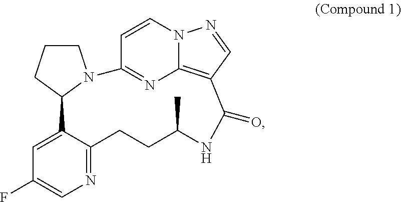 [SMILES: C[C@@H]1CCc2ncc(F)cc2[C@H]2CCCN2c2ccn3ncc(c3n2)C(=O)N1]

and a compounding agent as disclosed herein.

The present disclosure in some embodiments is directed to a pharmaceutical composition comprising a crystalline form of Compound 1, such as a crystalline form of Compound 1 having Form I, and a compounding agent as disclosed herein.

The present disclosure in some embodiments is directed to a pharmaceutical composition comprising a salt of Compound 1 and a compounding agent as disclosed herein.

In some embodiments of the pharmaceutical compositions disclosed herein, a pharmaceutical composition is a liquid oral pharmaceutical composition.

The present disclosure in some embodiments is directed to a pharmaceutical composition comprising a benzenesulfonic acid salt, citric acid salt, methanesulfonic acid salt, 1,2-ethane disulfonic acid salt, p-toluene sulfonic acid salt, oxalic acid salt, fumaric acid salt, L-malic acid salt, or succinic acid salt of Compound 1 and a compounding agent as disclosed herein. In some more particular embodiments, the benzenesulfonic acid salt, citric acid salt, methanesulfonic acid salt, 1,2-ethane disulfonic acid salt, p-toluene sulfonic acid salt, oxalic acid salt, fumaric acid salt, L-malic acid salt, or succinic acid salt is present as a crystalline form.

In some embodiments, the present disclosure is directed to a pharmaceutical composition comprising a crystalline form of Compound 1 besylate and a compounding agent as disclosed herein.

In some embodiments, the present disclosure is directed to a pharmaceutical composition comprising a crystalline form of Compound 1 citrate, such as crystalline Compound 1 citrate Form A, and a compounding agent as disclosed herein.

The present disclosure is further directed to the hydrochloric acid salt, sulfuric acid salt, naphthalene-2-sulphonic acid salt, 2-hydroxy ethanesulfonic acid salt, L-aspartic acid salt, maleic acid salt, phosphoric acid salt, ethanesulfonic acid salt, L-glutamic acid salt, L-tartaric acid salt, D-glucuronic acid salt, hippuric acid salt, D-gluconic acid salt, DL-lactic acid salt, L-ascorbic acid salt, benzoic acid salt, benzenesulfonic acid salt, citric acid salt, methanesulfonic acid salt, 1,2-ethane disulfonic acid salt, p-toluene sulfonic acid salt, oxalic acid salt, fumaric acid salt, L-malic acid salt, and succinic acid salt of Compound 1.

The present disclosure is further directed to a pharmaceutical composition comprising a hydrate or a solvate of Compound 1, or any one of the salts of Compound 1 described herein, and a compounding agent as disclosed herein. In some aspects, the hydrate or the solvate is crystalline.

The present disclosure is further directed to pharmaceutical compositions comprising any one of the crystalline forms, solid forms, solvates, hydrates, or salts described herein, and a compounding agent as disclosed herein.

In some embodiments, the present disclosure is directed to a pharmaceutical composition prepared by a process comprising mixing a compounding agent as disclosed herein with Compound 1, to form the pharmaceutical composition.

In some embodiments, the present disclosure is directed to a pharmaceutical composition prepared by a process comprising mixing a compounding agent as disclosed herein with any one of the crystalline forms, solid forms, solvates, hydrates, or salts described herein, to form the pharmaceutical composition.

In some embodiments, provided herein is a process for preparing a pharmaceutical composition comprising Compound 1, comprising mixing a compounding agent as disclosed herein with Compound 1, to form the pharmaceutical composition.

In some embodiments, provided herein is a process for preparing a pharmaceutical composition, comprising mixing a compounding agent as disclosed herein with any one of the crystalline forms, solid forms, solvates, hydrates, or salts described herein, to form the pharmaceutical composition.

In some embodiments, the present disclosure is directed to a pharmaceutical composition comprising Compound 1 and a sweetener as disclosed herein.

In some embodiments, the present disclosure is directed to a pharmaceutical composition prepared by a process comprising mixing a sweetener as disclosed herein with Compound 1, to form the pharmaceutical composition.

The present disclosure is further directed to a pharmaceutical composition prepared by a process comprising mixing a sweetener as disclosed herein with any one of the crystalline forms, solid forms, solvates, hydrates, or salts described herein, to form the pharmaceutical composition.

The present disclosure is further directed to therapeutic methods of using a pharmaceutical composition as described herein.

Also provided herein is a method of treating a subject having a cancer, the method comprising: (a) detecting a dysregulation of a NTRK gene, a Trk kinase, or the expression or activity or level of any of the same; (b) administering one or more doses of a first Trk inhibitor to the subject for a period of time; (c) after (a) and (b), determining whether (i) the cancer in the subject has relapsed during therapy with the first Trk inhibitor; and/or (ii) the cancer in the subject is not responding to therapy with the first Trk inhibitor; and/or (iii) the subject is intolerant to the first Trk inhibitor; and (d) administering a treatment including one or more doses of a second Trk inhibitor or a pharmaceutically acceptable salt thereof, to a subject in which (i) the cancer in the subject has relapsed during therapy with the first Trk inhibitor; and/or (ii) the cancer in the subject is not responding to therapy with the first Trk inhibitor; and/or (iii) the subject is intolerant to the first Trk inhibitor; or (e) administering additional doses of the first Trk inhibitor to a subject in which (i) the cancer has not relapsed during therapy with the first Trk inhibitor; and/or (ii) the cancer in the subject is responding to therapy with the first Trk inhibitor; and/or (iii) the subject is not intolerant to the first Trk inhibitor. In some embodiments, the second Trk inhibitor is Compound 1, or a pharmaceutically acceptable salt, amorphous, or polymorph form thereof. In some embodiments, step (a) is performed before step (b). In some embodiments, step (b) is performed before step (a). In some embodiments, step (d) further comprises administration of another anticancer agent or anticancer therapy. In some embodiments, detecting a dysregulation of a NTRK gene, a Trk kinase, or the expression or activity or level of any of the same comprises next generation sequencing, immunohistochemistry, fluorescence microscopy, break apart FISH analysis, and PCR-based amplification (e.g., RT-PCR and quantitative real-time RT-PCR). In some embodiments, the dysregulation of a NTRK gene, a Trk kinase, or the expression or activity or level of any of the same is at least one NTRK1, NTRK2, and/or NTRK3 fusion. In some embodiments, the at least one NTRK1, NTRK2, and/or NTRK3 fusion results in the expression of one or more of a TrkA fusion protein, and/or a TrkB fusion protein, and/or a TrkC fusion protein, wherein the TrkA fusion protein comprises one or more of the of the fusions selected from the group consisting of: TP53-TrkA, LMNA-TrkA, CD74-TrkA, TFG-TrkA, TPM3-TrkA, NFASC-TrkA, BCAN-TrkA, MPRIP-TrkA, TPR-TrkA, RFWD2-TrkA, IRF2BP2-TrkA, SQSTM1-TrkA, SSBP2-TrkA, RABGAP1L-TrkA, C18ORF8-TrkA, RNF213-TrkA, TBC1D22A-TrkA, C20ORF112-TrkA, DNER-TrkA, ARHGEF2-TrkA, CHTOP-TrkA, PPL-TrkA, PLEKHA6-TrkA, PEAR1-TrkA, MRPL24-TrkA, MDM4-TrkA, LRRC71-TrkA, GRIPAP1-TrkA, TAF-TrkA, EPS15-TrkA, DYNC2H1-TrkA, CEL-TrkA, EPHB2-TrkA, TGF-TrkA, NELL1-TrkA, EPL4-TrkA, CTNND2-TrkA, TCEANC2-TrkA, SCYL3-TrkA, AMOTL2-TrkA, MEF2D-TrkA, L7a-TrkA, ZBTB7B-TrkA, TRIM63-TrkA, DDR2-TrkAl, GON4L-TrkA, PDE4DIP-TrkA, NTRK1-P2RY8, CTRC-TrkA, and VANGL2-TrkA; and/or the TrkB fusion protein comprises one or more of the of the fusions selected from the group consisting of: NACC2-TrkB, QKI-TrkB, AFAP1-TrkB, PAN3-TrkB, SQSTM1-TrkB, TRIM24-TrkB, VCL-TrkB, AGBL4-TrkB, DAB2IP-TrkB, TrkB-TERT, ETV6-TrkB, NOS1AP-TrkB, GKAP1-TrkB, KCTD8-TrkB, TBC1D2-TrkB, VCAN-TrkB, SLMAP-TrkB, TLE4-TrkB, STRN3-TrkB, WNK2-TrkB, TrkB-BEND5, TrkB-TRAF2, Nav1-TrkB, and STRN-TrkB; and/or the TrkC fusion protein comprises one or more of the of the fusions selected from the group consisting of: ETV6-TrkC1, BTBD1-TrkC, LYN-TrkC, RBPMS-TrkC, EML4-TrkC, TrkC-HOMER2, TFG-TrkC, FAT1-TrkC, MYO5A-TrkC, MYH9-TrkC, KANK1-TrkC, SQSTM1-TrkC, UBE2R2-TrkC, HNRNPA2B1-TrkC, VPS18-TrkC, AKAP13-TrkC, TrkC-LOXL2, TrkC-PEAK1, ZNF710-TrkC, TPM4-TrkC, and LMNA-TrkC.

Also provided herein is a method of treating a subject having a cancer, the method comprising: (a) administering one or more doses of a first Trk inhibitor to the subject for a period of time; (b) after (a), determining whether (i) the cancer in the subject has relapsed during therapy with the first Trk inhibitor; and/or (ii) the cancer in the subject is not responding to therapy with the first Trk inhibitor; and/or (iii) the subject is intolerant to the first Trk inhibitor; and (c) administering a treatment including one or more doses of a second Trk inhibitor or a pharmaceutically acceptable salt thereof, to a subject in which (i) the cancer in the subject has relapsed during therapy with the first Trk inhibitor; and/or (ii) the cancer in the subject is not responding to therapy with the first Trk inhibitor; and/or (iii) the subject is intolerant to the first Trk inhibitor; or (d) administering additional doses of the first Trk inhibitor to a subject in which (i) the cancer has not relapsed during therapy with the first Trk inhibitor; and/or (ii) the cancer in the subject is responding to therapy with the first Trk inhibitor; and/or (iii) the subject is not intolerant to the first Trk inhibitor. In some embodiments, the second Trk inhibitor is Compound 1, or a pharmaceutically acceptable salt, amorphous, or polymorph form thereof. In some embodiments, step (c) further comprises administration of another anticancer agent or anticancer therapy. In some embodiments, the cancer is a Trk-associated cancer.

Also provided herein is a method of treating a subject having a cancer, the method comprising: (a) determining whether (i) the cancer in the subject has relapsed during therapy with a first Trk inhibitor; and/or (ii) the cancer in the subject is not responding to therapy with a first Trk inhibitor; and/or (iii) the subject is intolerant to a first Trk inhibitor; (b) administering a second Trk inhibitor or a treatment that does not include the first Trk inhibitor of step (a) as a monotherapy to a subject in which (i) the cancer in the subject has relapsed during therapy with the first Trk inhibitor; and/or (ii) the cancer in the subject is not responding to therapy with the first Trk inhibitor; and/or (iii) the subject is intolerant to the first Trk inhibitor; or (c) administering additional doses of the first Trk inhibitor of step (a) to a subject in which (i) the cancer has not relapsed during therapy with the first Trk inhibitor during therapy with the first Trk inhibitor; and/or (ii) the cancer in the subject is responding to therapy with the first Trk inhibitor; and/or (iii) the subject is not intolerant to the first Trk inhibitor. In some embodiments, step (b) comprises administering one or more doses of a second Trk inhibitor, wherein the second Trk inhibitor is Compound 1, or a pharmaceutically acceptable salt, amorphous, or polymorph form thereof. In some embodiments, step (b) further comprises administering another anticancer agent or anticancer therapy.

Also provided herein is a method of treating a subject having a cancer, the method comprising: identifying a subject in which (i) the cancer in the subject has relapsed during therapy with a first Trk inhibitor; and/or (ii) the cancer in the subject is not responding to therapy with a first Trk inhibitor; and/or (iii) the subject is intolerant to a first Trk inhibitor; and administering to the identified subject a treatment that does not include a first Trk inhibitor as a monotherapy. In some embodiments, the treatment that does not include a first Trk inhibitor as a monotherapy includes administering a therapeutically effective amount of Compound 1, or a pharmaceutically acceptable salt, amorphous, or polymorph form thereof.

Also provided herein is a method of treating a subject having a cancer, the method comprising: identifying a subject in which (i) the cancer in the subject has relapsed during therapy with a first Trk inhibitor; and/or (ii) the cancer in the subject is not responding to therapy with a first Trk inhibitor; and/or (iii) the subject is intolerant to a first Trk inhibitor; and administering to the identified subject a treatment that includes Compound 1, or a pharmaceutically acceptable salt, amorphous, or polymorph form thereof. In some embodiments, the method further comprises administration of another anticancer agent or anticancer therapy.

Also provided herein is a method of treating a subject identified as having a cancer in which (i) the cancer in the subject has relapsed during therapy with a first Trk inhibitor; and/or (ii) the cancer in the subject is not responding to therapy with a first Trk inhibitor; and/or (iii) the subject is intolerant to a first Trk inhibitor, the method comprising administering to the subject a treatment that does not include the first Trk inhibitor as a monotherapy. In some embodiments, the treatment that does not include a first Trk inhibitor as a monotherapy includes administering a therapeutically effective amount of Compound 1, or a pharmaceutically acceptable salt, amorphous, or polymorph form thereof.

Also provided herein is a method of treating a subject identified as having a cancer in which (i) the cancer in the subject has relapsed during therapy with a first Trk inhibitor; and/or (ii) the cancer in the subject is not responding to therapy with a first Trk inhibitor; and/or (iii) the subject is intolerant to a first Trk inhibitor, the method comprising administering to the subject a treatment that includes Compound 1, or a pharmaceutically acceptable salt, amorphous, or polymorph form thereof. In some embodiments, the method further comprises administration of another anticancer agent or anticancer therapy.

Also provided herein is a method of treating a subject, the method comprising administering a therapeutically effective amount of a treatment that does not include a first Trk inhibitor as a monotherapy, to a subject having a clinical record that indicates that (i) the cancer in the subject has relapsed during therapy with the first Trk inhibitor; and/or (ii) the cancer in the subject is not responding to therapy with the first Trk inhibitor; and/or (iii) the subject is intolerant to the first Trk inhibitor. In some embodiments, the treatment that does not include a first Trk inhibitor as a monotherapy includes administering a therapeutically effective amount of Compound 1, or a pharmaceutically acceptable salt, amorphous, or polymorph form thereof.

Also provided herein is a method of treating a subject, the method comprising administering a therapeutically effective amount of Compound 1, or a pharmaceutically acceptable salt, amorphous, or polymorph form thereof, to a subject having a clinical record that indicates that (i) the cancer in the subject has relapsed during therapy with a first Trk inhibitor; and/or (ii) the cancer in the subject is not responding to therapy with a first Trk inhibitor; and/or (iii) the subject is intolerant to a first Trk inhibitor. In some embodiments, the method further comprises administering another anticancer agent or anticancer therapy.

Also provided herein is a method of selecting a treatment for a subject having a cancer, the method comprising: identifying a subject in which (i) the cancer in the subject has relapsed during therapy with a first Trk inhibitor; and/or (ii) the cancer in the subject is not responding to therapy with a first Trk inhibitor; and/or (iii) the subject is intolerant to a first Trk inhibitor; and selecting a treatment for the identified subject that does not include a first Trk inhibitor as a monotherapy. In some embodiments, the treatment that does not include a first Trk inhibitor as a monotherapy includes administering a therapeutically effective amount of Compound 1, or a pharmaceutically acceptable salt, amorphous, or polymorph form thereof.

Also provided herein is a method of selecting a treatment for a subject having a cancer, the method comprising: identifying a subject in which (i) the cancer in the subject has relapsed during therapy with a first Trk inhibitor; and/or (ii) the cancer in the subject is not responding to therapy with the first Trk inhibitor; and/or (iii) the subject is intolerant to the first Trk inhibitor; and selecting a treatment for the identified subject that includes Compound 1, or a pharmaceutically acceptable salt, amorphous, or polymorph form thereof. In some embodiments, the selected treatment further comprises administering another anticancer agent or anticancer therapy.

Also provided herein is a method of selecting a treatment for a subject having a cancer, the method comprising: selecting a treatment that does not include a first Trk inhibitor as a monotherapy for a subject in which (i) the cancer in the subject has relapsed during therapy with the first Trk inhibitor; and/or (ii) the cancer in the subject is not responding to therapy with the first Trk inhibitor; and/or (iii) the subject is intolerant to the first Trk inhibitor. In some embodiments, the treatment that does not include a first Trk inhibitor as a monotherapy includes administering a therapeutically effective amount of Compound 1, or a pharmaceutically acceptable salt, amorphous, or polymorph form thereof.

Also provided herein is a method of selecting a treatment for a subject having a cancer, the method comprising: selecting a treatment that includes a pharmaceutical composition comprising Compound 1, or a pharmaceutically acceptable salt, amorphous, or polymorph form thereof; for a subject in which (i) the cancer in the subject has relapsed during therapy with a first Trk inhibitor; and/or (ii) the cancer in the subject is not responding to therapy with a first Trk inhibitor; and/or (iii) the subject is intolerant to a first Trk inhibitor. In some embodiments, the selected treatment further comprises administering another anticancer agent or anticancer therapy.

Also provided herein is a method of selecting a subject having a cancer for a treatment that does not include a first Trk inhibitor as a monotherapy, the method comprising: identifying a subject in which (i) the cancer in the subject has relapsed during therapy with the first Trk inhibitor; and/or (ii) the cancer in the subject is not responding to therapy with the first Trk inhibitor; and/or (iii) the subject is intolerant to the first Trk inhibitor; and selecting the identified subject for a treatment that does not include the first Trk inhibitor as a monotherapy. In some embodiments, the treatment that does not include a first Trk inhibitor as a monotherapy includes administering a therapeutically effective amount of Compound 1, or a pharmaceutically acceptable salt, amorphous, or polymorph form thereof.

Also provided herein is a method of selecting a subject having a cancer for a treatment that includes Compound 1, or a pharmaceutically acceptable salt, amorphous, or polymorph form thereof, the method comprising: identifying a subject in which (i) the cancer in the subject has relapsed during therapy with a first Trk inhibitor; and/or (ii) the cancer in the subject is not responding to therapy with a first Trk inhibitor; and/or (iii) the subject is intolerant to a first Trk inhibitor; and selecting the identified subject for a treatment that includes Compound 1, or a pharmaceutically acceptable salt, amorphous, or polymorph form thereof. In some embodiments, the selected treatment further comprises administration of another anticancer agent or anticancer therapy.

Also provided herein is a method of selecting a subject having a cancer for a treatment that does not include a first Trk inhibitor as a monotherapy, the method comprising: selecting a subject in which (i) the cancer in the subject has relapsed during therapy with a first Trk inhibitor; and/or (ii) the cancer in the subject is not responding to therapy with the first Trk inhibitor; and/or (iii) the subject is intolerant to the first Trk inhibitor. In some embodiments, the treatment that does not include a first Trk inhibitor as a monotherapy includes administering a therapeutically effective amount of Compound 1, or a pharmaceutically acceptable salt, amorphous, or polymorph form thereof.

Also provided herein is a method of selecting a subject having a cancer for a treatment that includes Compound 1, or a pharmaceutically acceptable salt, amorphous, or polymorph form thereof, the method comprising: selecting a subject in which (i) the cancer in the subject has relapsed during therapy with a first Trk inhibitor; and/or (ii) the cancer in the subject is not responding to therapy with a first Trk inhibitor; and/or (iii) the subject is intolerant to a first Trk inhibitor, for a treatment that includes a pharmaceutical composition comprising a compounding agent and Compound 1 or a solid form thereof, crystalline form thereof, or solvate or hydrate thereof, or a salt of Compound 1 or solid form thereof, crystalline form thereof, or solvate or hydrate thereof. In some embodiments, the treatment further comprises administration of another anticancer agent or anticancer therapy.

Also provided herein is a method of determining the likelihood that a subject having a cancer will have a positive response to therapy with a first Trk inhibitor as a monotherapy, the method comprising: determining whether (i) the cancer in the subject has relapsed during therapy with a first Trk inhibitor; and/or (ii) the cancer in the subject is not responding to therapy with the first Trk inhibitor; and/or (iii) the subject is intolerant to the first Trk inhibitor; and determining that a subject in which (i) the cancer in the subject has relapsed during therapy with a first Trk inhibitor; and/or (ii) the cancer in the subject is not responding to therapy with the first Trk inhibitor; and/or (iii) the subject is intolerant to the first Trk inhibitor, has a decreased likelihood of having a positive response to therapy with a first Trk inhibitor as a monotherapy.

Also provided herein is a method of determining the likelihood that a subject having a cancer will have a positive response to therapy that includes a pharmaceutical composition comprising Compound 1, or a pharmaceutically acceptable salt, amorphous, or polymorph form thereof, the method comprising: determining whether (i) the cancer in the subject has relapsed during therapy with a first Trk inhibitor; and/or (ii) the cancer in the subject is not responding to therapy with the first Trk inhibitor; and/or (iii) the subject is intolerant to the first Trk inhibitor; and determining that a subject in which (i) the cancer in the subject has relapsed during therapy with a first Trk inhibitor; and/or (ii) the cancer in the subject is not responding to therapy with the first Trk inhibitor; and/or (iii) the subject is intolerant to the first Trk inhibitor, has an increased likelihood of having a positive response to therapy that includes Compound 1, or a pharmaceutically acceptable salt, amorphous, or polymorph form thereof.

Also provided herein is a method of determining the likelihood that a subject having cancer will have a positive response to therapy with a first Trk inhibitor as a monotherapy, the method comprising: determining that a subject in which (i) the cancer in the subject has relapsed during therapy with a first Trk inhibitor; and/or (ii) the cancer in the subject is not responding to therapy with the first Trk inhibitor; and/or (iii) the subject is intolerant to the first Trk inhibitor, has a decreased likelihood of having a positive response to therapy with a first Trk inhibitor as a monotherapy.

Also provided herein is a method of determining the likelihood that a subject having cancer will have a positive response to therapy that includes Compound 1, or a pharmaceutically acceptable salt, amorphous, or polymorph form thereof, the method comprising: determining that a subject in which (i) the cancer in the subject has relapsed during therapy with a first Trk inhibitor; and/or (ii) the cancer in the subject is not responding to therapy with the first Trk inhibitor; and/or (iii) the subject is intolerant to the first Trk inhibitor, has an increased likelihood of having a positive response to therapy including Compound 1, or a pharmaceutically acceptable salt, amorphous, or polymorph form thereof.

Also provided herein is a method of predicting the efficacy of therapy with a first Trk inhibitor as a monotherapy in a subject having cancer, the method comprising: determining whether (i) the cancer in the subject has relapsed during therapy with the first Trk inhibitor; and/or (ii) the cancer in the subject is not responding to therapy with the first Trk inhibitor; and/or (iii) the subject is intolerant to the first Trk inhibitor; and determining that therapy with the first Trk inhibitor as a monotherapy is less likely to be more effective in a subject than administration of a second Trk inhibitor in which (i) the cancer in the subject has relapsed during therapy with the first Trk inhibitor; and/or (ii) the cancer in the subject is not responding to therapy with the first Trk inhibitor; and/or (iii) the subject is intolerant to the first Trk inhibitor.

Also provided herein is a method of predicting the efficacy of therapy including Compound 1, or a pharmaceutically acceptable salt, amorphous, or polymorph form thereof, in a subject having cancer, the method comprising: determining whether (i) the cancer in the subject has relapsed during therapy with a first Trk inhibitor; and/or (ii) the cancer in the subject is not responding to therapy with a first Trk inhibitor; and/or (iii) the subject is intolerant to a first Trk inhibitor; and determining that therapy including a pharmaceutical composition comprising Compound 1, or a pharmaceutically acceptable salt, amorphous, or polymorph form thereof, is more likely to be more effective than the first Trk inhibitor in the subject in which (i) the cancer in the subject has relapsed during therapy with the first Trk inhibitor; and/or (ii) the cancer in the subject is not responding to therapy with the first Trk inhibitor; and/or (iii) the subject is intolerant to the first Trk inhibitor.

Also provided herein is a method of predicting the efficacy of therapy with a first Trk inhibitor as a monotherapy in a subject having cancer, the method comprising: determining that therapy with the first Trk inhibitor as a monotherapy is less likely to be more effective in a subject than administration of a second Trk inhibitor in which (i) the cancer in the subject has relapsed during therapy with the first Trk inhibitor; and/or (ii) the cancer in the subject is not responding to therapy with the first Trk inhibitor; and/or (iii) the subject is intolerant to the first Trk inhibitor.

Also provided herein is a method of predicting the efficacy of therapy including Compound 1, or a pharmaceutically acceptable salt, amorphous, or polymorph form thereof, in a subject having cancer, the method comprising: determining that therapy including Compound 1, or a pharmaceutically acceptable salt, amorphous, or polymorph form thereof, is more likely to be more effective than administration of a first Trk inhibitor in the subject in which (i) the cancer in the subject has relapsed during therapy with a first Trk inhibitor; and/or (ii) the cancer in the subject is not responding to therapy with a first Trk inhibitor; and/or (iii) the subject is intolerant to a first Trk inhibitor.

Also provided herein is a method of treating a subject having a cancer, the method comprising: (a) detecting a dysregulation of a NTRK gene, a Trk kinase, or the expression or activity or level of any of the same; (b) administering one or more doses of a first Trk inhibitor to the subject for a period of time; (c) after (a) and (b), determining whether (i) the cancer in the subject has relapsed during therapy with the first Trk inhibitor; and/or (ii) the cancer in the subject is not responding to therapy with the first Trk inhibitor; and/or (iii) the subject is intolerant to the first Trk inhibitor; and (d) administering a second Trk inhibitor or a treatment that does not include the first Trk inhibitor of step (b) as a monotherapy to a subject in which (i) the cancer in the subject has relapsed during therapy with the first Trk inhibitor; and/or (ii) the cancer in the subject is not responding to therapy with the first Trk inhibitor; and/or (iii) the subject is intolerant to the first Trk inhibitor; or (e) administering additional doses of the Trk inhibitor of step (b) to a subject in which (i) the cancer has not relapsed during therapy with the first Trk inhibitor; and/or (ii) the cancer in the subject is responding to therapy with the first Trk inhibitor; and/or (iii) the subject is not intolerant to the first Trk inhibitor.

Also provided herein is a method of selecting a treatment for a subject having a cancer, the method comprising: (a) detecting a dysregulation of a NTRK gene, a Trk kinase, or the expression or activity or level of any of the same; (b) administering one or more doses of a first Trk inhibitor to the subject for a period of time; (c) after (a) and (b), determining whether (i) the cancer in the subject has relapsed during therapy with a first Trk inhibitor; and/or (ii) the cancer in the subject is not responding to therapy with a first Trk inhibitor; and/or (iii) the subject is intolerant to a first Trk inhibitor; and (d) selecting a second Trk inhibitor or a treatment that does not include the first Trk inhibitor of step (b) as a monotherapy for a subject in which (i) the cancer in the subject has relapsed during therapy with the first Trk inhibitor; and/or (ii) the cancer in the subject is not responding to therapy with the first Trk inhibitor; and/or (iii) the subject is intolerant to the first Trk inhibitor; or (e) selecting additional doses of the first Trk inhibitor of step (a) for a subject in which (i) the cancer has not relapsed during therapy with the first Trk inhibitor; and/or (ii) the cancer in the subject is responding to therapy with the first Trk inhibitor; and/or (iii) the subject is not intolerant to the first Trk inhibitor.

Also provided herein is a method of selecting a treatment for a subject having a cancer, the method comprising: (a) detecting a dysregulation of a NTRK gene, a Trk kinase, or the expression or activity or level of any of the same; (b) administering one or more doses of a first Trk inhibitor to the subject for a period of time; (c) after (a) and (b), determining whether (i) the cancer in the subject has relapsed during therapy with a first Trk inhibitor; and/or (ii) the cancer in the subject is not responding to therapy with a first Trk inhibitor; and/or (iii) the subject is intolerant to a first Trk inhibitor; and (d) selecting a treatment including a pharmaceutical composition comprising Compound 1, or a pharmaceutically acceptable salt, amorphous, or polymorph form thereof; for a subject in which (i) the cancer in the subject has relapsed during therapy with a first Trk inhibitor; and/or (ii) the cancer in the subject is not responding to therapy with the first Trk inhibitor; and/or (iii) the subject is intolerant to the first Trk inhibitor; or (e) selecting additional doses of the first Trk inhibitor for a subject in which (i) the cancer in the subject has not relapsed during therapy with a first Trk inhibitor; and/or (ii) the cancer in the subject is responding to therapy with the first Trk inhibitor; and/or (iii) the subject is not intolerant to the first Trk inhibitor.

Also provided herein is a method of selecting a treatment for a subject having a cancer, the method comprising: (a) detecting a dysregulation of a NTRK gene, a Trk kinase, or the expression or activity or level of any of the same; (b) administering one or more doses of a first Trk inhibitor to the subject for a period of time; (c) after (a) and (b), determining whether (i) the cancer in the subject has relapsed during therapy with a first Trk inhibitor; and/or (ii) the cancer in the subject is not responding to therapy with the first Trk inhibitor; and/or (iii) the subject is intolerant to the first Trk inhibitor; and (c) selecting a treatment including a Compound 1, or a pharmaceutically acceptable salt, amorphous, or polymorph form thereof; and another anticancer agent or anticancer therapy for a subject in which (i) the cancer in the subject has relapsed during therapy with the first Trk inhibitor; and/or (ii) the cancer in the subject is not responding to therapy with the first Trk inhibitor; and/or (iii) the subject is intolerant to the first Trk inhibitor; or (d) selecting additional doses of the first Trk inhibitor for a subject in which (i) the cancer has not relapsed during therapy with the first Trk inhibitor; and/or (ii) the cancer in the subject is responding to therapy with the first Trk inhibitor; and/or (iii) the subject is not intolerant to the first Trk inhibitor.

In some embodiments, step (a) is performed before step (b). In some embodiments, step (b) is performed before step (a).

In some embodiments, detecting a dysregulation of a NTRK gene, a Trk kinase, or the expression or activity or level of any of the same comprises next generation sequencing, immunohistochemistry, fluorescence microscopy, break apart FISH analysis, and PCR-based amplification (e.g., RT-PCR and quantitative real-time RT-PCR). In some embodiments, the dysregulation of a NTRK gene, a Trk kinase, or the expression or activity or level of any of the same is at least one NTRK1, NTRK2, and/or NTRK3 fusion. In some embodiments, the at least one NTRK1, NTRK2, and/or NTRK3 fusion results in the expression of one or more of a TrkA fusion protein, and/or a TrkB fusion protein, and/or a TrkC fusion protein, wherein the TrkA fusion protein comprises one or more of the of the fusions selected from the group consisting of: TP53-TrkA, LMNA-TrkA, CD74-TrkA, TFG-TrkA, TPM3-TrkA, NFASC-TrkA, BCAN-TrkA, MPRIP-TrkA, TPR-TrkA, RFWD2-TrkA, IRF2BP2-TrkA, SQSTM1-TrkA, SSBP2-TrkA, RABGAP1L-TrkA, C18ORF8-TrkA, RNF213-TrkA, TBC1D22A-TrkA, C20ORF112-TrkA, DNER-TrkA, ARHGEF2-TrkA, CHTOP-TrkA, PPL-TrkA, PLEKHA6-TrkA, PEAR1-TrkA, MRPL24-TrkA, MDM4-TrkA, LRRC71-TrkA, GRIPAP1-TrkA, TAF-TrkA, EPS15-TrkA, DYNC2H1-TrkA, CEL-TrkA, EPHB2-TrkA, TGF-TrkA, NELL1-TrkA, EPL4-TrkA, CTNND2-TrkA, TCEANC2-TrkA, SCYL3-TrkA, AMOTL2-TrkA, MEF2D-TrkA, L7a-TrkA, ZBTB7B-TrkA, TRIM63-TrkA, DDR2-TrkAl, GON4L-TrkA, PDE4DIP-TrkA, NTRK1-P2RY8, CTRC-TrkA, and VANGL2-TrkA; and/or the TrkB fusion protein comprises one or more of the of the fusions selected from the group consisting of: NACC2-TrkB, QKI-TrkB, AFAP1-TrkB, PAN3-TrkB, SQSTM1-TrkB, TRIM24-TrkB, VCL-TrkB, AGBL4-TrkB, DAB2IP-TrkB, TrkB-TERT, ETV6-TrkB, NOS1AP-TrkB, GKAP1-TrkB, KCTD8-TrkB, TBC1D2-TrkB, VCAN-TrkB, SLMAP-TrkB, TLE4-TrkB, STRN3-TrkB, WNK2-TrkB, TrkB-BEND5, TrkB-TRAF2, Nav1-TrkB, and STRN-TrkB; and/or the TrkC fusion protein comprises one or more of the of the fusions selected from the group consisting of: ETV6-TrkC1, BTBD1-TrkC, LYN-TrkC, RBPMS-TrkC, EML4-TrkC, TrkC-HOMER2, TFG-TrkC, FAT1-TrkC, MYO5A-TrkC, MYH9-TrkC, KANK1-TrkC, SQSTM1-TrkC, UBE2R2-TrkC, HNRNPA2B1-TrkC, VPS18-TrkC, AKAP13-TrkC, TrkC-LOXL2, TrkC-PEAK1, ZNF710-TrkC, TPM4-TrkC, and LMNA-TrkC.

Also provided herein is a method of selecting a treatment for a subject having a cancer, the method comprises: (a) determining whether, for a subject having a cancer and previously administered one or more doses of a first Trk inhibitor, (i) the cancer in the subject has relapsed during therapy with a first Trk inhibitor; and/or (ii) the cancer in the subject is not responding to therapy with a first Trk inhibitor; and/or (iii) the subject is intolerant to a first Trk inhibitor; (b) selecting a second Trk inhibitor or a treatment that does not include the first Trk inhibitor of step (a) as a monotherapy to a subject in which (i) the cancer in the subject has relapsed during therapy with the first Trk inhibitor; and/or (ii) the cancer in the subject is not responding to therapy with the first Trk inhibitor; and/or (iii) the subject is intolerant to the first Trk inhibitor; or (c) selecting additional doses of the first Trk inhibitor of step (a) to a subject in which (i) the cancer has not relapsed during therapy with a first Trk inhibitor; and/or (ii) the cancer in the subject is responding to therapy with the first Trk inhibitor; and/or (iii) the subject is not intolerant to the first Trk inhibitor.

In some embodiments, the second Trk inhibitor is Compound 1, or a pharmaceutically acceptable salt, amorphous, or polymorph form thereof.

Also provided herein is a method of selecting a treatment for a subject having a cancer, the method comprises: (a) determining whether, for a subject having a cancer and previously administered one or more doses of a first Trk inhibitor, (i) the cancer in the subject has relapsed during therapy with a first Trk inhibitor; and/or (ii) the cancer in the subject is not responding to therapy with a first Trk inhibitor; and/or (iii) the subject is intolerant to a first Trk inhibitor; (b) selecting a treatment that includes Compound 1, or a pharmaceutically acceptable salt, amorphous, or polymorph form thereof, for a subject in which (i) the cancer in the subject has relapsed during therapy with a first Trk inhibitor; and/or (ii) the cancer in the subject is not responding to therapy with the first Trk inhibitor; and/or (iii) the subject is intolerant to the first Trk inhibitor; or (c) selecting additional doses of the first Trk inhibitor to a subject in which (i) the cancer has not relapsed during therapy with a first Trk inhibitor; and/or (ii) the cancer in the subject is responding to therapy with the first Trk inhibitor; and/or (iii) the subject is not intolerant to the first Trk inhibitor.

Also provided herein is a method of selecting a treatment for a subject having a cancer, the method comprises: (a) determining whether, for a subject having a cancer and previously administered one or more doses of a first Trk inhibitor, (i) the cancer in the subject has relapsed during therapy with a first Trk inhibitor; and/or (ii) the cancer in the subject is not responding to therapy with a first Trk inhibitor; and/or (iii) the subject is intolerant to a first Trk inhibitor; (b) selecting a treatment that includes Compound 1, or a pharmaceutically acceptable salt, amorphous, or polymorph form thereof, and an another anticancer agent or anticancer therapy to a subject in which (i) the cancer in the subject has relapsed during therapy with a first Trk inhibitor; and/or (ii) the cancer in the subject is not responding to therapy with the first Trk inhibitor; and/or (iii) the subject is intolerant to the first Trk inhibitor; or (c) selecting additional doses of the first Trk inhibitor to a subject in which (i) the cancer has not relapsed during therapy with a first Trk inhibitor; and/or (ii) the cancer in the subject is responding to therapy with the first Trk inhibitor; and/or (iii) the subject is not intolerant to the first Trk inhibitor.

In some of any of the above embodiments, the first Trk inhibitor is selected from the group consisting of: entrectinib (N-[5-(3,5-difluoro-benzyl)-1H-indazol-3-yl]-4-(4-methylpiperazin-1-yl)-2-(tetrahydro-pyran-4-ylamino)-benzamide), (S)—N-(5-((R)-2-(2,5-difluorophenyl)pyrrolidin-1-yl)pyrazolo[1,5-a]pyrimidin-3-yl)-3-hydroxypyrrolidine-1-carboxamide sulfate, cabozantinib ((N-(4-((6,7-Dimethoxyquinolin-4-yl)oxy)phenyl)-N′-(4-fluorophenyl)cyclopropane-1,1-dicarboxamide)), dovitinib (4-amino-5-fluoro-3-[6-(4-methylpiperazin-1-yl)-1H-benzimidazol-2-yl]quinolin-2(1H)-one mono 2-hydroxypropanoate hydrate), belizatinib (4-fluoro-N-(6-((4-(2-hydroxypropan-2-yl)piperidin-1-yl)methyl)-1-((1s,4s)-4-(i sopropylcarbamoyl)cyclohexyl)-1H-benzo[d]imidazol-2-yl)benzamide), sitravatinib (N-(3-fluoro-4-((2-(5-(((2-methoxyethyl)amino)methyl)pyridin-2-yl)thieno[3,2-b]pyridin-7-yl)oxy)phenyl)-N-(4-fluorophenyl)cyclopropane-1,1-dicarboxamide), PLX7486, altiratinib (N-(4-((2-(cyclopropanecarboxamido)pyridin-4-yl)oxy)-2,5-difluorophenyl)-N-(4-fluorophenyl)cyclopropane-1,1-dicarboxamide), AZD7451 ((S)—N-(1-(5-fluoropyrimidin-2-yl)ethyl)-3-(5-isopropoxy-1H-pyrazol-3-yl)-3H-imidazo[4,5-b]pyridin-5-amine), (6R,15R)-9-fluoro-15-methyl-2,11,16,20,21,24-hexaazapentacyclo[16.5.2.02,6.07,12.021,25]pentacosa-1 (24),7,9, 11,18(25), 19,22-heptaen-17-one, a (R)-2-phenylpyrrolidine substituted imadazopyridazine, AZD6918, GNF-4256, GTx-186, GNF-5837, AZ623, AG-879, CT327, AR-772, AR-523, AR-786, AR-256, AR-618, AZ-23, CEP-701, CEP-751, PHA-739358, dovitinib, Go 6976, GW441756, MGCD516, ONO-5390556, PHA-848125AC, Regorafenib, Sorafenib, Sunitinib, TSR-011, VM-902A, K252a, a 4-aminopyrazolylpyrimidine, a substituted pyrazolo[1,5-a]pyrimidine compound, BMS-754807, ONO-7579, F17752, ANA-12, ONO-4474, GZ389988, or TPX-0005 ((7S,13R)-11-fluoro-7,13-dimethyl-6,7,13,14-tetrahydro-1,15-ethenopyrazolo[4,3-f][1,4,8,10]benzoxatriazacyclotridecin-4(5H)-one; repotrectinib). In some embodiments, the first Trk inhibitor is selected from the group consisting of: entrectinib (N-[5-(3,5-difluoro-benzyl)-1H-indazol-3-yl]-4-(4-methylpiperazin-1-yl)-2-(tetrahydro-pyran-4-ylamino)-benzamide); TPX-0005 ((7S,13R)-11-fluoro-7,13-dimethyl-6,7,13,14-tetrahydro-1,15-ethenopyrazolo[4,3-f][1,4,8,10]benzoxatriazacyclotridecin-4(5H)-one; repotrectinib); PLX7486; and (S)—N-(5-((R)-2-(2,5-difluorophenyl)pyrrolidin-1-yl)pyrazolo[1,5-a]pyrimidin-3-yl)-3-hydroxypyrrolidine-1-carboxamide sulfate.

In some of any of the above embodiments, the cancer is selected from the group consisting of: adenocarcinoma, adrenal gland cortical carcinoma, adrenal gland neuroblastoma, anus squamous cell carcinoma, appendix adenocarcinoma, bladder urothelial carcinoma, bile duct adenocarcinoma, bladder carcinoma, bladder urothelial carcinoma, bone chordoma, bone marrow leukemia lymphocytic chronic, bone marrow leukemia non-lymphocytic acute myelocytic, bone marrow lymph proliferative disease, bone marrow multiple myeloma, bone sarcoma, brain astrocytoma, brain glioblastoma, brain medulloblastoma, brain meningioma, brain oligodendroglioma, breast adenoid cystic carcinoma, breast carcinoma, breast ductal carcinoma in situ, breast invasive ductal carcinoma, breast invasive lobular carcinoma, breast metaplastic carcinoma, cervix neuroendocrine carcinoma, cervix squamous cell carcinoma, colon adenocarcinoma, colon carcinoid tumor, duodenum adenocarcinoma, endometrioid tumor, esophagus adenocarcinoma, eye intraocular melanoma, eye intraocular squamous cell carcinoma, eye lacrimal duct carcinoma, fallopian tube serous carcinoma, gallbladder adenocarcinoma, gallbladder glomus tumor, gastroesophageal junction adenocarcinoma, head and neck adenoid cystic carcinoma, head and neck carcinoma, head and neck neuroblastoma, head and neck squamous cell carcinoma, kidney chromophore carcinoma, kidney medullary carcinoma, kidney renal cell carcinoma, kidney renal papillary carcinoma, kidney sarcomatoid carcinoma, kidney urothelial carcinoma, leukemia lymphocytic, liver cholangiocarcinoma, liver hepatocellular carcinoma, lung adenocarcinoma, lung adenosquamous carcinoma, lung atypical carcinoid, lung carcinosarcoma, lung large cell neuroendocrine carcinoma, lung non-small cell lung carcinoma, lung sarcoma, lung sarcomatoid carcinoma, lung small cell carcinoma, lung small cell undifferentiated carcinoma, lung squamous cell carcinoma, lymph node lymphoma diffuse large B cell, lymph node lymphoma follicular lymphoma, lymph node lymphoma mediastinal B-cell, lymph node lymphoma plasmablastic lung adenocarcinoma, lymphoma follicular lymphoma, non-Hodgkin's lymphoma, nasopharynx and paranasal sinuses undifferentiated carcinoma, ovary carcinoma, ovary carcinosarcoma, ovary clear cell carcinoma, ovary epithelial carcinoma, ovary granulosa cell tumor, ovary serous carcinoma, pancreas carcinoma, pancreas ductal adenocarcinoma, pancreas neuroendocrine carcinoma, peritoneum mesothelioma, peritoneum serous carcinoma, placenta choriocarcinoma, pleura mesothelioma, prostate acinar adenocarcinoma, prostate carcinoma, rectum adenocarcinoma, rectum squamous cell carcinoma, skin adnexal carcinoma, skin basal cell carcinoma, skin melanoma, skin Merkel cell carcinoma, skin squamous cell carcinoma, small intestine adenocarcinoma, small intestine gastrointestinal stromal tumors (GISTs), soft tissue angiosarcoma, soft tissue Ewing sarcoma, soft tissue hemangioendothelioma, soft tissue inflammatory myofibroblastic tumor, soft tissue leiomyosarcoma, soft tissue liposarcoma, soft tissue neuroblastoma, soft tissue paraganglioma, soft tissue perivascular epitheliod cell tumor, soft tissue sarcoma, soft tissue synovial sarcoma, stomach adenocarcinoma, stomach adenocarcinoma diffuse-type, stomach adenocarcinoma intestinal type, stomach adenocarcinoma intestinal type, stomach leiomyosarcoma, thymus carcinoma, thymus thymoma lymphocytic, thyroid papillary carcinoma, unknown primary adenocarcinoma, unknown primary carcinoma, unknown primary malignant neoplasm, unknown primary melanoma, unknown primary sarcomatoid carcinoma, unknown primary squamous cell carcinoma, unknown undifferentiated neuroendocrine carcinoma, unknown primary undifferentiated small cell carcinoma, uterus carcinosarcoma, uterus endometrial adenocarcinoma, uterus endometrial adenocarcinoma endometrioid, uterus endometrial adenocarcinoma papillary serous, and uterus leiomyosarcoma. In some embodiments, the cancer is selected from the group consisting of: non-small cell lung carcinoma, thyroid neoplasms, sarcoma, GIST, malignant peripheral nerve sheath tumors, colorectal neoplasms, salivary gland neoplasms, biliary tract neoplasms, primary brain neoplasm, breast secretory carcinoma, melanoma, glioblastoma, bile duct neoplasms, astrocytoma, head and neck squamous cell carcinoma, pontine glioma, pancreatic neoplasms, ovarian neoplasms, uterine neoplasms, renal cell carcinoma, cholangiocarcinoma, skin carcinoma, bronchogenic carcinoma, bronchial neoplasms, lung neoplasms, respiratory tract neoplasms, thoracic neoplasms, nerve tissue neoplasms, nevi and melanomas, intestinal neoplasm, thyroid cancer, fibrosarcoma, infantile fibrosarcoma, congenital mesoblastic nephroma, and central nervous system neoplasms.

In some embodiments, the subject is previously identified or diagnosed as having the cancer.

In some embodiments, the cancer exhibits a TrkA fusion protein; and/or a TrkB fusion protein; and/or a TrkC fusion protein. In some embodiments, the TrkA fusion protein comprises one or more of the fusions selected from the group consisting of: TP53-TrkA, LMNA-TrkA, CD74-TrkA, TFG-TrkA, TPM3-TrkA, NFASC-TrkA, BCAN-TrkA, MPRIP-TrkA, TPR-TrkA, RFWD2-TrkA, IRF2BP2-TrkA, SQSTM1-TrkA, SSBP2-TrkA, RABGAP1L-TrkA, C18ORF8-TrkA, RNF213-TrkA, TBC1D22A-TrkA, C20ORF112-TrkA, DNER-TrkA, ARHGEF2-TrkA, CHTOP-TrkA, PPL-TrkA, PLEKHA6-TrkA, PEAR1-TrkA, MRPL24-TrkA, MDM4-TrkA, LRRC71-TrkA, GRIPAP1-TrkA, TAF-TrkA, EPS15-TrkA, DYNC2H1-TrkA, CEL-TrkA, EPHB2-TrkA, TGF-TrkA, NELL1-TrkA, EPL4-TrkA, CTNND2-TrkA, TCEANC2-TrkA, SCYL3-TrkA, AMOTL2-TrkA, MEF2D-TrkA, L7a-TrkA, ZBTB7B-TrkA, TRIM63-TrkA, DDR2-TrkAl, GON4L-TrkA, PDE4DIP-TrkA, TrkA-P2RY8, CTRC-TrkA, and VANGL2-TrkA; and/or the TrkB fusion protein comprises one or more of the of the fusions selected from the group consisting of: NACC2-TrkB, QKI-TrkB, AFAP1-TrkB, PAN3-TrkB, SQSTM1-TrkB, TRIM24-TrkB, VCL-TrkB, AGBL4-TrkB, DAB2IP-TrkB, TrkB-TERT, ETV6-TrkB, NOS1AP-TrkB, GKAP1-TrkB, KCTD8-TrkB, TBC1D2-TrkB, VCAN-TrkB, SLMAP-TrkB, TLE4-TrkB, STRN3-TrkB, WNK2-TrkB, TrkB-BEND5, TrkB-TRAF2, Nav1-TrkB, and STRN-TrkB; and/or the TrkC fusion protein comprises one or more of the of the fusions selected from the group consisting of: ETV6-TrkC1, BTBD1-TrkC, LYN-TrkC, RBPMS-TrkC, EML4-TrkC, TrkC-HOMER2, TFG-TrkC, FAT1-TrkC, MYO5A-TrkC, MYH9-TrkC, KANK1-TrkC, SQSTM1-TrkC, UBE2R2-TrkC, HNRNPA2B1-TrkC, VPS18-TrkC, AKAP13-TrkC, TrkC-LOXL2, TrkC-PEAK1, ZNF710-TrkC, TPM4-TrkC, and LMNA-TrkC.

Unless otherwise defined, all technical and scientific terms used herein have the same meaning as commonly understood by one of ordinary skill in the art to which the present application belongs. Methods and materials are described herein for use in the present application; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the present application will be apparent from the following detailed description and figures, and from the claims.

### DESCRIPTION OF DRAWINGS

FIG. 1 is a XRPD diffractogram of Compound 1 (Form I).

FIG. 2 is a TG/DTA thermogram of Compound 1 (Form I).

FIG. 3 is a DSC thermogram of Compound 1 (Form I).

FIG. 4 is a GVS isotherm plot of Compound 1 (Form I).

FIG. 5 is a GVS kinetic plot of Compound 1 (Form I).

FIG. 6 is a DVS isotherm plot of Compound 1 (Form I).

FIG. 7 is a DVS change in mass plot of Compound 1 (Form I).

FIG. 8 is an IR spectrum Compound 1 (Form I).

FIG. 9 is a ¹H NMR spectrum of Compound 1 (Form I).

FIG. 10 is an image showing a 3-D view of Compound 1 (Form I) with atom labels.

FIG. 11 is an image showing a ORTEP view of Compound 1 (Form I) with atom labels.

FIG. 12 is an image showing a 3-D view of Compound 1, acetonitrile solvate with atom labels.

FIG. 13 is an image showing a ORTEP view of Compound 1, acetonitrile solvate with atom labels.

FIG. 14 is a XRPD diffractogram of Compound 1 edisylate.

FIG. 15 is a XRPD diffractogram of Compound 1 tosylate.

FIG. 16 is a XRPD diffractogram of Compound 1 mesylate.

FIG. 17 is a XRPD diffractogram of Compound 1 besylate (pattern 1).

FIG. 18 is a XRPD diffractogram of Compound 1 besylate (pattern 2).

FIG. 19 is a XRPD diffractogram of Compound 1 oxalate.

FIG. 20 is a XRPD diffractogram of Compound 1 fumarate.

FIG. 21 is a XRPD diffractogram of Compound 1 citrate (Form A).

FIG. 22 is a XRPD diffractogram of Compound 1 L-malate.

FIG. 23 is a XRPD diffractogram of Compound 1 succinate.

FIG. 24 is a TG/DTA thermogram of Compound 1 tosylate.

FIG. 25 is a TG/DTA thermogram of Compound 1 mesylate.

FIG. 26 is a TG/DTA thermogram of Compound 1 oxalate.

FIG. 27 is a TG/DTA thermogram of Compound 1 fumarate.

FIG. 28 is a TG/DTA thermogram of Compound 1 L-malate.

FIG. 29 is a TG/DTA thermogram of Compound 1 succinate.

FIG. 30 is a XRPD diffractogram of Compound 1 mesylate acetone solvate.

FIG. 31 is a TG/DTA thermogram of Compound 1 mesylate acetone solvate.

FIG. 32 is a DSC thermogram of Compound 1 mesylate.

FIG. 33 is a GVS isotherm of Compound 1 mesylate acetone solvate.

FIG. 34 is a GVS kinetic plot of Compound 1 mesylate acetone solvate.

FIG. 35 is an IR spectrum of Compound 1 mesylate acetone solvate.

FIG. 36 is a ¹H NMR spectrum of Compound 1 mesylate acetone solvate.

FIG. 37 is a TG/DTA thermogram of Compound 1 besylate.

FIG. 38 is a DSC thermogram of Compound 1 besylate.

FIG. 39 is a DVS isotherm of Compound 1 besylate.

FIG. 40 is a DVS kinetic plot of Compound 1 besylate.

FIG. 41 is an IR spectrum of Compound 1 besylate.

FIG. 42 is ¹H NMR spectrum of Compound 1 besylate.

FIG. 43 is a TG/DTA thermogram of Compound 1 citrate (Form A).

FIG. 44 is a DSC thermogram of Compound 1 citrate (Form A).

FIG. 45 is a DVS isotherm of Compound 1 citrate (Form A).

FIG. 46 is a DVS kinetic plot of Compound 1 citrate (Form A).

FIG. 47 is an IR spectrum of Compound 1 citrate (Form A).

FIG. 48 is a ¹H-NMR spectrum of Compound 1 citrate (Form A).

FIG. 49 is a XRPD diffractogram of a Compound 1 citrate (Form B).

FIG. 50 is a sequence listing for an exemplary wildtype TrkA polypeptide (SEQ ID NO: 1).

FIG. 51 is a sequence listing for an exemplary wildtype TrkB polypeptide (SEQ ID NO: 5).

FIG. 52 is a sequence listing for an exemplary wildtype TrkC polypeptide (SEQ ID NO: 7).

### DETAILED DESCRIPTION

### Definitions

As used herein, the phrase “solid form” refers to Compound 1 or a salt of Compound 1 in either an amorphous state or a crystalline state (“crystalline form” or “crystalline solid”), whereby a compound in a crystalline state may optionally include solvent or water within the crystalline lattice, for example, to form a solvated or hydrated crystalline form.

The term “hydrated,” as used herein, is meant to refer to a crystalline form that includes water molecules in the crystalline lattice.

Different crystalline forms of compounds can be characterized by X-ray powder diffraction (XRPD), differential scanning calorimetry (DSC), differential thermal analysis (DTA), and/or thermogravimetric analysis (TGA). An X-ray powder diffraction (XRPD) pattern of reflections (peaks) is typically considered a fingerprint of a particular crystalline form. It is well known that the relative intensities of the XRPD peaks can widely vary depending on the sample preparation technique, crystal size distribution, various filters used, the sample mounting procedure, and the particular instrument employed. In some instances, new peaks may be observed or existing peaks may disappear depending on the type of instrument or the settings (for example, whether a Ni filter is used or not).

As used herein, the term “peak” refers to a reflection having a relative height/intensity of at least about 5% of the maximum peak height/intensity in the XPRD. Peak assignments, such as those reported herein, can vary by plus or minus 0.2° (2-theta), and the term “substantially” or “about” as used in the context of XRPD herein is meant to refer to the above-mentioned variations. Thus, for example, a 2-theta value of “about 9.1” means a 2-theta value of 9.1±0.2.

As described herein, temperature readings in connection with DSC, TGA, or other thermal experiments can vary by ±4° C. depending on the instrument, particular settings, sample preparation, etc. Accordingly, a crystalline form reported herein having a DSC thermogram “substantially” as shown in any of the Figures is understood to accommodate such variation. An endothermal or exothermic event at “about” a certain temperature is also understood to accommodate this variation.

As used herein, the term “melting point” refers to an endothermal event or endothermal event observed in, e.g., a DSC thermogram. An endothermal event is a process or reaction in which a sample absorbs energy from its surroundings in the form of e.g., heat as in a DSC experiment. An exothermic event is a process or reaction in which a sample releases energy. The process of heat absorption and release can be detected by DSC. In some embodiments, the term “melting point” is used to describe the major endothermal event on a DSC thermogram.

The terms “room temperature” or “ambient temperature” as used herein, are understood in the art, and refer generally to a temperature, e.g., a reaction temperature, that is about the temperature of the room in which the reaction is carried out, for example, a temperature from about 20° C. to about 30° C.

In some embodiments, the compounds, salts, and forms described herein are substantially isolated. By “substantially isolated” is meant that the compound, salt, or form is at least partially or substantially separated from the environment in which it was formed or detected. Partial separation can include, e.g., a composition enriched in the compound, salt or form. Substantial separation can include compositions containing at least about 50%, at least about 60%, at least about 70%, at least about 80%, at least about 90%, at least about 95%, at least about 97%, or at least about 99% by weight of the compound, salt or form.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “therapeutically effective amount” refers to the amount of active compound or pharmaceutical agent that elicits the biological or medicinal response in a tissue, system, animal, individual or human that is being sought by a researcher, veterinarian, medical doctor or other clinician.

As used herein, terms “treat” or “treatment” refer to therapeutic or palliative measures. Beneficial or desired clinical results include, but are not limited to, alleviation, in whole or in part, of symptoms associated with a disease or disorder or condition, diminishment of the extent of disease, stabilized (i.e., not worsening) state of disease, delay or slowing of disease progression, amelioration or palliation of the disease state (e.g., one or more symptoms of the disease), and remission (whether partial or total), whether detectable or undetectable. “Treatment” can also mean prolonging survival as compared to expected survival if not receiving treatment.

The term “therapy” refers to the administration of one or more doses of an active compound or pharmaceutical agent to a subject as part of a therapeutic regimen.

In one embodiment, the term “preventing” as used herein means the prevention of the onset, recurrence or spread, in whole or in part, of the disease or condition as described herein (e.g., multiple types of pain including inflammatory pain, neuropathic pain, and pain associated with cancer, surgery, and bone fracture), or a symptom thereof.

The term “progression” refers to cancer that becomes worse or spreads in the body, as defined by the National Cancer Institute (NCI Dictionary of Cancer Terms). For example, progression can include an increase in the number of cancer cells in the subject, an increase in the size of one or more tumors in the subject, an increase in tumor burden, an increase in the rate or extent of metastasis, worsening symptoms, in whole or in part, associated with the cancer, an increase in the extent of disease, and/or an acceleration of disease progression. “Progression” can also mean shortening survival as compared to expected survival if not receiving therapy. In some embodiments, the tumor burden can be assessed using RECIST (e.g., RECIST version 1 or version 1.1). See, for example, Eisenhauer et al., Eur. J. Cancer. 45(2):228-47 (2009), which is incorporated by reference in its entirety herein. In some embodiments, the cancer is glioma and the progression of the glioma is assessed by RANO. See, for example, Wen et al., J. Clin. Oncol. 28(11): 1963-72 (2010), which is incorporated by reference in its entirety herein.

The term “relapse” refers to the return of a disease or the signs and symptoms of a disease after a period of improvement, as defined by the National Cancer Institute (NCI Dictionary of Cancer Terms). For example, relapse can include detecting an increase in the number of cancer cells in the subject, an increase in the size of one or more tumors in the subject, an increase in tumor burden, an increase in the rate or extent of metastasis, worsening symptoms, in whole or in part, associated with the cancer, an increase in the extent of disease, and/or an acceleration of disease progression after a period of improvement. In some embodiments, relapse can include progression of the cancer after a period of improvement. In some embodiments, a period of improvement can include a decrease in the number of cancer cells in a subject, a decrease in the size of one or more tumors in the subject, a decrease in tumor burden, a decrease in the rate or extent of metastasis, improving symptoms, in whole or in part, associated with the cancer, a decrease in the extent of disease, and/or a slowing of disease progression. “Relapse” can also include “recurrence,” which the National Cancer institute defines as cancer that has recurred, usually after a period of time during which the cancer could not be detected. The cancer may come back to the same location in the body as the original (primary) tumor or to another location in the body (NCI Dictionary of Cancer Terms). In some embodiments, not detecting a cancer can include not detecting a cancer cells in the subject, not detecting a tumors in the subject, and/or no symptoms, in whole or in part, associated with the cancer.

As used herein, the terms “intolerance” and “intolerant” can refer to the occurrence of a severe, disabling, or life-threatening adverse event that leads to unplanned hospitalization during therapy, therapy discontinuation, and/or therapy dose reduction, functional decline attributed to therapy, and/or a decrease in performance status. In some embodiments, a decrease in performance status can be assessed using the Eastern Cooperative Oncology Group (ECOG) Scale of Performance Status (see, e.g., Oken et al. Am. J. Clin. Oncol. 5:649-655 (1982), which is incorporated by reference in its entirey herein). In some embodiments, a decrease in performance status can be assessed using the Karnofsky Performance Status (see, e.g., Peus et al., BMC Med. Inform. Decis. Mak. 13: 72 (2013), which is incorporated by reference in its entirey herein). In some embodiments, the subject is a pediatric patient and the performance status is assessed by the Lansky Performance Score (see, e.g., Lansky et al., Cancer. 60(7):1651-6 (1987), which is incorporated by reference in its entirey herein).

The terms “effective amount” and “therapeutically effective amount” refer to an amount of compound that, when administered to a mammal in need of such treatment, is sufficient to (i) treat or prevent a particular disease, condition, or disorder, (ii) attenuate, ameliorate, or eliminate one or more symptoms of the particular disease, condition, or disorder, or (iii) prevent or delay the onset of one or more symptoms of the particular disease, condition, or disorder described herein. The amount of a Compound 1, or salt thereof, that will correspond to such an amount will vary depending upon factors such as the particular compound, disease condition and its severity, the identity (e.g., weight) of the mammal in need of treatment, but can nevertheless be routinely determined by one skilled in the art.

The terms “individual” or “patient,” used interchangeably, refer to any animal, including mammals, and most preferably humans. As used herein, the term “mammal” refers to a warm-blooded animal that has or is at risk of developing a disease described herein and includes, but is not limited to, guinea pigs, dogs, cats, rats, mice, hamsters, primates, and humans.

Acute pain, as defined by the International Association for the Study of Pain, results from disease, inflammation, or injury to tissues. This type of pain generally comes on suddenly, for example, after trauma or surgery, and may be accompanied by anxiety or stress. The cause can usually be diagnosed and treated, and the pain is confined to a given period of time and severity. In some rare instances, it can become chronic.

Chronic pain, as defined by the International Association for the Study of Pain, is widely believed to represent disease itself. It can be made much worse by environmental and psychological factors. Chronic pain persists over a longer period than acute pain and is resistant to most medical treatments, generally over 3 months or more. It can and often does cause severe problems for patients.

The term “Trk-associated cancer” as used herein refers to cancers associated with or having a dysregulation of a NTRK gene, a Trk protein, or expression or activity, or level of any of the same. Exemplary Trk-associated cancers are provided herein.

The phrase “dysregulation of a NTRK gene, a Trk kinase, or the expression or activity or level of any of the same” refers to a genetic mutation (e.g., a NTRK gene translocation that results in the expression of a fusion protein, a deletion in a NTRK gene that results in the expression of a Trk protein that includes a deletion of at least one amino acid as compared to the wild-type Trk protein, a mutation in a NTRK gene that results in the expression of a Trk protein with one or more point mutations, or an alternative spliced version of a NTRK mRNA that results in a Trk protein having a deletion of at least one amino acid in the Trk protein as compared to the wild-type Trk protein) or a NTRK gene amplification that results in overexpression of a Trk protein or an autocrine activity resulting from the overexpression of a NTRK gene in a cell that results in a pathogenic increase in the activity of a kinase domain of a Trk protein (e.g., a constitutively active kinase domain of a Trk protein) in a cell. As another example, a dysregulation of a NTRK gene, a Trk protein, or expression or activity, or level of any of the same, can be a mutation in a NTRK gene that encodes a Trk protein that is constitutively active or has increased activity as compared to a protein encoded by a NTRK gene that does not include the mutation. For example, a dysregulation of a NTRK gene, a Trk protein, or expression or activity, or level of any of the same, can be the result of a gene or chromosome translocation which results in the expression of a fusion protein that contains a first portion of Trk that includes a functional kinase domain, and a second portion of a partner protein that is not Trk. In some examples, dysregulation of a NTRK gene, a Trk protein, or expression or activity or level of any of the same can be a result of a gene translocation of one NTRK gene with another non-NTRK gene. Non-limiting examples of fusion proteins are described in Tables 2, 5, and 8. Additional examples of Trk kinase protein mutations (e.g., point mutations) are Trk inhibitor resistance mutations.

The term “wildtype” or “wild-type” when referring to a Trk nucleic acid or protein describes a nucleic acid (e.g., a NTRK gene or a NTRK mRNA) or protein (e.g., a Trk protein) that is found in a subject that does not have a Trk-associated disease, e.g., a Trk-associated cancer (and optionally also does not have an increased risk of developing a Trk-associated disease and/or is not suspected of having a Trk-associated disease), or is found in a cell or tissue from a subject that does not have a Trk-associated disease, e.g., a Trk-associated cancer (and optionally also does not have an increased risk of developing a Trk-associated disease and/or is not suspected of having a Trk-associated disease).

As used herein, a “first Trk kinase inhibitor” or “first Trk inhibitor” is a Trk kinase inhibitor as defined herein, but which does not include Compound 1 or a pharmaceutically acceptable salt, amorphous, or polymorph form thereof, or a pharmaceutical composition thereof, as defined herein. As used herein, a “second Trk kinase inhibitor” or a “second Trk inhibitor” is a Trk kinase inhibitor as defined herein, but which can include Compound 1, or a pharmaceutically acceptable salt, amorphous, or polymorph form thereof, or a pharmaceutical composition thereof, as defined herein. When both a first and a second Trk inhibitor are present in a method provided herein, the first and second Trk kinase inhibitor are different.

### 1. Compound 1 and Pharmaceutical Compositions, Polymorphs, and Salts Thereof

Provided herein is Compound 1, (6R,15R)-9-fluoro-15-methyl-2,11,16,20,21,24-hexaazapentacyclo[16.5.2.0^{2,6}.0^{7,12}.0^{21,25}]pentacosa-1 (24),7,9,11,18(25), 19,22-heptaen-17-one having the following structural formula:

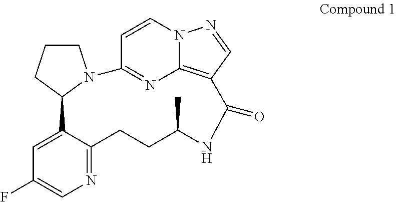 [SMILES: C[C@@H]1CCc2ncc(F)cc2[C@H]2CCCN2c2ccn3ncc(c3n2)C(=O)N1]

or a pharmaceutically acceptable salt, amorphous, or polymorph form thereof.

In one aspect, the present disclosure is directed to a pharmaceutical composition comprising Compound 1 and a compounding agent as disclosed herein.

In some embodiments of a pharmaceutical composition comprising Compound 1 and a compounding agent as disclosed herein, Compound 1 is present in a diastereomeric excess (d.e.) of at least 80% relative to the diastereomeric compound of formula I′:

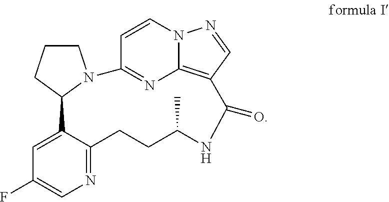 [SMILES: C[C@H]1CCc2ncc(F)cc2[C@H]2CCCN2c2ccn3ncc(c3n2)C(=O)N1]

In some embodiments, Compound 1 is present in a d.e. of at least 90% relative to the compound of formula I′. In some embodiments, Compound 1 is present in a d.e. of at least 92% relative to the compound of formula I′. In some embodiments, Compound 1 is present in a d.e. of at least 94% relative to the compound of formula I′. In some embodiments, Compound 1 is present in a d.e. of at least 96% relative to the compound of formula I′. In some embodiments, Compound 1 is present in a d.e. of at least 98% relative to the compound of formula I′.

In some embodiments, the compound of Formula I is prepared from a mixture of the compound of Formula I and the compound of formula I′ by separating the two compounds. In some embodiments, the two compounds are separated by chromatography.

In one aspect, the present disclosure is directed to a pharmaceutical composition comprising a form of (6R,15R)-9-fluoro-15-methyl-2,11,16,20,21,24-hexaazapentacyclo[16.5.2.0^{2,6}.0^{7,12}.0^{21,25}]pentacosa-1 (24),7,9,11,18(25), 19,22-heptaen-17-one (Compound 1), the structure of which is shown below:

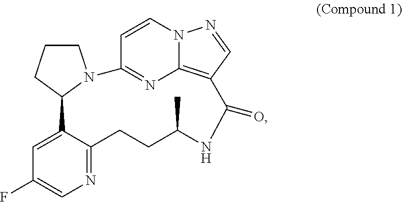 [SMILES: C[C@@H]1CCc2ncc(F)cc2[C@H]2CCCN2c2ccn3ncc(c3n2)C(=O)N1]

and a compounding agent as disclosed herein.

In another general aspect, the present disclosure is directed to a pharmaceutical composition comprising a salt of Compound 1.

In some embodiments, provided herein is a pharmaceutical composition comprising any one of the crystalline forms, solid forms, solvates, hydrates or salts of Compound 1 described herein, and a compounding agent as disclosed herein.

In some embodiments, provided herein is a pharmaceutical composition comprising Compound 1 and a compounding agent, wherein at least some of Compound 1 is present as any one of the crystalline forms, solid forms, solvates, hydrates, or salts described herein.

In some embodiments, provided herein is a pharmaceutical composition prepared by a process comprising mixing a compounding agent with Compound 1, to form the composition.

In some embodiments, provided herein is a pharmaceutical composition prepared by a process comprising mixing a compounding agent with any one of the crystalline forms, solid forms, solvates, hydrates, or salts described herein, to form the pharmaceutical composition.

In some embodiments, provided herein is a process for preparing a pharmaceutical composition comprising Compound 1, comprising mixing a compounding agent as disclosed herein with Compound 1, to form the pharmaceutical composition.

In some embodiments, provided herein is a process for preparing a pharmaceutical composition, comprising mixing a compounding agent as disclosed herein with any one of the crystalline forms, solid forms, solvates, hydrates, or salts described herein, to form the pharmaceutical composition.

In some embodiments, the pharmaceutical composition is a liquid oral pharmaceutical composition.

Pharmaceutical compositions comprising Compound 1, or pharmaceutical compositions comprising any one of the crystalline forms, solid forms, solvates, hydrates or salts described herein, can be prepared by intimately mixing, respectively, Compound 1 or the crystalline form, solid form, solvate, hydrate or salt described herein with a compounding agent as disclosed herein according to conventional pharmaceutical compounding techniques. For liquid oral compositions such as suspensions, elixirs and solutions, suitable compounding agents and additives comprise one or more of water, glycols, oils, alcohols, flavoring agents, preservatives, stabilizers, coloring agents, and the like. In some embodiments of liquid oral compositions, the compounding agent is a compounding agent as disclosed herein below.

In some embodiments, the compositions disclosed herein can further contain components that are conventional in pharmaceutical preparations, e.g., diluents, carriers, pH modifiers, sweeteners, bulking agents, and further active agents. Such compositions form a further aspect of the present disclosure.

In preparing liquid oral compositions, such as, for example, suspensions, elixirs, and solutions, suitable compounding agents comprise one or more of water, glycols, glycerols, oils, cyclodextrins, alcohols, e.g., ethanol, flavoring agents, preservatives, coloring agents, and the like.

In some embodiments, the compounding agent is an aqueous compounding agent.

In some embodiments, the compounding agent is an aqueous compounding agent comprising microcrystalline cellulose, carboxymethylcellulose sodium, xanthan gum, carrageenan, or a combination thereof. In some embodiments, the aqueous compounding agent comprises microcrystalline cellulose. In some embodiments, the aqueous compounding agent comprises colloidal microcrystalline cellulose. In some embodiments, the aqueous compounding agent comprises carboxymethylcellulose sodium. In some embodiments, the aqueous compounding agent comprises xanthan gum. In some embodiments, the aqueous compounding agent comprises carrageenan. In some embodiments, the aqueous compounding agent comprises microcrystalline cellulose and carboxymethylcellulose sodium. In some embodiments, the aqueous compounding agent comprises microcrystalline cellulose and carrageenan. In some embodiments, the aqueous compounding agent comprises microcrystalline cellulose and xanthan gum. In some embodiments, the aqueous compounding agent comprises carboxymethylcellulose sodium and carrageenan. In some embodiments, the aqueous compounding agent comprises carboxymethylcellulose sodium and xanthan gum. In some embodiments, the aqueous compounding agent comprises xanthan gum and carrageenan. In some embodiments, the aqueous compounding agent comprises microcrystalline cellulose, carboxymethylcellulose sodium, and xanthan gum. In some embodiments, the aqueous compounding agent comprises microcrystalline cellulose, carboxymethylcellulose sodium, and carrageenan. In some embodiments, the aqueous compounding agent comprises microcrystalline cellulose, xanthan gum, and carrageenan. In some embodiments, the aqueous compounding agent comprises carboxymethylcellulose sodium, xanthan gum, and carrageenan. In some embodiments, the aqueous compounding agent comprises microcrystalline cellulose, carboxymethylcellulose sodium, xanthan gum, and carrageenan. In some embodiments, the aqueous compounding agent comprises colloidal microcrystalline cellulose, carboxymethylcellulose sodium, xanthan gum, and carrageenan.

In some embodiments, the compounding agent is an aqueous compounding agent comprising microcrystalline cellulose, xanthan gum, carrageenan, calcium sulfate, or a combination thereof. In some embodiments, the aqueous compounding agent comprises calcium sulfate. In some embodiments, the compounding agent comprises microcrystalline cellulose and calcium sulfate. In some embodiments, the compounding agent comprises xanthan gum and calcium sulfate. In some embodiments the compounding agent comprises carrageenan and calcium sulfate. In some embodiments, the compounding agent comprises microcrystalline cellulose, xanthan gum, and calcium sulfate. In some embodiments, the compounding agent comprises microcrystalline cellulose, carrageenan, and calcium sulfate. In some embodiments, the compounding agent comprises xanthan gum, carrageenan, and calcium sulfate. In some embodiments, the compounding agent comprises microcrystalline cellulose, xanthan gum, carrageenan, and calcium sulfate. In some embodiments, the compounding agent comprises colloidal microcrystalline cellulose, xanthan gum, carrageenan, and calcium sulfate.

The pharmaceutical composition comprising the compounding agent can further comprise at least one of citric acid, a citrate, a lactate, a phosphate, a maleate, a tartrate, a succinate, a sulfate, or an acetate. In some embodiments, the composition comprises at least one of lithium lactate, sodium lactate, potassium lactate, calcium lactate, lithium phosphate, trisodium phosphate, sodium phosphate, potassium phosphate, calcium phosphate, lithium maleate, sodium maleate, potassium maleate, calcium maleate, lithium tartarate, sodium tartarate, potassium tartarate, calcium tartarate, lithium succinate, sodium succinate, potassium succinate, calcium succinate, lithium acetate, sodium acetate, potassium acetate, or calcium acetate. The composition can comprise a citrate. The citrate can be at least one of lithium citrate monohydrate, sodium citrate monohydrate, potassium citrate monohydrate, calcium citrate monohydrate, lithium citrate dihydrate, sodium citrate dihydrate, potassium citrate dihydrate, calcium citrate dihydrate, lithium citrate trihydrate, sodium citrate trihydrate, potassium citrate trihydrate, calcium citrate trihydrate, lithium citrate tetrahydrate, sodium citrate tetrahydrate, potassium citrate tetrahydrate, calcium citrate tetrahydrate, lithium citrate pentahydrate, sodium citrate pentahydrate, potassium citrate pentahydrate, calcium citrate pentahydrate, lithium citrate hexahydrate, sodium citrate hexahydrate, potassium citrate hexahydrate, calcium citrate hexahydrate, lithium citrate heptahydrate, sodium citrate heptahydrate, potassium citrate heptahydrate, or calcium citrate heptahydrate. In some embodiments, the composition comprises at least one of sodium citrate monohydrate, potassium citrate monohydrate, calcium citrate monohydrate, sodium citrate dihydrate, potassium citrate dihydrate, calcium citrate dihydrate, sodium citrate trihydrate, potassium citrate trihydrate, calcium citrate trihydrate, sodium citrate tetrahydrate, potassium citrate tetrahydrate, calcium citrate tetrahydrate, sodium citrate pentahydrate, potassium citrate pentahydrate, calcium citrate pentahydrate, sodium citrate hexahydrate, potassium citrate hexahydrate, calcium citrate hexahydrate, sodium citrate heptahydrate, potassium citrate heptahydrate, or calcium citrate heptahydrate. In some embodiments, the composition includes sodium citrate dihydrate.

In some embodiments, the composition comprises citric acid.

In some embodiments, the composition comprises sulfate. In some embodiments, the sulfate can be lithium sulfate, sodium sulfate, potassium sulfate, magnesium sulfate, or calcium sulfate. In some embodiments, the sulfate can be calcium sulfate.

In some embodiments, the composition comprises trisodium phosphate, sodium phosphate, citric acid, calcium sulfate, or a combination thereof. In some embodiments, the composition comprises trisodium phosphate. In some embodiments, the composition comprises sodium phosphate. In some embodiments, the composition comprises citric acid. In some embodiments, the composition comprises calcium sulfate. In some embodiments, the composition comprises trisodium phosphate and sodium phosphate. In some embodiments, the composition comprises trisodium phosphate and citric acid. In some embodiments, the composition comprises sodium phosphate and citric acid. In some embodiments, the composition comprises calcium sulfate and sodium phosphate. In some embodiments, the composition comprises calcium sulfate and trisodium phosphate. In some embodiments, the composition comprises calcium sulfate and citric acid. In some embodiments, the composition comprises trisodium phosphate, sodium phosphate, and citric acid. In some embodiments, the composition comprises calcium sulfate, sodium phosphate, and citric acid. In some embodiments, the composition comprises calcium sulfate, trisodium phosphate, and citric acid. In some embodiments, the composition comprises calcium sulfate, trisodium phosphate, and sodium phosphate. In some embodiments, the composition comprises calcium sulfate, trisodium phosphate, sodium phosphate, and citric acid.

In some embodiments, the composition has a pH of about 3 to about 8. In some embodiments, the composition has a pH of about 4 to about 7. In some embodiments, the composition has a pH of about 5 to 6. In some embodiments, the composition has a pH of about 5.3. In some embodiments, the composition has a pH of about 5.4. In some embodiments, the composition has a pH of about 5.5. In some embodiments, the composition has a pH of about 5.6.

In some embodiments, the compounding agent and at least one of citric acid, a citrate, a lactate, a phosphate, a maleate, a tartrate, a succinate, a sulfate, or an acetate is Ora-Plus®.

In some embodiments of the pharmaceutical composition comprising Compound 1 and a compounding agent, Compound 1 is present in a concentration of about 5 mg/mL to about 40 mg/mL.

In some embodiments, the pharmaceutical composition comprising the compounding agent can further comprise a sweetener.

In some embodiments, the pharmaceutical composition comprising the compounding agent and the sweetener is an aqueous composition.

In some embodiments, provided herein is a pharmaceutical composition comprising Compound 1 and a sweetener.

In some embodiments, provided herein is a pharmaceutical composition comprising any one of the crystalline forms, solid forms, solvates, hydrates, or salts described herein, and a sweetener.

In some embodiments, provided herein is a pharmaceutical composition comprising Compound 1 and a sweetener, wherein at least some of Compound 1 is present as any one of the crystalline forms, solid forms, solvates, hydrates, or salts described herein.

In some embodiments, provided herein is a pharmaceutical composition prepared by a process comprising mixing a sweetener with the crystalline form of Compound 1, to form the pharmaceutical composition.

In some embodiments, the pharmaceutical composition comprising the sweetener is an aqueous pharmaceutical composition.

In some embodiments, the sweetener in a composition as disclosed herein comprises a sugar or a sugar substitute. In some embodiments, the sweetener comprises sucrose, saccharin, mannitol, sorbitol, dextrose, acesulfame, aspartame, fructose, maltitol, sucralose, or a combination thereof, wherein the sweetener or at least one sweetener in a combination of sweeteners is optionally in a salt form. In some embodiments, the sweetener comprises sucrose. In some embodiments, the sweetener comprises saccharin. In some embodiments, the sweetener comprises saccharin sodium. In some embodiments, the sweetener comprises saccharin sodium dihydrate. In some embodiments, the sweetener comprises saccharin calcium. In some embodiments, the sweetener comprises mannitol. In some embodiments, the sweetener comprises sorbitol. In some embodiments, the sweetener comprises dextrose. In some embodiments, the sweetener comprises anhydrous dextrose. In some embodiments, the sweetener comprises dextrose monohydrate. In some embodiments, the sweetener comprises acesulfame. In some embodiments, the sweetener comprises acesulfame potassium. In some embodiments, the sweetener comprises aspartame. In some embodiments, the sweetener comprises fructose. In some embodiments, the sweetener comprises maltitol. In some embodiments, the sweetener comprises sucralose.

In some embodiments, the sweetener is present in an amount of about 0.01 wt. % to about 1 wt. % in the pharmaceutical composition. In some embodiments, the sweetener is present in an amount of about 0.05 wt. % to about 0.75 wt. % in the pharmaceutical composition. In some embodiments, the sweetener is present in an amount of about 0.1 wt. % to about 0.5 wt. % in the pharmaceutical composition. In some embodiments, the sweetener is present in an amount of about 0.2 wt. % to about 0.4 wt. % in the pharmaceutical composition. In some embodiments, the sweetener is present in an amount of about 0.3 wt. % in the pharmaceutical composition.

In some embodiments, the sweetener is Ora-Sweet®.

In some embodiments, provided herein is a kit comprising
a) a pharmaceutical composition comprising Compound 1 and a compounding agent;
   and
b) a pharmaceutical composition comprising Compound 1 and a sweetener.

In some embodiments, provided herein is a kit comprising
a) a pharmaceutical composition comprising any one of the crystalline forms, solid forms, solvates, hydrates, or salts described herein, and a compounding agent;
   and
b) a pharmaceutical composition comprising any one of the crystalline forms, solid forms, solvates, hydrates, or salts described herein, and a sweetener.

In some embodiments, provided herein is a pharmaceutical composition comprising:
Compound 1;
a compounding agent comprising microcrystalline cellulose, carboxymethylcellulose sodium, xanthan gum, carrageenan, or a combination thereof;
at least one of citric acid, a citrate, a lactate, a phosphate, a maleate, a tartrate, a succinate, a sulfate, or an acetate;
and optionally a sweetener;
wherein the composition has a pH of about 3 to about 8.

In some embodiments, the pharmaceutical composition comprises:
Compound 1;
about 0.1 wt. % to about 2.0 wt. % of microcrystalline cellulose;
about 0.1 wt. % to about 1.0 wt. % of xanthan gum;
about 0.01 wt. % to about 1.0 wt. % of carrageenan;
and
about 0.01 wt. % to about 1.0 wt. % of CaSO₄.

In some embodiments, provided herein is a pharmaceutical composition comprising:
Form I of Compound 1;
a compounding agent comprising microcrystalline cellulose, carboxymethylcellulose sodium, xanthan gum, carrageenan, or a combination thereof;
at least one of citric acid, a citrate, a lactate, a phosphate, a maleate, a tartrate, a succinate, a sulfate, or an acetate;
and optionally a sweetener;
wherein the composition has a pH of about 3 to about 8.

In some embodiments, the pharmaceutical composition comprises:
Form I of Compound 1;
about 0.1 wt. % to about 2.0 wt. % of microcrystalline cellulose;
about 0.1 wt. % to about 1.0 wt. % of xanthan gum;
about 0.01 wt. % to about 1.0 wt. % of carrageenan;
and
about 0.01 wt. % to about 1.0 wt. % of CaSO₄.

### Compound 1, Forms Thereof, and Salts Thereof

In some embodiments, the compositions disclosed herein comprise Compound 1, or any one of the crystalline forms, solid forms, solvates, hydrates, or salts described herein of Compound 1, and a compounding agent as disclosed herein.

Compound 1 may be referred to herein as “Compound 1 free base”. In some embodiments, Compound 1 provided herein is a solid form. In some embodiments, the solid form is crystalline (e.g., Form I).

In some embodiments, the salt of the pharmaceutical composition of the present disclosure is a benzenesulfonic acid salt of Compound 1, which is referred to herein as “Compound 1 besylate”. In some embodiments, Compound 1 besylate has the following structure:

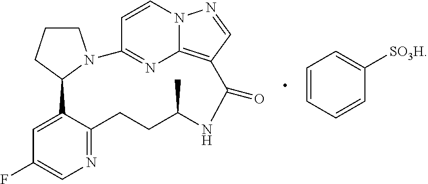 [SMILES: C[C@@H]1CCc2ncc(F)cc2[C@H]2CCCN2c2ccn3ncc(c3n2)C(=O)N1 ; O=S(=O)(O)c1ccccc1]

In some embodiments, the salt of the pharmaceutical composition of the present disclosure is a citric acid salt of Compound 1, which is referred to herein as “Compound 1 citrate”. In some embodiments, the Compound 1 citrate has the following structure:

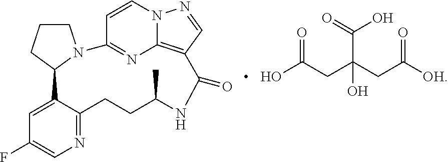 [SMILES: C[C@@H]1CCc2ncc(F)cc2[C@H]2CCCN2c2ccn3ncc(c3n2)C(=O)N1 ; O=C(O)CC(O)(CC(=O)O)C(=O)O]

In some embodiments, the salt of the pharmaceutical composition of the present disclosure is a methanesulfonic acid salt of Compound 1, which is referred to herein as “Compound 1 mesylate”. In some embodiments, the Compound 1 mesylate has the following structure:

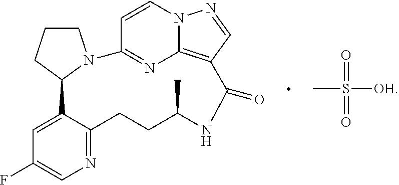

In some embodiments, the salt of the pharmaceutical composition of the present disclosure is a 1,2-ethane disulfonic acid salt of Compound 1, which is referred to herein as “Compound 1 edisylate”. In some embodiments, the Compound 1 edisylate has the following structure:

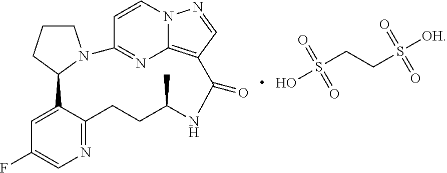 [SMILES: C[C@@H]1CCc2ncc(F)cc2[C@H]2CCCN2c2ccn3ncc(c3n2)C(=O)N1 ; O=S(=O)(O)CCS(=O)(=O)O]

In some embodiments, the salt of the pharmaceutical composition of the present disclosure is a p-toluene sulfonic acid salt of Compound 1, which is referred to herein as “Compound 1 tosylate”. In some embodiments, the Compound 1 tosylate has the following structure:

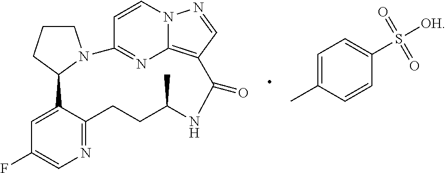 [SMILES: C[C@@H]1CCc2ncc(F)cc2[C@H]2CCCN2c2ccn3ncc(c3n2)C(=O)N1 ; Cc1ccc(S(=O)(=O)O)cc1]

In some embodiments, the salt of the pharmaceutical composition of the present disclosure is an oxalic acid salt of Compound 1, which is referred to herein as “Compound 1 oxalate”. In some embodiments, the Compound 1 oxalate has the following structure:

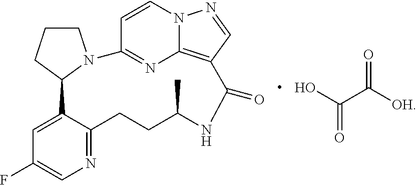 [SMILES: C[C@@H]1CCc2ncc(F)cc2[C@H]2CCCN2c2ccn3ncc(c3n2)C(=O)N1 ; O=C(O)C(=O)O]

In some embodiments, the salt of the pharmaceutical composition of the present disclosure is a fumaric acid salt of Compound 1, which is referred to herein as “Compound 1 fumarate”. In some embodiments, the Compound 1 fumarate has the following structure:

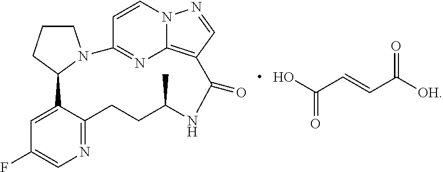 [SMILES: C[C@@H]1CCc2ncc(F)cc2[C@H]2CCCN2c2ccn3ncc(c3n2)C(=O)N1 ; O=C(O)/C=C/C(=O)O]

In some embodiments, the salt of the pharmaceutical composition of the present disclosure is a L-malic acid salt of Compound 1, which is referred to herein as “Compound 1 L-malate”. In some embodiments, the Compound 1 L-malate has the following structure:

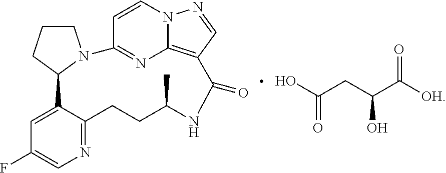 [SMILES: C[C@@H]1CCc2ncc(F)cc2[C@H]2CCCN2c2ccn3ncc(c3n2)C(=O)N1 ; O=C(O)C[C@H](O)C(=O)O]

In some embodiments, the salt of the pharmaceutical composition of the present disclosure is a succinic acid salt of Compound 1, which is referred to herein as “Compound 1 succinate”. In some embodiments, the Compound 1 succinate has the following structure:

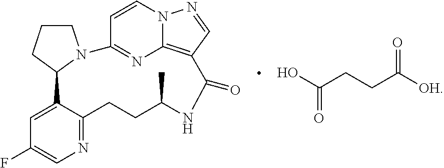 [SMILES: C[C@@H]1CCc2ncc(F)cc2[C@H]2CCCN2c2ccn3ncc(c3n2)C(=O)N1 ; O=C(O)CCC(=O)O]

In some embodiments, the salt of the pharmaceutical composition of the present disclosure is a hydrochloric acid salt of Compound 1, which is referred to herein as “Compound 1 hydrochloride”. In some embodiments, the Compound 1 hydrochloride has the following structure:

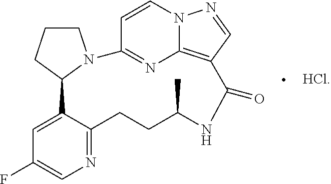 [SMILES: C[C@@H]1CCc2ncc(F)cc2[C@H]2CCCN2c2ccn3ncc(c3n2)C(=O)N1]

In some embodiments, the salt of the pharmaceutical composition of the present disclosure is a sulfuric acid salt of Compound 1, which is referred to herein as “Compound 1 sulfate”. In some embodiments, the Compound 1 sulfate has the following structure:

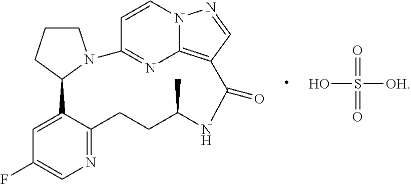 [SMILES: C[C@@H]1CCc2ncc(F)cc2[C@H]2CCCN2c2ccn3ncc(c3n2)C(=O)N1 ; O=S(=O)(O)O]

In some embodiments, the salt of the pharmaceutical composition of the present disclosure is a naphthalene-2-sulphonic acid salt of Compound 1, which is referred to herein as “Compound 1 2-naphthalenesulfonate”. In some embodiments, the Compound 1 2-naphthalenesulfonate has the following structure:

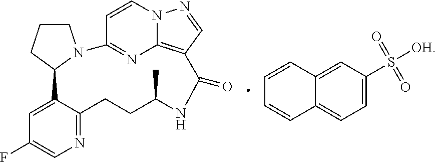

In some embodiments, the salt of the pharmaceutical composition of the present disclosure is a 2-hydroxy ethanesulfonic acid salt of Compound 1, which is referred to herein as “Compound 1 isethionate”. In some embodiments, the Compound 1 isethionate has the following structure:

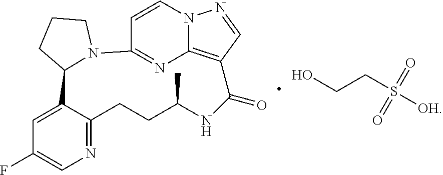 [SMILES: C[C@@H]1CCc2ncc(F)cc2[C@H]2CCCN2c2ccn3ncc(c3n2)C(=O)N1 ; O=S(=O)(O)CCO]

In some embodiments, the salt of the pharmaceutical composition of the present disclosure is a L-aspartic salt of Compound 1, which is referred to herein as “Compound 1 L-aspartate”. In some embodiments, the Compound 1 L-aspartate has the following structure:

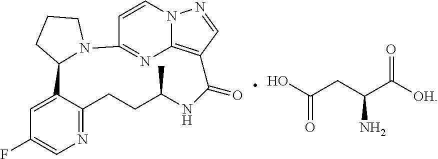 [SMILES: C[C@@H]1CCc2ncc(F)cc2[C@H]2CCCN2c2ccn3ncc(c3n2)C(=O)N1 ; N[C@@H](CC(=O)O)C(=O)O]

In some embodiments, the salt of the pharmaceutical composition of the present disclosure is a maleic acid salt of Compound 1, which is referred to herein as “Compound 1 maleate”. In some embodiments, the Compound 1 maleate has the following structure:

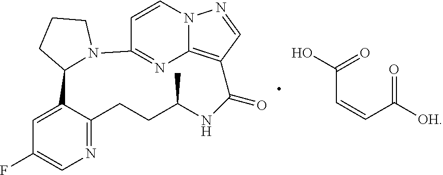 [SMILES: C[C@@H]1CCc2ncc(F)cc2[C@H]2CCCN2c2ccn3ncc(c3n2)C(=O)N1 ; O=C(O)/C=C\C(=O)O]

In some embodiments, the salt of the pharmaceutical composition of the present disclosure is a phosphoric acid salt of Compound 1, which is referred to herein as “Compound 1 phosphate”. In some embodiments, the Compound 1 phosphate has the following structure:

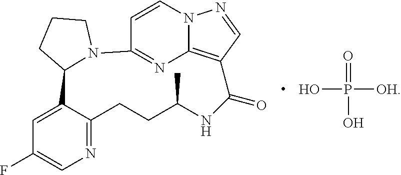 [SMILES: C[C@@H]1CCc2ncc(F)cc2[C@H]2CCCN2c2ccn3ncc(c3n2)C(=O)N1 ; O=P(O)(O)O]

In some embodiments, the salt of the pharmaceutical composition of the present disclosure is a ethanesulfonic acid salt of Compound 1, which is referred to herein as “Compound 1 esylate”. In some embodiments, the Compound 1 esylate has the following structure:

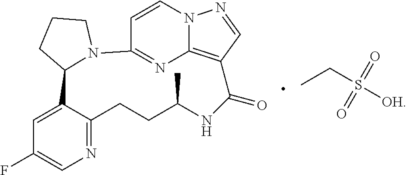

In some embodiments, the salt of the pharmaceutical composition of the present disclosure is a L-glutamic acid salt of Compound 1, which is referred to herein as “Compound 1 L-glutamate”. In some embodiments, the Compound 1 L-glutamate has the following structure:

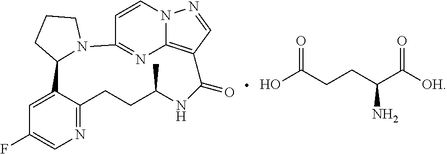 [SMILES: C[C@@H]1CCc2ncc(F)cc2[C@H]2CCCN2c2ccn3ncc(c3n2)C(=O)N1 ; N[C@@H](CCC(=O)O)C(=O)O]

In some embodiments, the salt of the pharmaceutical composition of the present disclosure is a L-tartaric acid salt of Compound 1, which is referred to herein as “Compound 1 L-tartrate”. In some embodiments, the Compound 1 L-tartrate has the following structure:

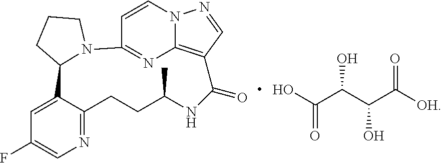 [SMILES: C[C@@H]1CCc2ncc(F)cc2[C@H]2CCCN2c2ccn3ncc(c3n2)C(=O)N1 ; O=C(O)[C@H](O)[C@@H](O)C(=O)O]

In some embodiments, the salt of the pharmaceutical composition of the present disclosure is a D-glucuronic acid salt of Compound 1, which is referred to herein as “Compound 1 D-glucuronate”. In some embodiments, the Compound 1 D-glucuronate has the following structure:

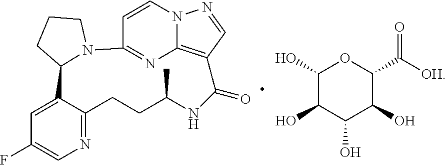 [SMILES: C[C@@H]1CCc2ncc(F)cc2[C@H]2CCCN2c2ccn3ncc(c3n2)C(=O)N1 ; O=C(O)[C@H]1O[C@@H](O)[C@H](O)[C@@H](O)[C@@H]1O]

In some embodiments, the salt of the pharmaceutical composition of the present disclosure is a hippuric acid salt of Compound 1, which is referred to herein as “Compound 1 hippurate”. In some embodiments, the Compound 1 hippurate has the following structure:

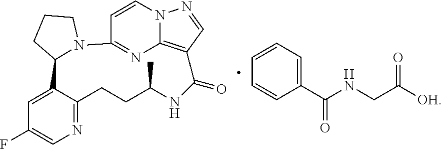

In some embodiments, the salt of the pharmaceutical composition of the present disclosure is a D-gluconic acid salt of Compound 1, which is referred to herein as “Compound 1 D-gluconate”. In some embodiments, the Compound 1 D-gluconate has the following structure:

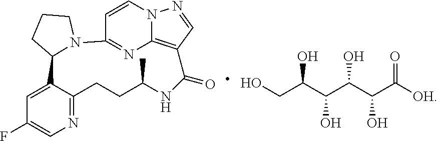 [SMILES: C[C@@H]1CCc2ncc(F)cc2[C@H]2CCCN2c2ccn3ncc(c3n2)C(=O)N1 ; O=C(O)[C@H](O)[C@@H](O)[C@H](O)[C@H](O)CO]

In some embodiments, the salt of the pharmaceutical composition of the present disclosure is a DL-lactic acid salt of Compound 1, which is referred to herein as “Compound 1 lactate”. In some embodiments, the Compound 1 lactate has the following structure:

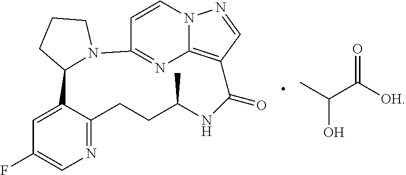

In some embodiments, the salt of the pharmaceutical composition of the present disclosure is a L-ascorbic acid salt of Compound 1, which is referred to herein as “Compound 1 L-ascorbate”. In some embodiments, the Compound 1 L-ascorbate has the following structure:

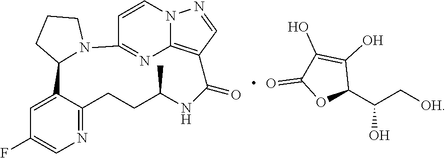

In some embodiments, the salt of the pharmaceutical composition of the present disclosure is a benzoic acid salt of Compound 1, which is referred to herein as “Compound 1 benzoate”. In some embodiments, the Compound 1 benzoate has the following structure:

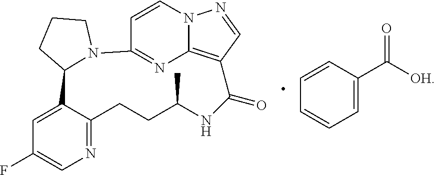

The salts of the pharmaceutical composition of the present application can be isolated as one or more solid forms. The solid forms, crystalline forms, solvated forms, hydrated forms of the Compound 1 and the salts of Compound 1 are described below, along with the methods of making the same and using the same for therapeutic purposes.

### Compound 1 Free Base

In some embodiments of the pharmaceutical compositions disclosed herein, a pharmaceutical composition comprises Compound 1 free base. In some embodiments, Compound 1 is at least 50%, at least 60%, at least 70%, at least 80%, at least 90%, or at least 99% crystalline.

In some embodiments, Form I is substantially free of other forms of Compound 1. In some embodiments, Form I contains less than 10%, such as less than 5%, such as less than 3%, such as less than 1% of other forms of Compound 1. In some embodiments, Form I is substantially free of the amorphous form of Compound 1. In some embodiments, Form I contains less than 10%, such as less than 5%, such as less than 3%, such as less than 1%, of the amorphous form of Compound 1.

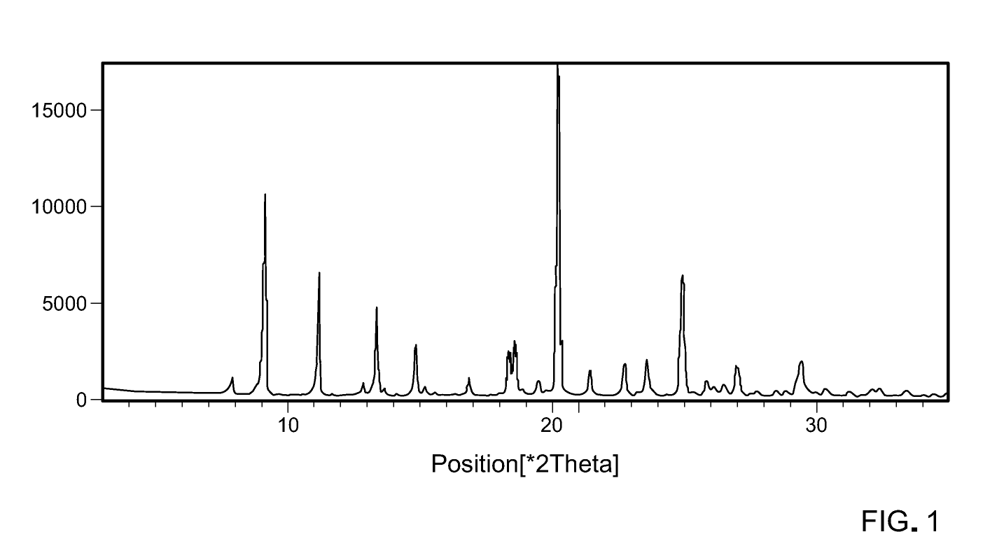

In some embodiments, Form I is substantially free of other stereoisomers of Compound 1. In some embodiments, Form I contains less than 10%, such as less than 5%, such as less than 3%, such as less than 1% of other stereoisomers of Compound 1. In some embodiments, Form I has an XRPD pattern substantially as depicted in FIG. 1. In some embodiments, Form I has a XRPD peak, in terms of 2-theta, at about 20.2 degrees. In some embodiments, Form I has XRPD peaks, in terms of 2-theta, at about 9.1, about 20.2 and about 24.9. In some embodiments, Form I has XRPD peaks, in terms of 2-theta, at about 9.1, about 11.2, about 20.2 and about 24.9. In some embodiments, Form I has XRPD peaks, in terms of 2-theta, at about 9.1, about 11.2, about 13.4, about 14.8, about 20.2, and about 29.4. In some embodiments, Form I has XRPD peaks, in terms of 2-theta, at about 9.1, about 11.2, about 13.4, about 14.8, about 18.3, about 18.6, about 20.2, about 23.6, about 24.9, and about 29.4.

In some embodiments, Form I has a XRPD peak, in terms of 2-theta, at 20.2±0.2 degrees. In some embodiments, Form I has XRPD peaks, in terms of 2-theta, at 9.1±0.2, 20.2±0.2 and 24.9±0.2 degrees. In some embodiments, Form I has XRPD peaks, in terms of 2-theta, at 9.1±0.2, 11.2±0.2, 20.2±0.2 and 24.9±0.2 degrees. In some embodiments, Form I has XRPD peaks, in terms of 2-theta, at 9.1±0.2, 11.2±0.2, 13.4±0.2, 14.8±0.2, 20.2±0.2, and 29.4±0.2 degrees. In some embodiments, Form I has XRPD peaks, in terms of 2-theta, at 9.1±0.2, 11.2±0.2, 13.4±0.2, 14.8±0.2, 18.3±0.2, 18.6±0.2, 20.2±0.2, 23.6±0.2, 24.9±0.2, and 29.4±0.2 degrees.

In some embodiments, Form I has at least one, at least two or at least three XRPD peaks, in terms of 2-theta, selected from about 9.1, about 11.2, about 13.4, about 20.2, and about 24.9 degrees. In some embodiments, Form I has at least one, at least two or at least three XRPD peaks, in terms of 2-theta, selected from about 9.1, about 11.2, about 13.4, about 14.8, about 16.8, about 18.3, about 18.6, about 20.2, about 21.4, about 22.7, about 23.6, about 24.9, and about 29.4. In some embodiments, Form I has at least one, at least two or at least three XRPD peaks, in terms of 2-theta, selected from about 9.1, about 11.2, about 13.4, about 14.8, about 18.3, about 18.6, about 20.2, about 23.6, about 24.9, and about 29.4. In some embodiments, Form I has at least one, at least two or at least three XRPD peaks, in terms of 2-theta, selected from about 9.1, about 11.2, about 13.4, about 14.8, about 20.2, and about 29.4.

In some embodiments, Form I has at least one, at least two or at least three XRPD peaks, in terms of 2-theta, selected from 9.1±0.2, 11.2±0.2, 13.4±0.2, 20.2±0.2, and 24.9±0.2 degrees. In some embodiments, Form I has at least one, at least two or at least three XRPD peaks, in terms of 2-theta, selected from 9.1±0.2, 11.2±0.2, 13.4±0.2, 14.8±0.2, 16.8±0.2, 18.3±0.2, 18.6±0.2, 20.2±0.2, 21.4±0.2, 22.7±0.2, 23.6±0.2, 24.9±0.2, and 29.4±0.2 degrees. In some embodiments, Form I has at least one, at least two or at least three XRPD peaks, in terms of 2-theta, selected from 9.1±0.2, 11.2±0.2, 13.4±0.2, 14.8±0.2, 18.3±0.2, 18.6±0.2, 20.2±0.2, 23.6±0.2, 24.9±0.2, and 29.4±0.2 degrees. In some embodiments, Form I has at least one, at least two or at least three XRPD peaks, in terms of 2-theta, selected from 9.1±0.2, 11.2±0.2, 13.4±0.2, 14.8±0.2, 20.2±0.2, and 29.4±0.2 degrees.

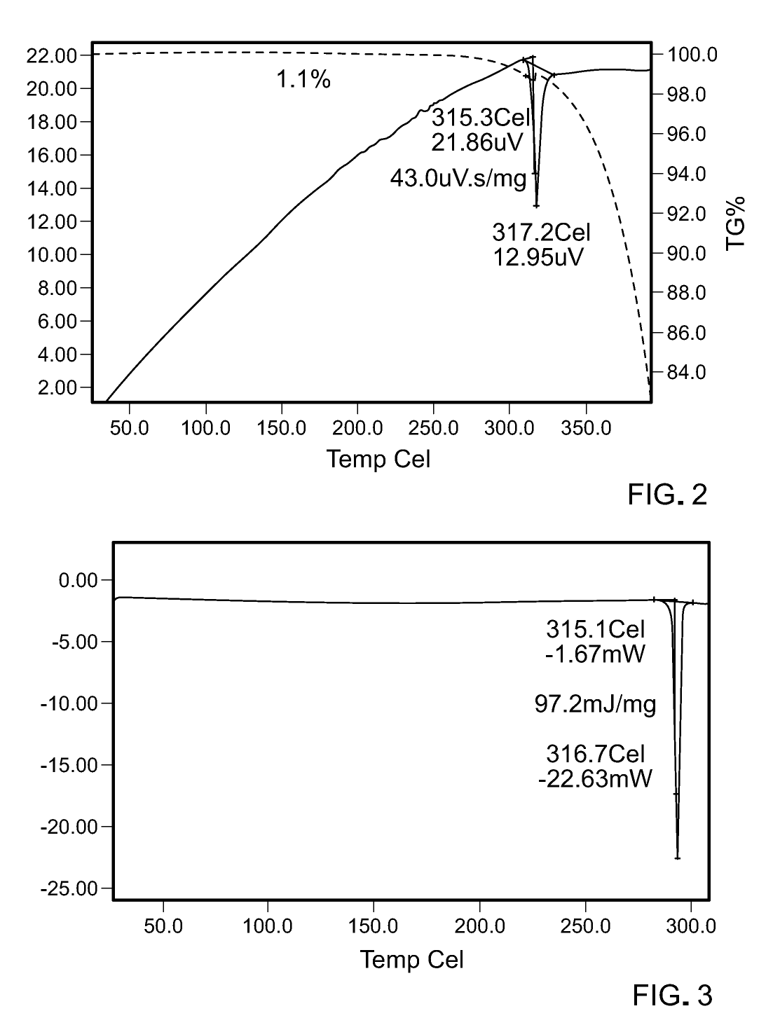

In some embodiments, Form I has a DTA thermogram substantially as depicted in FIG. 2. In some embodiments, Form I has a DTA thermogram characterized by an endothermal event at about 317° C. In some embodiments, Form I has a DSC thermogram substantially as depicted in FIG. 3. In some embodiments, Form I has a DSC thermogram characterized by an endothermal event at about 317° C. In some aspects of the aforementioned embodiments, the endothermal event is a melting point. In some embodiments, Form I has a DSC thermogram characterized by an endothermal event at about 124° C. (e.g., at the second heating cycle). In some aspects of these embodiments, the endothermal event at about 124° C. is a glass transition temperature.

Form I of Compound 1 is substantially anhydrous (Form I is not hydrated) and is substantially free of organic solvents (Form I is not solvated).

In some embodiments, Form I has hygroscopicity characterized by a mass uptake of about 0.3% at 90% RH as determined by GVS analysis. In other embodiments, Form I has hygroscopicity characterized by a mass uptake of about 0.7% at 90% RH as determined by DVS analysis. In some embodiments, Form I is substantially pure (e.g., the purity of the compound is at least about 90 wt. %, about 95 wt. %, about 98 wt. %, or about 99 wt. %). Purity values indicate the percentage of the amount of sample that is Form I. Purity values can be determined, for example, by HPLC/UV methods. In some embodiments, Form I is substantially free of impurities, such as organic impurities (e.g., process intermediates), inorganic impurities, and/or residual solvents.

In some embodiments, the crystalline form of Compound 1 exhibits the following single crystal X-ray crystallographic parameters at 120K:

| Crystal system | orthorhombic |
|---|---|
| Space group | P2₁2₁2₁ |
| a/Å | 6.91792(3) |
| b/Å | 13.74742(3) |
| c/Å | 19.22580(5) |
| α/° | 90.00 |
| β/° | 90.00 |
| γ/° | 90.00 |
| Volume/Å³ | 1828.442(10) |
| Z, Z′ | 4 |
| ρcalc g/cm³ | 1.382 |

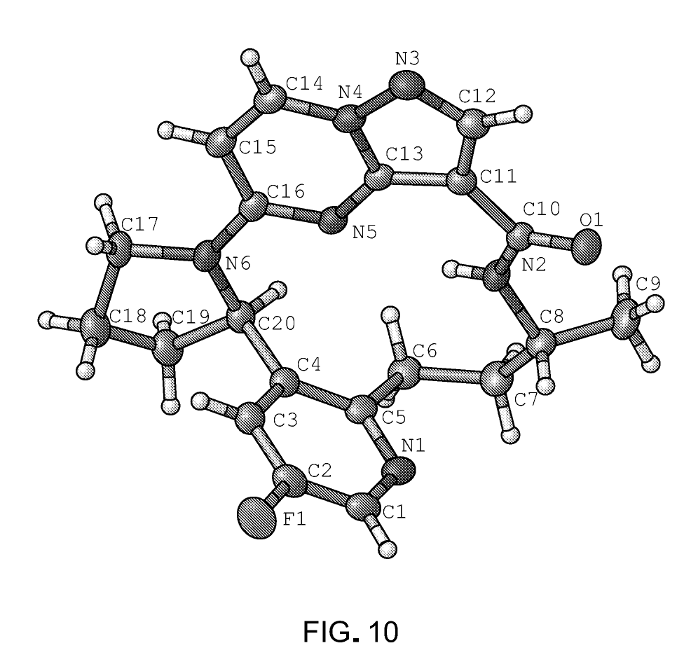

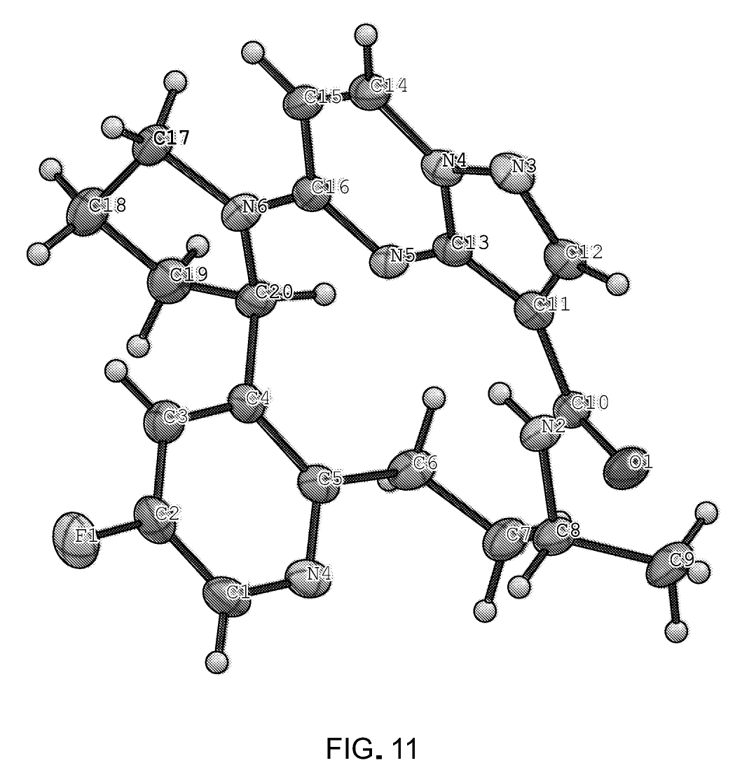

In some embodiments, the crystalline form of Compound 1 is substantially as shown in FIGS. 10 and 11.

In some embodiments, Compound 1 forms a solvate with acetonitrile solvent. In some embodiments, the acetonitrile solvate of Compound 1 is crystalline. In some embodiments, the crystalline form of acetonitrile solvate of Compound 1 exhibits the following single crystal X-ray crystallographic parameters at 120K:

| Crystal system | orthorhombic |
|---|---|
| Space group | P2₁2₁2₁ |
| a/Å | 6.03307(4) |
| b/Å | 16.10794(9) |
| c/Å | 23.72624(13) |
| α/° | 90.00 |
| β/° | 90.00 |
| γ/° | 90.00 |
| Volume/Å³ | 2305.73(2) |
| Z, Z′ | 4 |
| ρcalc g/cm³ | 1.332 |

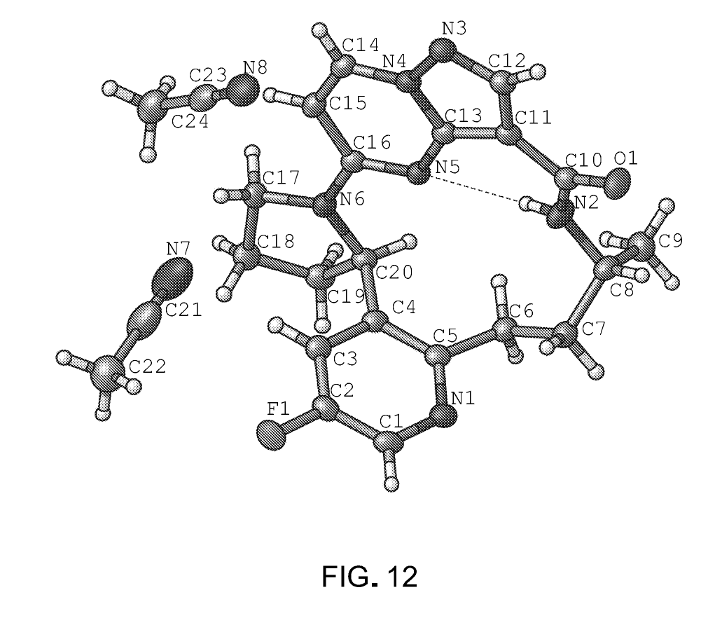

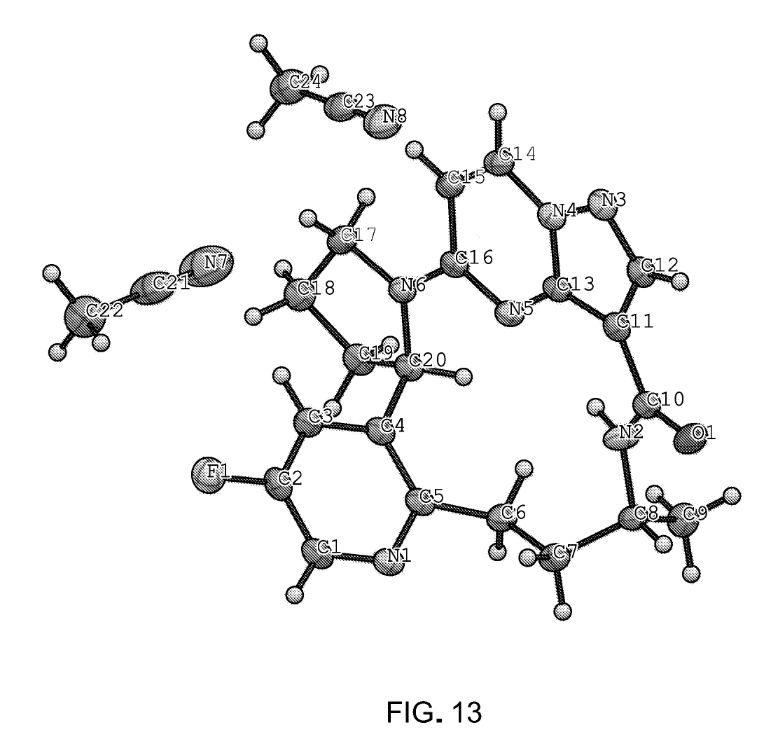

In some embodiments, the crystalline form of acetonitrile solvate is substantially as shown in FIGS. 12 and 13. In some embodiments, the crystalline form of acetonitrile solvate readily desolvates at room temperature to yield the crystalline Form I of Compound 1.

In some embodiments, the present disclosure provides crystalline Form I of Compound 1 prepared as disclosed herein. In one example, the disclosure provides the Form I of Compound 1 prepared by precipitating the solid crystalline form of Compound 1 from a saturated solution of Compound 1 in 1-propanol at about 2° C.

### Compound 1 Benzenesulfonic Acid Salt

In some embodiments the pharmaceutical compositions disclosed herein comprises Compound 1 besylate. In some embodiments, Compound 1 besylate is at least 50%, at least 60%, at least 70%, at least 80%, at least 90%, or at least 99% crystalline solid. In some embodiments, crystalline Compound 1 besylate is substantially free of other forms of Compound 1 besylate. In some embodiments, crystalline Compound 1 besylate contains less than 10%, such as less than 5%, such as less than 3% of other forms of Compound 1 besylate. In some embodiments, the crystalline Compound 1 besylate is substantially free of the amorphous form of Compound 1 besylate. In some embodiments, the crystalline Compound 1 besylate contains less than 10%, less than 5%, or less than 3% of the amorphous form of Compound 1 besylate.

In some embodiments, the molar ratio of Compound 1 to the benzenesulfonic acid in the besylate is about 1:1. In some embodiments, Compound 1 besylate is a monobesylate.

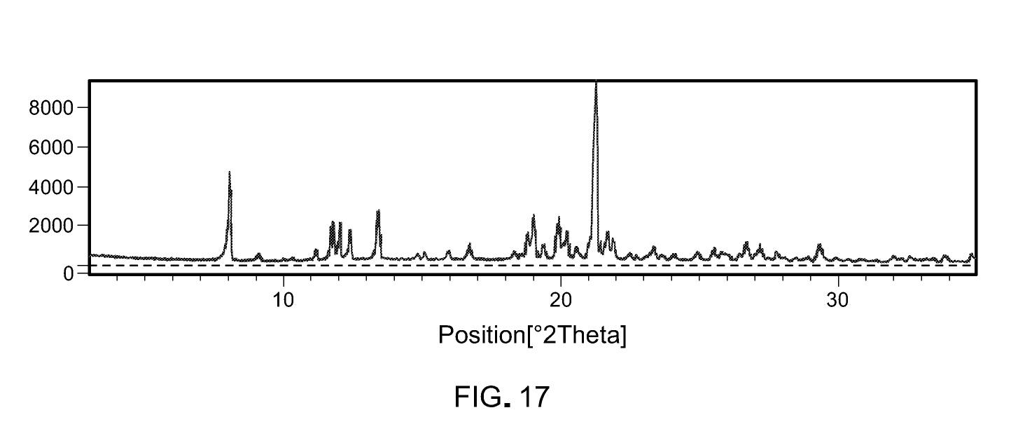

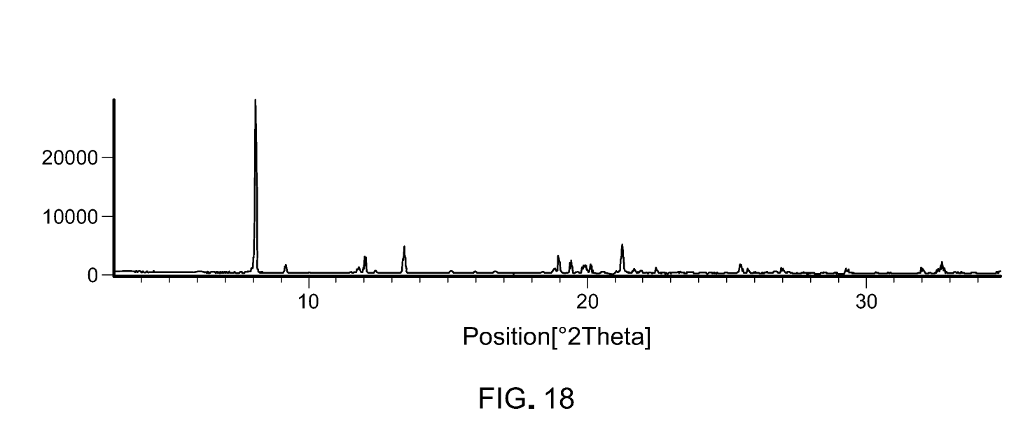

In some embodiments, the crystalline Compound 1 besylate has an XRPD pattern substantially as depicted in FIG. 17. In other embodiments, the crystalline Compound 1 besylate has an XRPD pattern substantially as depicted in FIG. 18.

In some embodiments, the crystalline Compound 1 besylate has a XRPD peak, in terms of 2-theta, at about 8.1 degrees. In some embodiments, the crystalline Compound 1 besylate has XRPD peaks, in terms of 2-theta, at about 8.1, about 13.4, and about 21.2. In some embodiments, the crystalline Compound 1 besylate has XRPD peaks, in terms of 2-theta, at about 8.1, about 12.0, about 13.4, about 19.0, about 19.4, and about 21.2. In some embodiments, the crystalline Compound 1 besylate has XRPD peaks, in terms of 2-theta, at about 8.1, about 12.0, about 13.4, about 19.0, about 19.4, about 19.9, about 20.1, about 21.2, about 25.5, and about 32.7.

In some embodiments, the crystalline Compound 1 besylate has a XRPD peak, in terms of 2-theta, at 8.1±0.2 degrees. In some embodiments, the crystalline Compound 1 besylate has XRPD peaks, in terms of 2-theta, at 8.1±0.2, 13.4±0.2, and 21.2±0.2. In some embodiments, the crystalline Compound 1 besylate has XRPD peaks, in terms of 2-theta, at 8.1±0.2, 12.0±0.2, 13.4±0.2, 19.0±0.2, 19.4±0.2, and 21.2±0.2 degrees. In some embodiments, the crystalline Compound 1 besylate has XRPD peaks, in terms of 2-theta, at 8.1±0.2, 12.0±0.2, 13.4±0.2, 19.0±0.2, 19.4±0.2, 19.9±0.2, 20.1±0.2, 21.2±0.2, 25.5±0.2, and 32.7±0.2 degrees.

In some embodiments, the crystalline Compound 1 besylate has a XRPD peak, in terms of 2-theta, at about 8.1, about 13.4, or about 21.2. In some embodiments, the crystalline Compound 1 besylate has at least one, at least two, or at least three XRPD peaks, in terms of 2-theta, selected from about 8.1, about 9.2, about 12.0, about 13.4, about 19.0, about 19.4, about 19.9, about 20.1, about 21.2, about 25.5, about 27.0, about 32.0, and about 32.7. In some embodiments, the crystalline Compound 1 besylate has at least one, at least two, or at least three XRPD peaks, in terms of 2-theta, selected from about 8.1, about 12.0, about 13.4, about 19.0, about 19.4, and about 21.2. In some embodiments, the crystalline Compound 1 besylate has at least one, at least two, or at least three XRPD peaks, in terms of 2-theta, selected from about 8.1, about 12.0, about 13.4, about 19.0, about 19.4, about 19.9, about 20.1, about 21.2, about 25.5, and about 32.7.

In some embodiments, the crystalline Compound 1 besylate has a XRPD peak, in terms of 2-theta, at 8.1±0.2, 13.4±0.2, or 21.2±0.2. In some embodiments, the crystalline Compound 1 besylate has at least one, at least two, or at least three XRPD peaks, in terms of 2-theta, selected from 8.1±0.2, 9.2±0.2, 12.0±0.2, 13.4±0.2, 19.0±0.2, 19.4±0.2, 19.9±0.2, 20.1±0.2, 21.2±0.2, 25.5±0.2, 27.0±0.2, 32.0±0.2, and 32.7±0.2 degrees. In some embodiments, the crystalline Compound 1 besylate has at least one, at least two, or at least three XRPD peaks, in terms of 2-theta, selected from 8.1±0.2, 12.0±0.2, 13.4±0.2, 19.0±0.2, 19.4±0.2, and 21.2±0.2 degrees. In some embodiments, the crystalline Compound 1 besylate has at least one, at least two, or at least three XRPD peaks, in terms of 2-theta, selected from 8.1±0.2, 12.0±0.2, 13.4±0.2, 19.0±0.2, 19.4±0.2, 19.9±0.2, 20.1±0.2, 21.2±0.2, 25.5±0.2, and 32.7±0.2 degrees.

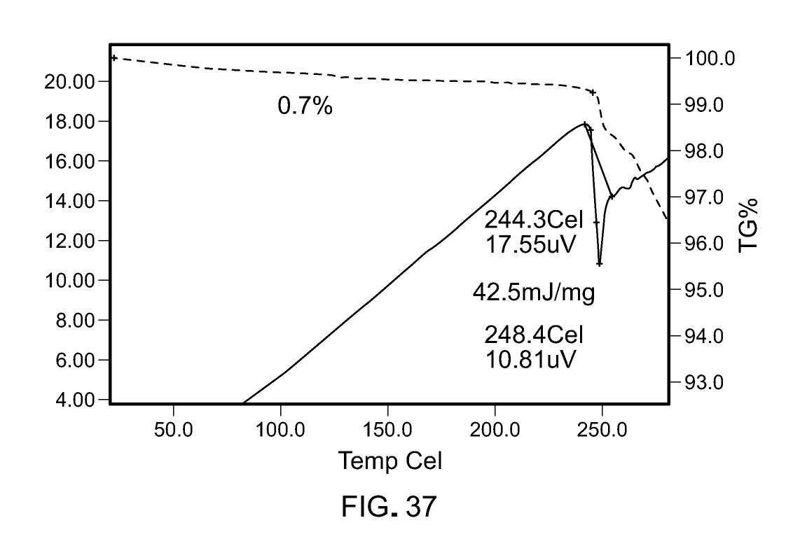

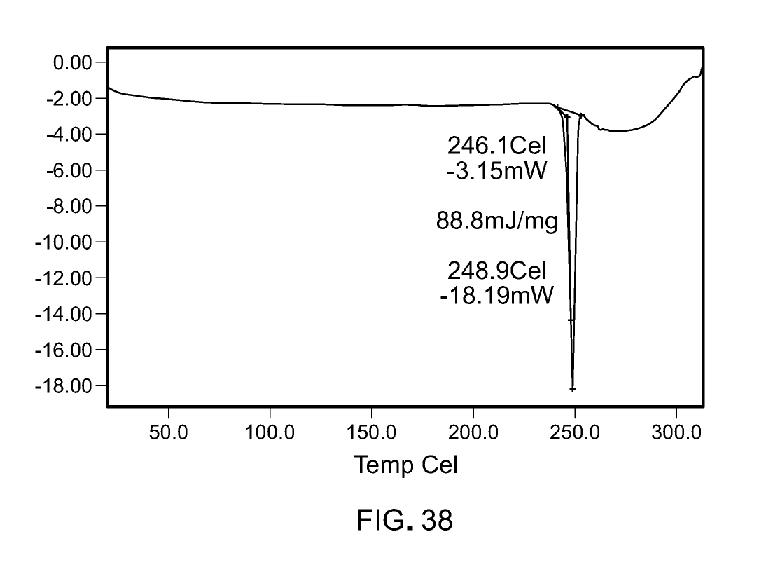

In some embodiments, the crystalline Compound 1 besylate has a DTA thermogram substantially as depicted in FIG. 37. In some embodiments, the crystalline Compound 1 besylate has a DTA thermogram characterized by an endothermal event at about 248° C. In some aspects of these embodiments, the endothermal event is a melting point. In some embodiments, the crystalline Compound 1 besylate has a DSC thermogram substantially as depicted in FIG. 38. In some embodiments, the crystalline Compound 1 besylate has a DSC thermogram characterized by an endothermal event at about 249° C.

In some embodiments, the crystalline Compound 1 besylate has hygroscopicity characterized by a mass uptake of about 0.7% at 90% RH as determined by DVS analysis. The crystalline Compound 1 besylate is substantially anhydrous (the crystalline form of the besylate is not hydrated) and is substantially free of organic solvents (the crystalline form of the besylate is not solvated).

In some embodiments, the crystalline Compound 1 besylate is substantially pure (e.g., free of organic, inorganic or other impurities). In some embodiments, the purity of the crystalline Compound 1 besylate is 90 wt. % or more, 95 wt. % or more, or 99 wt. % or more. In some embodiments, the crystalline Compound 1 besylate is substantially free of other crystalline forms of Compound 1 besylate.

In some embodiments, the benzenesulfonic acid salt of Compound 1 may form a hydrate. In some aspects of these embodiments, the hydrate is crystalline.

In some embodiments, the present disclosure provides a crystalline Compound 1 besylate prepared as disclosed herein. In one example, the application provides the crystalline Compound 1 besylate prepared by precipitating the solid crystalline form of Compound 1 besylate from a mixture of Compound 1 besylate with THF (e.g., a solution of Compound 1 besylate in THF). In another example, the application provides the crystalline Compound 1 besylate prepared by precipitating the crystalline form of Compound 1 besylate from a mixture of Compound 1 besylate with ethanol (e.g., a solution of Compound 1 besylate in ethanol).

### Compound 1 Citric Acid Salt

In some embodiments the pharmaceutical compositions disclosed herein comprises Compound 1 citrate. In some embodiments, Compound 1 citrate is at least 50%, at least 60%, at least 70%, at least 80%, at least 90%, or at least 99% crystalline solid. In some embodiments, crystalline Compound 1 citrate is substantially free of other forms of Compound 1 citrate. In some embodiments, crystalline Compound 1 citrate contains less than 10%, such as less than 5%, such as less than 3% of other forms of Compound 1 citrate. In some embodiments, the crystalline form of Compound 1 citrate is substantially free of the amorphous form of Compound 1 citrate. In some embodiments, the crystalline form of Compound 1 citrate contains less than 10%, less than 5%, or less than 3% of the amorphous form of compound 1 citrate.

In some embodiments, the molar ratio of Compound 1 to the citric acid in the citrate is about 1:1. In some embodiments, Compound 1 citrate is a monocitrate.

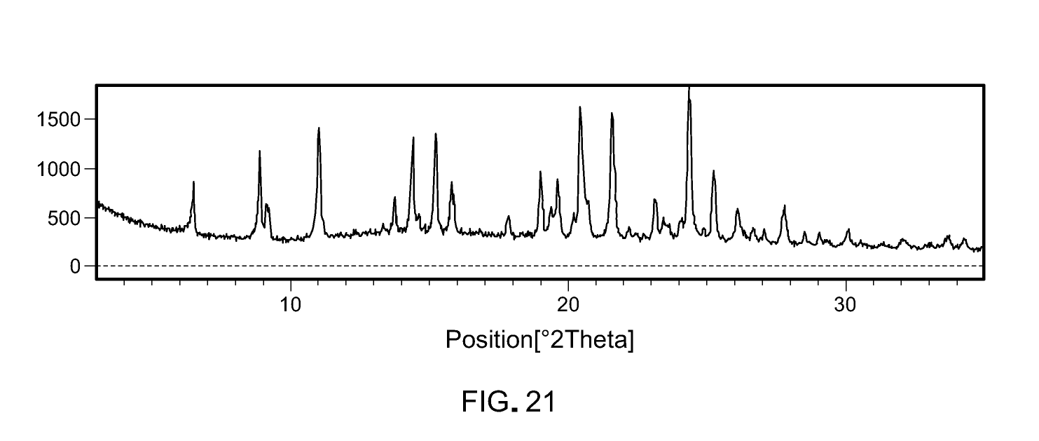

In some embodiments, crystalline Compound 1 citrate has Form A, which is described below in the Examples. In some embodiments, the Compound 1 citrate Form A has an XRPD pattern substantially as depicted in FIG. 21.

In some embodiments, the Compound 1 citrate Form A has a XRPD peak, in terms of 2-theta, at about 20.7 degrees. In some embodiments, Compound 1 citrate Form A has XRPD peaks, in terms of 2-theta, at about 20.7, about 21.6, and about 24.8. In some embodiments, Compound 1 citrate Form A has XRPD peaks, in terms of 2-theta, at about 8.9, about 11.1, about 14.4, about 15.4, about 20.7, about 21.6, and about 24.8. In some embodiments, Compound 1 citrate Form A has XRPD peaks, in terms of 2-theta, at about 8.9, about 11.1, about 13.9, about 14.4, about 15.4, about 19.2, about 20.7, about 21.6, about 24.8, and about 25.6.

In some embodiments, the Compound 1 citrate Form A has a XRPD peak, in terms of 2-theta, at 20.7±0.2 degrees. In some embodiments, Compound 1 citrate Form A has XRPD peaks, in terms of 2-theta, at 20.7±0.2, 21.6±0.2, and 24.8±0.2 degrees. In some embodiments, Compound 1 citrate Form A has XRPD peaks, in terms of 2-theta, at 8.9±0.2, 11.1±0.2, 14.4±0.2, 15.4±0.2, 20.7±0.2, 21.6±0.2, and 24.8±0.2 degrees. In some embodiments, Compound 1 citrate Form A has XRPD peaks, in terms of 2-theta, at 8.9±0.2, 11.1±0.2, 13.9±0.2, 14.4±0.2, 15.4±0.2, 19.2±0.2, 20.7±0.2, 21.6±0.2, 24.8±0.2, and 25.6±0.2 degrees.

In some embodiments, Compound 1 citrate Form A has at least one, at least two, or at least three XRPD peaks, in terms of 2-theta, selected from about 6.5, about 8.9, about 9.2, about 11.1, about 13.9, about 14.4, about 15.4, about 15.9, about 18.0, about 19.2, about 19.6, about 20.7, about 21.6, about 22.7, about 23.3, about 23.7, about 24.2, about 24.8, about 25.6, about 26.3, about 26.5, about 26.8, about 27.9, about 28.9, about 29.1, about 30.2, about 32.5, and about 33.7. In some embodiments, Compound 1 citrate Form A has at least one, at least two, or at least three XRPD peaks, in terms of 2-theta, selected from about 6.5, about 8.9, about 9.2, about 11.1, about 13.9, about 14.4, about 15.4, about 15.9, about 18.0, about 19.2, about 19.6, about 20.7, about 21.6, about 23.3, about 23.7, about 24.2, about 24.8, about 25.6, about 26.5, and about 27.9. In some embodiments, Compound 1 citrate Form A has at least one, at least two, or at least three XRPD peaks, in terms of 2-theta, selected from about 8.9, about 11.1, about 14.4, about 15.4, about 19.2, about 20.7, about 21.6, about 24.8, and about 25.6.

In some embodiments, Compound 1 citrate Form A has at least one, at least two, or at least three XRPD peaks, in terms of 2-theta, selected from about 6.5, about 8.9, about 9.2, about 11.1, about 13.9, about 14.4, about 15.4, about 15.9, about 18.0, about 19.2, about 19.6, about 20.7, about 21.6, about 22.3, about 22.7, about 23.3, about 23.7, about 24.2, about 24.8, about 25.6, about 26.3, about 26.5, about 26.8, about 27.9, about 28.9, about 29.1, about 30.2, about 30.6, about 31.8, about 32.5, about 33.1, about 33.7, about 34.3, and about 34.5.

In some embodiments, Compound 1 citrate Form A has at least one, at least two, or at least three XRPD peaks, in terms of 2-theta, selected from 6.5±0.2, 8.9±0.2, 9.2±0.2, 11.1±0.2, 13.9±0.2, 14.4±0.2, 15.4±0.2, 15.9±0.2, 18.0±0.2, 19.2±0.2, 19.6±0.2, 20.7±0.2, 21.6±0.2, 22.7±0.2, 23.3±0.2, 23.7±0.2, 24.2±0.2, 24.8±0.2, 25.6±0.2, 26.3±0.2, 26.5±0.2, 26.8±0.2, 27.9±0.2, 28.9±0.2, 29.1±0.2, 30.2±0.2, 32.5±0.2, and 33.7±0.2 degrees. In some embodiments, Compound 1 citrate Form A has at least one, at least two, or at least three XRPD peaks, in terms of 2-theta, selected from 6.5±0.2, 8.9±0.2, 9.2±0.2, 11.1±0.2, 13.9±0.2, 14.4±0.2, 15.4±0.2, 15.9±0.2, 18.0±0.2, 19.2±0.2, 19.6±0.2, 20.7±0.2, 21.6±0.2, 23.3±0.2, 23.7±0.2, 24.2±0.2, 24.8±0.2, 25.6±0.2, 26.5±0.2, and 27.9±0.2 degrees. In some embodiments, Compound 1 citrate Form A has at least one, at least two, or at least three XRPD peaks, in terms of 2-theta, selected from 8.9±0.2, 11.1±0.2, 14.4±0.2, 15.4±0.2, 19.2±0.2, 20.7±0.2, 21.6±0.2, 24.8±0.2, and 25.6±0.2 degrees.

In some embodiments, Compound 1 citrate Form A has at least one, at least two, or at least three XRPD peaks, in terms of 2-theta, selected from 6.5±0.2, 8.9±0.2, 9.2±0.2, 11.1±0.2, 13.9±0.2, 14.4±0.2, 15.4±0.2, 15.9±0.2, 18.0±0.2, 19.2±0.2, 19.6±0.2, 20.7±0.2, 21.6±0.2, 22.3±0.2, 22.7±0.2, 23.3±0.2, 23.7±0.2, 24.2±0.2, 24.8±0.2, 25.6±0.2, 26.3±0.2, 26.5±0.2, 26.8±0.2, 27.9±0.2, 28.9±0.2, 29.1±0.2, 30.2±0.2, 30.6±0.2, 31.8±0.2, 32.5±0.2, 33.1±0.2, 33.7±0.2, 34.3±0.2, and 34.5±0.2 degrees.

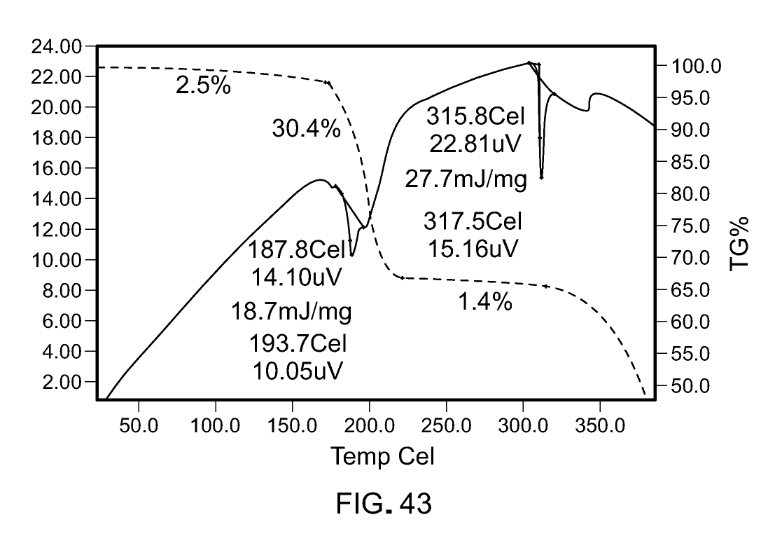

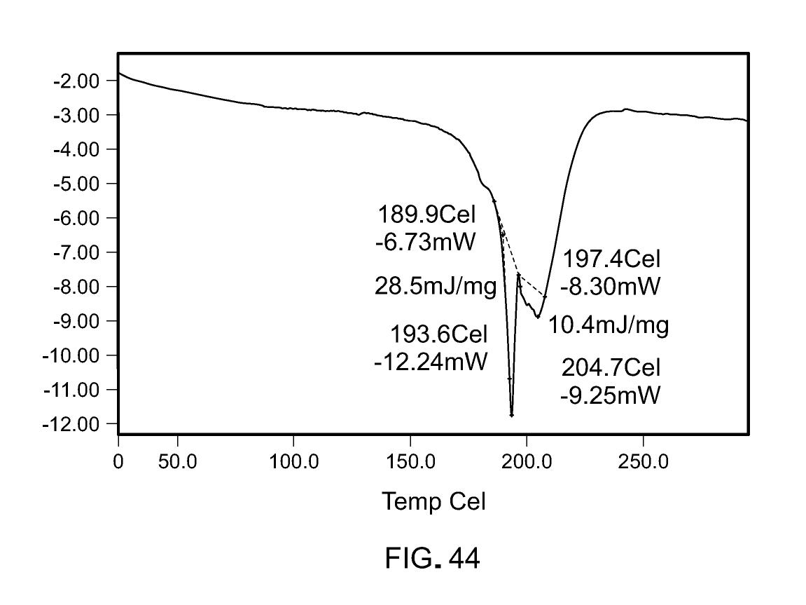

In some embodiments, Compound 1 citrate Form A has a DTA thermogram substantially as depicted in FIG. 43. In some embodiments, Compound 1 citrate Form A has a DTA thermogram characterized by an endothermal event at about 194° C. In some embodiments, Compound 1 citrate Form A has a DTA thermogram characterized by an endothermal event at about 318° C. In some embodiments, Compound 1 citrate Form A has a DTA thermogram characterized by an endothermal event at about 194° C. and an endothermal event at about 318° C. In some embodiments, Compound 1 citrate Form A has a DSC thermogram substantially as depicted in FIG. 44. In some embodiments, Compound 1 citrate Form A has a DSC thermogram characterized by an endothermal event at about 205° C. In some embodiments, Compound 1 citrate Form Ahas a DSC thermogram characterized by an endothermal event at about 194° C. and an endothermal event at about 205° C. In some aspects of these embodiments, the endothermal events are overlapping.

In some embodiments, Compound 1 citrate Form A has hygroscopicity characterized by a mass uptake of around 1.8% at 90% RH as determined by DVS analysis. Compound 1 citrate Form A is substantially anhydrous (Form A is not hydrated) and is substantially free of organic solvents (Form A is not solvated).

In some embodiments, Compound 1 citrate Form A is substantially pure (e.g., free of organic, inorganic or other impurities). In some embodiments, the purity of Compound 1 citrate Form A is 90 wt. % or more, 95 wt. % or more, or 99 wt. % or more. In some embodiments, Compound 1 citrate Form A is substantially free of other crystalline forms of Compound 1 citrate. For example, Compound 1 citrate Form A is substantially free of Compound 1 citrate Form B.

In some embodiments, the citric acid salt of Compound 1 may form a hydrate. In some aspects of these embodiments, the hydrate is crystalline.

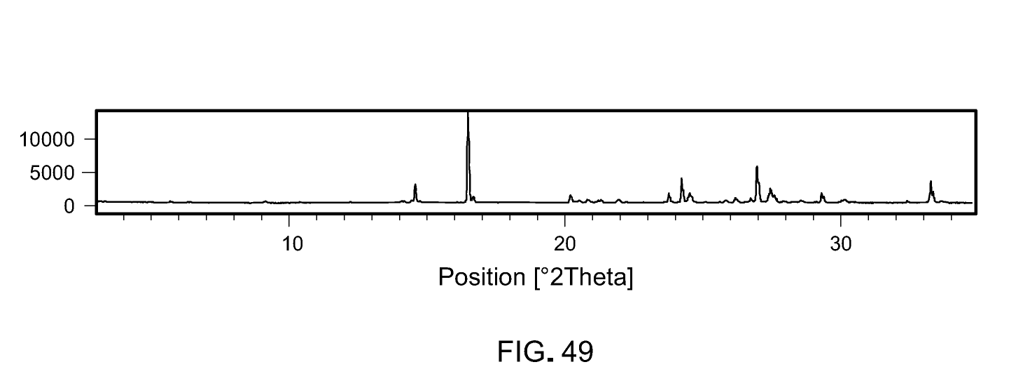

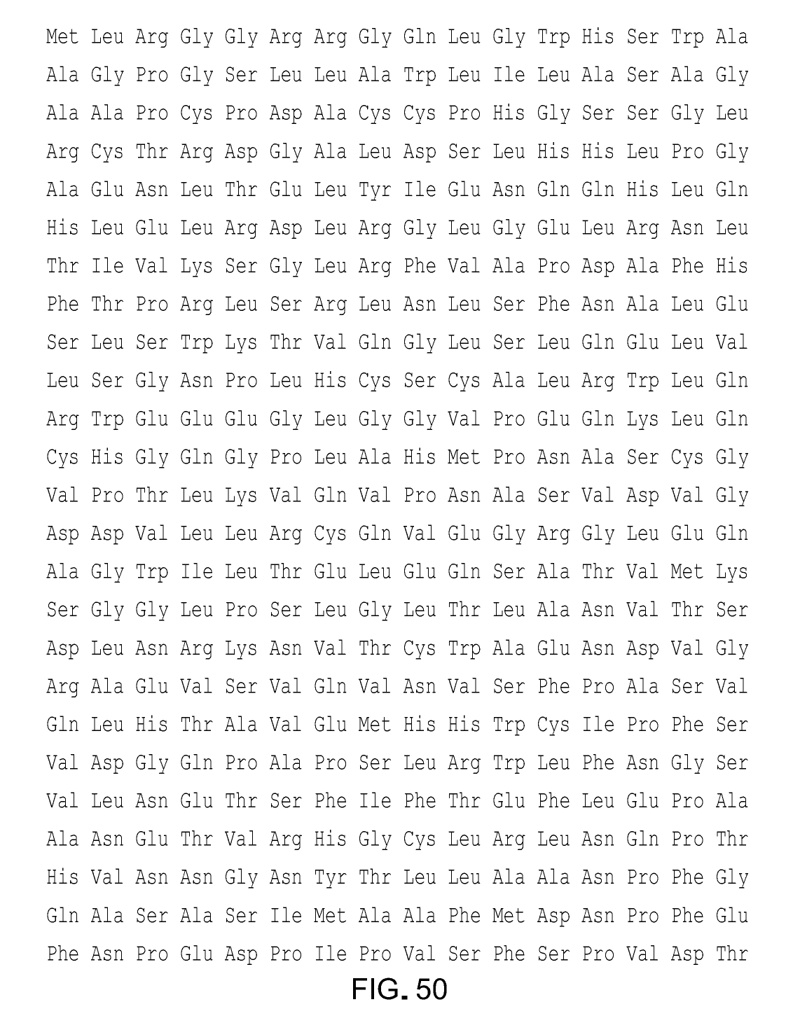

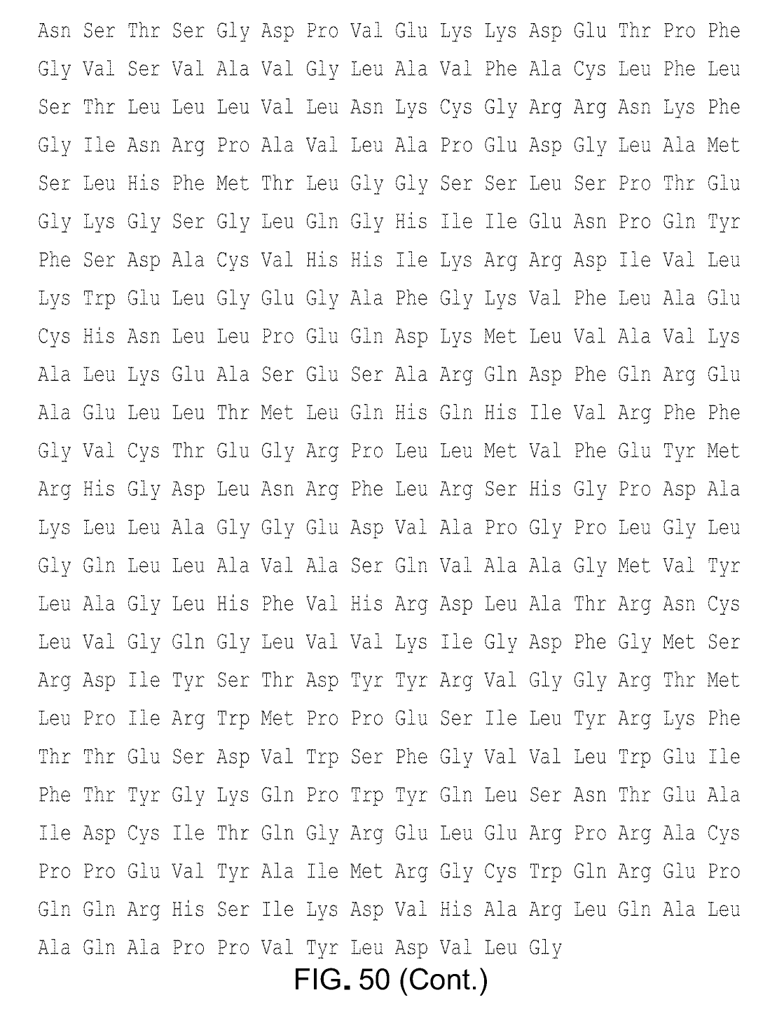

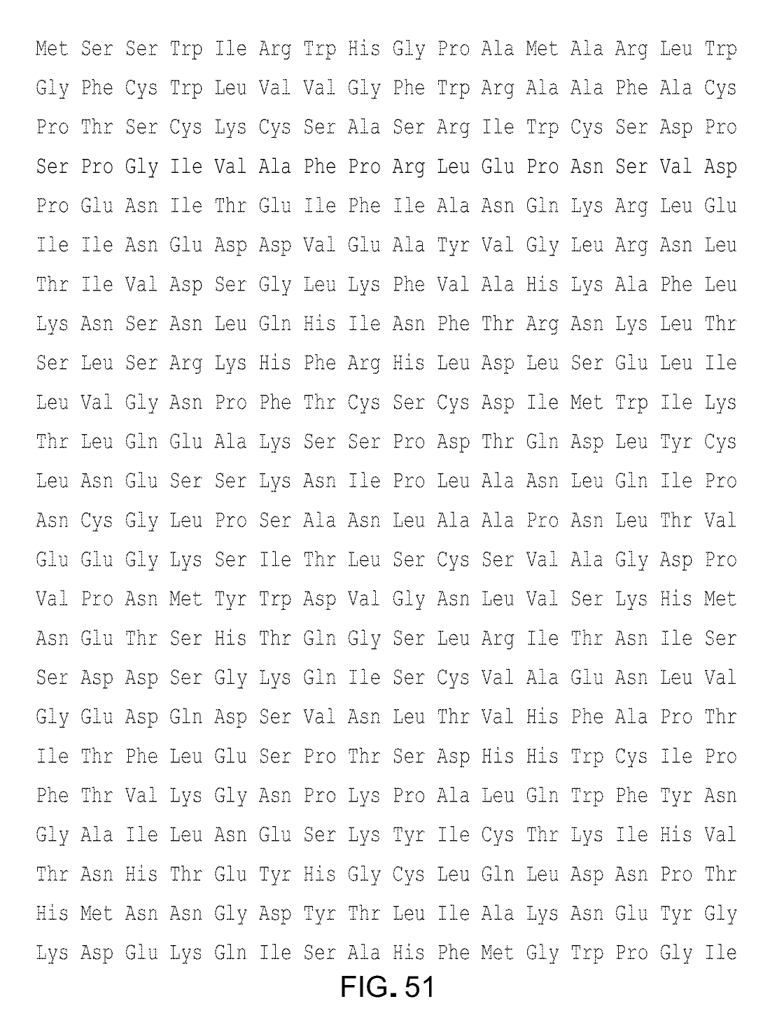

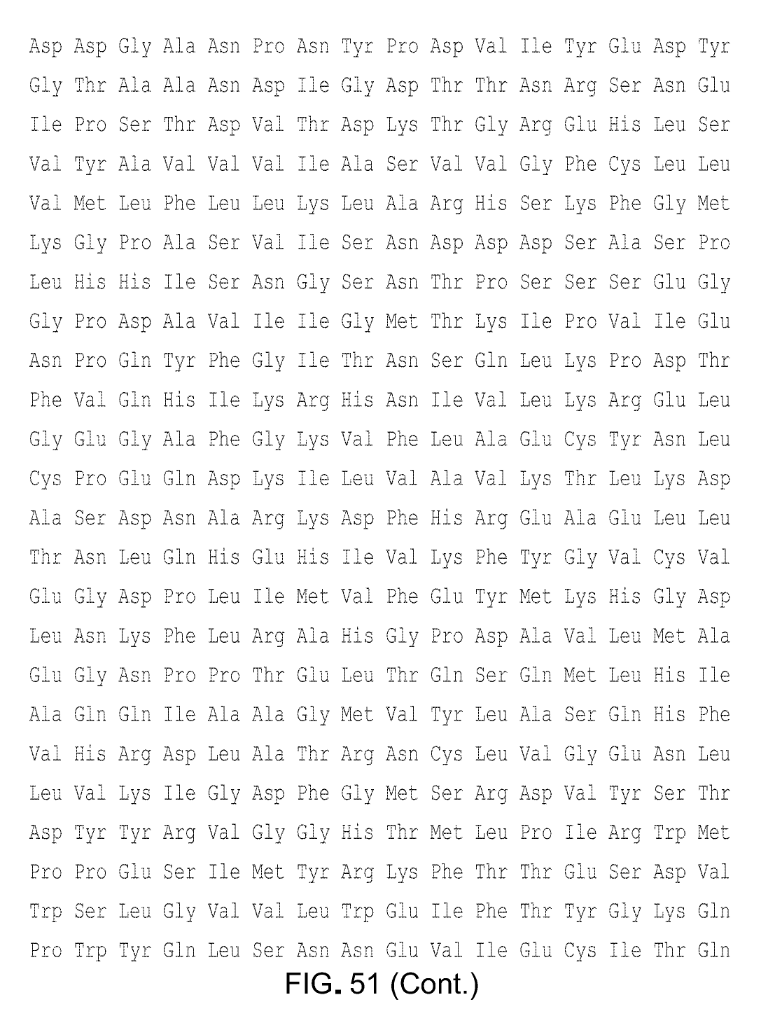

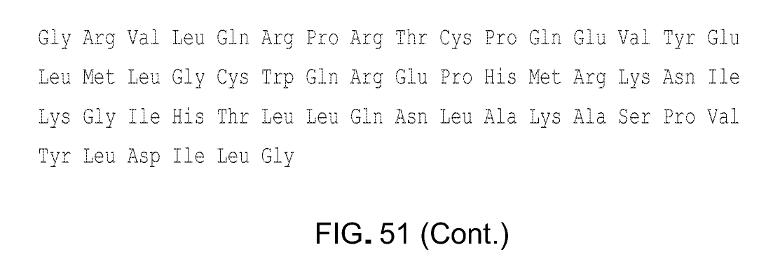

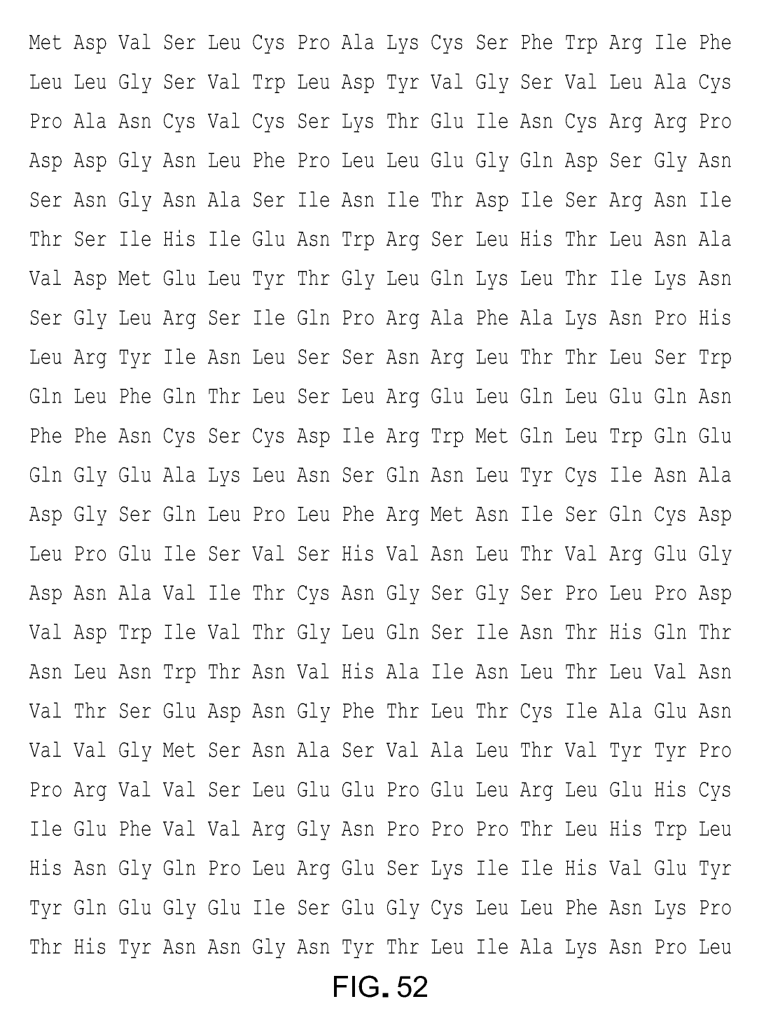

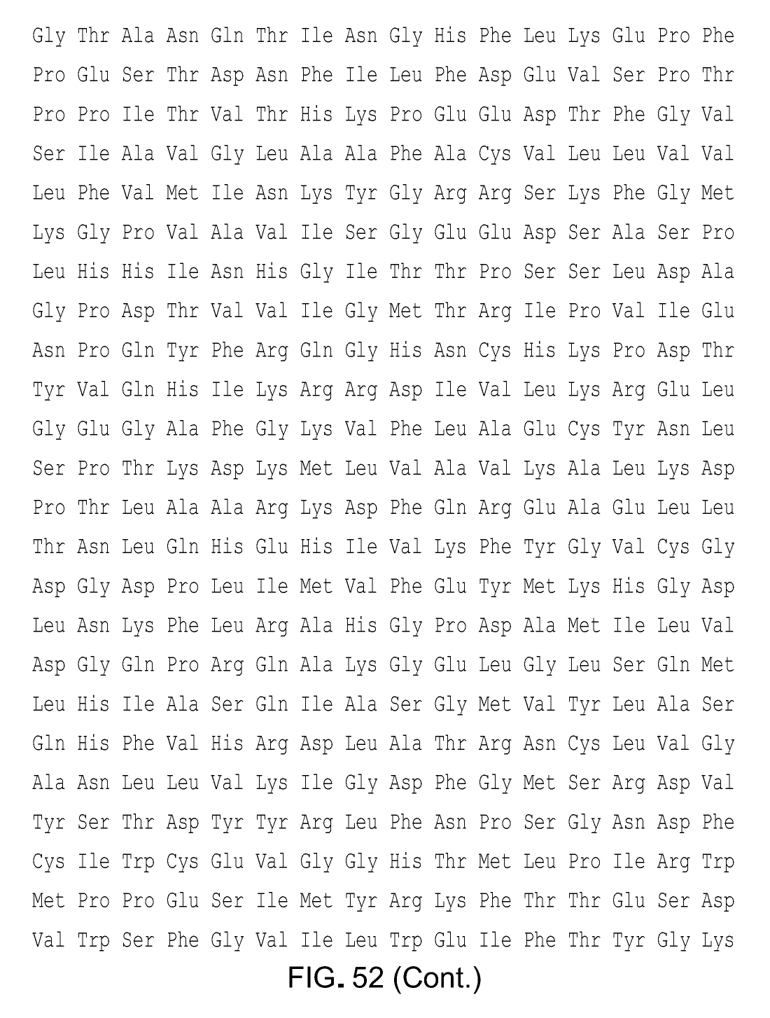

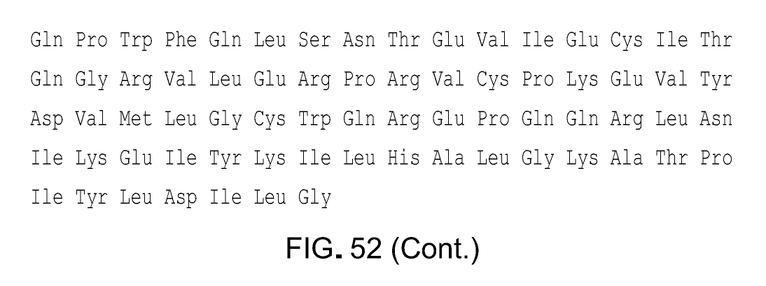

In some embodiments, the crystalline Compound 1 citrate has Form B, which has an XRPD pattern substantially as depicted in FIG. 49.

In some embodiments, the present disclosure provides a crystalline form of Compound 1 citrate prepared as disclosed herein. In one example, the present application provides Compound 1 citrate Form A prepared by precipitating Form A from a mixture of Compound 1 citrate with acetone (e.g., a solution of Compound 1 in acetone).

### Compound 1 Methanesulfonic Acid Salt

In some embodiments the pharmaceutical compositions disclosed herein comprises Compound 1 mesylate. In some embodiments, the Compound 1 mesylate is at least 50%, at least 60%, at least 70%, at least 80%, at least 90%, or at least 99% crystalline solid. In some embodiments, the crystalline form of Compound 1 mesylate is substantially free of the amorphous form of Compound 1 mesylate. In some embodiments, the crystalline form of Compound 1 mesylate contains less than 10%, less than 5%, or less than 3% of the amorphous form of compound 1 mesylate.

In some embodiments, the molar ratio of the Compound 1 to the methanesulfonic acid in the mesylate is about 1:1. In some embodiments, the Compound 1 mesylate is a monomesylate.

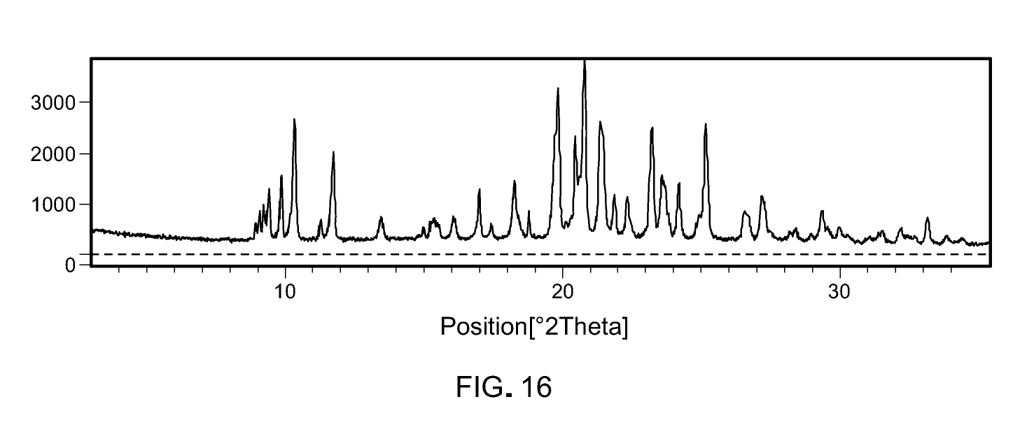

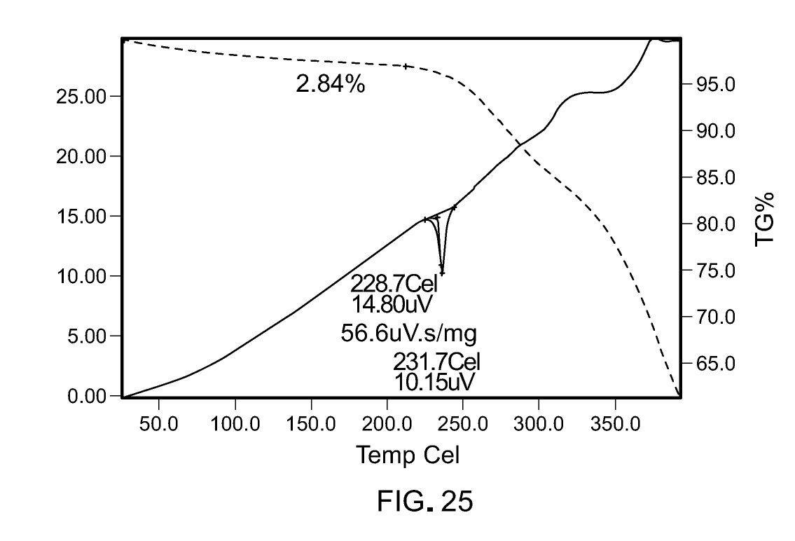

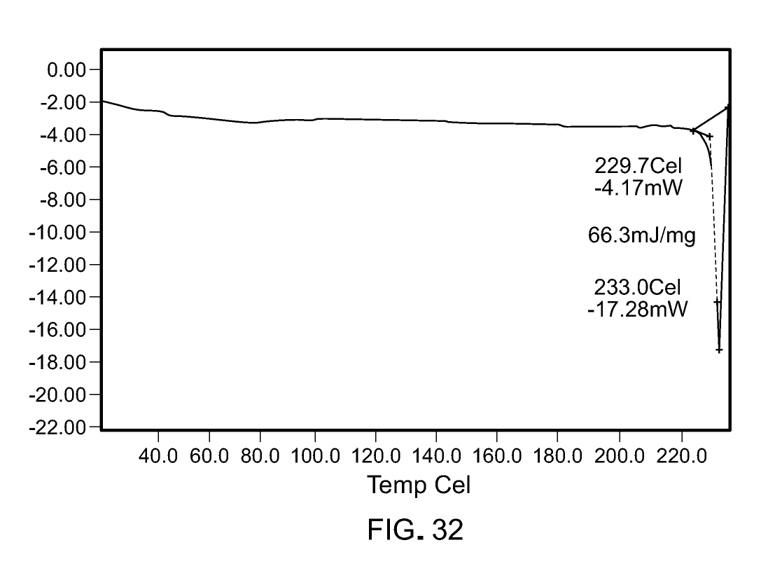

In some embodiments, the crystalline form of Compound 1 mesylate has an XRPD pattern substantially as depicted in FIG. 16. In some embodiments, the crystalline solid of the Compound 1 mesylate has a DTA thermogram substantially as depicted in FIG. 25. In some embodiments, the crystalline solid of the Compound 1 mesylate has a DTA thermogram characterized by an endothermal event at about 232° C. (e.g., a melting point of the mesylate). In some embodiments, the crystalline Compound 1 mesylate has a DSC thermogram substantially as depicted in FIG. 32. In some embodiments, the crystalline Compound 1 mesylate has a DSC thermogram characterized by an endothermal event at about 233° C. The crystalline form of the mesylate is substantially anhydrous (the crystalline form is not hydrated) and is substantially free of organic solvents (the crystalline form is not solvated). In some embodiments, the crystalline form of the mesylate is substantially pure (e.g., purity is 90 wt. % or more, 95 wt. % or more, or 99 wt. % or more). In some embodiments, the crystalline form of Compound 1 mesylate is substantially free of other crystalline forms of Compound 1 mesylate.

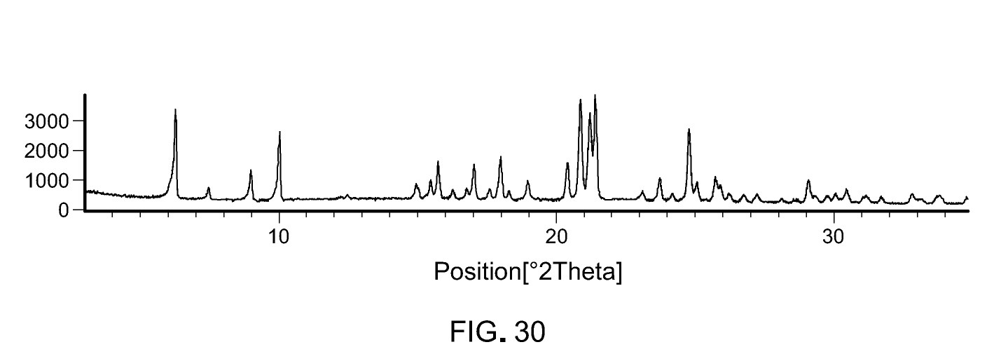

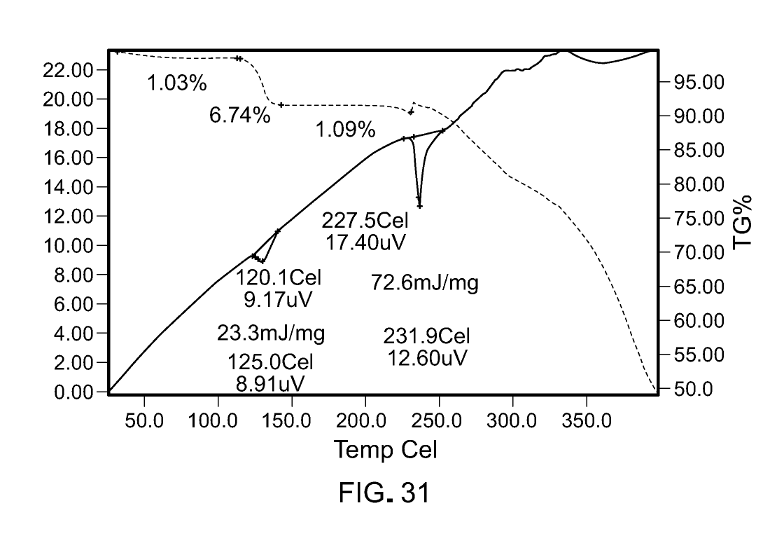

Compound 1 mesylate can be prepared as an acetone solvate. In some embodiments, the acetone solvate of the mesylate is a solid form (e.g., an amorphous solid, a crystalline solid, or a mixture thereof). In some embodiments, the acetone solvate of the mesylate is crystalline. In some embodiments, the crystalline form of the acetone solvate of the mesylate salt of Compound 1 is has an XRPD pattern substantially as depicted in FIG. 30. In some embodiments, the crystalline acetone solvate has a DTA thermogram substantially as depicted in FIG. 31. In some embodiments, the crystalline acetone solvate has a DTA thermogram characterized by an endothermal event at about 125° C. and an endothermal event at about 232° C. (melting point). The endothermal event at about 125° C. is likely associated with the desolvation of the material. In some embodiments, the crystalline acetone solvate has a DSC thermogram characterized by an endothermal event at about 233° C. at the first heating cycle, a solidification event at about 181° C. at the first cooling cycle, and an endothermal event at about 229° C. at the second heating cycle. In some embodiments, the acetone solvate readily desolvates upon heating to produce crystalline form of the Compound 1 mesylate.

In some embodiments, the present disclosure provides a crystalline form of Compound 1 mesylate prepared as disclosed herein. In one example, the application provides the crystalline form of Compound 1 mesylate prepared by precipitating the solid crystalline form of Compound 1 mesylate from a mixture of Compound 1 mesylate in 2-propanol (e.g., a solution of Compound 1 in isopropanol).

### Other Salts

In some embodiments the pharmaceutical compositions disclosed herein comprises Compound 1 edisylate, Compound 1 tosylate, Compound 1 oxalate, Compound 1 fumarate, Compound 1 L-malate or Compound 1 succinate. In some embodiments, each of Compound 1 edisylate, Compound 1 tosylate, Compound 1 oxalate, Compound 1 fumarate, Compound 1 L-malate and or Compound 1 succinate can be prepared as a solid form, e.g., as an amorphous solid, as a crystalline solid, or as a mixture thereof. In some aspects of these embodiments, any of the aforementioned salts of Compound 1 is at least 50%, at least 60%, at least 70%, at least 80%, at least 90%, or at least 99% crystalline solid. In other aspects of these embodiments, the crystalline salt of Compound 1 is substantially free of the amorphous form of the salt. For example, Compound 1 salt contains less than 10%, less than 5%, or less than 3% of the amorphous form of the salt.

In some embodiments, the present disclosure provides a pharmaceutical composition comprising a crystalline form of Compound 1 edisylate, Compound 1 tosylate, Compound 1 oxalate, Compound 1 fumarate, Compound 1 L-malate or Compound 1 succinate prepared as disclosed herein.

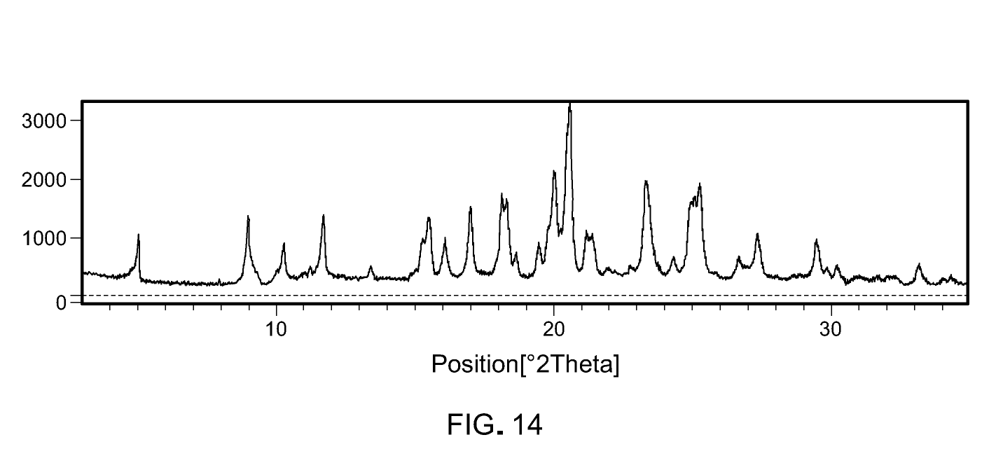

In some embodiments, the crystalline Compound 1 edisylate has an XRPD pattern substantially as depicted in FIG. 14.

In some embodiments, the crystalline Compound 1 edisylate has XRPD peaks, in terms of 2-theta, at about 20.0, about 20.6, and about 23.3. In some embodiments, the crystalline Compound 1 edisylate has XRPD peaks, in terms of 2-theta, at about 18.1, about 18.3, about 20.0, about 20.6, about 23.3, and about 25.3. In some embodiments, the crystalline Compound 1 edisylate has XRPD peaks, in terms of 2-theta, at about 11.6, about 15.5, about 17.0, about 18.1, about 18.3, about 20.0, about 20.6, about 23.3, about 24.9, and about 25.3.

In some embodiments, the crystalline Compound 1 edisylate has XRPD peaks, in terms of 2-theta, at 20.0±0.2, 20.6±0.2, and 23.3±0.2 degrees. In some embodiments, the crystalline Compound 1 edisylate has XRPD peaks, in terms of 2-theta, at 18.1±0.2, 18.3±0.2, 20.0±0.2, 20.6±0.2, 23.3±0.2, and 25.3±0.2 degrees. In some embodiments, the crystalline Compound 1 edisylate has XRPD peaks, in terms of 2-theta, at 11.6±0.2, 15.5±0.2, 17.0±0.2, 18.1±0.2, 18.3±0.2, 20.0±0.2, 20.6±0.2, 23.3±0.2, 24.9±0.2, and 25.3±0.2 degrees.

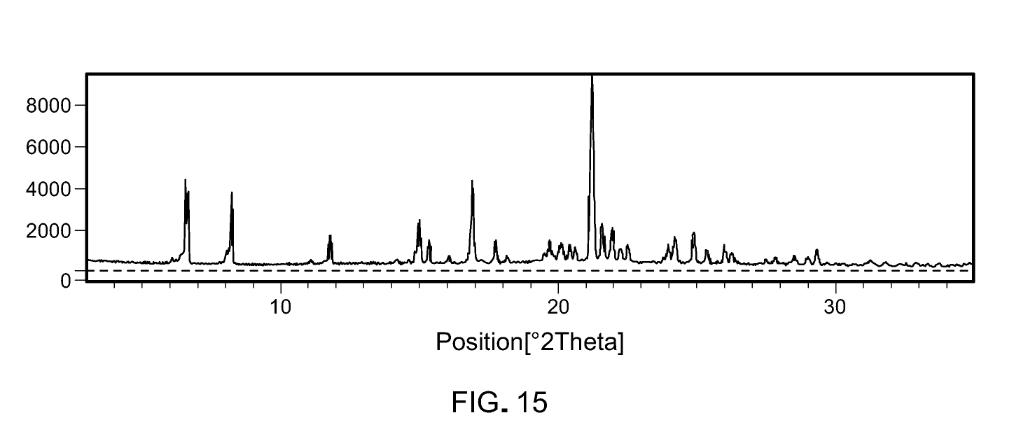

In some embodiments, the crystalline Compound 1 tosylate has an XRPD pattern substantially as depicted in FIG. 15.

In some embodiments, the crystalline Compound 1 tosylate has XRPD peaks, in terms of 2-theta, at about 6.6, about 16.9, and about 21.2. In some embodiments, the crystalline Compound 1 tosylate has XRPD peaks, in terms of 2-theta, at about 6.6, about 8.2, about 15.0, about 16.9, about 21.2, and about 21.6. In some embodiments, the crystalline Compound 1 tosylate has XRPD peaks, in terms of 2-theta, at about 6.6, about 8.2, about 11.8, about 15.0, about 16.9, about 21.2, about 21.6, about 21.9, about 24.2 and about 24.9.

In some embodiments, the crystalline Compound 1 tosylate has XRPD peaks, in terms of 2-theta, at 6.6±0.2, 16.9±0.2, and 21.2±0.2 degrees. In some embodiments, the crystalline Compound 1 tosylate has XRPD peaks, in terms of 2-theta, at 6.6±0.2, 8.2±0.2, 15.0±0.2, 16.9±0.2, 21.2±0.2, and 21.6±0.2 degrees. In some embodiments, the crystalline Compound 1 tosylate has XRPD peaks, in terms of 2-theta, at 6.6±0.2, 8.2±0.2, 11.8±0.2, 15.0±0.2, 16.9±0.2, 21.2±0.2, 21.6±0.2, 21.9±0.2, 24.2±0.2, and 24.9±0.2 degrees.

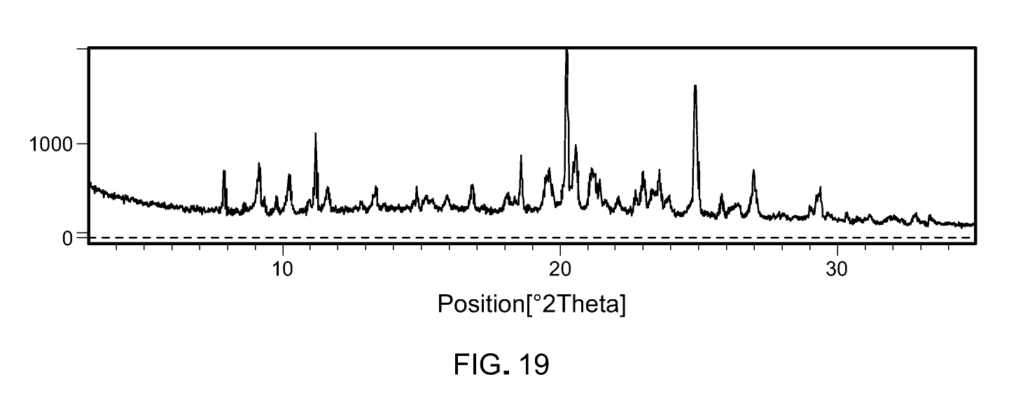

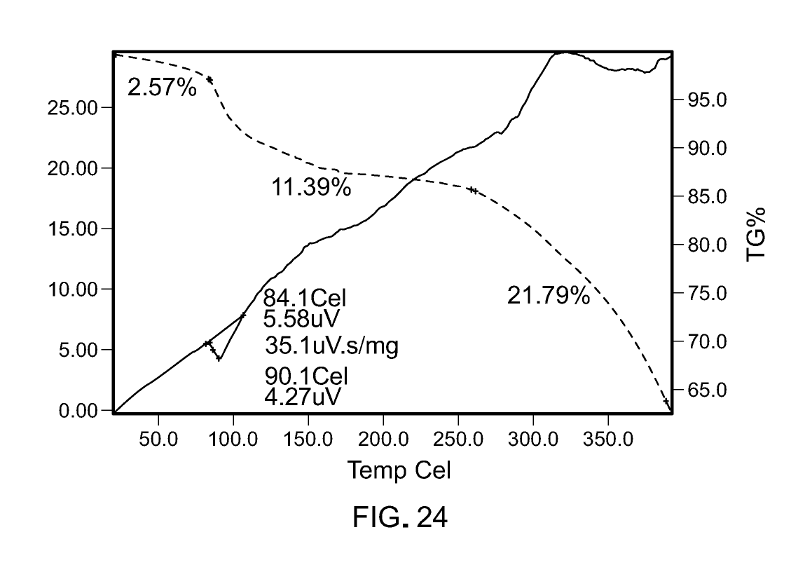

In some embodiments, the crystalline Compound 1 tosylate has a DTA thermogram substantially as depicted in FIG. 24. In some embodiments, the crystalline Compound 1 tosylate has a DTA thermogram characterized by an endothermal event at about 90° C. In some embodiments, the crystalline Compound 1 oxalate has an XRPD pattern substantially as depicted in FIG. 19.

In some embodiments, the crystalline Compound 1 oxalate has XRPD peaks, in terms of 2-theta, at about 20.2, about 20.5, and about 24.9. In some embodiments, the crystalline Compound 1 oxalate has XRPD peaks, in terms of 2-theta, at about 11.2, about 18.6, about 20.2, about 20.5, about 23.5, and about 24.9. In some embodiments, the crystalline Compound 1 oxalate has XRPD peaks, in terms of 2-theta, at about 11.2, about 18.6, about 20.0, about 20.2, about 20.5, about 21.1, about 22.9, about 23.5, about 24.9, and about 27.0.

In some embodiments, the crystalline Compound 1 oxalate has XRPD peaks, in terms of 2-theta, at 20.2±0.2, 20.5±0.2, and 24.9±0.2 degrees. In some embodiments, the crystalline Compound 1 oxalate has XRPD peaks, in terms of 2-theta, at 11.2±0.2, 18.6±0.2, 20.2±0.2, 20.5±0.2, 23.5±0.2, and 24.9±0.2 degrees. In some embodiments, the crystalline Compound 1 oxalate has XRPD peaks, in terms of 2-theta, at 11.2±0.2, 18.6±0.2, 20.0±0.2, 20.2±0.2, 20.5±0.2, 21.1±0.2, 22.9±0.2, 23.5±0.2, 24.9±0.2, and 27.0±0.2 degrees.

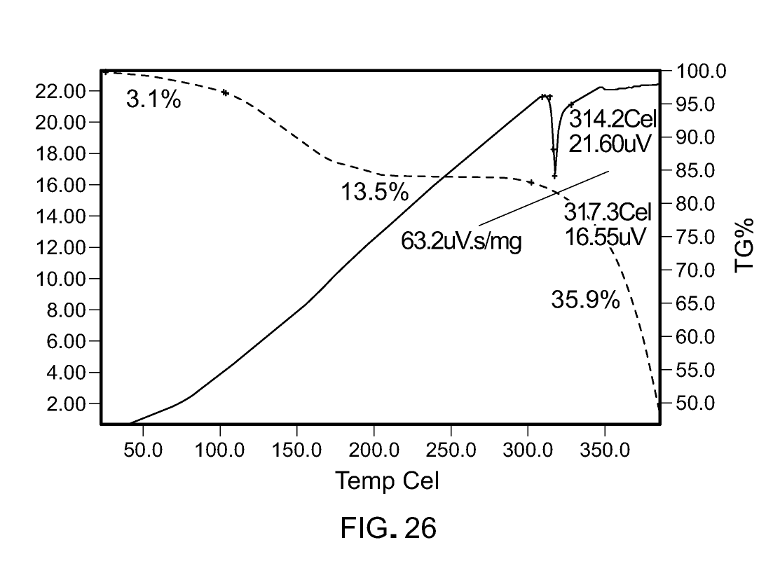

In some embodiments, the crystalline Compound 1 oxalate has a DTA thermogram substantially as depicted in FIG. 26. In some embodiments, the crystalline Compound 1 oxalate has a DTA thermogram characterized by an endothermal event at about 317° C. (a melting point).

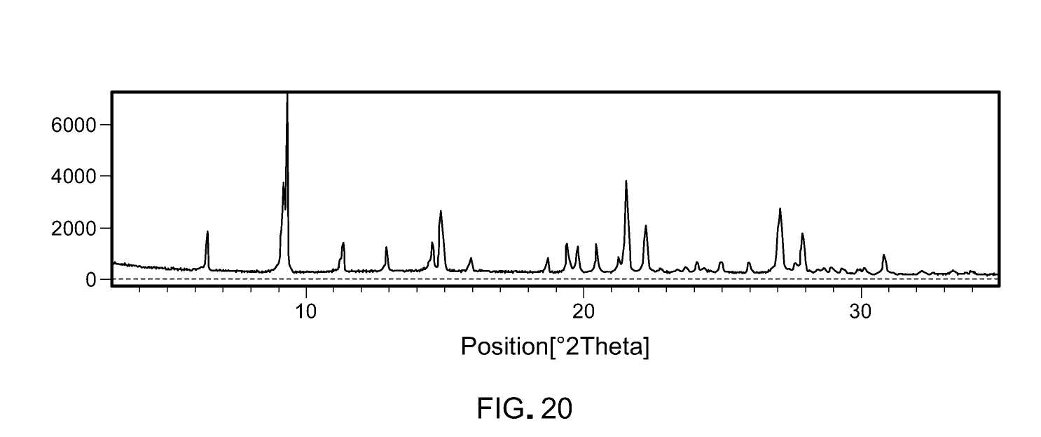

In some embodiments, the crystalline Compound 1 fumarate has an XRPD pattern substantially as depicted in FIG. 20.

In some embodiments, the crystalline Compound 1 fumarate has XRPD peaks, in terms of 2-theta, at about 9.3, about 21.6, and about 27.1. In some embodiments, the crystalline Compound 1 fumarate has XRPD peaks, in terms of 2-theta, at about 9.3, about 14.8, about 21.6, about 22.2, about 27.1, and about 27.9. In some embodiments, the crystalline Compound 1 fumarate has XRPD peaks, in terms of 2-theta, at about 6.4, about 9.3, about 14.8, about 19.4, about 19.8, about 20.4, about 21.6, about 22.2, about 27.1, and about 27.9.

In some embodiments, the crystalline Compound 1 fumarate has XRPD peaks, in terms of 2-theta, at 9.3±0.2, 21.6±0.2, and 27.1±0.2 degrees. In some embodiments, the crystalline Compound 1 fumarate has XRPD peaks, in terms of 2-theta, at 9.3±0.2, 14.8±0.2, 21.6±0.2, 22.2±0.2, 27.1±0.2, and 27.9±0.2 degrees. In some embodiments, the crystalline Compound 1 fumarate has XRPD peaks, in terms of 2-theta, at 6.4±0.2, 9.3±0.2, 14.8±0.2, 19.4±0.2, 19.8±0.2, 20.4±0.2, 21.6±0.2, 22.2±0.2, 27.1±0.2, and 27.9±0.2 degrees.

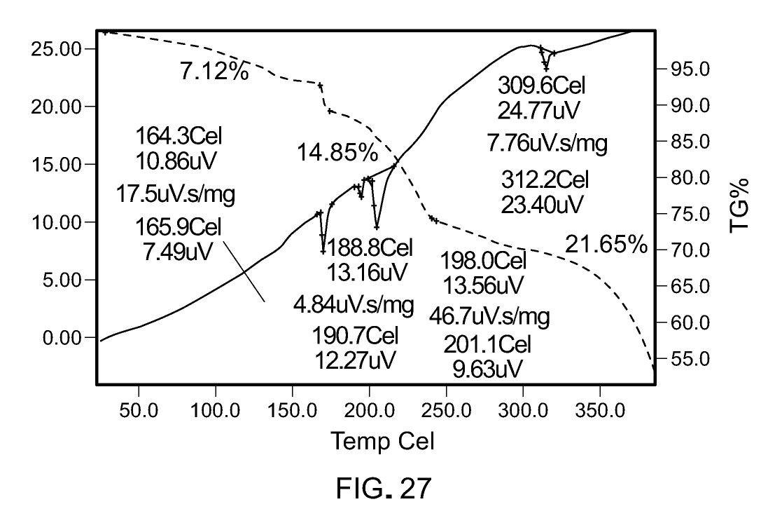

In some embodiments, the crystalline Compound 1 fumarate has a DTA thermogram substantially as depicted in FIG. 27. In some embodiments, the crystalline Compound 1 fumarate has a DTA thermogram characterized by an endothermal event at about 166° C. In some embodiments, the crystalline Compound 1 fumarate has a DTA thermogram characterized by an endothermal event at about 191° C. In some embodiments, the crystalline Compound 1 fumarate has a DTA thermogram characterized by an endothermal event at about 201° C. In some embodiments, the crystalline Compound 1 fumarate has a DTA thermogram characterized by an endothermal event at about 312° C. In some embodiments, the crystalline Compound 1 fumarate has a DTA thermogram characterized by an endothermal event at about 166° C., an endothermal event at about 191° C., an endothermal event at about 201° C., and an endothermal event at about 312° C.

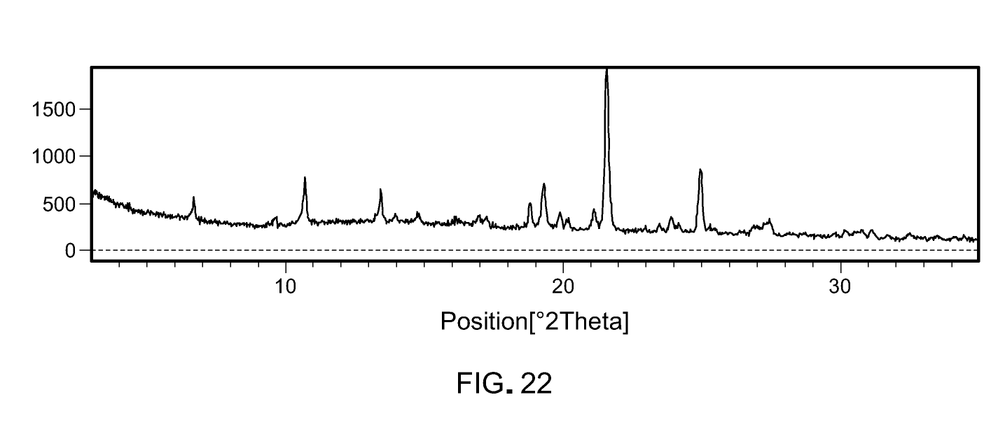

In some embodiments, the crystalline Compound 1 L-malate has an XRPD pattern substantially as depicted in FIG. 22.

In some embodiments, the crystalline Compound 1 malate has XRPD peaks, in terms of 2-theta, at about 19.3, about 21.6, and about 24.9. In some embodiments, the crystalline Compound 1 malate has XRPD peaks, in terms of 2-theta, at about 10.7, about 13.4, about 18.8, about 19.3, about 21.6, and about 24.9. In some embodiments, the crystalline Compound 1 malate has XRPD peaks, in terms of 2-theta, at about 6.7, about 10.7, about 13.4, about 18.8, about 19.3, about 19.9, about 21.1, about 21.6, about 23.9, and about 24.9.

In some embodiments, the crystalline Compound 1 malate has XRPD peaks, in terms of 2-theta, at 19.3±0.2, 21.6±0.2, and 24.9±0.2 degrees. In some embodiments, the crystalline Compound 1 malate has XRPD peaks, in terms of 2-theta, at 10.7±0.2, 13.4±0.2, 18.8±0.2, 19.3±0.2, 21.6±0.2, and 24.9±0.2 degrees. In some embodiments, the crystalline Compound 1 malate has XRPD peaks, in terms of 2-theta, at 6.7±0.2, 10.7±0.2, 13.4±0.2, 18.8±0.2, 19.3±0.2, 19.9±0.2, 21.1±0.2, 21.6±0.2, 23.9±0.2, and 24.9±0.2 degrees.

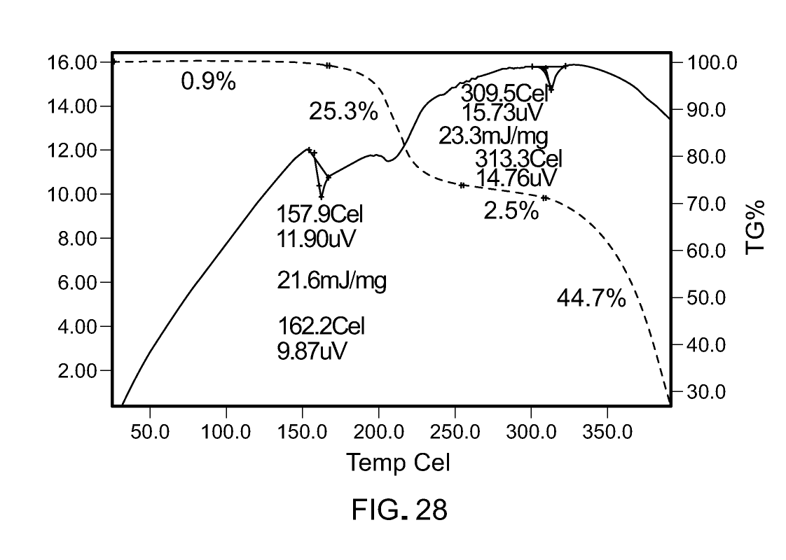

In some embodiments, the crystalline Compound 1 L-malate has a DTA thermogram substantially as depicted in FIG. 28. In some embodiments, the crystalline Compound 1 L-malate has a DTA thermogram characterized by an endothermal event at about 162° C. In some embodiments, the crystalline Compound 1 L-malate has a DTA thermogram characterized by an endothermal event at about 313° C. In some embodiments, the crystalline Compound 1 L-malate has a DTA thermogram characterized by an endothermal event at about 162° C. and an endothermal event at about 313° C.

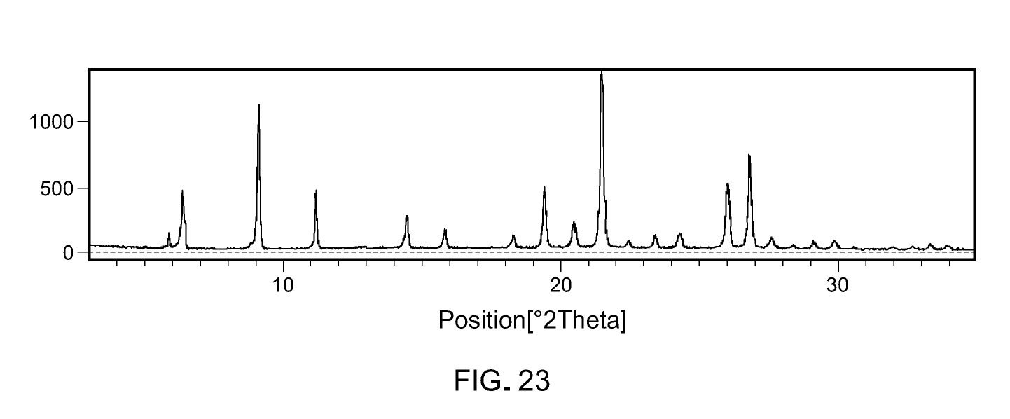

In some embodiments, the crystalline form of Compound 1 succinate has pattern 1. In some embodiments, the crystalline Compound 1 succinate has an XRPD pattern substantially as depicted in FIG. 23.

In some embodiments, the crystalline Compound 1 succinate has XRPD peaks, in terms of 2-theta, at about 9.1, about 21.5, and about 26.8. In some embodiments, the crystalline Compound 1 succinate has XRPD peaks, in terms of 2-theta, at about 9.1, about 11.2, about 19.4, about 21.5, about 26.0, and about 26.8. In some embodiments, the crystalline Compound 1 succinate has XRPD peaks, in terms of 2-theta, at about 6.4, about 9.1, about 11.2, about 14.5, about 15.8, about 19.4, about 20.5, about 21.5, about 26.0, about 26.8.

In some embodiments, the crystalline Compound 1 succinate has XRPD peaks, in terms of 2-theta, at 9.1±0.2, 21.5±0.2, and 26.8±0.2 degrees. In some embodiments, the crystalline Compound 1 succinate has XRPD peaks, in terms of 2-theta, at 9.1±0.2, 11.2±0.2, 19.4±0.2, 21.5±0.2, 26.0±0.2, and 26.8±0.2 degrees. In some embodiments, the crystalline Compound 1 succinate has XRPD peaks, in terms of 2-theta, at 6.4±0.2, 9.1±0.2, 11.2±0.2, 14.5±0.2, 15.8±0.2, 19.4±0.2, 20.5±0.2, 21.5±0.2, 26.0±0.2, 26.8±0.2 degrees.

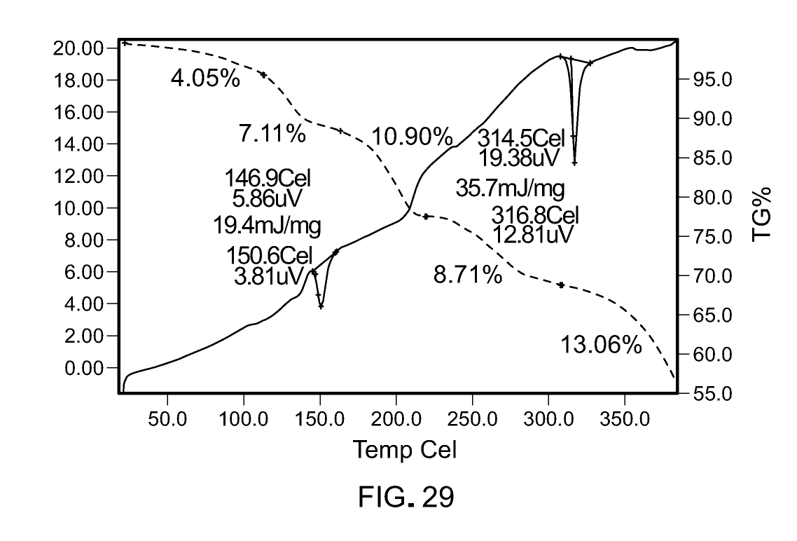

In some embodiments, the crystalline Compound 1 succinate has a DTA thermogram substantially as depicted in FIG. 29. In some embodiments, the crystalline Compound 1 oxalate has a DTA thermogram characterized by an endothermal event at about 151° C. In some embodiments, the crystalline Compound 1 oxalate has a DTA thermogram characterized by an endothermal event at about 315° C. In some embodiments, the crystalline Compound 1 oxalate has a DTA thermogram characterized by an endothermal event at about 151° C. and an endothermal event at about 315° C.

Compound 1 hydrochloride, Compound 1 sulfate, Compound 1 2-naphthalenesulfonate, Compound 1 isethionate, Compound 1 L-aspartate, Compound 1 maleate, Compound 1 phosphate, Compound 1 esylate, Compound 1 glutamate, Compound 1 L-tartrate, Compound 1 D-glucuronate, Compound 1 hippurate, Compound 1 D-gluconate, Compound 1 lactate, Compound 1 L-ascorbate, Compound 1 benzoate are provided herein and each of these salts can be prepared by treating Compound 1 with the corresponding acid.

### Synthetic Preparations

### Compound 1 and its Forms

In some embodiments, Compound 1 (free base) may be prepared as described, for example, in the U.S. provisional application No. 62/524,801, which is incorporated by reference herein in its entirety. The crystalline form of Compound 1 (e.g., Form I as described herein) may be prepared by the method comprising precipitating the crystalline form from a mixture comprising Compound 1 (free base). In some embodiments, the mixture further comprises a solvent. In some embodiments, the method comprises obtaining a mixture of Compound 1 with a solvent. In some embodiments, the mixture is a solution of Compound 1 in a solvent. In some embodiments, the solution is saturated. The solvent may be selected from acetone, acetonitrile, 2-butanone, cyclopropylmethyl ether, 1,2-dimethoxyethane, 1,4-dioxane, ethanol, ethyl acetate, 2-ethoxy ethanol, isobutyl acetate, isopropyl acetate, methanol, MIBK, 2-propanol, 1-propanol and THF.

In some embodiments, the precipitating is carried out at a temperature above 0° C. (e.g., 5° C., 10° C., 20° C., or 30° C.). In some embodiments, the precipitating is carried out below room temperature. In some aspects of these embodiments, the precipitating is carried out below 10° C. In some embodiments, the precipitating is carried out at about 2° C. In some aspects of these embodiments, the solution comprises 2-propanol (e.g., Compound 1 is precipitated from the solution in 2-propanol).

In some embodiments, the precipitating is carried out at a temperature below 0° C. (e.g., −5° C., −10° C., −20° C., or −30° C.). In some aspects of these embodiments, the precipitating is carried out at about −18° C. In other aspects of these embodiments, the solution comprises a solvent selected from 1-butanol, ethanol, 2-propanol and 1-propanol. For example, the Form I of Compound 1 may be precipitated by cooling a saturated solution of Compound 1 in, e.g., 1-butanol, and further collecting the resultant solid.

In some embodiments, the precipitating is carried out for a time period from about 24 hours to about 72 hours (e.g., cooled solution of Compound 1 may be stored at the specified temperature for 24-72 hours).

In some embodiments, the precipitating comprises adding an anti-solvent to the solution of Compound 1. In some aspects of these embodiments, the anti-solvent is miscible with the solvent in which Compound 1 is dissolved. For example, the anti-solvent may be selected from heptane and t-butylmethyl ether (herein also referred to as TBME). In some embodiments, the precipitating is carried out at or above room temperature. In some aspects of these embodiments, the solvent may be acetone, acetonitrile, 2-butanone, 1,2-dimethoxyethane, 1,4-dioxane and ethanol. For instance, a MTBE may be added to the solution of Compound 1 in acetone at room temperature, followed by collection of the precipitated Form I. In other aspects of these embodiments, the precipitating is carried out below room temperature (e.g., at 0° C., 5° C., or 10° C.). In one example, the precipitating is carried out at about 2° C. In some aspects of these embodiments, the solvent may be selected from acetone, acetonitrile, 1-butanol, 2-butanone, 1,2-dimethoxyethane, 1,4-dioxane, ethanol, ethyl acetate, MIBK, 1-propanol and THF. For instance, a heptane may be added to the solution of Compound 1 in ethyl acetate at about 2° C., followed by collection of the precipitated Form I.

In some embodiments, the precipitating may be carried out by evaporating the solvent. In some aspects of these embodiments, the evaporating may be carried out at about room temperature. In other aspects of these embodiments, the solvent is selected from acetone, acetonitrile, 2-butanone, cyclopropylmethyl ether, 1,2-dimethoxyethane, 1,4-dioxane, ethanol, ethyl acetate, 2-ethoxy ethanol, isobutyl acetate, isopropyl acetate, methanol, MIBK, 2-propanol, 1-propanol and THF.

### Compound 1 Salts and Crystalline Forms

Generally, the salts of the Compound 1 can be prepared by combining (6R,15R)-9-fluoro-15-methyl-2,11,16,20,21,24-hexaazapentacyclo[16.5.2.0^{2,6}.0^{7,12}.0^{21,25}]pentacosa-1(24),7,9,11,18(25),19,22-heptaen-17-one (Compound 1 free base) with an acid. That is, any one of the salts of Compound 1 described herein may be prepared by combining the Compound 1 with a benzenesulfonic acid, a citric acid, a methanesulfonic acid, a 1,2-ethane disulfonic acid, a p-toluene sulfonic acid, an oxalic acid, a fumaric acid, a L-malic acid, a hydrochloric acid, a sulfuric acid, a naphthalene-2-sulfonic acid, a 2-hydroxy ethanesulfonic acid, a L-aspartic acid, a maleic acid, a phosphoric acid, a ethanesulfonic acid, a L-glutamic acid, a L-tartaric acid, a D-glucuronic acid, a hippuric acid, a D-gluconic acid, a DL-lactic acid, a L-ascorbic acid, or a benzoic acid. In some embodiments, the combining may be carried out in the presence of a solvent, such as, for example, acetone, ethanol, methanol, 2-propanol, TBME or THF. In some embodiments, Compound 1 is combined with a solvent to obtain the first solution, an acid is separately combined with a solvent to obtain the second solution, and the salt of Compound 1 is obtained by combining the first solution with the second solution. In some embodiments, the combining is carried out with the acid in molar excess with respect to the Compound 1 free base. In some aspects of these embodiments, the molar ratio of the acid to the Compound 1 is from about 1:1 to about 1.1:1 (e.g., about 1.05:1). In some embodiments, the combining is carried out from about room temperature to about 40° C. (e.g., the combining is carried out by cycling the temperature between ambient and 40° C. in 4 hour cycles). In some embodiments, the combining is carried out for a time period from 24 hours to 72 hours.

Generally, any one of the crystalline forms of the salts of Compound 1 may be obtained by precipitating the crystalline form from a mixture of the salt with a solvent (e.g., precipitating the crystalline compound from a mixture, such as precipitating the crystalline compound from a solution). In some embodiments, the precipitating is carried out by temperature cycling the reaction mixture from about room temperature to about 40° C. (e.g., 4 hour cycles between room temperature and 40° C.). In some embodiments, the precipitating is carried out by evaporating the solvent from the mixture (e.g., by evaporating the solvent from the solution of Compound 1). In some embodiments, the precipitating is carried out by adding an anti-solvent (e.g., heptane of MTBE) to the solution of Compound 1 in a solvent.

In some embodiments, crystalline Compound 1 besylate may be obtained by precipitating the crystalline form from a mixture of Compound 1 besylate with a solvent selected from THF and t-BME. In some aspects of these embodiments, the mixture is a solution of Compound 1 besylate in THF or t-BME.

In some embodiments, crystalline Compound 1 besylate may be prepared by precipitating the crystalline form from a mixture of Compound 1 besylate with ethanol. In some aspects of these embodiments, the mixture is a solution of Compound 1 besylate in ethanol.

In some embodiments, crystalline Compound 1 citrate Form A may be prepared by precipitating Form A from a mixture of Compound 1 citrate with a solvent selected from acetone and t-BME. In some aspects of these embodiments, the mixture is a solution of Compound 1 citrate in acetone or t-BME.

In some embodiments, crystalline form of Compound 1 mesylate may be prepared by precipitating the crystalline form from a mixture of Compound 1 mesylate with a solvent selected from acetone, methanol and 2-propanol. In some aspects of these embodiments, the mixture is a solution of Compound 1 mesylate in acetone, methanol or 2-propanol. In some embodiments, crystalline form of Compound 1 edisylate may be prepared by precipitating the crystalline form from a mixture of Compound 1 edisylate with 2-propanol. In some aspects of these embodiments, the mixture is a solution of Compound 1 edisylate in 2-propanol.

In some embodiments, crystalline form of Compound 1 tosylate may be prepared by precipitating the crystalline form from a mixture of Compound 1 tosylate with a solvent selected from acetone and THF. In some aspects of these embodiments, the mixture is a solution of Compound 1 tosylate in acetone or THF.

In some embodiments, crystalline form of Compound 1 oxalate may be prepared by precipitating the crystalline form from a mixture of Compound 1 oxalate with a solvent selected from ethanol and methanol. In some aspects of these embodiments, the mixture is a solution of Compound 1 oxalate in ethanol or methanol.

In some embodiments, a crystalline form of Compound 1 fumarate may be prepared by precipitating the crystalline form from a mixture of Compound 1 fumarate with acetone. In some aspects of these embodiments, the mixture is a solution of Compound 1 fumarate in ethanol or methanol.

In some embodiments, crystalline form of Compound 1 L-malate may be prepared by precipitating the crystalline form from a mixture of Compound 1 L-malate with TBME. In some aspects of these embodiments, the mixture is a solution of Compound 1 L-malate in TBME.

In some embodiments, crystalline form of Compound 1 succinate may be prepared by precipitating the crystalline form from a mixture of Compound 1 succinate with acetone. In some aspects of these embodiments, the mixture is a solution of Compound 1 succinate in acetone.

### 2. Methods of Use

### Tropomyosin Receptor Kinases (Trks)

Three different NTRK genes have been implicated as having a role in cancer (e.g., through discovery of chromosome translocations resulting in constitutively active Trk fusion proteins): NTRK1, NTRK2, and NTRK3. The NTRK1, NTRK2, and NTRK3 genes encode TrkA, TrkB, and TrkC, respectively.

Non-limiting exemplary amino acid and cDNA sequences for wild-type TrkA are provided below. The exemplary wild-type protein and cDNA sequences provided below can be used to identify a point mutation in a NTRK1 gene or can be used to determine mutation in a TrkA protein caused by a point mutation in a NTRK1 gene, respectively. Additional wild-type protein and cDNA sequences for TrkA are known in the art.

The amino acid positions used to describe the TrkA substitutions herein are based on the wild-type sequence of TrkA of SEQ ID NO: 1. The corresponding amino acid position in the wild-type sequence of another isoform of TrkA (SEQ ID NO: 3) can be identified by performing a sequence alignment between SEQ ID NO: 1 and SEQ ID NO: 3. A similar method (e.g., alignment of SEQ ID NO: 1 to the amino acid sequence of any other isoform of TrkA) can be used to match the amino acid positions of the substitutions 46040-0009001/125-33-US in TrkA described herein to the corresponding amino acid position in other isoforms of TrkA known in the art.

### Wildtype Human TrkA Protein Isoform A (NP_002520) (SEQ ID NO: 1)

Wildtype Human TrkA cDNA Isoform A (NM_002529) (SEQ ID NO: 2)

### Wildtype Human TrkA Protein Isoform B (NP_001007793) (SEQ ID NO: 3)

Wildtype Human TrkA cDNA Isoform B (NM_001007792) (SEQ ID NO: 4)
Alignment of TrkA isoforms (SEQ ID NO: 1 and SEQ ID NO: 3)

| | | | |
|---|---|---|---|
| S1 | 68 | LTELYIENQQHLQHLELRDLRGLGELRNLTIVKSGLRFVAPDAFHFTPRLSRLNLSFNAL | 127 |
| | | L YIENQQHLQHLELRDLRGLGELRNLTIVKSGLRFVAPDAFHFTPRLSRLNLSFNAL | |
| S3 | 38 | LAASYIENQQHLQHLELRDLRGLGELRNLTIVKSGLRFVAPDAFHFTPRLSRLNLSFNAL | 97 |
| S1 | 128 | ESLSWKTVQGLSLQELVLSGNPLHCSCALRWLQRWEEEGLGGVPEQKLQCHGQGPLAHMP | 187 |
| | | ESLSWKTVQGLSLQELVLSGNPLHCSCALRWLQRWEEEGLGGVPEQKLQCHGQGPLAHMP | |
| S3 | 98 | ESLSWKTVQGLSLQELVLSGNPLHCSCALRWLQRWEEEGLGGVPEQKLQCHGQGPLAHMP | 157 |
| S1 | 188 | NASCGVPTLKVQVPNASVDVGDDVLLRCQVEGRGLEQAGWILTELEQSATVMKSGGLPSL | 247 |
| | | NASCGVPTLKVQVPNASVDVGDDVLLRCQVEGRGLEQAGWILTELEQSATVMKSGGLPSL | |
| S3 | 158 | NASCGVPTLKVQVPNASVDVGDDVLLRCQVEGRGLEQAGWILTELEQSATVMKSGGLPSL | 217 |
| S1 | 248 | GLTLANVTSDLNRKNVTCWAENDVGRAEVSVQVNVSFPASVQLHTAVEMHHWCIPFSVDG | 307 |
| | | GLTLANVTSDLNRKNVTCWAENDVGRAEVSVQVNVSFPASVQLHTAVEMHHWCIPFSVDG | |
| S3 | 218 | GLTLANVTSDLNRKNVTCWAENDVGRAEVSVQVNVSFPASVQLHTAVEMHHWCIPFSVDG | 277 |
| S1 | 308 | QPAPSLRWLFNGSVLNETSFIFTEFLEPAANETVRHGCLRLNQPTHVNNGNYTLLAANPF | 367 |
| | | QPAPSLRWLFNGSVLNETSFIFTEFLEPAANETVRHGCLRLNQPTHVNNGNYTLLAANPF | |
| S3 | 278 | QPAPSLRWLFNGSVLNETSFIFTEFLEPAANETVRHGCLRLNQPTHVNNGNYTLLAANPF | 337 |
| S1 | 368 | GQASASIMAAFMDNPFEFNPEDPIPVSFSPVDTNSTSGDPVEKKDETPFGVSVAVGLAVF | 427 |
| | | GQASASIMAAFMDNPFEFNPEDPIP DTNSTSGDPVEKKDETPFGVSVAVGLAVF | |
| S3 | 338 | GQASASIMAAFMDNPFEFNPEDPIP------DTNSTSGDPVEKKDETPFGVSVAVGLAVF | 391 |
| S1 | 428 | ACLFLSTLLLVLNKCGRRNKFGINRPAVLAPEDGLAMSLHFMTLGGSSLSPTEGKGSGLQ | 487 |
| | | ACLFLSTLLLVLNKCGRRNKFGINRPAVLAPEDGLAMSLHFMTLGGSSLSPTEGKGSGLQ | |
| S3 | 392 | ACLFLSTLLLVLNKCGRRNKFGINRPAVLAPEDGLAMSLHFMTLGGSSLSPTEGKGSGLQ | 451 |
| S1 | 488 | GHIIENPQYFSDACVHHIKRRDIVLKWELGEGAFGKVFLAECHNLLPEQDKMLVAVKALK | 547 |
| | | GHIIENPQYFSDACVHHIKRRDIVLKWELGEGAFGKVFLAECHNLLPEQDKMLVAVKALK | |
| S3 | 452 | GHIIENPQYFSDACVHHIKRRDIVLKWELGEGAFGKVFLAECHNLLPEQDKMLVAVKALK | 511 |
| S1 | 548 | EASESARQDFQREAELLTMLQHQHIVRFFGVCTEGRPLLMVFEYMRHGDLNRFLRSHGPD | 607 |
| | | EASESARQDFQREAELLTMLQHQHIVRFFGVCTEGRPLLMVFEYMRHGDLNRFLRSHGPD | |
| S3 | 512 | EASESARQDFQREAELLTMLQHQHIVRFFGVCTEGRPLLMVFEYMRHGDLNRFLRSHGPD | 571 |
| S1 | 608 | AKLLAGGEDVAPGPLGLGQLLAVASQVAAGMVYLAGLHFVHRDLATRNCLVGQGLVVKIG | 667 |
| | | AKLLAGGEDVAPGPLGLGQLLAVASQVAAGMVYLAGLHFVHRDLATRNCLVGQGLVVKIG | |
| S3 | 572 | AKLLAGGEDVAPGPLGLGQLLAVASQVAAGMVYLAGLHFVHRDLATRNCLVGQGLVVKIG | 631 |
| S1 | 668 | DEGMSRDIYSTDYYRVGGRTMLPIRWMPPESILYRKFTTESDVWSEGVVLWEIFTYGKQP | 727 |
| | | DEGMSRDIYSTDYYRVGGRTMLPIRWMPPESILYRKFTTESDVWSEGVVLWEIFTYGKQP | |
| S3 | 632 | DEGMSRDIYSTDYYRVGGRTMLPIRWMPPESILYRKFTTESDVWSEGVVLWEIFTYGKQP | 691 |
| S1 | 728 | WYQLSNTEAIDCITQGRELERPRACPPEVYAIMRGCWQREPQQRHSIKDVHARLQALAQA | 787 |
| | | WYQLSNTEAIDCITQGRELERPRACPPEVYAIMRGCWQREPQQRHSIKDVHARLQALAQA | |
| S3 | 692 | WYQLSNTEAIDCITQGRELERPRACPPEVYAIMRGCWQREPQQRHSIKDVHARLQALAQA | 751 |
| S1 | 788 | PPVYLDVLG | 796 |
| | | PPVYLDVLG | |
| S3 | 752 | PPVYLDVLG | 760 |

Non-limiting exemplary amino acid and cDNA sequences for wildtype TrkB are provided below. The exemplary wildtype protein and cDNA sequences provided below can be used to identify a point mutation in a NTRK2 gene or can be used to determine mutation in a TrkB protein caused by a point mutation in a NTRK2 gene, respectively. Additional wildtype protein and cDNA sequences for TrkB are known in the art.

The amino acid positions used to describe the TrkB substitutions herein are based on the wildtype sequence of TrkB of SEQ ID NO: 5. The corresponding amino acid position in the wildtype sequence of another isoform of TrkB can be identified by performing a sequence alignment between SEQ ID NO: 5 and the amino acid sequence of the other isoform of TrkB.

### Wildtype Human TrkB Protein Isoform A (AAB33109.1) (SEQ ID NO: 5)

Wildtype Human TrkB cDNA Isoform A (S76473.1) (SEQ ID NO: 6)

Non-limiting exemplary amino acid and cDNA sequences for wildtype TrkC are provided below. The exemplary wildtype protein and cDNA sequences provided below can be used to identify a point mutation in a NTRK3 gene or can be used to determine mutation in a TrkC protein caused by a point mutation in a NTRK3 gene, respectively. Additional wildtype protein and cDNA sequences for TrkC are known in the art.

The amino acid positions used to describe the TrkC substitutions herein are based on the wildtype sequence of TrkC of SEQ ID NO: 7. The corresponding amino acid position in the wildtype sequence of another isoform of TrkC can be identified by performing a sequence alignment between SEQ ID NO: 7 and the amino acid sequence of the other isoform of TrkC.

### Wildtype Human TrkC Protein (AAB33111.1) (SEQ ID NO: 7)

Wildtype Human TrkC cDNA (S76475.1) (SEQ ID NO: 8)

### Trk Inhibitors

A variety of Trk inhibitors are known in the art. The ability of a Trk inhibitor to act as a Trk inhibitor may be tested using one or both of the assays described in Examples A and B in U.S. Pat. No. 8,513,263, which is incorporated herein by reference.

A Trk inhibitor can bind to one or more of the sites on TrkA: the extracellular cysteine-rich region (domain 1), the extracellular leucine rich region (domain 2), the extracellular cysteine-rich region (domain 3), the extracellular immunoglobulin-like region (domain 4), the extracellular immunoglobulin-like region (domain 5), the transmembrane region, the intracellular kinase domain, an amino acid in the active site, the ATP-binding pocket, the tyrosine substrate binding site, the activation loop (e.g., the DFG motif of the activation loop), the kinase insert domain (KID) region (e.g., amino acids 603 to 623), the hinge region of the kinase, the α-C helix in the catalytic domain, the N-lobe lysine responsible for the stabilization of the α phosphate of the ATP substrate, the C-terminus (see, e.g., Bertrand et al., J. Mol. Biol. 423:439-453, 2012), the α-D helix in the C-terminus, the α-E helix in the C-terminus, an amino acid in the kinase domain that interacts with a ligand in the ATP binding site (see, e.g., Cherry et al., Curr. Med. Chem. 11:663-673, 2004). For example, a Trk inhibitor can bind to domain 5 or the intracellular kinase domain of a TrkA.

Non-limiting examples of Trk inhibitors include: entrectinib (N-[5-(3,5-difluoro-benzyl)-1H-indazol-3-yl]-4-(4-methylpiperazin-1-yl)-2-(tetrahydro-pyran-4-ylamino)-benzamide), (S)—N-(5-((R)-2-(2,5-difluorophenyl)pyrrolidin-1-yl)pyrazolo[1,5-a]pyrimidin-3-yl)-3-hydroxypyrrolidine-1-carboxamide sulfate, cabozantinib ((N-(4-((6,7-Dimethoxyquinolin-4-yl)oxy)phenyl)-N′-(4-fluorophenyl)cyclopropane-1,1-dicarboxamide)), dovitinib (4-amino-5-fluoro-3-[6-(4-methylpiperazin-1-yl)-1H-benzimidazol-2-yl]quinolin-2(1H)-one mono 2-hydroxypropanoate hydrate), belizatinib (4-fluoro-N-(6-((4-(2-hydroxypropan-2-yl)piperidin-1-yl)methyl)-1-((1s,4s)-4-(i sopropylcarbamoyl)cyclohexyl)-1H-benzo[d]imidazol-2-yl)benzamide), sitravatinib (N-(3-fluoro-4-((2-(5-(((2-methoxyethyl)amino)methyl)pyridin-2-yl)thieno[3,2-b]pyridin-7-yl)oxy)phenyl)-N-(4-fluorophenyl)cyclopropane-1,1-dicarboxamide), PLX7486, altiratinib (N-(4-((2-(cyclopropanecarboxamido)pyridin-4-yl)oxy)-2,5-difluorophenyl)-N-(4-fluorophenyl)cyclopropane-1,1-dicarboxamide), AZD7451 ((S)—N-(1-(5-fluoropyrimidin-2-yl)ethyl)-3-(5-isopropoxy-1H-pyrazol-3-yl)-3H-imidazo[4,5-b]pyridin-5-amine), (6R,15R)-9-fluoro-15-methyl-2,11,16,20,21,24-hexaazapentacyclo[16.5.2.0^{2,6}.0^{7,12}.0^{21,25}]pentacosa-1(24),7,9, 11,18(25), 19,22-heptaen-17-one, a (R)-2-phenylpyrrolidine substituted imadazopyridazine, AZD6918, GNF-4256, GTx-186, GNF-5837, AZ623, AG-879, CT327, AR-772, AR-523, AR-786, AR-256, AR-618, AZ-23, CEP-701, CEP-751, PHA-739358, dovitinib, Go 6976, GW441756, MGCD516, ONO-5390556, PHA-848125AC, Regorafenib, Sorafenib, Sunitinib, TSR-011, VM-902A, K252a, a 4-aminopyrazolylpyrimidine, a substituted pyrazolo[1,5-a]pyrimidine compound, BMS-754807, ONO-7579, F17752, ANA-12, ONO-4474, GZ389988, and TPX-0005 ((7S,13R)-11-fluoro-7,13-dimethyl-6,7,13,14-tetrahydro-1,15-ethenopyrazolo[4,3-f][1,4,8,10]benzoxatriazacyclotridecin-4(5H)-one; repotrectinib).

Non-limiting examples of receptor tyrosine kinase (e.g., Trk) targeted therapeutic agents, include afatinib, cabozantinib, cetuximab, crizotinib, dabrafenib, entrectinib, erlotinib, gefitinib, imatinib, lapatinib, lestaurtinib, nilotinib, pazopanib, panitumumab, pertuzumab, sunitinib, trastuzumab, 1-((3 S,4R)-4-(3-fluorophenyl)-1-(2-methoxyethyl)pyrrolidin-3-yl)-3-(4-methyl-3-(2-methylpyrimidin-5-yl)-1-phenyl-1H-pyrazol-5-yl)urea, AG 879, AR-772, AR-786, AR-256, AR-618, AZ-23, AZ623, DS-6051, Go 6976, GNF-5837, GTx-186, GW 441756, LOXO-101, MGCD516, PLX7486, RXDX101, TPX-0005 ((7S,13R)-11-fluoro-7,13-dimethyl-6,7,13,14-tetrahydro-1,15-ethenopyrazolo[4,3-f][1,4,8,10]benzoxatriazacyclotridecin-4(5H)-one; repotrectinib), and TSR-011. Additional Trk targeted therapeutic agents include those described in U.S. Pat. Nos. 8,450,322; 8,513,263; 8,933,084; 8,791,123; 8,946,226; 8,450,322; 8,299,057; and 8,912,194; U.S. Publication No. 2016/0137654; 2015/0166564; 2015/0051222; 2015/0283132; and 2015/0306086; International Publication No. WO 2010/033941; WO 2010/048314; WO 2016/077841; WO 2011/146336; WO 2011/006074; WO 2010/033941; WO 2012/158413; WO 2014078454; WO 2014078417; WO 2014078408; WO 2014078378; WO 2014078372; WO 2014078331; WO 2014078328; WO 2014078325; WO 2014078323; WO 2014078322; WO 2015175788; WO 2009/013126; WO 2013/174876; WO 2015/124697; WO 2010/058006; WO 2015/017533; WO 2015/112806; WO 2013/183578; and WO 2013/074518, all of which are hereby incorporated by reference in their entireties.

Further examples of Trk inhibitors can be found in U.S. Pat. No. 8,637,516, International Publication No. WO 2012/034091, U.S. Pat. No. 9,102,671, International Publication No. WO 2012/116217, U.S. Publication No. 2010/0297115, International Publication No. WO 2009/053442, U.S. Pat. No. 8,642,035, International Publication No. WO 2009092049, U.S. Pat. No. 8,691,221, International Publication No. WO2006131952, all of which are incorporated by reference in their entireties herein. Exemplary Trk inhibitors include GNF-4256, described in Cancer Chemother. Pharmacol. 75(1):131-141, 2015; and GNF-5837 (N-[3-[[2,3-dihydro-2-oxo-3-(1H-pyrrol-2-ylmethylene)-1H-indol-6-yl]amino]-4-methylphenyl]-N′-[2-fluoro-5-(trifluoromethyl)phenyl]-urea), described in ACS Med. Chem. Lett. 3(2):140-145, 2012, each of which is incorporated by reference in its entirety herein.

Additional examples of Trk inhibitors include those disclosed in U.S. Publication No. 2010/0152219, U.S. Pat. No. 8,114,989, and International Publication No. WO 2006/123113, all of which are incorporated by reference in their entireties herein. Exemplary Trk inhibitors include AZ623, described in Cancer 117(6):1321-1391, 2011; AZD6918, described in Cancer Biol. Ther. 16(3):477-483, 2015; AZ64, described in Cancer Chemother. Pharmacol. 70:477-486, 2012; AZ-23 ((S)-5-Chloro-N2-(1-(5-fluoropyridin-2-yl)ethyl)-N4-(5-isopropoxy-1H-pyrazol-3-yl)pyrimidine-2,4-diamine), described in Mol. Cancer Ther. 8:1818-1827, 2009; and AZD7451; each of which is incorporated by reference in its entirety.

A Trk inhibitor can include those described in U.S. Pat. Nos. 7,615,383; 7,384,632; 6,153,189; 6,027,927; 6,025,166; 5,910,574; 5,877,016; and 5,844,092, each of which is incorporated by reference in its entirety.

Further examples of Trk inhibitors include CEP-751, described in Int. J. Cancer 72:672-679, 1997; CT327, described in Acta Derm. Venereol. 95:542-548, 2015; compounds described in International Publication No. WO 2012/034095; compounds described in U.S. Pat. No. 8,673,347 and International Publication No. WO 2007/022999; compounds described in U.S. Pat. No. 8,338,417; compounds described in International Publication No. WO 2016/027754; compounds described in U.S. Pat. No. 9,242,977; compounds described in U.S. Publication No. 2016/0000783; sunitinib (N-(2-diethylaminoethyl)-5-[(Z)-(5-fluoro-2-oxo-1H-indol-3-ylidene)methyl]-2,4-dimethyl-1H-pyrrole-3-carboxamide), as described in PLoS One 9:e95628, 2014; compounds described in International Publication No. WO 2011/133637; compounds described in U.S. Pat. No. 8,637,256; compounds described in Expert. Opin. Ther. Pat. 24(7):731-744, 2014; compounds described in Expert Opin. Ther. Pat. 19(3):305-319, 2009; (R)-2-phenylpyrrolidine substituted imidazopyridazines, e.g., GNF-8625, (R)-1-(6-(6-(2-(3-fluorophenyl)pyrrolidin-1-yl)imidazo[1,2-b]pyridazin-3-yl)-[2,4′-bipyridin]-2′-yl)piperidin-4-ol as described in ACS Med. Chem. Lett. 6(5):562-567, 2015; GTx-186 and others, as described in PLoS One 8(12):e83380, 2013; K252a ((9S-(9α,10β,12α))-2,3,9,10,11,12-hexahydro-10-hydroxy-10-(methoxycarbonyl)-9-methyl-9,12-epoxy-1H-diindolo[1,2,3-fg:3′,2′,1′-kl]pyrrolo[3,4-i][1,6]benzodiazocin-1-one), as described in Mol. Cell Biochem. 339(1-2):201-213, 2010; 4-aminopyrazolylpyrimidines, e.g., AZ-23 (((S)-5-chloro-N2-(1-(5-fluoropyridin-2-yl)ethyl)-N4-(5-isopropoxy-1H-pyrazol-3-yl)pyrimidine-2,4-diamine)), as described in J Med. Chem. 51(15):4672-4684, 2008; PHA-739358 (danusertib), as described in Mol. Cancer Ther. 6:3158, 2007; Go 6976 (5,6,7,13-tetrahydro-13-methyl-5-oxo-12H-indolo[2,3-a]pyrrolo[3,4-c]carbazole-12-propanenitrile), as described in J. Neurochem. 72:919-924, 1999; GW441756 ((3Z)-3-[(1-methylindol-3-yl)methylidene]-1H-pyrrolo[3,2-b]pyridin-2-one), as described in IJAE 115:117, 2010; milciclib (PHA-848125AC), described in J. Carcinog. 12:22, 2013; AG-879 ((2E)-3-[3,5-Bis(1,1-dimethylethyl)-4-hydroxyphenyl]-2-cyano-2-propenethioamide); altiratinib (N-(4-((2-(cyclopropanecarboxamido)pyridin-4-yl)oxy)-2,5-difluorophenyl)-N-(4-fluorophenyl)cyclopropane-1,1-dicarboxamide); cabozantinib (N-(4-((6,7-Dimethoxyquinolin-4-yl)oxy)phenyl)-N′-(4-fluorophenyl)cyclopropane-1,1-dicarboxamide); lestaurtinib ((5 S,6S,8R)-6-Hydroxy-6-(hydroxymethyl)-5-methyl-7,8,14,15-tetrahydro-5H-16-oxa-4b,8a, 14-triaza-5,8-methanodibenzo[b,h]cycloocta[jkl]cyclopenta[e]-as-indacen-13(6H)-one); dovatinib (4-amino-5-fluoro-3-[6-(4-methylpiperazin-1-yl)-1H-benzimidazol-2-yl]quinolin-2(1H)-one mono 2-hydroxypropanoate hydrate); sitravatinib (N-(3-fluoro-4-((2-(5-(((2-methoxyethyl)amino)methyl)pyridin-2-yl)thieno[3,2-b]pyridin-7-yl)oxy)phenyl)-N-(4-fluorophenyl)cyclopropane-1,1-dicarboxamide); ONO-5390556; regorafenib (4-[4-({[4-Chloro-3-(trifluoromethyl)phenyl]carbamoyl}amino)-3-fluorophenoxy]-N-methylpyridine-2-carboxamide hydrate); and VSR-902A; all of the references above are incorporated by reference in their entireties herein.

Trk inhibitors are also described in U.S. Pat. Nos. 9,670,207, 9,701,681, and 9,346,788 and U.S. patent application Ser. No. 14/883,072 and are incorporated herein by reference in their entireties.

In some embodiments, the Trk inhibitor is selected from the group consisting of: (S)—N-(5-((R)-2-(2,5-difluorophenyl)pyrrolidin-1-yl)pyrazolo[1,5-a]pyrimidin-3-yl)-3-hydroxypyrrolidine-1-carboxamide sulfate; (R)—N-cyclopropyl-5-(2-(5-fluoropyridin-3-yl)pyrrolidin-1-yl)pyrazolo[1,5-a]pyrimidine-3-carboxamide; (6R,13 S)-9-fluoro-13-methyl-2,11,15,19,20,23-hexaazapentacyclo[15.5.2.17,11.02,6.020,24]pentacosa-1(23),7,9, 17(24), 18,21-hexaene-16,25-dione; and (6R)-9-fluoro-15-methyl-2,11,16,20,21,24-hexaazapentacyclo [16.5.2.02,6.07,12.021,25]pentacosa-1(24),7,9,11,18(25),19,22-heptaen-17-one.

Non-limiting examples of Trk inhibitors are described in U.S. Pat. No. 8,513,263 and International Publication No. WO 2010/048314 both of which are incorporated by reference in their entireties herein, and include a compound of Formula I:

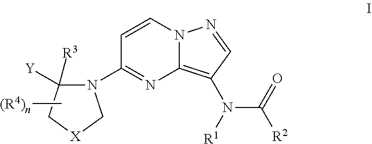

or a pharmaceutically acceptable salt thereof. For example, a Trk inhibitor can include one or more compounds selected from the group consisting of:
(R)—N-(5-(2-(2,5-difluorophenyl)pyrrolidin-1-yl)pyrazolo[1,5-a]pyrimidin-3-yl)-3-hydroxyazetidine-1-carboxamide;
N-(5-(2-(3-fluorophenyl)-2-methylpyrrolidin-1-yl)pyrazolo[1,5-a]pyrimidin-3-yl)-3-hydroxyazetidine-1-carboxamide;
(R)-1-(5-(2-(2,5-difluorophenyl)pyrrolidin-1-yl)pyrazolo[1,5-a]pyrimidin-3-yl)-3-phenylurea;
(R)—N-(5-(2-(2-(difluoromethyl)-5-fluorophenyl)pyrrolidin-1-yl)pyrazolo[1,5-a]pyrimidin-3-yl)-3-hydroxyazetidine-1-carboxamide;
(R)—N-(5-(2-(3-fluorophenyl)pyrrolidin-1-yl)pyrazolo[1,5-a]pyrimidin-3-yl)-1-methyl-6-oxo-1,6-dihydropyridazine-3-carboxamide;
(S)—N-(5-((R)-2-(2,5-difluorophenyl)pyrrolidin-1-yl)pyrazolo[1,5-a]pyrimidin-3-yl)-3-hydroxypyrrolidine-1-carboxamide;
(3R,4R)—N-(5-((R)-2-(2,5-difluorophenyl)pyrrolidin-1-yl)pyrazolo[1,5-a]pyrimidin-3-yl)-3,4-dihydroxypyrrolidine-1-carboxamide;
(S)—N-(5-((R)-2-(2-chloro-5-fluorophenyl)pyrrolidin-1-yl)pyrazolo[1,5-a]pyrimidin-3-yl)-3-methylpiperazine-1-carboxamide;
(R)—N-(5-(2-(2,5-difluorophenyl)pyrrolidin-1-yl)pyrazolo[1,5-a]pyrimidin-3-yl)-3-hydroxy-3-methyl azetidine-1-carboxamide;
(R)—N-(5-(2-(3-fluorophenyl)pyrrolidin-1-yl)pyrazolo[1,5-a]pyrimidin-3-yl)-3-hydroxyazetidine-1-carboxamide; and
(R)-1-(4-chlorophenyl)-3-(5-(2-(2,5-difluorophenyl)pyrrolidin-1-yl)pyrazolo[1,5-a]pyrimidin-3-yl)urea,
   or a pharmaceutically acceptable salt thereof.

Additional examples of Trk inhibitors are the substituted pyrazolo[1,5-a]pyrimidine compounds described in U.S. Pat. No. 8,791,123 and International Publication No. WO 2011/006074, both of which are herein incorporated by reference in their entireties. For example, Trk inhibitors that are substituted pyrazolo[1,5-a]pyrimidine compounds can have the general formula II:

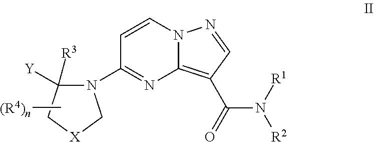

or a salt thereof. For example, a Trk inhibitor can include one or more compounds selected from the group consisting of:
(R)-5-(2-(2,5-difluorophenyl)pyrrolidin-1-yl)-N-(pyridin-2-yl)pyrazolo[1,5-a]pyrimidine-3-carboxamide;
(R)-5-(2-(2,5-difluorophenyl)pyrrolidin-1-yl)-N-(2-morpholinoethyl)pyrazolo[1,5-a]pyrimidine-3-carboxamide;
N-((2S)-bicyclo[2.2.1]heptan-2-yl)-5-((R)-2-(2,5-difluorophenyl)pyrrolidin-1-yl)pyrazolo[1,5-a]pyrimidine-3-carboxamide;
(R)-5-(2-(2,5-difluorophenyl)pyrrolidin-1-yl)-N-(2-(2-oxoimidazolidin-1-yl)ethyl)pyrazole[1,5-a]pyrimidine-3-carboxamide;
5-((R)-2-(2,5-difluorophenyl)pyrrolidin-1-yl)-N—((R)-2,3-dihydroxypropyl)pyrazolo[1,5-a]pyrimidine-3-carboxamide;
(R)—N-cyclopropyl-5-(2-(5-fluoropyridin-3-yl)pyrrolidin-1-yl)pyrazolo[1,5-a]pyrimidine-3-carboxamide;
(R)—N-tert-butyl-5-(2-(5-fluoropyridin-3-yl)pyrrolidin-1-yl)pyrazolo[1,5-a]pyrimidine-3-carboxamide;
(R)-5-(2-(2,5-difluorophenyl)pyrrolidin-1-yl)pyrazolo[1,5-a]pyrimidine-3-carboxamide;
(R)-5-(2-(5-fluoropyridin-3-yl)pyrrolidin-1-yl)-N-(1-methylcyclobutyl)pyrazolo[1,5-a]pyrimidine-3-carboxamide; and
5-((R)-2-(5-fluoropyridin-3-yl)pyrrolidin-1-yl)-N—((S)-1,1,1-trifluoropropan-2-yl)pyrazolo[1,5-a]pyrimidine-3-carboxamide;
   or a pharmaceutically acceptable salt thereof.

Additional examples of Trk inhibitors are the macrocyclic compounds described in U.S. Pat. No. 8,933,084 and International Publication No. WO 2011/146336, both of which are herein incorporated by reference in their entireties. For example, Trk inhibitors that are macrocyclic compounds can have the general formula III:

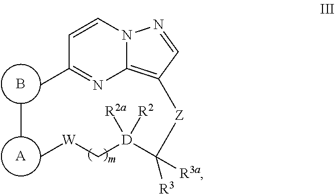

or a pharmaceutically acceptable salt thereof. For example, a Trk inhibitor can include one or more compounds selected from the group consisting of:
(6R)-9-fluoro-13-oxa-2,11,17,21,22,25-hexaazapentacyclo[17.5.2.0^{2,6}.0^{7,12}.0^{22,26}]hexacosa-1(25),7,9,11,19(26),20,23-heptaen-18-one;
(6R)-9-fluoro-15-hydroxy-13-oxa-2,11,17,21,22,25-hexaazapentacyclo[17.5.2.0^{2,6}.0^{7,12}.0^{22,26}] hexacosa-1(25),7,9,11,19(26),20,23-heptaen-18-one;
(6R,15R)-9-fluoro-15-hydroxy-13-oxa-2,11,17,21,22,25-hexaazapentacyclo-[17.5.2.0^{2,6}.0^{7,12}.0^{22,26}] hexacosa-1(25),7,9,11,19(26),20,23-heptaen-18-one;
(6R)-9-fluoro-13-oxa-2,11,16,20,21,24-hexaazapentacyclo[16.5.2.0^{2,6}.0^{7,12}.0^{21,25}]pentacosa-1 (24),7,9,11,18(25), 19,22-heptaen-17-one;
(6R)-9-fluoro-13-oxa-2,11,18,22,23,26-hexaazapentacyclo[18.5.2.0^{2,6}.0^{7,12}.0^{23,27}]heptacosa-1 (26),7,9,11,20(27),21,24-heptaen-19-one;
(6R,13S)-9-fluoro-13-methyl-2,11,15,19,20,23-hexaazapentacyclo [15.5.2.1^{7,11}.0^{2,6}.0^{20,24}]pentacosa-1 (23),7,9,17(24),18,21-hexaene-16,25-dione;
(6R)-9-fluoro-2,11,13,16,20,21,24-heptaazapentacyclo[16.5.2.0^{2,6}.0^{7,12}.0^{21,25}]pentacosa-1(24),7,9,11,18(25), 19,22-heptaen-17-one;
(6R)-9-fluoro-2,11,13,17,21,22,25-heptaazapentacyclo[17.5.2.0^{2,6}.0^{7,12}.0^{22,26}]hexacosa-1 (25),7,9,11,19(26),20,23-heptaen-18-one;
(6R)-9-fluoro-17-methyl-13-oxa-2,11,17,21,22,25-hexaazapentacyclo[17.5.2.0^{2,6}.0^{7,12}.0^{22,26}] hexacosa-1(25),7,9,11,19(26),20,23-heptaen-18-one;
(6R)-9,15,15-trifluoro-13-oxa-2,11,17,21,22,25-hexaazapentacyclo[17.5.2.0^{2,6}.0^{7,12}.0^{22,26}]hexacosa-1(25),7,9,11,19(26),20,23-heptaen-18-one;
(6R)-9-fluoro-2,11,16,20,21,24-hexaazapentacyclo[16.5.2.0^{2,6}.0^{7,12}.0^{21,25}]pentacosa-1 (24),7,9,11,18(25), 19,22-heptaen-17-one;
(6R)-9-fluoro-15-methyl-2,11,16,20,21,24-hexaazapentacyclo[16.5.2.0^{2,6}.0^{7,12}.0^{21,25}]pentacosa-1 (24),7,9,11,18(25), 19,22-heptaen-17-one;
(6R)-9-fluoro-(15R)-methyl-2,11,16,20,21,24-hexaazapentacyclo[16.5.2.0^{2,6}.0^{7,12}.0^{21,25}]pentacosa-1 (24),7,9,11,18(25), 19,22-heptaen-17-one;
(6R)-9-fluoro-15-methyl-2,11,16,20,21,24-hexaazapentacyclo[16.5.2.0^{2,6}.0^{7,12}.0^{21,25}]pentacosa-1 (24),7,9,11,18(25), 19,22-heptaen-17-one;
(6R)-9-fluoro-15,15-dimethyl-13-oxa-2,11,17,21,22,25-hexaazapentacyclo [17.5.2.0^{2,6}.0^{7,12}.0^{22,26}] hexacosa-1(25),7,9,11,19(26),20,23-heptaen-18-one; and
(6R)-9-fluoro-15,15-dimethyl-2,11,16,20,21,24-hexaazapentacyclo[16.5.2.0^{2,6}.0^{7,12}.0^{21,25}]pentacosa-1 (24),7,9,11,18(25), 19,22-heptaen-17-one;
   or a pharmaceutically acceptable salt thereof.

Additional examples of Trk inhibitors are the substituted imidazo[1,2-b]pyridazine compounds described in U.S. Pat. No. 8,450,322 and International Publication No. WO 2010/033941, both of which are herein incorporated by reference in their entireties. For example, Trk inhibitors that are substituted imidazo[1,2B]pyridazine compounds can have the general formula IV:

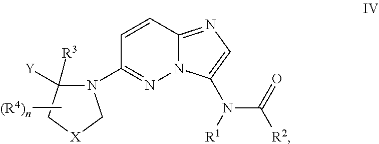

or a pharmaceutically acceptable salt thereof.

Additional examples of Trk inhibitors are the substituted pyrazolo[1,5-a]pyrimidine compounds described in WO 10/048314, herein incorporated by reference in its entirety. For example, Trk inhibitors that are substituted pyrazolo[1,5-a]pyrimidine compounds can have the general formula V:

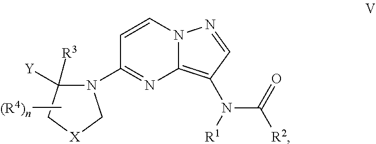

or a pharmaceutically acceptable salt thereof.

For example, a Trk inhibitor can include one or more compounds selected from the group consisting of:
(R)-5-(2-(2,5-difluorophenyl)pyrrolidin-1-yl)-N-(pyridin-2-yl)pyrazolo[1,5-a]pyrimidine-3-carboxamide;
(R)-5-(2-(2,5-difluorophenyl)pyrrolidin-1-yl)-N-(2-morpholinoethyl)pyrazolo[1,5-a]pyrimidine-3-carboxamide;
N-((2S)-bicyclo[2.2.1]heptan-2-yl)-5-((R)-2-(2,5-difluorophenyl)pyrrolidin-1-yl)pyrazolo[1,5-a]pyrimidine-3-carboxamide;
(R)-5-(2-(2,5-difluorophenyl)pyrrolidin-1-yl)-N-(2-(2-oxoimidazolidin-1-yl)ethyl)pyrazole[1,5-a]pyrimidine-3-carboxamide;
5-((R)-2-(2,5-difluorophenyl)pyrrolidin-1-yl)-N—((R)-2,3-dihydroxypropyl)pyrazolo[1,5-a]pyrimidine-3-carboxamide;
(R)—N-cyclopropyl-5-(2-(5-fluoropyridin-3-yl)pyrrolidin-1-yl)pyrazolo[1,5-a]pyrimidine-3-carboxamide;
(R)—N-tert-butyl-5-(2-(5-fluoropyridin-3-yl)pyrrolidin-1-yl)pyrazolo[1,5-a]pyrimidine-3-carboxamide;
(R)-5-(2-(2,5-difluorophenyl)pyrrolidin-1-yl)pyrazolo[1,5-a]pyrimidine-3-carboxamide;
(R)-5-(2-(5-fluoropyridin-3-yl)pyrrolidin-1-yl)-N-(1-methylcyclobutyl)pyrazolo[1,5-a]pyrimidine-3-carboxamide; and
5-((R)-2-(5-fluoropyridin-3-yl)pyrrolidin-1-yl)-N—((S)-1,1,1-trifluoropropan-2-yl)pyrazolo[1,5-a]pyrimidine-3-carboxamide;
   or a pharmaceutically acceptable salt thereof.

Additional Trk inhibitors can be found in U.S. Publication No. 2015/0166564 and WO 2012/158413, both of which are incorporated by reference in their entireties herein. For example, a Trk inhibitor can be a compound of Formula I:

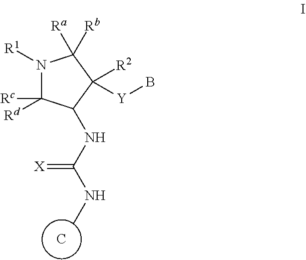

or stereoisomers, tautomers, or pharmaceutically acceptable salts, solvates or prodrugs thereof.

Further examples of Trk inhibitors can be found in International Publication No. WO 2014078454, which is incorporated by reference in its entirety herein. For example, a Trk inhibitor can be a compound of Formula I:

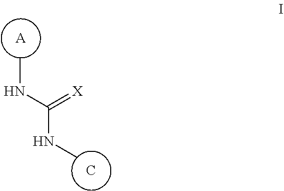

or stereoisomers, tautomers, or pharmaceutically acceptable salts, or solvates thereof.

Further examples of Trk inhibitors can be found in International Publication No. WO 2014078417, which is incorporated by reference in its entirety herein. For example, a Trk inhibitor can be a compound of Formula I:

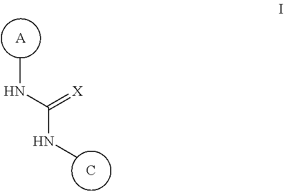

or stereoisomers, tautomers, or pharmaceutically acceptable salts, solvates or prodrugs thereof.

Further examples of Trk inhibitors can be found in International Publication No. WO 2014078378, which is incorporated by reference in its entirety herein. For example, a Trk inhibitor can be a compound of Formula I:

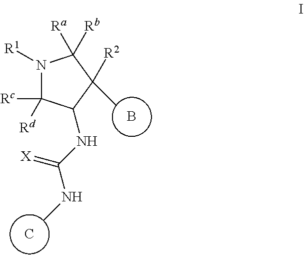

or stereoisomers, tautomers, or pharmaceutically acceptable salts, solvates or prodrugs thereof.

Additional examples of Trk inhibitors can be found in International Publication No. 5 WO 2014078372, which is incorporated by reference in its entirety herein. For example, a Trk inhibitor can be a compound of Formula I:

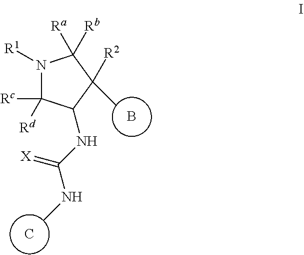

or stereoisomers, tautomers, or pharmaceutically acceptable salts, solvates or prodrugs thereof.

Additional examples of Trk inhibitors can be found in International Publication No. WO 2014078328, which is incorporated by reference in its entirety herein. For example, a Trk inhibitor can be a compound of Formula I-1:

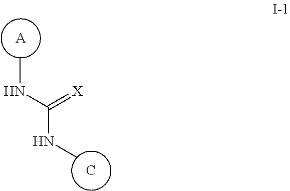

or stereoisomers, tautomers, or pharmaceutically acceptable salts, solvates or prodrugs thereof.

Further examples of Trk inhibitors can be found in International Publication No. WO 2014078325, which is incorporated by reference in its entirety herein. For example, a Trk inhibitor can be a compound of Formula I:

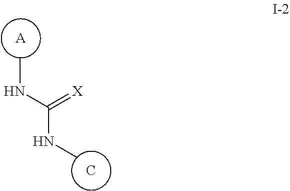

or a stereoisomer, tautomer, or pharmaceutically acceptable salt, solvate or prodrug thereof.

Additional examples of Trk inhibitors can be found in International Publication No. WO 2014078323, which is incorporated by reference in its entirety herein. For example, a Trk inhibitor can be a compound of Formula I:

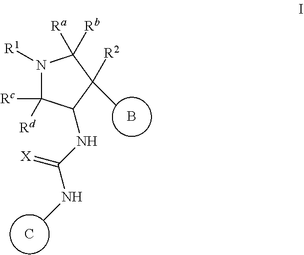

or stereoisomers, tautomers, or pharmaceutically acceptable salts, solvates or prodrugs.

Additional examples of Trk inhibitors can be found in International Publication No. WO 2014078322, which is incorporated by reference in its entirety herein. For example, a Trk inhibitor can be a compound of Formula I:

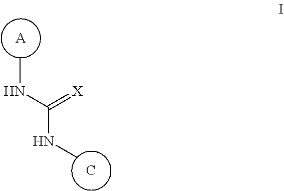

or stereoisomers, tautomers, or pharmaceutically acceptable salts, solvates or prodrugs thereof.

Exemplary Trk inhibitors include AR-772, AR-786, AR-256, and AR-618.

Non-limiting examples of Trk inhibitors can be found in U.S. Pat. No. 8,299,057 and International Publication No. WO 2009/013126 both of which are incorporated by reference in their entireties. For example, a Trk inhibitor can be a compound of Formula (I):

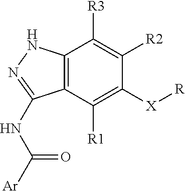

or optical isomers, tautomers or pharmaceutically acceptable salt thereof.

For example, a Trk inhibitor can be entrectinib (N-[5-(3,5-difluoro-benzyl)-1H-indazol-3-yl]-4-(4-methyl-piperazin-1-yl)-2-(tetrahydro-pyran-4-ylamino)-benzamide), or a pharmaceutically acceptable salt thereof. For example, a Trk inhibitor can be a polymorph such as those described in U.S. Publication No. 2015/0051222 or International Publication No. WO 2013/174876, both of which are incorporated by reference in their entireties herein. In some embodiments, a Trk inhibitor can be any disclosed in U.S. Publication No. 2015/0283132, International Publication No. WO 2015/124697, U.S. Pat. No. 8,946,226, International Publication No. WO 2010/012733, U.S. Pat. No. 8,912,194, and International Publication No. WO 2010/058006, all of which are incorporated by reference in their entireties herein.

Additional examples of Trk inhibitors can be found in U.S. Publication No. International Publication No. WO 2015/017533, which is incorporated by reference in its entirety herein.

Further examples of Trk inhibitors can be found in U.S. Publication No. 2016/0272725 and International Publication No. WO 2015/112806, both of which are incorporated by reference in their entirety herein. For example, a Trk inhibitor can be a compound of Formula (I-A):

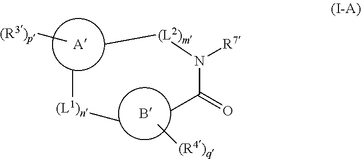

or a pharmaceutically acceptable salt thereof; or a pharmaceutically acceptable salt thereof. Exemplary Trk inhibitors include TPX-0005 (repotrectinib).

A Trk inhibitor can be one found in U.S. Pat. No. 9,187,489 and International Publication No. WO 2013/183578, both of which are incorporated by reference in their entireties herein. Exemplary Trk inhibitors include PLX7486 and DS-6051.

Non-limiting examples of Trk inhibitors can be found in U.S. Publication No. 2015/0306086 and International Publication No. WO 2013/074518, both of which are incorporated by reference in their entireties herein. Exemplary Trk inhibitors include TSR-011.

Further examples of Trk inhibitors can be found in U.S. Pat. No. 8,637,516, International Publication No. WO 2012/034091, U.S. Pat. No. 9,102,671, International Publication No. WO 2012/116217, U.S. Publication No. 2010/0297115, International Publication No. WO 2009/053442, U.S. Pat. No. 8,642,035, International Publication No. WO 2009092049, U.S. Pat. No. 8,691,221, International Publication No. WO2006131952, all of which are incorporated by reference in their entireties herein. Exemplary Trk inhibitors include GNF-4256, described in Cancer Chemother. Pharmacol. 75(1):131-141, 2015; and GNF-5837 (N-[3-[[2,3-dihydro-2-oxo-3-(1H-pyrrol-2-ylmethylene)-1H-indol-6-yl]amino]-4-methylphenyl]-N′-[2-fluoro-5-(trifluoromethyl)phenyl]-urea), described in ACS Med. Chem. Lett. 3(2):140-145, 2012, each of which is incorporated by reference in its entirety herein.

Additional examples of Trk inhibitors include those disclosed in U.S. Publication No. 2010/0152219, U.S. Pat. No. 8,114,989, and International Publication No. WO 2006/123113, all of which are incorporated by reference in their entireties herein. Exemplary Trk inhibitors include AZ623, described in Cancer 117(6):1321-1391, 2011; AZD6918, described in Cancer Biol. Ther. 16(3):477-483, 2015; AZ64, described in Cancer Chemother. Pharmacol. 70:477-486, 2012; AZ-23 ((S)-5-Chloro-N2-(1-(5-fluoropyridin-2-yl)ethyl)-N4-(5-isopropoxy-1H-pyrazol-3-yl)pyrimidine-2,4-diamine), described in Mol. Cancer Ther. 8:1818-1827, 2009; and AZD7451; each of which is incorporated by reference in its entirety.

A Trk inhibitor can include those described in U.S. Pat. Nos. 7,615,383; 7,384,632; 6,153,189; 6,027,927; 6,025,166; 5,910,574; 5,877,016; and 5,844,092, each of which is incorporated by reference in its entirety.

Further examples of Trk inhibitors include CEP-751, described in Int. J. Cancer 72:672-679, 1997; CT327, described in Acta Derm. Venereol. 95:542-548, 2015; compounds described in International Publication No. WO 2012/034095; compounds described in U.S. Pat. No. 8,673,347 and International Publication No. WO 2007/022999; compounds described in U.S. Pat. No. 8,338,417; compounds described in International Publication No. WO 2016/027754; compounds described in U.S. Pat. No. 9,242,977; compounds described in U.S. Publication No. 2016/0000783; sunitinib (N-(2-diethylaminoethyl)-5-[(Z)-(5-fluoro-2-oxo-1H-indol-3-ylidene)methyl]-2,4-dimethyl-1H-pyrrole-3-carboxamide), as described in PLoS One 9:e95628, 2014; compounds described in International Publication No. WO 2011/133637; compounds described in U.S. Pat. No. 8,637,256; compounds described in Expert. Opin. Ther. Pat. 24(7):731-744, 2014; compounds described in Expert Opin. Ther. Pat. 19(3):305-319, 2009; (R)-2-phenylpyrrolidine substituted imadizopyridazines, e.g., (4-((5-chloro-4-(methylamino)-7H-pyrrolo[2,3-d]pyrimidin-2-yl)amino)-3-methoxyphenyl)(morpholino)methanone as described in ACS Med. Chem. Lett. 6(5):562-567, 2015; GTx-186 and others, as described in PLoS One 8(12):e83380, 2013; K252a ((9S-(9α,10β,12α))-2,3,9,10,11,12-hexahydro-10-hydroxy-10-(methoxycarbonyl)-9-methyl-9,12-epoxy-1H-diindolo[1,2,3-fg:3′,2′,1′-kl]pyrrolo[3,4-i][1,6]benzodiazocin-1-one), as described in Mol. Cell Biochem. 339(1-2):201-213, 2010; 4-aminopyrazolylpyrimidines, e.g., AZ-23 (((S)-5-chloro-N2-(1-(5-fluoropyridin-2-yl)ethyl)-N4-(5-isopropoxy-1H-pyrazol-3-yl)pyrimidine-2,4-diamine)), as described in J. Med. Chem. 51(15):4672-4684, 2008; PHA-739358 (danusertib), as described in Mol. Cancer Ther. 6:3158, 2007; Go 6976 (5,6,7,13-tetrahydro-13-methyl-5-oxo-12H-indolo[2,3-a]pyrrolo[3,4-c]carbazole-12-propanenitrile), as described in J. Neurochem. 72:919-924, 1999; GW441756 ((3Z)-3-[(1-methylindol-3-yl)methylidene]-1H-pyrrolo[3,2-b]pyridin-2-one), as described in IJAE 115:117, 2010; milciclib (PHA-848125AC), described in J. Carcinog. 12:22, 2013; AG-879 ((2E)-3-[3,5-Bis(1,1-dimethylethyl)-4-hydroxyphenyl]-2-cyano-2-propenethioamide); altiratinib (N-(4-((2-(cyclopropanecarboxamido)pyridin-4-yl)oxy)-2,5-difluorophenyl)-N-(4-fluorophenyl)cyclopropane-1,1-dicarboxamide); cabozantinib (N-(4-((6,7-Dimethoxyquinolin-4-yl)oxy)phenyl)-N′-(4-fluorophenyl)cyclopropane-1,1-dicarboxamide); lestaurtinib ((5 S,6S,8R)-6-Hydroxy-6-(hydroxymethyl)-5-methyl-7,8,14,15-tetrahydro-5H-16-oxa-4b,8a, 14-triaza-5,8-methanodibenzo[b,h]cycloocta[jkl]cyclopenta[e]-as-indacen-13(6H)-one); dovatinib (4-amino-5-fluoro-3-[6-(4-methylpiperazin-1-yl)-1H-benzimidazol-2-yl]quinolin-2(1H)-one mono 2-hydroxypropanoate hydrate); sitravatinib (N-(3-fluoro-4-((2-(5-(((2-methoxyethyl)amino)methyl)pyridin-2-yl)thieno[3,2-b]pyridin-7-yl)oxy)phenyl)-N-(4-fluorophenyl)cyclopropane-1,1-dicarboxamide); ONO-5390556; regorafenib (4-[4-({[4-Chloro-3-(trifluoromethyl)phenyl]carbamoyl}amino)-3-fluorophenoxy]-N-methylpyridine-2-carboxamide hydrate); VSR-902A; all of the references above are incorporated by reference in their entireties herein.

In some embodiments, a Trk inhibitor is one or more compounds of Table 1, or a pharmaceutically acceptable salt thereof.

TABLE 1
Exemplary Trk inhibitors

| Compound No. | Compound Structure | Compound Name |
|---|---|---|
| 1 | 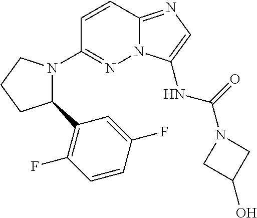 [SMILES: O=C(Nc1cnc2ccc(N3CCC[C@@H]3c3cc(F)ccc3F)nn12)N1CC(O)C1] | (R)-N-(6-(2-(2,5-difluorophenyl)pyrrolidin-1- yl)imidazo[1,2-b]pyridazin-3-yl)-3- hydroxyazetidine-1-carboxamide |
| 2 | 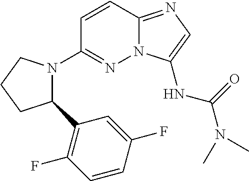 [SMILES: CN(C)C(=O)Nc1cnc2ccc(N3CCC[C@@H]3c3cc(F)ccc3F)nn12] | (R)-3-(6-(2-(2,5-difluorophenyl)pyrrolidin-1- yl)imidazo[1,2-b]pyridazin-3-yl)-1,1-dimethylurea |
| 3 | 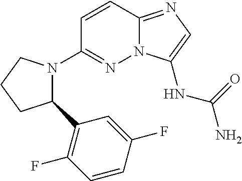 [SMILES: NC(=O)Nc1cnc2ccc(N3CCC[C@@H]3c3cc(F)ccc3F)nn12] | (R)-1-(6-(2-(2,5-difluorophenyl)pyrrolidin-1- yl)imidazo[1,2-b]pyridazin-3-yl)urea |
| 4 | 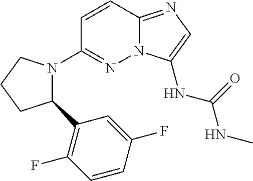 [SMILES: CNC(=O)Nc1cnc2ccc(N3CCC[C@@H]3c3cc(F)ccc3F)nn12] | (R)-1-(6-(2-(2,5-difluorophenyl)pyrrolidin-1- yl)imidazo[1,2-b]pyridazin-3-yl)-3-methylurea |
| 5 | 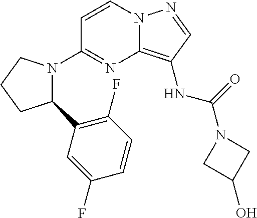 [SMILES: O=C(Nc1cnn2ccc(N3CCC[C@@H]3c3cc(F)ccc3F)nc12)N1CC(O)C1] | (R)-N-(5-(2-(2,5-difluorophenyl)pyrrolidin-1- yl)pyrazolo[1,5-a]pyrimidin-3-yl)-3- hydroxyazetidine-1-carboxamide |
| 6 | 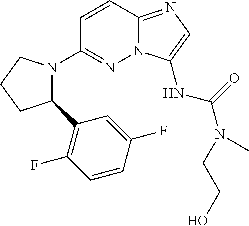 [SMILES: CN(CCO)C(=O)Nc1cnc2ccc(N3CCC[C@@H]3c3cc(F)ccc3F)nn12] | (R)-3-(6-(2-(2,5-difluorophenyl)pyrrolidin-1- yl)imidazo[1,2-b]pyridazin-3-yl)-1-(2- hydroxyethyl)-1-methylurea |
| 7 | 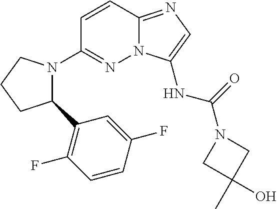 [SMILES: CC1(O)CN(C(=O)Nc2cnc3ccc(N4CCC[C@@H]4c4cc(F)ccc4F)nn23)C1] | (R)-N-(6-(2-(2,5-difluorophenyl)pyrrolidin-1- yl)imidazo[1,2-b]pyridazin-3-yl)-3-hydroxy-3- methylazetidine-1-carboxamide |
| 8 | 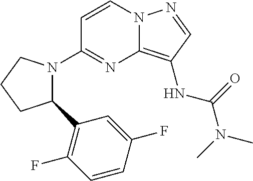 [SMILES: CN(C)C(=O)Nc1cnn2ccc(N3CCC[C@@H]3c3cc(F)ccc3F)nc12] | (R)-3-(5-(2-(2,5-difluorophenyl)pyrrolidin-1- yl)pyrazolo[1,5-a]pyrimidin-3-yl)-1,1-dimethylurea |
| 9 | 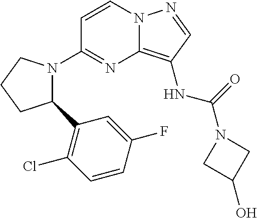 [SMILES: O=C(Nc1cnn2ccc(N3CCC[C@@H]3c3cc(F)ccc3Cl)nc12)N1CC(O)C1] | (R)-N-(5-(2-(2-chloro-5-fluorophenyl)pyrrolidin-1- yl)pyrazolo[1,5-a]pyrimidin-3-yl)-3- hydroxyazetidine-1-carboxamide |
| 10 | 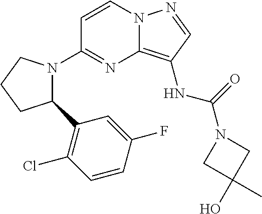 [SMILES: CC1(O)CN(C(=O)Nc2cnn3ccc(N4CCC[C@@H]4c4cc(F)ccc4Cl)nc23)C1] | (R)-N-(5-(2-(2-chloro-5-fluorophenyl)pyrrolidin-1- yl)pyrazolo[1,5-a]pyrimidin-3-yl)-3-hydroxy-3- methylazetidine-1-carboxamide |
| 11 | 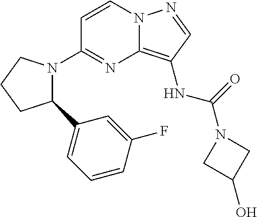 [SMILES: O=C(Nc1cnn2ccc(N3CCC[C@@H]3c3cccc(F)c3)nc12)N1CC(O)C1] | (R)-N-(5-(2-(3-fluorophenyl)pyrrolidin-1- yl)pyrazolo[1,5-a]pyrimidin-3-yl)-3- hydroxyazetidine-1-carboxamide |
| 12 | 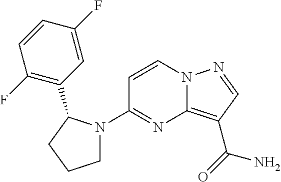 [SMILES: NC(=O)c1cnn2ccc(N3CCC[C@@H]3c3cc(F)ccc3F)nc12] | (R)-5-(2-(2,5-difluorophenyl)pyrrolidin-1- yl)pyrazolo[1,5-a]pyrimidine-3-carboxamide |
| 13 | 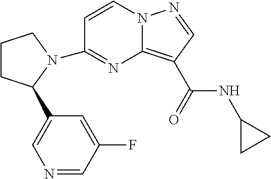 [SMILES: O=C(NC1CC1)c1cnn2ccc(N3CCC[C@@H]3c3cncc(F)c3)nc12] | (R)-N-cyclopropyl-5-(2-(5-fluoropyridin-3- yl)pyrrolidin-1-yl)pyrazolo[1,5-a]pyrimidine-3- carboxamide |
| 14 | 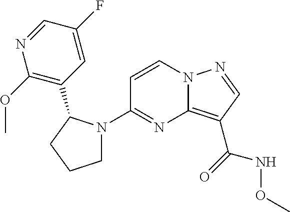 [SMILES: CONC(=O)c1cnn2ccc(N3CCC[C@@H]3c3cc(F)cnc3OC)nc12] | (R)-5-(2-(5-fluoro-2-methoxypyridin-3- yl)pyrrolidin-1-yl)-N-methoxypyrazolo[1,5- a]pyrimidine-3-carboxamide |
| 15 | 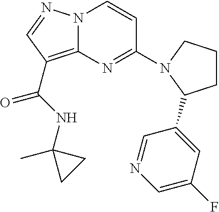 | (R)-5-(2-(5-fluoropyridin-3-yl)pyrrolidin-1-yl)-N- (1-methylcyclopropyl)pyrazolo[1,5-a]pyrimidine-3- carboxamide |
| 16 | 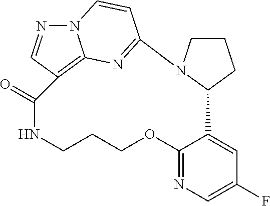 [SMILES: O=C1NCCCOc2ncc(F)cc2[C@H]2CCCN2c2ccn3ncc1c3n2] | (6R)-9-fluoro-13-oxa-2,11,17,21,22,25- hexaazapentacyclo[17.5.2.0^{2,6}.0^{7,12}.0^{22,26}]-hexacosa- 1(25),7,9,11,19(26),20,23-heptaen-18-one |
| 17 | 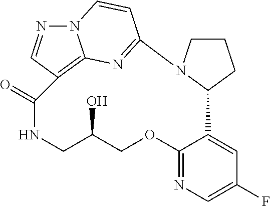 [SMILES: O=C1NC[C@@H](O)COc2ncc(F)cc2[C@H]2CCCN2c2ccn3ncc1c3n2] | (6R,15R)-9-fluoro-15-hydroxy-13-oxa- 2,11,17,21,22,25-hexaazapentacyclo- [17.5.2.0^{2,6}.0^{7,12}.0^{22,26}]-hexacosa- 1(25),7,9,11,19(26),20,23-heptaen-18-one |
| 18 | 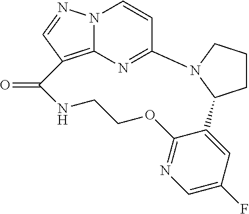 [SMILES: O=C1NCCOc2ncc(F)cc2[C@H]2CCCN2c2ccn3ncc1c3n2] | (6R)-9-fluoro-13-oxa-2,11,18,22,23,26- hexaazapentacyclo[18.5.2.0^{2,6}.0^{7,12}l.0^{23,27}]- heptacosa-1(26),7,9,11,20(27),21,24-heptaen-19- one |
| 19 | 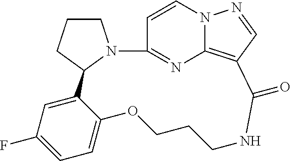 [SMILES: O=C1NCCCOc2ccc(F)cc2[C@H]2CCCN2c2ccn3ncc1c3n2] | (6R)-9-fluoro-13-oxa-2,17,21,22,25- pentaazapentacyclo[17.5.2.0^{2,6}.0^{7,12}.0^{22,26}]hexacosa- 1(25),7,9,11,19(26),20,23-heptaen-18-one |
| 20 | 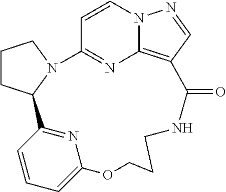 [SMILES: O=C1NCCCOc2cccc(n2)[C@H]2CCCN2c2ccn3ncc1c3n2] | (6R)-12-oxa-2,16,20,21,24,26- hexaazapentacyclo[16.5.2.^{17,11}.0^{2,6}.0^{21,21}]-hexacosa- 1(24),7(26),8,10,18(25),19,22-heptaen-17-one |
| 21 | 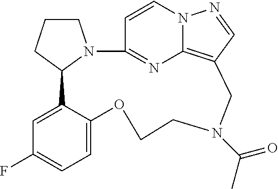 [SMILES: CC(=O)N1CCOc2ccc(F)cc2[C@H]2CCCN2c2ccn3ncc(c3n2)C1] | 1-[(6R)-9-fluoro-13-oxa-2,16,20,21,24- pentaazapentacyclo[16.5.2.0^{2,6}.0^{7,12}.0^{21,25}]pentacosa- 1(24),7,9,11,18(25),19,22-heptaen-16-yl]ethan-1- one |
| 22 | 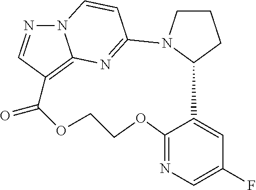 [SMILES: O=C1OCCOc2ncc(F)cc2[C@H]2CCCN2c2ccn3ncc1c3n2] | (6R)-9-fluoro-13,16-dioxa-2,11,20,21,24- pentaazapentacyclo[16.5.2.0^{2,6}.0^{7,12}.0^{21,25}]- pentacosa-1(24),7,9,11,18(25),19,22-heptaen-17- one |
| 23 | 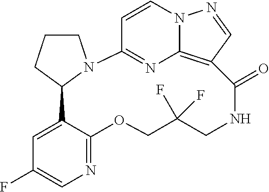 [SMILES: O=C1NCC(F)(F)COc2ncc(F)cc2[C@H]2CCCN2c2ccn3ncc1c3n2] | (6R)-9,15,15-trifluoro-13-oxa-2,11,17,21,22,25- hexaazapentacyclo[17.5.2.0^{2,6}.0^{7,12}.0^{22,26}]hexacosa- 1(25),7,9,11,19(26),20,23-heptaen-18-one |
| 24 | 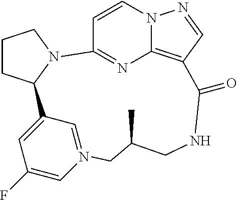 | (6R,13S)-9-fluoro-13-methyl-2,11,15,19,20,23- hexaazapentacyclo[15.5.2.^{17,11}.0^{2,6}.0^{20,24}]pentacosa- 1(23),7,9,17(24),18,21-hexaene-16,25-dione |
| 25 | 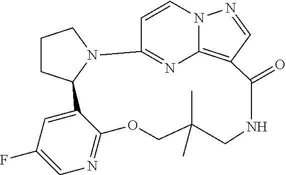 [SMILES: CC1(C)CNC(=O)c2cnn3ccc(nc23)N2CCC[C@@H]2c2cc(F)cnc2OC1] | (6R)-9-fluoro-15,15-dimethyl-13-oxa- 2,11,17,21,22,25- hexaazapentacyclo[17.5.2.0^{2,6}.0^{7,12}.0^{22,26}]hexacosa- 1(25),7,9,11,19(26),20,23-heptaen-18-one |
| 26 | 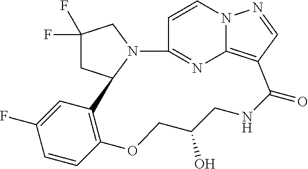 [SMILES: O=C1NC[C@H](O)COc2ccc(F)cc2[C@H]2CC(F)(F)CN2c2ccn3ncc1c3n2] | (15S)-4,4,9-trifluoro-15-hydroxy-13-oxa- 2,17,21,22.25- pentaazapentacyclo[17.5.2.0^{2,6}.0^{7,12}.0^{22,26}]hexacosa- 1(25),7(12),8,10,19(26),20,23-heptaen-18-one |
| 27 | 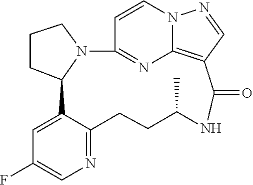 [SMILES: C[C@H]1CCc2ncc(F)cc2[C@H]2CCCN2c2ccn3ncc(c3n2)C(=O)N1] | (6R,15S)-9-fluoro-15-methyl-2,11,16,20,21,24- hexaazapentacyclo[16.5.2.0^{2,6}.0^{7,12}.0^{21,25}]pentacosa- 1(24),7,9,11,18(25),19,22-heptaen-17-one |
| 28 | 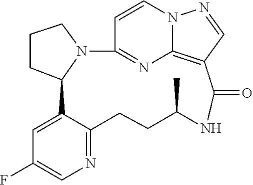 [SMILES: C[C@@H]1CCc2ncc(F)cc2[C@H]2CCCN2c2ccn3ncc(c3n2)C(=O)N1] | (6R,15R)-9-fluoro-15-methyl-2,11,16,20,21,24- hexaazapentacyclo[16.5.2.0^{2,6}.0^{7,12}.0^{21,25}]pentacosa- 1(24),7,9,11,18(25),19,22-heptaen-17-one |

Additional examples of Trk inhibitors are described in U.S. Patent Application Ser. No. 62/080,374, International Application Publication Nos. WO 11/006074, WO 11/146336, WO 10/033941, and WO 10/048314, and U.S. Pat. Nos. 8,933,084, 8,791,123, 8,637,516, 8,513,263, 8,450,322, 7,615,383, 7,384,632, 6,153,189, 6,027,927, 6,025,166, 5,910,574, 5,877,016, and 5,844,092, each of which is herein incorporated by reference in its entirety. Additional Trk inhibitors are known in the art.

In some embodiments, a Trk inhibitor is selected from the group consisting of: entrectinib (N-[5-(3,5-difluoro-benzyl)-1H-indazol-3-yl]-4-(4-methylpiperazin-1-yl)-2-(tetrahydro-pyran-4-ylamino)-benzamide); (S)—N-(5-((R)-2-(2,5-difluorophenyl)pyrrolidin-1-yl)pyrazolo[1,5-a]pyrimidin-3-yl)-3-hydroxypyrrolidine-1-carboxamide sulfate; cabozantinib ((N-(4-((6,7-Dimethoxyquinolin-4-yl)oxy)phenyl)-N′-(4-fluorophenyl)cyclopropane-1,1-dicarboxamide)); dovatinib (4-amino-5-fluoro-3-[6-(4-methylpiperazin-1-yl)-1H-benzimidazol-2-yl]quinolin-2(1H)-one mono 2-hydroxypropanoate hydrate); belizatinib (4-fluoro-N-(6-((4-(2-hydroxypropan-2-yl)piperidin-1-yl)methyl)-1-((1s,4s)-4-(isopropylcarbamoyl)cyclohexyl)-1H-benzo[d]imidazol-2-yl)benzamide); sitravatinib (N-(3-fluoro-4-((2-(5-(((2-methoxyethyl)amino)methyl)pyridin-2-yl)thieno[3,2-b]pyridin-7-yl)oxy)phenyl)-N-(4-fluorophenyl)cyclopropane-1,1-dicarboxamide); PLX7486; altiratinib (N-(4-((2-(cyclopropanecarboxamido)pyridin-4-yl)oxy)-2,5-difluorophenyl)-N-(4-fluorophenyl)cyclopropane-1,1-dicarboxamide); and AZD7451 ((S)—N-(1-(5-fluoropyrimidin-2-yl)ethyl)-3-(5-isopropoxy-1H-pyrazol-3-yl)-3H-imidazo[4,5-b]pyridin-5-amine)). For example, a first Trk inhibitor can be entrectinib, TPX-0005, PLX7486, or (S)—N-(5-((R)-2-(2,5-difluorophenyl)pyrrolidin-1-yl)pyrazolo[1,5-a]pyrimidin-3-yl)-3-hydroxypyrrolidine-1-carboxamide sulfate (or a polymorph thereof).

### Immunotherapy

The term “immunotherapy” refers to an agent that modulates the immune system. In some embodiments, an immunotherapy can increase the expression and/or activity of a regulator of the immune system. In some embodiments, an immunotherapy can decrease the expression and/or activity of a regulator of the immune system. In some embodiments, an immunotherapy can recruit and/or enhance the activity of an immune cell.

In some embodiments, the immunotherapy is a cellular immunotherapy (e.g., adoptive T-cell therapy, dendritic cell therapy, natural killer cell therapy). In some embodiments, the cellular immunotherapy is sipuleucel-T (APC8015; Provenge™; Plosker (2011) Drugs 71(1): 101-108). In some embodiments, the cellular immunotherapy includes cells that express a chimeric antigen receptor (CAR). In some embodiments, the cellular immunotherapy is a CAR-T cell therapy. In some embodiments, the CAR-T cell therapy is tisagenlecleucel (Kymriah™).

In some embodiments, the immunotherapy is an antibody therapy (e.g., a monoclonal antibody, a conjugated antibody). In some embodiments, the antibody therapy is bevacizumab (Mvasti™, Avastin®), trastuzumab (Herceptin®), avelumab (Bavencio®), rituximab (MabThera™, Rituxan®), edrecolomab (Panorex), daratumuab (Darzalex®), olaratumab (Lartruvo™), ofatumumab (Arzerra®), alemtuzumab (Campath®), cetuximab (Erbitux®), oregovomab, pembrolizumab (Keytruda®), dinutiximab (Unituxin®), obinutuzumab (Gazyva®), tremelimumab (CP-675,206), ramucirumab (Cyramza®), ublituximab (TG-1101), panitumumab (Vectibix®), elotuzumab (Empliciti™), avelumab (Bavencio®), necitumumab (Portrazza™), cirmtuzumab (UC-961), ibritumomab (Zevalin®), isatuximab (SAR650984), nimotuzumab, fresolimumab (GC1008), lirilumab (INN), mogamulizumab (Poteligeo®), ficlatuzumab (AV-299), denosumab (Xgeva®), ganitumab, urelumab, pidilizumab or amatuximab.

In some embodiments, the immunotherapy is an antibody-drug conjugate. In some embodiments, the antibody-drug conjugate is gemtuzumab ozogamicin (Mylotarg™), inotuzumab ozogamicin (Besponsa®), brentuximab vedotin (Adcetris®), ado-trastuzumab emtansine (TDM-1; Kadcyla®), mirvetuximab soravtansine (IMGN853) or anetumab ravtansine

In some embodiments, the immunotherapy includes blinatumomab (AMG103; Blincyto®) or midostaurin (Rydapt).

In some embodiments, the immunotherapy includes a toxin. In some embodiments, the immunotherapy is denileukin diftitox (Ontak®).

In some embodiments, the immunotherapy is a cytokine therapy. In some embodiments, the cytokine therapy is an interleukin 2 (IL-2) therapy, an interferon alpha (IFNα) therapy, a granulocyte colony stimulating factor (G-CSF) therapy, an interleukin 12 (IL-12) therapy, an interleukin 15 (IL-15) therapy, an interleukin 7 (IL-7) therapy or an erythropoietin-alpha (EPO) therapy. In some embodiments, the IL-2 therapy is aldesleukin (Proleukin®). In some embodiments, the IFNα therapy is interferon alfa-2b (e.g., IntronA®) or interferon alfa-2a (e.g., Roferon-A®). In some embodiments, the G-CSF therapy is filgrastim (Neupogen®).

In some embodiments, the immunotherapy is an immune checkpoint inhibitor. In some embodiments, the immunotherapy includes one or more immune checkpoint inhibitors. In some embodiments, the immune checkpoint inhibitor is a CTLA-4 inhibitor, a PD-1 inhibitor or a PD-L1 inhibitor. In some embodiments, the CTLA-4 inhibitor is ipilimumab (Yervoy®) or tremelimumab (CP-675,206). In some embodiments, the PD-1 inhibitor is pembrolizumab (Keytruda®) or nivolumab (Opdivo®). In some embodiments, the PD-L1 inhibitor is atezolizumab (Tecentriq®), avelumab (Bavencio®) or durvalumab (Imfinzi™).

In some embodiments, the immunotherapy is mRNA-based immunotherapy. In some embodiments, the mRNA-based immunotherapy is CV9104 (see, e.g., Rausch et al. (2014) Human Vaccin Immunother 10(11): 3146-52; and Kubler et al. (2015) J. Immunother Cancer 3:26).

In some embodiments, the immunotherapy is bacillus Calmette-Guerin (BCG) therapy.

In some embodiments, the immunotherapy is an oncolytic virus therapy. In some embodiments, the oncolytic virus therapy is talimogene alherparepvec (T-VEC; Imlygic®).

In some embodiments, the immunotherapy is a cancer vaccine. In some embodiments, the cancer vaccine is a human papillomavirus (HPV) vaccine. In some embodiments, the HPV vaccine is a recombinant human papillomavirus vaccine [types 6, 11, 16, and 18] (Gardasil®); a recombinant human papillomavirus vaccine [types 6, 11, 16, 18, 31, 33, 45, 52, and 58] (Gardasil9®); or a recombinant human papillomavirus vaccine [types 16 and 18] (Cervarix®). In some embodiments, the cancer vaccine is a hepatitis B virus (HBV) vaccine. In some embodiments, the HBV vaccine is Engerix-B®, Recombivax HB® or GS-4774 (GI-13020 or Tarmogen®). In some embodiments, the cancer vaccine is a combination Hepatitis A and Hepatitis B vaccine (e.g., Twinrix®) or a combination diphtheria, tetanus, pertussis, hepatitis B virus, and poliomyelitis vaccine (e.g., Pediarix®). In some embodiments, the cancer vaccine is dasiprotimut-T (BiovaxID®), an HSPPC-96 vaccine (e.g., Oncophage®), GVAX, ADXS11-001, ALVAC-CEA, rilimogene galvacirepvec/rilimogene glafolivec (PROSTVAC®), CDX-110 (Rindopepimut®), CimaVax-EGF, lapuleucel-T (APC8024; Neuvenge™), GRNVAC1, GRNVAC2, GRN-1201, hepcortespenlisimut-L (Hepko-V5), a dendritic cell vaccine (e.g., DCVax-L®, ICT-107), SCIB1, BMT CTN 1401, PrCa VBIR, PANVAC, a prostate cancer vaccine (e.g., ProstAtak®), DPX-Survivac, or viagenpumatucel-L (HS-110).

In some embodiments, the immunotherapy is a peptide vaccine. In some embodiments, the peptide vaccine is nelipepimut-S (E75) (NeuVax™), IMA901, or SurVaxM (SVN53-67). In some embodiments, the cancer vaccine is an immunogenic personal neoantigen vaccine (see, e.g., Ott et al. (2017) Nature 547: 217-221; Sahin et al. (2017) Nature 547: 222-226). In some embodiments, the cancer vaccine is RGSH4K, or NEO-PV-01.

In some embodiments, the cancer vaccine is a DNA-based vaccine. In some embodiments, the DNA-based vaccine is a mammaglobin-A DNA vaccine (see, e.g., Kim et al. (2016) Oncolmmunology 5(2): e1069940).

### Methods of Treating Cancer

Compound 1, or a pharmaceutically acceptable salt, amorphous, or polymorph form thereof, or a pharmaceutical composition thereof, as described herein, is also useful for treating cancer in a mammal. Particular examples include neuroblastoma, ovarian, pancreatic, colorectal, and prostate cancer.

Another embodiment of the present disclosure provides a method of treating or preventing cancer in a mammal, comprising administering to said mammal a pharmaceutical composition comprising a compounding agent as disclosed herein and Compound 1 or a solid form thereof, crystalline form thereof, or solvate or hydrate thereof, or a salt of Compound 1 or solid form thereof, crystalline form thereof, or solvate or hydrate thereof, as described herein, in an amount effective to treat or prevent the cancer. In one embodiment, the cancer is neuroblastoma. In one embodiment, the cancer is ovarian cancer. In one embodiment, the cancer is pancreatic cancer. In one embodiment, the cancer is colorectal cancer. In one embodiment, the cancer is prostate cancer. In one embodiment, the method comprises treating the cancer in a subject. In one embodiment, the method comprises preventing the cancer in a subject.

Pharmaceutical compositions comprising a compounding agent as disclosed herein and Compound 1 or a solid form thereof, crystalline form thereof, or solvate or hydrate thereof, or a salt of Compound 1 or solid form thereof, crystalline form thereof, or solvate or hydrate thereof, as described herein, may be administered alone as a sole therapy or can be administered in addition with one or more other substances and/or treatments that work by the same or a different mechanism of action. Examples include anti-inflammatory compounds, steroids (e.g., dexamethasone, cortisone and fluticasone), analgesics such as NSAIDs (e.g., aspirin, ibuprofen, indomethacin, and ketoprofen), and opioids (such as morphine), and chemotherapeutic agents. These agents may be administered with a pharmaceutical composition comprising a compounding agent as disclosed herein and Compound 1 or a solid form thereof, crystalline form thereof, or solvate or hydrate thereof, or a salt of Compound 1 or solid form thereof, crystalline form thereof, or solvate or hydrate thereof, as described herein, as part of the same or separate dosage forms, via the same or different routes of administration, and on the same or different administration schedules according to standard pharmaceutical practice known to one skilled in the art.

In the field of medical oncology, it is normal practice to use a combination of different forms of treatment to treat each patient with cancer. In medical oncology the other component(s) of such conjoint treatment in addition to compositions of the present disclosure may be, for example, surgery, radiotherapy, chemotherapy, signal transduction inhibitors and/or immunotherapy (e.g., monoclonal antibodies).

Accordingly, a pharmaceutical composition comprising a compounding agent as disclosed herein and Compound 1 or a solid form thereof, crystalline form thereof, or solvate or hydrate thereof, or a salt of Compound 1 or solid form thereof, crystalline form thereof, or solvate or hydrate thereof, as described herein, may be administered in combination with one or more agents selected from mitotic inhibitors, alkylating agents, anti-metabolites, antisense DNA or RNA, intercalating antibiotics, growth factor inhibitors, signal transduction inhibitors, cell cycle inhibitors, enzyme inhibitors, retinoid receptor modulators, proteasome inhibitors, topoisomerase inhibitors, biological response modifiers, anti-hormones, angiogenesis inhibitors, cytostatic agents anti-androgens, targeted antibodies, HMG-CoA reductase inhibitors, and prenyl-protein transferase inhibitors. These agents may be administered with one or more Compound 1, its solid form, crystalline form, solvate or hydrate, or a salt of Compound 1, or solid form, crystalline form, solvate or hydrate of the salt as described herein, as part of the same or separate dosage forms, via the same or different routes of administration, and on the same or different administration schedules according to standard pharmaceutical practice known to one skilled in the art.

In some embodiments, provided herein is a method for treating a patient diagnosed with a Trk-associated cancer, comprising administering to the patient a therapeutically effective amount of one or more Trk inhibitors and optionally an immunotherapy agent. The Trk family of neurotrophin receptors, TrkA, TrkB, and TrkC (encoded by NTRK1, NTRK2, and NTRK3 genes, respectively) and their neurotrophin ligands regulate growth, differentiation and survival of neurons. Dysregulation in a NTRK gene, a Trk protein, or expression or activity, or level of the same, such as translocations involving the NTRK kinase domain, mutations involving the Trk ligand-binding site, amplifications of a NTRK gene, Trk mRNA splice variants, and Trk autocrine/paracrine signaling are described in a diverse number of tumor types and may contribute to tumorigenesis. Recently NTRK1 fusions were described in a subset of adenocarcinoma lung cancer patients. Translocations in NTRK1, NTRK2, and NTRK3 that lead to the production of constitutively-active TrkA, TrkB, and TrkC fusion proteins are oncogenic and prevalent in a wide array of tumor types, including lung adenocarcinoma, thyroid, head and neck cancer, glioblastoma, and others.

In some embodiments, the dysregulation in a NTRK gene, a Trk protein, or expression or activity, or level of the same, includes overexpression of wild-type TrkA, TrkB, or TrkC (e.g., leading to autocrine activation). In some embodiments, the dysregulation in a NTRK gene, a Trk protein, or expression or activity, or level of the same, includes overexpression, activation, amplification or mutation in a chromosomal segment comprising the NTRK1, NTRK2, or NTRKR3 gene or a portion thereof. In some embodiments, the dysregulation of a NTRK gene, a Trk protein, or expression or activity, or level of the same, includes one or more chromosome translocations or inversions resulting in NTRK1, NTRK2, or NTRK3 gene fusions, respectively. In some embodiments, the dysregulation of a NTRK gene, a Trk protein, or expression or activity, or level of the same, is a result of genetic translocations in which the expressed protein is a fusion protein containing residues from a non-TrkA partner protein and TrkA, a non-TrkB partner protein and TrkB, or a non-TrkC partner protein and TrkC proteins, and include a minimum of a functional TrkA, TrkB, or TrkC kinase domain, respectively.

In some embodiments, a TrkA fusion protein is one of the TrkA fusion proteins shown in Table 2.

TABLE 2
Exemplary TrkA Fusion Proteins and Cancers

| | | Non-limiting Exemplary TrkA |
|---|---|---|
| | | Fusions and Synonyms of |
| Fusion Protein | Non-TrkA Fusion Partner | Associated Cancer(s) |
| TP53-TrkA^{1, 2} | Tumor Protein P53 | Spitzoid Neoplasms³, Spitz |
| | | Tumors¹, Pediatric High-Grade |
| | | Glioma² |
| LMNA-TrkA¹⁷ | Lamin A/C | Spitzoid Neoplasms¹, Spitz |
| | | Tumors⁴, Sarcoma⁶³ (e.g., Adult |
| | | Soft Tissue Sarcoma¹², Spindle |
| | | Cell Sarcoma including Uterine |
| | | Spindle Cell Sarcoma⁶ and |
| | | Paediatric Haemangiopericytoma- |
| | | Like Sarcoma⁵), Congenital |
| | | Infantile Fibrosarcoma^{7, 65}, |
| | | Colorectal Cancer^{8, 18}, Pediatric |
| | | Soft Tissue Tumor⁹, Soft Tissue |
| | | Primitive Neuroectodermal |
| | | Tumor⁶⁴, Lipofibromatosis-like |
| | | Neural Tumor (LPF-NT)^{10, 11}, |
| | | Histiocytic Neoplasms^{13, 14} (e.g., |
| | | Non-Langerhan Histocytosis¹⁵), |
| | | Melanoma¹⁷ (e.g., Skin Cutaneous |
| | | Melanoma⁵⁷) |
| CD74-TrkA¹⁹ | MHC class II invariant chain | Lung Adenocarcinoma²⁰ |
| TFG-TrkA (TRK- | TRK-Fused Gene | Papillary Thyroid Carcinoma |
| T3)²¹ | | (PTC)^{22, 54}, Histiocytic |
| | | Neoplasms¹⁴, Thyroid |
| | | Carcinoma⁵⁷ |
| TPM3-TrkA²¹ | Tropomyosin 3 | Non-Small Cell Lung Cancer⁶³, |
| | | Papillary Thyroid Carcinoma |
| | | (PTC)^{21, 53}, Sarcoma^{37, 57} (e.g., |
| | | Spindle Cell Uterine Sarcoma⁶, |
| | | Pediatric Spindle Cell Sarcoma²⁴, |
| | | Uterine Leiomyosarcoma |
| | | (LMS)²⁵, Spindle Cell Sarcoma |
| | | with a Prominent |
| | | Myopericytic/Haemangiopericytic |
| | | Pattern⁵), Glioblastoma⁶³, |
| | | Colorectal Cancer (CRC)^{8, 16, 51, 56}, |
| | | Soft Tissue Schwannoma¹², |
| | | Spitzoid Melanocytic Tumors²³, |
| | | Lipofibromatosis-Like Neural |
| | | Tumors (LPF-NT)¹¹, |
| | | Lipofibromatosis (LPF)²⁶, |
| | | Bladder Urothelial Carcinoma⁵⁷, |
| | | Gall Bladder Cancer⁶³, |
| | | Cholangiocarcinoma⁶³ |
| NFASC-TrkA³⁰ | Neurofascin | Gliobastoma multiforme |
| | | (GBM)^{27, 30, 60} |
| BCAN-TrkA²⁷ | Brevican | Glioma (e.g., Glioblastoma |
| | | Multiforme (GBM)²⁷, High-Grade |
| | | Glioma²⁸, Glioneuronal Tumor^{29, 61}, |
| | | Pilocytic Astrocytoma³¹) |
| MPRIP-TrkA^{19, 32} | Myosin Phosphatase Rho | Lung Adenocarcinoma^{15, 33} |
| | Interacting Protein or Rho | |
| | Interacting Protein 3 | |
| TPR-TrkA (e.g., | Translocated Promoter Region, | Papillary Thyroid Carcinoma |
| TRK-T1 or TRK- | Nuclear Basket Protein | (PTC)^{62, 67}, Post-Chernobyl |
| T2)²⁷ | | Radiation-Induced Thyroid |
| | | cancer⁴⁵, Colorectal Cancer |
| | | (CRC)³⁴, LPF-Like Neural |
| | | Tumors², Sporadic Pediatric |
| | | Differentiated Thyroid |
| | | Carcinomas (DTC)³⁵, Spindle |
| | | Cell Uterine Sarcoma⁶, |
| | | Myofibroma/Myofibromatosis²⁶, |
| | | Dendritic Cell Neoplasm¹⁴ |
| RFWD2-TrkA³⁶ | Ring Finger and WD Repeat | Large Cell Neuroendrocine |
| | Domain 2 | Cancer (LCNEC)³⁶ |
| IRF2BP2-TrkA⁴⁴ | Interferon Regulatory Factor 2 | Thyroid Gland Carcinoma^{44, 59}, |
| | Binding Protein 2 | Thyroid Carcinoma⁵⁷, Non-Small |
| | | Cell Lung Cancer⁶³ |
| SQSTM1-TrkA⁴⁴ | Sequestosome 1 | Thyroid Cancer (e.g., Papillary |
| | | Thyroid Cancer⁶³, Thyroid Gland |
| | | Carcinoma⁵⁹), Soft Tissue |
| | | Fibrosarcoma¹², Non-Small Cell |
| | | Lung Cancer^{38, 39}, Lung |
| | | Adenocarcinoma⁵⁸ |
| SSBP2-TrkA⁴⁴ | Single-Stranded DNA Binding | Thyroid Cancer⁵⁷ (e.g., Papillary |
| | Protein 2 | Thyroid Cancer); Thyroid Gland |
| | | Carcinoma⁵⁹ |
| RABGAP1L- | RAB GTPase Activating Protein | Intrahepatic Cholangiocarcinoma |
| TrkA⁴¹ | 1-Like | (ICC)⁴¹ |
| C18ORF8-TrkA⁴⁷ | Chromosome 18 Open Reading | Non-Small Cell Lung Cancer |
| | Frame 8 | (NSCLC)⁴⁷ |
| RNF213-TrkA⁴⁷ | Ring Finger Protein 213 | Non-Small Cell Lung Cancer |
| | | (NSCLC)⁴⁷ |
| TBC1D22A- | TBC1 Domain Family, Member | Non-Small Cell Lung Cancer |
| TrkA⁴⁷ | 22A | (NSCLC)⁴⁷ |
| C20ORF112- | Chromosome 20 Open Reading | Non-Small Cell Lung Cancer |
| TrkA⁴⁷ | Frame 112 | (NSCLC)⁴⁷ |
| DNER-TrkA⁴⁷ | Delta/Notch-Like EGF Repeat | Non-Small Cell Lung Cancer |
| | Containing | (NSCLC)⁴⁷ |
| ARHGEF2- | Rho Guanine Nucleotide | Glioblastoma^{42, 43}, Sarcoma⁵⁷ |
| TrkA^{42, 57} | Exchange Factor 2 | |
| CHTOP-TrkA⁴² | Chromatin Target of PRMT1 | Glioblastoma⁴² |
| PPL-TrkA⁴² | Periplakin | Thyroid Carcinoma⁴² |
| PLEKHA6-TrkA | Pleckstrin Homology Domain- | |
| | Containing Family A Member 6 | |
| PEARl-TrkA⁶³ | Platelet Endothelial Aggregation | Sarcoma⁶³, Breast Cancer⁶³ |
| | Receptor 1 | |
| MRPL24-TrkA⁶³ | 39S Ribosomal Protein L24, | Non-Small Cell Lung Cancer⁶³ |
| | Mitochondrial | |
| MDM4-TrkA⁶³ | Human Homolog of Mouse | Breast Cancer⁶³ |
| | Double Minute 4 | |
| LRRC71-TrkA⁶³ | Leucine Rich Repeat Containing | Uterus Carcinoma⁶³ |
| | 71 | |
| GRIPAP1-TrkA⁶³ | GRIP1 Associated Protein 1 | Non-Small Cell Lung Cancer⁶³ |
| TAF-TrkA⁶³ | | Papillary Thyroid Carcinoma⁶³ |
| EPS15-TrkA | Epidermal Growth Factor | |
| | Receptor Substrate 15 | |
| DYNC2H1- | Dynein, Cytoplasmic 2, Heavy | Sarcoma |
| TrkA⁴⁴ | Chain 1 | |
| CEL-TrkA⁵⁷ | Carboxyl Ester Lipase | Pancreatic adenocarcinoma |
| | | sample⁵⁷ |
| EPHB2-TrkA⁴⁴ | EPH Receptor B2 | Lower Grade Glioma^{44, 57} |
| TGF-TrkA⁴⁶ | Transforming Growth Factor | Papillary Thyroid Cancer (PTC) |
| NELL1-TrkA⁴⁷ | Cytoplasmic Protein That | Non-Small Cell Lung Cancer |
| | Contains Epidermal Growth | (NSCLC)⁴⁷ |
| | Factor (Egf)-Like Repeats | |
| EPL4-TrkA⁴⁷ | EPH-Related Receptor Tyrosine | Non-Small Cell Lung Cancer |
| | Kinase Ligand 4/Ephrin-A4 | (NSCLC)⁴⁷ |
| | Protein | |
| CTNND2-TrkA⁴⁷ | Catenin (Cadherin-Associated | Non-Small Cell Lung Cancer |
| | Protein), Delta 2 | (NSCLC)⁴⁷ |
| TCEANC2- | Transcription Elongation Factor A | Non-Small Cell Lung Cancer |
| TrkA⁴⁷ | (Sll) N-Terminal And Central | (NSCLC)⁴⁷ |
| | Domain | |
| SCYL3-TrkA⁴⁸ | SCY1 Like Pseudokinase 3 | Colorectal Cancer |
| AMOTL2-TrkA⁴⁹ | | Non-small cell lung cancer |
| MEF2D-TrkA⁵⁰ | | |
| L7a-TrkA⁵⁵ (Trk- | | Breast Carcinoma (human cell |
| 2h) | | line)⁵⁵ |
| ZBTB7B-TrkA⁵⁷ | | Bladder Urothelial Carcinoma⁵⁷ |
| TRIM63-TrkA⁶⁶ | | Non-Spitzoid Metastasizing |
| | | Melanomas⁶⁶ |
| DDR2-TrkA⁶⁶ | | Non-Spitzoid Metastasizing |
| | | Melanomas⁶⁶ |
| GON4L-TrkA⁶⁶ | | Non-Spitzoid Metastasizing |
| | | Melanomas⁶⁶ |
| PDE4DIP-TrkA | | Soft Tissue Sarcoma |
| | | (Myopericytoma) |
| NTRK1- | | |
| P2RY8⁵²* | | |
| CTRC-TrkA | Chymotrypsin C | Pancreatic cancer |
| VANGL2-TrkA⁶⁸ | | Non-Small Cell Lung Cancer⁶⁸ |

*The transcript of this fusion was not detected.
¹Wiesner et al., Nature Comm. 5: 3116, 2014.
²Wu et al., Nat. Genet. 46: 444-450, 2014.
³U.S. Patent Application Publication No. 2016/0010068.
⁴P.C.T. Patent Application Publication No. WO 2013/059740.
⁵Haller et al., J. Path. 238(5): 700-10, 2016.
⁶Chiang et al., Am. J. Surg. Pathol. doi: 10.1097/PAS.0000000000001055, 2018.
⁷Wong et al., J. Natl. Cancer Inst. 108(1): doi: 10.1093/jnci/djv307, 2016.
⁸Park et al., Oncotarget. 7(7): 8399-412, 2016.
⁹Kohsaka et al., Hum. Pathol. 72: 167-173, 2017.
¹⁰Bartenstein et al., JAAD Case Reports. 4(2): 185-188, 2018.
¹¹Agaram et al., Am. J. Surg. Pathol. 40(10): 1407-1416, 2016.
¹²Doebele et al., Cancer Discov. 5(10): 1049-1057, 2015.
¹³Durham et al. Blood. 126(23): 481, 2015.
¹⁴Taylor et al., Abstract Number: 794. Meeting Info: 59th Annual Meeting of the American Society of Hematology, ASH 2017. Atlanta, GA, United States, 2017.
¹⁵U.S. Patent Application Publication No. 2016/0009785.
¹⁶Sartore-Bianchi et al., J. Natl. Cancer Inst. 108(1). doi: 10.1093/jnci/djv306, 2015.
¹⁷U.S. Patent Application Publication No. 2014/0336236.
¹⁸P.C.T. Patent Application Publication No. WO 2015/064621.
¹⁹Vaishnavi et al., Nature Med. 19: 1469-1472, 2013.
²⁰Doebele et al., Abstract Number: 8023. Meeting Info: 2013 Annual Meeting of the American Society of Clinical Oncology, ASCO. Chicago, IL, United States, 2013.
²¹Greco et al., Mol. Cell. Endocrinol. 28: 321, 2010.
²²Greco et al., Mol. Cell. Biol. 15(11): 6118-6127, 1995.
²³Wu et al., Mod Pathol. 29(4): 359-69, 2016.
²⁴Kao et al., Am. J. Surg. Pathol. 42(1): 28-38, 2018.
²⁵Elvin et al., Abstract Number: 319. Meeting Info: 26 EORTC - NCI - AACR Symposium on Molecular Targets and Cancer Therapeutics. Barcelona, Spain, 2014.
²⁶Agaram, et al., Abstract Number: 33. Meeting Info: 105th Annual Meeting of the United States and Canadian Academy of Pathology, USCAP 2016. Seattle, WA, United States, 2016.
²⁷Kim et al., PloS ONE. 9(3): e91940, 2014.
²⁸Cook et al., Nat. Comm. 8(15987). DOI 10.1038/ncomms15987, 2017.
²⁹Alvarez-Breckenridge et al., NPJ Precision Oncology. 1(5) doi: 10.1038/s41698-017-0009-y, 2017.
³⁰U.S. Patent Application Publication No. 2016/0108380.
³¹Subramaniam et al., Meeting Info: 2017 Annual Meeting of the American Society of Clinical Oncology, ASCO. Chicago, IL, United States, 2017.
³²U.S. Patent Application Publication No. 2015/0073036.
³³U.S. Patent Application Publication No. 2015/0218652.
³⁴Créancier et al., Cancer Lett. 365(1): 107-111, 2015.
³⁵Picarsic et al., Pediatr. Dev. Pathol. 19(2): 115-22, 2016.
³⁶Femandez-Cuesta et al., Abstract Number: 1531. Meeting Info: 105th Annual Meeting of the American Association for Cancer Research, AACR 2014. San Diego, CA, United States, 2014.
³⁷Stransky et al., Nature Comm. 5: 4846, 2014.
³⁸Drilon et al., Abstract Number: CT007; 107^{th} Annual Meeting of the American Association for Cancer Research, AACR 2016. New Orleans, LA, 2016.
³⁹Farago et al., Abstract Number: MINI30.09. Meeting Info: 16th World Conference on Lung Cancer. Denver, CO, United States, 2015.
⁴¹Ross et al., Oncologist 19: 235-242, 2014.
⁴²Zheng et al., Nat. Med. 20(12): 1479-84, 2014.
⁴³P.C.T. Patent Application Publication No. WO 2015/039006.
⁴⁴U.S. Patent Application Publication No. 2015/0315657.
⁴⁵Ricarte-Filho et al., J. Clin. Invest. 123(11): 4935-44, 2013.
⁴⁶U.S. Patent Application Publication No. 2015/0283132.
⁴⁷U.S. Patent Application Publication No. 2017/0114415.
⁴⁸Milione et al., Oncotarget, 8(33): 55353-55360, 2017.
⁴⁹Chen et al., Abstract Number: 40. Meeting Info: 3rd Molecular Analysis for Personalised Therapy Conference, MAP 2017. Zurich, Switzerland, 2017.
⁵⁰Gatalica et al., Abstract Number: A047. Meeting Info: AACR-NCI-EORTC International Conference: Molecular Targets and Cancer Therapeutics 2017. Philadelphia, PA, United States, 2017.
⁵¹Martin-Zanca et al., Nature. 319(6056): 743-8, 1986.
⁵²Hechtman et al., Mol. Cancer Res. 14(3): 296-301, 2016.
⁵³Butti et al., Genomics. 28(1): 15-24, 1995.
⁵⁴Brzezianska et al., Neuro. Endocrinol. Lett. 28(3): 221-9, 2007.
⁵⁵Ziemiecki et al., EMBO J. 9(1): 191-6, 1990.
⁵⁶Ardini et al., Mol. Oncol. 8(8): 1495-1507, 2014.
⁵⁷Gao et al., Cell Rep. 23(1): 227-238.e3, 2018.
⁵⁸Farago et al., J. Thorac Oncol. 10(12): 1670-1674, 2015.
⁵⁹U.S. Patent Application Publication No. 2014/0315199.
⁶⁰Frattini et al., Nat. Genet. 45(10): 1141-1149, 2013.
⁶¹Bastianos et al., Abstract Number: OS06.4. Meeting Info: 5th Quadrennial Meeting of the World Federation of Neuro-Oncology Societies, WFNOS. Zurich, Switzerland, 2017.
⁶²Greco et al., Oncogene. 7(2): 237-42, 1992.
⁶³Wei et al., Abstract Number: 78. Meeting Info: 28th EORTC-NCI-AACR Symposium on Molecular Targets and Cancer Therapeutics. Munich, Germany, 2016.
⁶⁴Pavlick et al., Pediatr. Blood Cancer. 64(8). 2017.
⁶⁵Wong et al., J. Natl. Cancer Inst. 108(1), 2015.
⁶⁶ezcano et al., Am. J. Surg. Pathol. doi: 10.1097/PAS.0000000000001070, 2018.
⁶⁷Greco et al., Genes Chromosomes Cancer. 19(2): 112-23, 1997.
⁶⁸Zehir et al., Nat. Med. 23(6): 703-713, 2017.

In some embodiments, the dysregulation of a NTRK gene, a Trk protein, or expression or activity or level of any of the same, includes at least one point mutation in a NTRK gene that results in the production of a TrkA protein that has one or more amino acid substitutions, insertions, or deletions as compared to the wildtype TrkA protein (see, for example, the point mutations listed in Table 3). An exemplary wildtype TrkA polypeptide is SEQ ID NO: 1, an exemplary wildtype TrkB polypeptide is SEQ ID NO: 5, and an exemplary TrkC polypeptide is SEQ ID NO: 7.

TABLE 3
TrkA Kinase Protein Amino Acid
Substitutions/Insertions/Deletions^{A}

Amino acid position 6 (e.g., R6W³)
Amino acid position 33 (e.g., R33W⁴)
Amino acid position 336 (e.g., A336E)
Amino acid position 337 (e.g., A337T)
Amino acid position 324 (e.g., R324Q, R324W)
Amino acid position 420 (e.g., V420M)
Amino acid position 444 (e.g., R444Q, R444W)
Amino acid position 517 (e.g., G517R, G517V)
Amino acid position 538 (e.g., K538A)
Amino acid position 542 (e.g., A542V)
Amino acid position 564 (e.g., L564H²)
Amino acid position 568 (e.g., Q568x)
Amino acid position 573 (e.g., V573M⁵)
Amino acid position 583 (e.g., R583H³)
Amino acid position 589 (e.g., F589L⁵, F589C)
Amino acid position 595 (e.g., G595S, G595R¹, G595L²)
Amino acid position 597* (e.g., Q597X⁷)
Amino acid position 598 (e.g., F598L⁵)
Amino acid position 599 (e.g., D596V)
Amino acid position 600 (e.g., F600L)
Amino acid position 602 (e.g., R602x)
Amino acid position 627* (e.g., Q627X⁷)
Amino acid position 633* (e.g., Q633X⁷)
Amino acid position 646 (e.g., F646V, F646I²)
Amino acid position 649 (e.g., R649W, R649L)
Amino acid position 656 (e.g., C656Y, C656F)
Amino acid position 657 (e.g., L657V)
Amino acid position 667 (e.g., G667C¹, G667S)
Amino acid position 676 (e.g., Y676S)
Amino acid position 679 (e.g., D679G²)
Amino acid position 682 (e.g., R682S)
Amino acid position 683 (e.g., V683G)
Amino acid position 699 (e.g., 1699V⁶)
Amino acid position 702 (e.g., R702C)
Amino acid position 744 (e.g., R744H³)
^{A}The TrkA kinase mutations shown above may be activating mutations and/or may confer increased resistance of the TrkA kinase to a TrkA inhibitor e.g., as compared to a wildtype TrkA kinase.
*Q627XC, Q597XC, and Q633XC are from NP_001012331.1G⁸, NP_001007793.1F⁹, and the Reference TrkA sequence¹⁰, respectively.
¹Russo et al., Acquired Resistance to the TRK Inhibitor Entrectinib in Colorectal Cancer, Cancer Discov., Jan; 6(1): 36-44, 2016.
²Fuse et al., Mechanisms of Resistance to NTRK Inhibitors and Therapeutic Strategies in NTRK1-Rearranged Cancers, Mol. Cancer Ther.,. Jan; 6(1): 36-44, 2016.
³Iniguez-Ariza et al., Journal of Clinical Oncology, (20 Jun. 2017) Vol. 35, No. 15, Supp. 1, 2017 Annual Meeting of the American Society of Clinical Oncology, ASCO, 2017.
⁴Zhang et al., Blood 124(21): 1682, 2014. Mutation found in T-cell prolymphocytic leukemia.
⁵PCT Application No. WO2016196141A1.
⁶Deihimi et al., Oncotarget. Jun 20; 8(25): 39945-39962. doi: 10.18632/oncotarget.18098, 2017.
⁷Park et al., Proc. Natl. Acad. Sci. U.S.A. 112(40): 12492-12497, 2015. Mutation found in colorectal cancer.
⁸www.ncbi.nlm.nih.gov/protein/59889558
⁹www.ncbi.nlm.nih.gov/protein/56118210?report=genbank&log$=protalign&blast_rank=3&RID=0
¹⁰Reference TrkA sequence is UniProtKB/Swiss-Prot: P04629.4, and can be found at URL: www.ncbi.nlm.nih.gov/protein/94730402?report=genbank&log$=protalign&blast_rank=0&RID=0

In some embodiments, the dysregulation of a NTRK gene, a Trk protein, or expression or activity, or level of the same, includes one or more deletions, insertions, or point mutation(s) in a TrkA protein. In some embodiments, the dysregulation of a NTRK gene, a Trk protein, or expression or activity, or level of the same, includes a deletion of one or more residues from the TrkA protein, resulting in constitutive activity of the TrkA kinase domain. In some embodiments, the deletion includes a deletion of amino acids 303-377 in TrkA isoform 2.

In some embodiments, the dysregulation of a NTRK gene, a Trk protein, or expression or activity, or level of the same, includes at least one point mutation in a NTRK1 gene that results in the production of a TrkA protein that has one or more amino acid substitutions as compared to the wildtype TrkA protein. In some embodiments, the at least one or more amino acid substitutions are activating mutations (see, for example, the point mutations listed in Table 4 and Table 4a).

TABLE 4
Activating TrkA Point Mutations¹

| | | Exemplary Isoform in |
|---|---|---|
| | | which Mutation is Present |
| Point Mutation | Rationale | (if known) |
| R6W^{I} | | |
| R33W² | | NP_001007793.1⁶ |
| A336E | Near NGF Binding Site | Reference TrkA sequence |
| A337T | Near NGF Binding Site | Reference TrkA sequence |
| R324Q or R324W | Near NGF Binding Site | Unknown |
| V420M | Close to Membrane | Reference TrkA sequence |
| R444Q or R444W | Close to Membrane | Reference TrkA sequence |
| G517R or G517V | P-Loop | Reference TrkA sequence |
| K538A | Activating | Reference TrkA sequence |
| R583H⁹ | | |
| F598L⁵ | | Unknown |
| R649W or R649L | Arginine may stabilize auto- | Reference TrkA sequence |
| | inhibited conformation. | |
| G667C⁴ | Catalytic Domain | Reference TrkA sequence |
| R682S | Activation Loop | Reference TrkA sequence |
| V683G | Activation Loop | Reference TrkA sequence |
| I699V⁸ | | |
| Q627X³, Q597X³, | | NP_001012331.1⁷, |
| Q633X³ | | NP_001007793.1⁶, and |
| | | Reference TrkA sequence, |
| | | respectively |
| R702C | Exposed, may form face-to-face | Reference TrkA sequence |
| | disulfide linked dimer | |
| R744H⁹ | | |

¹Reference TrkA sequence is UniProtKB/Swiss-Prot: P04629.4, and can be found at URL:
www.ncbi.nlm.nih.gov/protein/94730402?report=genbank&log$=protalign&blast_rank=0&RID=0
²Zhang et al., Blood 124(21): 1682, 2014. Mutation found in T-cell prolymphocytic leukemia.
³Park et al., Proc. Natl. Acad. Sci. U.S.A. 112(40): 12492-12497, 2015. Mutation found in colorectal cancer.
⁴Russo et al., Cancer Discov. Jan; 6(1): 36-44, 2016.
⁵PCT Application No. WO2016196141A1.
⁶www.ncbi.nlm.nih.gov/protein/56118210?report=genbank&log$=protalign&blast_rank=3&RID=0
⁷www.ncbi.nlm.nih.gov/protein/59889558
⁸Deihimi et al., Oncotarget. Jun 20; 8(25): 39945-39962. doi: 10.18632/oncotarget.18098, 2017.
⁹Iniguez-Ariza et al., Journal of Clinical Oncology, (20 Jun. 2017) Vol. 35, No. 15, Supp. 1, 2017 Annual Meeting of the American Society of Clinical Oncology, ASCO, 2017.

TABLE 4a
Activating TrkA Point Mutations

| Mutation | | Pediatric Cancer | Reference |
|---|---|---|---|
| C6773T, C7232T, | TrkA | neuroblastoma | Scaruffi et al., |
| C7301T | | | Int. J. Oncol. |
| | | | 14: 935-938, |
| | | | 1999 |

In some embodiments, the dysregulation of a NTRK gene, a Trk protein, or expression or activity, or level of the same, includes a splice variation in a TrkA mRNA which results in an expressed protein that is an alternatively spliced variant of TrkA having at least one residue deleted (as compared to a wild-type TrkA protein) resulting in constitutive activity of the TrkA kinase domain. In some embodiments, an alternatively spliced form of TrkA with constitutive activity has deletions of exons 8, 9, and 11 resulting in an expressed protein missing residues 192-284 and 393-398 relative to TrkA Isoform 2, has a deletion of exon 10 in TrkA, or has a deletion in a NTRK1 gene that encodes a TrkA protein with a 75 amino acid deletion in the transmembrane domain (Reuther et al., Mol. Cell Biol. 20:8655-8666, 2000).

Cancers identified as having dysregulation of a NTRK gene, a Trk protein, or expression or activity, or level of the same, (see references cited herein and also the www.cancer.gov and www.nccn.org websites) include:

(A) Cancers wherein the dysregulation of a NTRK gene, a Trk protein, or expression or activity, or level of the same, includes one or more chromosome translocations or inversions resulting in TrkA fusion proteins, e.g., including:

| Cancer | Standard of Care |
|---|---|
| Non-Small Cell | radiotherapy (e.g., radioiodide therapy, external-beam radiation, |
| Lung Cancer² | or radium 223 therapy), chemotherapeutics as single agents (e.g., |
| | afatinib dimaleate, bevacizumab, carboplatin, cetuximab, |
| | cisplatin, crizotinib, erlotinib, gefitinib, gemcitabine, |
| | methotrexate, paclitaxel, or pemetrexed) or combinations (e.g., |
| | carboplatin-paclitaxel, gemcitabine-paclitaxel, or |
| | chemoradiation) |
| Papillary Thyroid | Radiotherapies (e.g., radioiodide therapy or external-beam |
| Carcinoma¹⁴ | radiation) and chemotherapeutics (e.g., sorafenib, sunitinib, or |
| | pazopanib) |
| Glioblastoma | Chemotherapeutics (e.g., bevacizumab, everolimus, lomustine, or |
| Multiforme¹⁵ | temozolomide) |
| Colorectal | Chemotherapeutics as single agents (e.g., aflibercept, |
| Carcinoma¹⁶ | bevacizumab, capecitabine, cetuximab, fluorouracil, irinotecan, |
| | leucovorin, oxaliplatin, panitumumab, or regorafenib) or |
| | combinations (e.g., folfox, folfiri, capox, folfiri-bevacizumab, |
| | folfiri-cetuximab, or xelox) |
| Melanoma¹² | Chemotherapeutics (e.g., aldesleukin, dabrafenib, dacarbazine, |
| | interferon alfa-2b, ipilimumab, peginterferon alfa-2b, trametinib, |
| | or vemurafenib) |

(B) Cancers wherein the dysregulation of a NTRK gene, a Trk protein, or expression or activity, or level of the same, includes one or more deletions, insertions, or mutations in the TrkA protein, e.g., including:

| Cancer | Standard of care |
|---|---|
| Acute Myeloid | Chemotherapeutics as single agents (e.g., arsenic trioxide, |
| leukemia^{17, 18} | cyclophosphamide, cytarabine, daunorubicin, doxorubicin, or |
| | vincristine) or combinations (e.g., ADE) |
| Large Cell | Radiotherapy (e.g., radioiodide therapy, external-beam |
| Neuroendocrine | radiation, or radium 223 therapy) and/or chemotherapeutics |
| Carcinoma¹⁹ | (e.g., cisplatin, carboplatin, or etoposide) |
| Neuroblastoma²⁰ | Chemotherapeutics (e.g., cyclophosphamide, doxorubicin, or |
| | vincristine) |

(C) Cancers wherein the dysregulation of a NTRK gene, a Trk protein, or expression or activity, or level of the same, includes overexpression of wildtype TrkA (autocrine activation), e.g., including:

| Cancer | Standard of care |
|---|---|
| Prostate | Radiotherapy (e.g., radium 223 therapy) or chemotherapeutics |
| Carcinoma^{21, 22} | (e.g. abiraterone, cabazitaxel, degarelix, denosumab, docetaxel, |
| | enzalutamide, leuprolide, prednisone, or sipuleucel-T) |
| Neuroblastoma²³ | Chemotherapeutics (e.g., cyclophosphamide, doxorubicin, or |
| | vincristine) |
| Pancreatic | Chemotherapeutics as single agents (e.g., erlotinib, fluorouracil, |
| Carcinoma²⁴ | gemcitabine, or mitomycin C) or combinations (e.g., |
| | gemcitabine-oxaliplatin) |
| Melanoma²⁵ | Chemotherapeutics (e.g., aldesleukin, dabrafenib, dacarbazine, |
| | interferon alfa-2b, ipilimumab, peginterferon alfa-2b, trametinib, |
| | or vemurafenib) |
| Head and Neck | Radiotherapy and/or chemotherapeutics (e.g., bleomycin, |
| Squamous Cell | cetuximab, cisplatin, docetaxel, fluorouracil, or methotrexate) |
| Carcinoma²⁶ | |
| Gastric | Chemotherapeutics (e.g., docetaxel, doxorubucin, fluorouracil, |
| Carcinoma²⁷ | mitomycin C, or trastuzumab) |

In some embodiments, the dysregulation of a NTRK gene, a Trk protein, or expression or activity, or level of the same, includes a translocation that results in the expression of a TrkB fusion protein, e.g., one of the TrkB fusion proteins shown in Table 5.

TABLE 5
Exemplary TrkB Fusion Proteins and Cancers

| | | Non-limiting Exemplary TrkB |
|---|---|---|
| | | Fusions and Synonyms of |
| Fusion Protein | Non-TrkB Fusion Partner | Associated Cancer(s) |
| NACC2-TrkB¹ | NACC Family Member 2, | Pilocytic Astrocytoma¹ |
| | BEN and BTB (POZ) Domain | |
| | Containing | |
| QKI-TrkB^{1, 11} | QKI, KH Domain Containing, | Pilocytic Astrocytoma¹ |
| | RNA Binding | |
| AFAP1-TrkB² | Actin Filament Associated | Lower-Grade Glioma^{2, 5}, Pilocytic |
| | Protein 1 | Astrocytoma with Anaplasia |
| | | (PAA)⁴, In vitro (Murine Ba/F3 |
| | | cells)³ |
| PAN3-TrkB² | PAN3 Poly(A) Specific | Head and Neck Squamous Cell |
| | Ribonuclease Subunit | Carcinoma² |
| SQSTM1-TrkB² | Sequestosome 1 | Lower-Grade Glioma², |
| | | Glioblastoma¹² |
| TRIM24-TrkB² | Tripartite Motif Containing 24 | Lung adenocarcinoma², Non- |
| | | Small Cell Lung Cancer¹⁷ |
| VCL-TrkB⁶ | Vinculin | Pediatric gliomas (e.g., pediatric |
| | | high-grade glioma⁶) |
| AGBL4-TrkB⁶ | ATP/GTP Binding Protein- | Pediatric gliomas (e.g., pediatric |
| | Like 4 | high-grade glioma⁶) |
| DAB2IP-TrkB¹⁷ | Disabled Homolog 2- | Colorectal Cancer¹⁷ |
| | Interacting Protein | |
| TrkB-TERT⁷ | Telomerase Reverse | Thyroid Cancer^{7, 8} |
| | Transcriptase | |
| TEL-TrkB⁹ | ETS Variant 6 | In vitro (murine Ba/F3 cells)⁹, |
| (ETV6) | | Acute Myeloid Leukemia |
| | | (AML)¹⁰, Pediatric |
| | | Glioblastoma²¹ |
| NOS1AP-TrkB¹² | | Anaplastic Astrocytoma¹² |
| GKAP1-TrkB¹² | | Glioblastoma¹² |
| KCTD8-TrkB¹² | | Glioblastoma¹² |
| TBClD2-TrkB¹² | | Glioblastoma¹² |
| VCAN-TrkB¹² | | Grade II Astrocytoma¹² |
| SLMAP-TrkB¹⁸ | | Ganglioma¹³ |
| TLE4-TrkB¹⁴ | | Ganglioma¹⁴ |
| STRN3-TrkB¹⁵ | Striatin | Ganglioglioma¹⁵ |
| WNK2-TrkB¹⁵ | | Complex Glioneuronal Tumor¹⁵ |
| TrkB-BEND5¹⁶ | | Malignant Epithelioid |
| | | Glioneuronal Tumor (MEGNT)¹⁶ |
| TrkB-TRAF2¹⁹ | | Melanoma¹⁹ |
| Nav1-TrkB²⁰ | | Oligoastrocytoma²⁰ |
| STRN-TrkB | | Salivary Gland Cancer |

¹Jones et al., Nature Genetics 45: 927-932, 2013.
²Stransky et al., Nature Comm. 5: 4846, 2014.
³Drilon et al., Ann Oncol. 27(5): 920-6, 2016.
⁴Lin et al., Abstract Number: HG-48. 17^{th} International Symposium on Pediatric Neuro-Oncology, ISPNO 2016. Liverpool, UK, 2016.
⁵ U.S. Patent Application No. 2016/0272725.
⁶Wu et al., Nature Genetics 46: 444-450, 2014.
⁷P.C.T. Patent Application Publication No. WO 2015/183836.
⁸ P.C.T. Patent Application Publication No. WO 2015/183837.
⁹Yuzugullu et al., Cell Discov. 2: 16030, 2016.
¹⁰Taylor et al. Abstract Number: 794. Meeting Info: 59th Annual Meeting of the American Society of Hematology, ASH 2017. Atlanta, GA, United States, 2017.
¹¹ Ni et al., Neuro Oncol. 19(1): 22-30, 2017.
¹²Subramaniam et al., 2017 Annual Meeting of the American Society of Clinical Oncology, ASCO. Chicago, IL, United States, 2017.
¹³Ellison et al., Abstract Number: O13. 117th Meeting of the British Neuropathological Society, Royal College of Physicians. London, United Kingdom, 2017.
¹⁴Prabhakaran et al., Neuropathology. E-ISSN: 1440-1789. L-ISSN: 0919-6544, 2018.
¹⁵Alvarez-Breckenridge et al., NPJ Precision Oncology. 1(5) doi: 10.1038/s41698-017-0009-y, 2017.
¹⁶Bavle et al., Abstract Number: GENE-04. Meeting Info: 4th Biennial Conference on Pediatric Neuro-Oncology Basic and Translational Research. New York City, NY, United States, 2017.
¹⁷Wei et al., Abstract Number: 78. Meeting Info: 28th EORTC-NCI-AACR Symposium on Molecular Targets and Cancer Therapeutics. Munich, Germany, 2016.
¹⁸Qaddoumi et al., Acta Neuropathol. 131(6): 833-45, 2016.
¹⁹Lezcano et al., Am. J. Surg. Pathol. doi: 10.1097/PAS.0000000000001070, 2018.
²⁰Zhang et al., Nat. Genet. 45(6): 602-612, 2013.
²¹Bender et al., Abstract Number: HG-024. Meeting Info: 16th International Symposium on Pediatric Neuro-Oncology in Conjunction with the 8th St. Jude-VIVA Forum. Singapore, Singapore, 2014.

In some embodiments, the dysregulation of a NTRK gene, a Trk protein, or expression or activity or level of any of the same, includes at least one point mutation in a NTRK gene that results in the production of a TrkB protein that has one or more amino acid substitutions, insertions, or deletions as compared to the wildtype TrkB protein (see, for example, the point mutations listed in Table 6).

TABLE 6
TrkB Kinase Protein Amino Acid
Substitutions/Insertions/Deletions^{A}

Amino acid position 13 (e.g., A13T²)
Amino acid position 142 (e.g., E142K²)
Amino acid position 136 (e.g., R136H²)
Amino acid position 167 (e.g., S167Y³)
Amino acid position 545 (e.g., G545R)
Amino acid position 570 (e.g., A570V)
Amino acid position 596 (e.g., Q596E, Q596P)
Amino acid position 601 (e.g., V601G)
Amino acid position 617 (e.g., F617L, F617C, F617I)
Amino acid position 619 (e.g., V619M⁴)
Amino acid position 623 (e.g., G623S, G623R)
Amino acid position 624 (e.g., D624V)
Amino acid position 628 (e.g., F628x)
Amino acid position 630 (e.g., R630K)
Amino acid position 633 (e.g., F633L⁴)
Amino acid position 639 (e.g., G639R¹)
Amino acid position 672 (e.g., F672x)
Amino acid position 682 (e.g., C682Y, C682F)
Amino acid position 683 (e.g., L683V)
Amino acid position 693 (e.g., G693S)
Amino acid position 702 (e.g., Y702x)
Amino acid position 709 (e.g., G709C, G709A, G709S⁴)
Amino acid position 716 (e.g., P716S⁵)
^{A}The TrkB kinase mutations shown above may be activating mutations and/or may confer increased resistance of the TrkB kinase to a TrkB inhibitor e.g., as compared to a wildtype TrkB kinase.
¹PCT Application No. WO2017155018A1.
²Bonanno et al., Journal of Thoracic Oncology, Vol. 11, No. 4, Supp. Suppl. 1, pp S67. Abstract Number: 28P; 6th European Lung Cancer Conference, ELCC 2016, Geneva, Switzerland.
³Iniguez-Ariza et al., Journal of Clinical Oncology, (20 Jun. 2017) Vol. 35, No. 15, Supp. 1, 2017 Annual Meeting of the American Society of Clinical Oncology, ASCO, 2017.
⁴PCT Application No. WO2016196141A1.
⁵Deihimi et al., Oncotarget. Jun 20; 8(25): 39945-39962. doi: 10.18632/oncotarget.18098, 2017.

In some embodiments, the dysregulation of a NTRK gene, a Trk protein, or expression or activity, or level of the same, includes at least one point mutation in a NTRK2 gene that results in the production of a TrkB protein that has one or more amino acid substitutions as compared to the wildtype TrkB protein. In some embodiments, the at least one or more amino acid substitutions are activating mutations (see, for example, the point mutations listed in Table 7).

TABLE 7
Activating TrkB Point Mutations¹

| | | |
|---|---|---|
| | | Exemplary Isoform in which |
| | | Mutation is Present (if |
| Point Mutation | Rationale | known) |

| | |
|---|---|
| A13T² | Reference TrkB sequence |
| E142K² | Reference TrkB sequence |
| R136H² | Reference TrkB sequence |
| S167Y³ | |
| P716S⁴ | |

¹Reference TrkB sequence is UniProtKB/Swiss-Prot: Q16620.1, and can be found at URL: www.ncbi.nlm.nih.gov/protein/2497560?report=genbank&log$=protalign&blast_rank=0&RID=0
²Bonanno et al., Journal of Thoracic Oncology, Vol. 11, No. 4, Supp. Suppl. 1, pp S67. Abstract Number: 28P; 6^{th} European Lung Cancer Conference, ELCC 2016, Geneva, Switzerland.
³Iniguez-Ariza et al., Journal of Clinical Oncology, (20 Jun. 2017) Vol. 35, No. 15, Supp. 1, 2017 Annual Meeting of the American Society of Clinical Oncology, ASCO, 2017.
⁴Deihimi et al., Oncotarget. Jun 20; 8(25): 39945-39962. doi: 10.18632/oncotarget.18098, 2017.

In some embodiments, the dysregulation of a NTRK gene, a Trk protein, or expression or activity, or level of the same, includes a translocation which results in the expression of a TrkC fusion protein, e.g., one of the TrkC fusion proteins shown in Table 8.

TABLE 8
Exemplary TrkC Fusion Proteins and Cancers

| | | Non-limiting Exemplary |
|---|---|---|
| | Non-TrkC Fusion | TrkC Fusions and Synonyms |
| Fusion Protein | Partner | of Associated Cancer(s) |
| ETV6-TrkC¹ | ETS Variant 6 | Fibrosarcoma (e.g., Infantile or |
| (TEL; e.g., | | Congenital Fibrosarcoma (IFS, |
| chromosomal | | CFS, or CIFS)^{6, 7, 29, 30}), |
| translocation | | Nephroma (e.g., Congenital |
| t(12; 15) (p13; q25)² | | Mesoblastic Nephroma^{3, 60}), |
| t(12; 15)(p13; q26), | | Melanoma (e.g., Skin |
| ins(12; 15)(p13; q22 | | Cutaneous Melanoma⁵⁶), |
| q26)³, or | | Colorectal Cancer (CRC)^{33, 58} |
| t(12; 15)(p13; q25)⁴) | | (colon adenocarcinoma⁵⁶), |
| | | Breast Cancer⁵⁶, |
| | | Gastrointestinal Stromal Tumor |
| | | (GIST)²⁸ (e.g., c-kit-Negative |
| | | GIST²⁸), Pediatric Gliomas |
| | | (e.g., Pediatric High-Grade |
| | | Gliomas^{1, 8}, Desmoplastic |
| | | Infantile Ganglioglioma¹¹), |
| | | Medulloblastoma¹, Thyroid |
| | | Cancer (e.g., Papillary Thyroid |
| | | Cancer^{12, 56, 59}, Sporadic |
| | | Pediatric Differentiated Thyroid |
| | | Carcinoma (DTC)¹³ Post- |
| | | Chernobyl PTCs³¹), Soft Tissue |
| | | Hemangioma³⁴, Mammary |
| | | Analogue Secretory Carcinoma |
| | | (MASC)^{14, 61}, Secretory Breast |
| | | Carcinoma (SBSC)^{10, 27, 57}), |
| | | Primary Thyroid Gland |
| | | Secretory Carcinoma¹⁵, Acinic |
| | | cell carcinoma (AcCC)¹⁶, |
| | | Polymorphous Low-Grade |
| | | Adenocarcinoma¹⁷, Sinonasal |
| | | Low-Grade Non-Intestinal- |
| | | Type Adenocarcinoma⁶², ALK- |
| | | Negative Inflammatory |
| | | Myofibroblastic Tumors |
| | | (IMT)^{18, 19}, Acute Myeloid (or |
| | | Myelogenous) Leukemia |
| | | (AML)³², Promyelocytic |
| | | Leukemia²⁶, Acute |
| | | Lymphoblastic Leukemia |
| | | (ALL) (e.g., Ph-like ALL^{5, 22}), |
| | | Chronic Eosinophilic |
| | | Leukemia²³, Relapsed Pediatric |
| | | B-ALL⁵³, Angiomatoid Fibrous |
| | | Histiocytoma²⁴, |
| | | Neuroendricrine Tumor²⁵ |
| BTBD1-TrkC¹ | BTB (POZ) Domain | Pediatric Gliomas (e.g., high- |
| | Containing 1 | grade gliomas¹) |
| LYN-TrkC³⁵ | V-Yes-1 Yamaguchi | Head and Neck Squamous Cell |
| | Sarcoma Viral Related | Carcinoma⁶³ |
| | Oncogene Homolog (also | |
| | known as Lck/Yes-Related | |
| | Novel Protein Tyrosine | |
| | Kinase) | |
| RBPMS-TrkC³⁵ | RNA Binding Protein with | Thyroid Cancer⁵⁶ (e.g., |
| | Multiple Splicing | Papillary Thyroid Cancer⁶³), |
| | | Uterine Spindle Cell Sarcoma³⁶ |
| EML4-TrkC³⁷ | Echinoderm Microtubule- | Fibrosarcoma (e.g., Pediatric |
| (eg., | Associated Protein-Like 4 | Fibrosarcoma³⁹ or Infantile |
| t(2; 15)(2p21; 15q25))³⁸ | | Fibrosarcoma^{9, 37, 45, 64}), |
| | | Glioblastoma^{40, 20}, Colon |
| | | Cancer⁴¹, Mesenchymal |
| | | Tumor⁴², Thyroid Cancer⁴³, |
| | | Congenital Mesoblastic |
| | | Nephroma⁴⁴, Pancreatic |
| | | adenocarcinoma⁵⁶ |
| TrkC-HOMER2 | Homer Protein Homolog 2 | Soft Tissue Sarcoma³⁴ |
| TFG-TrkC | TRK-Fused Gene | Soft Tissue Solitary Fibrous |
| | | Tumor³⁴ |
| FAT1-TrkC⁴⁶ | FAT Atypical Cadherin 1 | Cervical Squamous Cell |
| | | Carcinoma^{46, 56} |
| MYO5A-TrkC⁴⁹ | Myosin VA | Melanocytic Tumor⁴⁹ (e.g., |
| | | Spitz tumor⁴⁷), Melanoma⁴⁸ |
| MYH9-TrkC⁴⁷ | Myosin Heavy Chain 9 | Spitz Tumor⁴⁷ |
| KANK1-TrkC²¹ | KANK1 | Renal Metanephric Adenoma |
| (eg., | | (MA)²¹ |
| t(9; 15)(p24; q24))⁵⁰ | | |
| SQSTM1-TrkC⁵¹ | Sequestosome 1 | Papillary Thyroid Carcinoma, |
| | | thyroid carcinoma^{55, 56} |
| UBE2R2-TrkC | Ubiquitin Conjugating | Multiple Myeloma⁵² |
| | Enzyme E2 R2 | |
| HNRNPA2B1- | | Multiple Myeloma⁵² |
| TrkC | | |
| VPS18-NTRK3⁵⁶ | | Colon Adenocarcinoma⁵⁶ |
| AKAP13-NTRK3⁵⁶ | | Lower Grade Glioma⁵⁶ |
| NTRK3-LOXL2⁵⁶ | | Lower Grade Glioma⁵⁶ |
| NTRK3-PEAK1⁵⁶ | | Lower Grade Glioma⁵⁶ |
| ZNF710-TrkC*^{54, 58} | | |
| TPM4-TrkC | | Soft Tissue Sarcoma |
| LMNA-TrkC | | Soft Tissue Sarcoma |

*The transcript of this fusion was not detected.
¹Wu et al., Nature. Genet. 46: 444-450, 2014.
²Skalova et al., Mod. Pathol. 30: S27-S43, 2017.
³Watanbe et al., Cancer Genet. Cytogenet. 136(1): 10-16, 2002.
⁴Eguchi et al., Blood. 93: 1355-1363, 1999.
⁵Roberts et al., Abstract Number: 278, 58th Annual Meeting of the American Society of Hematology, ASH 2016. San Diego, CA, United States, 2016.
⁶Knezevich et al., Nat. Genet. 18(2): 184-7, 1998.
⁷Pavlick et al., Pediatr. Blood Cancer. doi: 10.1002/pbc.26433, 2017.
⁸Hover et al., Abstract Number: TMOD-07. Meeting Info: 4th Biennial Conference on Pediatric Neuro-Oncology Basic and Translational Research. New York City, NY, United States, 2017.
⁹Church et al., Mod. Pathol. 31(3), 463-473, 2018.
¹⁰Arce et al., World J. Surg. Oncol. 3: 35, 2005.
¹¹Carvalho et al., Abstract Number: HG-09. Meeting Info: 3rd Biennial Conference on Pediatric Neuro-Oncology Basic and Translational Research. San Diego, CA, United States, 2015.
¹²Otsubo et al., J. Pediatr.. Endocrinol.. Metab. 28; 31(4): 461-467, 2018.
¹³Picarsic et al., Pediatr.. Dev. Pathol. 19(2): 115-22, 2016.
¹⁴Skalova et al., Am. J. Surg. Pathol. 42(2): 234-246, 2018.
¹⁵Farhat et al., Am. J. Clin. Pathol., 148(3): 251-258, 2017.
¹⁶Chintakuntlawar et al., Oral Surg. Oral Med. Oral Pathol. Oral Radiol. 121(5): 542-549.e1, 2016.
¹⁷Montalli et al., J. Oral Pathol. Med. doi: 10.1111/jop.12491, 2016.
¹⁸Alassiri et al., Am. J. Surg. Pathol. 40(8): 1051-61, 2016.
¹⁹Yamamoto et al., Histopathology. 69(1): 72-83, 2016.
²⁰Subramaniam et al., 2017 Annual Meeting of the American Society of Clinical Oncology, ASCO. Chicago, IL, United States, 2017.
²¹Catic et al., Cancer Genet. 214-215: 9-15, doi: 10.1016/j.cancergen.2017.03.001, 2017.
²²Reshmi et al., Abstract Number: 477. Meeting Info: 59th Annual Meeting of the American Society of Hematology, ASH 2017. Atlanta, GA, United States, 2017.
²³Forghieri et al., Abstract Number: P137. Meeting Info: 11th Congress of the Italian Society of Experimental Hematology. Turin, Italy, 2010.
²⁴Walther et al., Cancer Genet. 206(7-8), 299-303, 2013.
²⁵Sigal, et al., J. Natl. Compr. Canc. Netw. 15(11): 1317-1322, 2017.
²⁶Macleod, et al., Abstract Number: 0294. Meeting Info: 14th Congress of the European Hematology Association. Berlin, Germany, 2009.
²⁷Tognon et al., Cancer Cell. 2(5): 367-376, 2002.
²⁸Brenca et al., J. Pathol. 238(4): 543-549, 2016.
²⁹Rossi et al., Abstract Number: 84. Meeting Info: 105th Annual Meeting of the United States and Canadian Academy of Pathology, USCAP 2016. Seattle, WA, United States, 2016.
³⁰Sheng et al., Am. J. Clin. Pathol. 115(3): 348-355, 2001.
³¹Leeman-Neill et al., Cancer. 120(6): 799-807, 2014.
³²Kralik et al., Diagn. Pathol. 6: 19, 2011.
³³U.S. Patent Application No. 2016/0305943.
³⁴Doebele et al., Cancer Discov. 5(10): 1049-1057, 2015.
³⁵Stransky et al., Nature Comm. 5: 4846, 2014.
³⁶Chiang et al., Am. J. Surg. Pathol. doi: 10.1097/PAS.0000000000001055, 2018.
³⁷Tannenbaum et al., Cold Spring Harb. Mol. Case Stud. 1: a000471, 2015.
³⁸Tannenbaum, et al., Abstract Number: 749. Meeting Info: 2015 American Society of Pediatric Hematology/Oncology, ASPHO 2015. Phoenix, AZ, United States, 2015.
³⁹Sims et al., Abstract Number: P280; 31^{st} Annual Meeting and Associated Programs of the Society for Immunotherapy of Cancer, SITC 2016. National Harbor, MD, United States, 2016.
⁴⁰Schram et al., Cancer Research. Abstract Number: LB-302, American Association for Cancer Research Annual Meeting, Washington, DC, United States, 2017.
⁴¹Coebergh et al., Cancer Research. Abstract Number: 490, American Association for Cancer Research Annual Meeting, Washington, DC, United States, 2017.
⁴²Davis et al., Pediatr. Dev. Pathol. 21(1): 68-78, 2018.
⁴³Nikiforova et al., Abstract Number: 5. Meeting Info: 84th Annual Meeting of the American Thyroid Association. Coronado, CA, United States, 2014.
⁴⁴Church et al., Mod. Pathol. 31(3), 463-473, 2018.
⁴⁵Church et al., Abstract Number: ST16. Meeting Info: 2015 Annual Meeting of the Association for Molecular Pathology, AMP 2015. Austin, TX, United States, 2015.
⁴⁶U.S. Patent Application Publication No. 2015/0315657.
⁴⁷Yeh et al., J Pathol. 240(3): 282-90, 2016.
⁴⁸Leyvraz et al., Abstract Number: 897. Meeting Info: 33. Deutscher Krebskongress, DKK. Berlin, Germany, 2018.
⁴⁹Wang et al., J. Mol. Diagn. 19(3): 387-396, 2017.
⁵⁰Catic et al., Meeting Info: 2017 Annual Meeting of the American Society of Clinical Oncology, ASCO. Chicago, IL, United States, 2017.
⁵¹Lu et al., Oncotarget. 8(28): 45784-45792, 2017.
⁵²Taylor et al., Abstract Number: 794. Meeting Info: 59th Annual Meeting of the American Society of Hematology, ASH 2017. Atlanta, GA, United States, 2017.
⁵³Baughn et al., Abstract Number: 5115. Meeting Info: 59th Annual Meeting of the American Society of Hematology, ASH 2017. Atlanta, GA, United States, 2017.
⁵⁴Hechtman et al., Abstract Number: 1837. Meeting Info: 106th Annual Meeting of the United States and Canadian Academy of Pathology, USCAP 2017. San Antonio, TX, United States, 2017.
⁵⁵Iyama et al., Thyroid. 27(6): 811-818, 2017.
⁵⁶Gao et al., Cell Rep. 23(1): 227-238.e3, 2018.
⁵⁷Zheng et al., Nat Med. 20(12): 1479-84, 2014.
⁵⁸Hechtman et al., Mol. Cancer Res. 14(3): 296-301, 2016.
⁵⁹Ricarte-Filho et al., J. Clin. Invest. 123(11): 4935-44, 2013.
⁶⁰Rubin et al., Am. J. Pathol. 153(5): 1451-8, 1998.
⁶¹Skálová et al., Am. J. Surg. Pathol. 2016 Jan; 40(1): 3-13.
⁶²Andreason et al., Am. J. Surg. Pathol. 41(11): 1552-1560, 2017.
⁶³Wei et al., Abstract Number: 78. Meeting Info: 28th EORTC-NCI-AACR Symposium on Molecular Targets and Cancer Therapeutics. Munich, Germany, 2016.
⁶⁴Kao et al., Am. J. Surg. Pathol. 42(1): 28-38, 2018.

In some embodiments, the dysregulation of a NTRK gene, a Trk protein, or expression or activity or level of any of the same, includes at least one point mutation in a NTRK gene that results in the production of a TrkC protein that has one or more amino acid substitutions, insertions, or deletions as compared to the wildtype TrkC protein (see, for example, the point mutations listed in Table 9).

TABLE 9
TrkC Kinase Protein Amino Acid
Substitutions/Insertions/Deletions^{A}

Amino acid position 545 (e.g., G545R)
Amino acid position 570 (e.g., A570V)
Amino acid position 596 (e.g., Q596x)
Amino acid position 601 (e.g., V601x)
Amino acid position 603 (e.g., V603M²)
Amino acid position 617 (e.g., F617x, F617L²)
Amino acid position 623 (e.g., G623R¹)
Amino acid position 624 (e.g., D624V)
Amino acid position 628 (e.g., F628x)
Amino acid position 630 (e.g., R630x)
Amino acid position 675 (e.g., F675x)
Amino acid position 685 (e.g., C685Y, C685F)
Amino acid position 686 (e.g., L686V)
Amino acid position 696 (e.g., G696x, G696C, G696A², G696S²)
Amino acid position 705 (e.g., Y705x)
Amino acid position 745 (e.g., R745L³)
Amino acid position 749 (e.g., I749M⁴)
^{A}The TrkC kinase mutations shown above may be activating mutations and/or may confer increased resistance of the TrkC kinase to a TrkC inhibitor e.g., as compared to a wildtype TrkC kinase.
¹Drilon et al., What hides behind the MASC: clinical response and acquired resistance to entrectinib after ETV6-NTRK3 identification in a mammary analogue secretory carcinoma (MASC), Ann Oncol. 2016 May; 27(5): 920-6. doi: 10.1093/annonc/mdw042. Epub 2016 Feb. 15.
²PCT Application No. WO2016196141A1.
³Deihimi et al., Oncotarget. Jun 20; 8(25): 39945-39962. doi: 10.18632/oncotarget.18098, 2017.
⁴Iniguez-Ariza et al., Journal of Clinical Oncology, (20 Jun. 2017) Vol. 35, No. 15, Supp. 1, 2017 Annual Meeting of the American Society of Clinical Oncology, ASCO, 2017.

In some embodiments, the dysregulation of a NTRK gene, a Trk protein, or expression or activity, or level of the same, includes at least one point mutation in a NTRK3 gene that results in the production of a TrkC protein that has one or more amino acid substitutions as compared to the wildtype TrkC protein. In some embodiments, the at least one or more amino acid substitutions are activating mutations (see, for example, the point mutations listed in Table 10).

TABLE 10
Activating TrkC Point Mutations¹

| | | Exemplary Isoform in |
|---|---|---|
| | | which Mutation is Present |
| Point Mutation | Rationale | (if known) |
| G623R² | Steric Hinderance | Reference TrkC sequence |
| R745L³ | | |
| I749M⁴ | | |

¹Reference TrkC sequence is UniProtKB/Swiss-Prot: Q16288.2, and can be found at URL: www.ncbi.nlm.nih.gov/protein/134035335?report=genbank&log$=protalign&blast_rank=0&RID=0
²Drilon et al., Ann Oncol. 2016 May; 27(5): 920-6. doi: 10.1093/annonc/mdw042.Epub 2016 Feb. 15.
³Deihimi et al., Oncotarget. Jun 20; 8(25): 39945-39962. doi: 10.18632/oncotarget.18098, 2017.
⁴Iniguez-Ariza et al., Journal of Clinical Oncology, (20 Jun. 2017) Vol. 35, No. 15, Supp. 1, 2017 Annual Meeting of the American Society of Clinical Oncology, ASCO, 2017.

In some embodiments, the dysregulation of a NTRK gene, a Trk protein, or expression or activity, or level of the same, includes at least one point mutation in a NTRK gene that results in the production of a Trk protein that has one or more amino acid substitutions as compared to the wildtype Trk protein. For example, a mutation can include one or more of a solvent front mutation (e.g., TrkA G595R), an xDFG mutation (e.g., TrkA G667S), or a gatekeeper mutation (e.g., TrkC F617L). In some embodiments, these mutations are associated with resistance (e.g., acquired resistance) to one or more Trk kinase inhibitors.

In some embodiments, a Trk-associated cancer has been identified as having one or more Trk inhibitor resistance mutations (that result in an increased resistance to a Trk inhibitor. Non-limiting examples of Trk inhibitor resistance mutations are listed in Tables 11-13.

TABLE 11
Exemplary TrkA Resistance Mutations

Amino acid position 517 (e.g., G517R)
Amino acid position 542 (e.g., A542V)
Amino acid position 564 (e.g., L564H²)
Amino acid position 568 (e.g., Q568x)
Amino acid position 573 (e.g., V573M)
Amino acid position 589 (e.g., F589L, F589C)
Amino acid position 595 (e.g., G595S, G595R¹, G595L²)
Amino acid position 599 (e.g., D596V)
Amino acid position 600 (e.g., F600L)
Amino acid position 602 (e.g., R602x)
Amino acid position 646 (e.g., F646V, F646I²)
Amino acid position 656 (e.g., C656Y, C656F)
Amino acid position 657 (e.g., L657V)
Amino acid position 667 (e.g., G667A³, G667C¹, G667S³)
Amino acid position 676 (e.g., Y676S)
Amino acid position 679 (e.g., D679G²)
¹Russo et al., Acquired Resistance to the TRK Inhibitor Entrectinib in Colorectal Cancer, Cancer Discov., Jan; 6(1): 36-44, 2016.
²Fuse et al., Mechanisms of Resistance to NTRK Inhibitors and Therapeutic Strategies in NTRK1-Rearranged Cancers, Mol. Cancer Ther.,. Jan; 6(1): 36-44, 2016.
³PCT Application No. WO2016196141A1.

The letter “x” when used to describe a mutation of an amino acid at a specific amino acid position means (i) a substitution of the amino acid present at the same amino acid position in the corresponding wild-type protein with a different naturally-occurring amino acid, or (ii) a deletion of the amino acid present at the same amino acid position in the corresponding wild-type protein.

Non-limiting examples of the specific amino acid positions discovered to have mutations (e.g., substitutions or deletions) in TrkA in Trk inhibitor-resistant cancer cells having a NTRK1 point mutation are listed below. Also listed below are the different specific amino acid mutations (e.g., substitutions) present in TrkA proteins present in Trk inhibitor resistant cancer cells having a NTRK1 point mutation.

Trk inhibitor-resistant cancer cells were discovered to have point mutations in a NTRK1 gene that result in a TrkA protein that includes one or more (e.g., two, three, four, five, six, seven, eight, nine, ten, eleven, twelve, thirteen, fourteen, or fifteen) amino acid substitutions or deletions at amino acid positions: 517, 542, 568, 573, 589, 595, 599, 600, 602, 646, 656, 657, 667, and 676 (e.g., amino acid positions corresponding to those in wild-type sequence NP_002520 (SEQ ID NO: 9)). Different specific amino acid substitutions present in a TrkA protein generated in a Trk inhibitor-resistant cancer cell include one or more (e.g., two, three, four, five, six, seven, eight, nine, ten, eleven, or twelve) of the following: G517R, A542V, V573M, F589L, F589C, G595S, G595R, D596V, F600L, F646V, C656Y, C656F, L657V, G667S, G667C, and Y676S (e.g., as compared to the wild-type sequence NP_002520 (SEQ ID NO: 9)).

TABLE 12
Exemplary TrkB Resistance Mutations

Amino acid position 545 (e.g., G545R)
Amino acid position 570 (e.g., A570V)
Amino acid position 596 (e.g., Q596E, Q596P)
Amino acid position 601 (e.g., V601G)
Amino acid position 617 (e.g., F617L, F617C, F617I)
Amino acid position 619 (e.g., V619M)²
Amino acid position 623 (e.g., G623S, G623R)
Amino acid position 624 (e.g., D624V)
Amino acid position 628 (e.g., F628x)
Amino acid position 630 (e.g., R630K)
Amino acid position 633 (e.g., F633L²)
Amino acid position 639 (e.g., G639R¹)
Amino acid position 672 (e.g., F672x)
Amino acid position 682 (e.g., C682Y, C682F)
Amino acid position 683 (e.g., L683V)
Amino acid position 693 (e.g., G693S)
Amino acid position 702 (e.g., Y702x)
Amino acid position 709 (e.g., G709C², G709A², G709S²)
¹PCT Application No. WO2017155018A1.
²PCT Application No. WO2016196141A1.

The letter “x” when used to describe a mutation of an amino acid at a specific amino acid position means (i) a substitution of the amino acid present at the same amino acid position in the corresponding wild-type protein with a different naturally-occurring amino acid, or (ii) a deletion of the amino acid present at the same amino acid position in the corresponding wild-type protein.

Trk inhibitor-resistant cancer cells were discovered to have point mutations in a NTRK2 gene that result in a TrkB protein that includes one or more (e.g., two, three, four, five, six, seven, eight, nine, ten, eleven, twelve, thirteen, fourteen, or fifteen) amino acid substitutions or deletions at amino acid positions: 545, 570, 596, 601, 617, 623, 624, 628, 630, 672, 682, 683, 693, and 702 (e.g., amino acid positions corresponding to those in wild-type sequence AAB33109.1 (SEQ ID NO: 10)). Different specific amino acid substitutions present in a TrkB protein generated in a Trk inhibitor-resistant cancer cell include one or more (e.g., two, three, four, five, six, seven, eight, nine, eleven, or twelve) of the following: G545R, A570V, Q596E, Q596P, V601G, F617L, F617C, F6171, G623S, G623R, D624V, R630K, C682Y, C682F, L683V, G693S, and G713S (e.g., as compared to the wild-type sequence AAB33109.1 (SEQ ID NO: 10)).

TABLE 13
Exemplary TrkC Resistance Mutations

Amino acid position 545 (e.g., G545R)
Amino acid position 570 (e.g., A570V)
Amino acid position 596 (e.g., Q596x)
Amino acid position 601 (e.g., V601x)
Amino acid position 603 (e.g., V603M²)
Amino acid position 617 (e.g., F617x, F617L²)
Amino acid position 623 (e.g., G623R¹)
Amino acid position 624 (e.g., D624V)
Amino acid position 628 (e.g., F628x)
Amino acid position 630 (e.g., R630x)
Amino acid position 675 (e.g., F675x)
Amino acid position 685 (e.g., C685Y, C685F)
Amino acid position 686 (e.g., L686V)
Amino acid position 696 (e.g., G696x, G696A², G696C²,
G696S²)
Amino acid position 705 (e.g., Y705x)
¹Drilon et al., What hides behind the MASC: clinical response and acquired resistance to entrectinib after ETV6-NTRK3 identification in a mammary analogue secretory carcinoma (MASC), Ann Oncol. 2016 May; 27(5): 920-6. doi: 10.1093/annonc/mdw042. Epub 2016 Feb. 15.
²PCT Application No. WO2016196141A1.

The letter “x” when used to describe a mutation of an amino acid at a specific amino acid position means (i) a substitution of the amino acid present at the same amino acid position in the corresponding wild-type protein with a different naturally-occurring amino acid, or (ii) a deletion of the amino acid present at the same amino acid position in the corresponding wild-type protein.

Trk inhibitor-resistant cancer cells were discovered to have point mutations in a NTRK3 gene that result in a TrkC protein that includes one or more (e.g., two, three, four, five, six, seven, eight, nine, ten, eleven, twelve, thirteen, fourteen, or fifteen) amino acid substitutions or deletions at amino acid positions: 545, 570, 596, 601, 617, 623, 624, 628, 630, 675, 685, 686, 696, and 705 (e.g., amino acid positions corresponding to those in a wild-type sequence (SEQ ID NO: 11)). Different specific amino acid substitutions present in a TrkC protein generated in a Trk inhibitor-resistant cancer cell include one or more (e.g., two, three, four, five, six, or seven, or eight) of the following: G545R, A570V, F617L, G623R, D624V, C685Y, C685F, L686V, and G696A (e.g., as compared to the wild-type sequence (SEQ ID NO: 11)).

As one skilled in the art can appreciate, the specific substitutions listed above are exemplary. For example, when a naturally-occurring amino acid at an amino acid position is substituted with a different amino acid, it is understood that an amino acid having a chemically-related amino acid side chain may also be substituted (and detected in a cancer cell). Amino acids that have chemically-related amino acid side chains are listed in Table 14.

TABLE 14
Chemically Related Amino Acid Side Chains

| | |
|---|---|
| Positively-Charged Side Chains | Lysine, Arginine, Histidine |
| Negatively-Charged Side Chains | Glutamate and Aspartate |
| Nonpolar and/or Aliphatic Side | Glycine, Alanine, Valine, Leucine, Isoleucine, |
| Groups | and Proline |
| Polar, Uncharged Side Groups | Serine, Threonine, Cysteine, Methionine, |
| | Asparagine, Glutamine |
| Aromatic Side Chains | Phenylalanine, Tyrosine, and Tryptophan |

In some embodiments, the dysregulation of a NTRK gene, a Trk protein, or expression or activity, or level of the same, includes a splice variation in a TrkA mRNA which results in an expressed protein that is an alternatively spliced variant of TrkA having at least one residue deleted (as compared to a wild-type TrkA protein) resulting in constitutive activity of the TrkA kinase domain. In some embodiments, an alternatively spliced form of TrkA with constitutive activity is the TrkAIII splice variant and, e.g., is associated with neuroectodermal-derived tumors including Wilm's tumor, neuroblastoma, and medulloblastoma (see, e.g., U.S. Patent Publication No. 2015/0218132).

Overexpression or increased expression of a NTRK gene (e.g., as compared to a control non-cancerous cell of the same cell type) is another type of dysregulation of a NTRK gene that is associated with a variety of different pediatric cancers. For example, overexpression of a Trk receptor has been observed in neuroectodermal-derived tumors including Wilm's tumor, neuroblastoma, and medulloblastoma (see, e.g., U.S. Patent Application Publication No. 2015/0218132), overexpression of NTRK2 in pediatric colorectal cancer subjects indicates poor prognosis in subjects (see, e.g., Tanaka et al., PLoS One 9:E96410, 2014), overexpression of NTRK2 has been observed in medulloblastoma and neuroblastoma in pediatric subjects (see, e.g., Evans et al., Clin. Cancer Res. 5:3592-3602, 1999; Geiger et al., J Cancer Res. 65:7033, 2005). Decreased NTRK1 expression has been detected in bilateral stage IV adrenal neuroblastoma with multiple skin metastases in a neonate (see, e.g., Yanai et al., J. Pediatr. Surg. 39:1782-1783, 2004).

In some embodiments, a Trk-associated cancer is advanced solid and primary central nervous system tumors (e.g., advanced solid and primary central nervous system tumors that are refractory to standard therapy). In some embodiments, the cancer is a solid or central nervous system tumors (e.g., advanced solid or primary central nervous system tumor) that is refractory to standard therapy.

Cancers identified as having dysregulation of a NTRK gene, a Trk protein, or expression or activity, or level of the same (see references cited herein and also the www.cancer.gov and www.nccn.org websites) include:

(A) Cancers wherein the dysregulation of a NTRK gene, a Trk protein, or expression or activity, or level of the same, includes one or more chromosome translocations or inversions resulting in Trk fusion proteins;

(B) Cancers wherein the dysregulation of a NTRK gene, a Trk protein, or expression or activity, or level of the same, includes one or more deletions, insertions, or mutations in the Trk protein;

(C) Cancers wherein the dysregulation of a NTRK gene, a Trk protein, or expression or activity, or level of the same, includes overexpression of wildtype Trk (e.g., leading to autocrine activation of a Trk);

In some embodiments, the dysregulation of a NTRK gene, a Trk protein, or expression or activity, or level of the same, includes a translocation that results in the expression of a TrkA, TrkB, or TrkC fusion protein, e.g., one of the TrkA, TrkB, or TrkC fusion proteins shown in Tables 2, 5, and 8.

In some embodiments, provided herein is a method for treating a patient diagnosed with a Trk-associated cancer, comprising administering to the patient a therapeutically effective amount of one or more Trk inhibitors as provided herein and optionally an immunotherapy agent. For example, the Trk-associated cancer can be selected from the group of: non-small cell lung cancer, papillary thyroid carcinoma (e.g., recurrent papillary thyroid cancer; younger papillary thyroid cancer), glioblastoma multiforme, acute myeloid leukemia, colorectal carcinoma, large cell neuroendocrine carcinoma, prostate cancer, neuroblastoma, pancreatic carcinoma, melanoma, head and neck squamous cell carcinoma, gastric carcinoma, Spitz cancer, papillary thyroid carcinoma, colon cancer, acute myeloid leukemia, gastrointestinal stromal tumor (GIST) (e.g., GIST testing wild type for KIT/PDGFR/BRAF/SDH), sarcoma, glioma (e.g., pediatric glioma), intrahepatic cholangicarcinoma, pilocytic astrocytoma, lower grade glioma, lung adenocarcinoma, salivary gland cancer, secretory breast cancer, fibrosarcoma, nephroma, and breast cancer.

Non-limiting examples of Trk-associated cancers include: Spitzoid melanoma, Spitz tumors (e.g., metastatic Spitz tumors), non-small cell lung cancer (NSCLC), thyroid carcinoma (e.g., papillary thyroid carcinoma (PTC)), acute myeloid leukemia (AML), sarcoma (e.g., undifferentiated sarcoma or adult soft tissue sarcoma), hepatobiliary cancer, glioma (e.g., pediatric gliomas), colorectal cancer (CRC), gliobastoma multiforme (GBM), large cell neuroendocrine cancer (LCNEC), thyroid cancer, intrahepatic cholangicarcinoma (ICC), pilocytic astrocytoma, lower-grade glioma, head and neck squamous cell carcinoma, adenocarcinoma (e.g., lung adenocarcinoma), salivary gland cancer, secretory breast carcinoma, breast cancer, acute myeloid leukemia, fibrosarcoma, nephroma, melanoma, bronchogenic carcinoma, B-cell cancer, bronchus cancer, cancer of the oral cavity or pharynx, cancer of hematological tissues, cervical cancer, gastric cancer, kidney cancer, liver cancer, multiple myeloma, ovarian cancer, pancreatic cancer, salivary gland cancer, small bowel or appendix cancer, testicular cancer, urinary bladder cancer, uterine or endrometrial cancer, inflammatory myofibroblastic tumors, gastrointestinal stromal tumor, non-Hodgkin's lymphoma, neuroblastoma, small cell lung cancer, squamous cell carcinoma, esophageal-gastric cancer, skin cancer, neoplasm (e.g., a melanocystic neoplasm), Spitz nevi, astrocytoma, medulloblastoma, glioma, large cell neuroendocrine tumors, mammary analogue secretory carconioma (e.g., MASC; mammary analogue secretory carcinoma of the salivary gland), nonparotid acinic cell carcinoma, bone cancer, and rectum carcinoma.

In some embodiments, the Trk-associated cancer is selected from the group consisting of: non-small cell lung carcinoma, thyroid neoplasms, sarcoma, GIST, malignant peripheral nerve sheath tumors, colorectal neoplasms, salivary gland neoplasms, biliary tract neoplasms, primary brain neoplasm, breast secretory carcinoma, melanoma, glioblastoma, bile duct neoplasms, astrocytoma, head and neck squamous cell carcinoma, pontine glioma, pancreatic neoplasms, ovarian neoplasms, uterine neoplasms, renal cell carcinoma, cholangiocarcinoma, skin carcinoma, bronchogenic carcinoma, bronchial neoplasms, lung neoplasms, respiratory tract neoplasms, thoracic neoplasms, nerve tissue neoplasms, nevi and melanomas, intestinal neoplasm, thyroid cancer, fibrosarcoma, infantile fibrosarcoma, congenital mesoblastic nephroma, and central nervous system neoplasms.

In some embodiments, provided herein is a method for treating a patient (e.g., a pediatric patient) diagnosed with a Trk-associated cancer, comprising administering to the patient a therapeutically effective amount of the compound of one or more Trk inhibitors as provided herein and optionally an immunotherapy agent. For example, the Trk-associated cancer can be selected from the group consisting of: pediatric nephroma, congenital fibrosarcoma (CFS), pediatric high-grade glioma (HGG), mesenchymal cancers (infant fibrosarcoma (IF), congenital mesoblastic nephroma, congenital infantile fibrosarcoma (CIFS); locally advanced infantile fibrosarcoma, pilocytic astrocytoma, brain tumors (e.g., glioglastomas), pediatic acute leukemia, Ph-like acute lymphoblastic leukemia, cellular congenital mesoblastic nephroma (CMN); infantile fibrosarcoma, pediatric high-grade glioma (HGG), diffuse intrinsic pontine gliomas (DIPGs), non-brainstem HGGs (NBS-HGGs), anaplastic large cell lymphoma (ALCL), non-Hodgkin's lymphoma (NHL), pediatric papillary thyroid carcinoma, secretory breast cancer, soft tissue sarcoma, hepatobiliary cancer, non-rhabdomyosarcoma soft tissue sarcomas (NRSTS), spitzoid melanoma, pediatric hemangiopericytoma-like sarcoma, spindle cell sarcoma, NOS with myo/haemangiopericytic growth pattern, advanced pediatric solid tumors, neuroectodermal-derived tumors (e.g., Wilm's tumor, neuroblastoma, and medulloblastoma), pediatric colorectal cancer, adrenal neuroblastoma, and central nervous system tumors (e.g., advanced solid and primary central nervous system tumors that are refractory to standard therapy).

In some embodiments, the cancer can be a fibrosarcoma. For example, the cancer can be infantile fibrosarcoma. In some embodiments, the subject is an infant and the fibrosarcoma is infantile fibrosarcoma. In some embodiments, the cancer is locally advanced infantile fibrosarcoma that would necessitate disfiguring surgery or amputation to achieve complete surgical resection. In some embodiments, the cancer is a myofibroblastic/fibroblastic tumor. The cancer can be a solid tumor or a primary CNS tumor. The cancer can also be a congenital mesoblastic nephroma.

In some embodiments, one or more Trk inhibitors as provided herein and optionally an immunotherapy agent are useful for treating Trk-associated cancers in pediatric patients. For example, the one or more Trk inhibitors as provided herein and optionally an immunotherapy agent can be used to treat infantile sarcoma, glioma (e.g., pediatric gliomas), neuroblastoma, congenital mesoblastic nephroma, brain low-grade glioma, and pontine glioma.

In some embodiments, the Trk-associated cancer is a glioma. For example, the Trk-associated cancer is selected from the group consisting of: pediatric high-grade glioma (HGG), diffuse intrinsic pontine gliomas (DIPGs), and on-brainstem HGGs (NBS-HGGs). In some embodiments, the cancer is an extracranial solid tumor. For example, the pediatric cancer is selected from the group consisting of: neuroblastoma, nephroblastoma (e.g., Wilm's tumor), rhabdomyosarcoma and hepatoblastoma.

In some embodiments, the fibrosarcoma is infantile fibrosarcoma.

In some embodiments, the Trk-associated cancer is LMNA-NTRK1 fusion soft tissue sarcoma or EVT6-NTRK3 fusion papillary thyroid cancer.

In one embodiment, a pharmaceutical composition comprising a compounding agent as disclosed herein and Compound 1 or a solid form thereof, crystalline form thereof, or solvate or hydrate thereof, or a salt of Compound 1 or solid form thereof, crystalline form thereof, or solvate or hydrate thereof, as described herein, are useful for treating diseases and disorders which can be treated with a Trk inhibitor. Non-limiting examples of cancer (e.g., a Trk-associated cancer) include adenocarcinoma, adrenal gland cortical carcinoma, adrenal gland neuroblastoma, anus squamous cell carcinoma, appendix adenocarcinoma, bladder urothelial carcinoma, bile duct adenocarcinoma, bladder carcinoma, bladder urothelial carcinoma, bone chordoma, bone marrow leukemia lymphocytic chronic, bone marrow leukemia non-lymphocytic acute myelocytic, bone marrow lymph proliferative disease, bone marrow multiple myeloma, bone sarcoma, sarcoma, primary brain neoplasm, brain astrocytoma, brain glioblastoma, brain medulloblastoma, brain meningioma, brain oligodendroglioma, pontine glioma, breast adenoid cystic carcinoma, malignant peripheral nerve sheath tumor, breast carcinoma, breast ductal carcinoma in situ, breast invasive ductal carcinoma, breast invasive lobular carcinoma, breast metaplastic carcinoma, cervix neuroendocrine carcinoma, cervix squamous cell carcinoma, colon adenocarcinoma, colon carcinoid tumor, duodenum adenocarcinoma, endometrioid tumor, esophagus adenocarcinoma, eye intraocular melanoma, eye intraocular squamous cell carcinoma, eye lacrimal duct carcinoma, fallopian tube serous carcinoma, gallbladder adenocarcinoma, gallbladder glomus tumor, gastroesophageal junction adenocarcinoma, head and neck adenoid cystic carcinoma, head and neck carcinoma, head and neck neuroblastoma, head and neck squamous cell carcinoma, kidney chromophore carcinoma, kidney medullary carcinoma, kidney renal cell carcinoma, kidney renal papillary carcinoma, kidney sarcomatoid carcinoma, kidney urothelial carcinoma, leukemia lymphocytic, liver cholangiocarcinoma, liver hepatocellular carcinoma, lung adenocarcinoma, lung adenosquamous carcinoma, lung atypical carcinoid, lung carcinosarcoma, lung large cell neuroendocrine carcinoma, non-small cell lung carcinoma, lung sarcoma, lung sarcomatoid carcinoma, lung small cell carcinoma, lung small cell undifferentiated carcinoma, lung squamous cell carcinoma, lymph node lymphoma diffuse large B cell, lymph node lymphoma follicular lymphoma, lymph node lymphoma mediastinal B-cell, lymph node lymphoma plasmablastic lung adenocarcinoma, lymphoma follicular lymphoma, lymphoma, non-Hodgkin's lymphoma, nasopharynx and paranasal sinuses undifferentiated carcinoma, ovary carcinoma, ovary carcinosarcoma, ovary clear cell carcinoma, ovary epithelial carcinoma, ovary granulosa cell tumor, ovary serous carcinoma, pancreas carcinoma, pancreas ductal adenocarcinoma, pancreas neuroendocrine carcinoma, peritoneum mesothelioma, peritoneum serous carcinoma, placenta choriocarcinoma, pleura mesothelioma, prostate acinar adenocarcinoma, prostate carcinoma, rectum adenocarcinoma, rectum squamous cell carcinoma, skin carcinoma, skin adnexal carcinoma, skin basal cell carcinoma, skin melanoma, skin Merkel cell carcinoma, skin squamous cell carcinoma, biliary tract neoplasm, a bile duct neoplasm, small intestine adenocarcinoma, a gastrointestinal stromal tumor (GISTs), a small intestine gastrointestinal stromal tumor (a small intestine GIST), soft tissue angiosarcoma, soft tissue Ewing sarcoma, soft tissue hemangioendothelioma, soft tissue inflammatory myofibroblastic tumor, soft tissue leiomyosarcoma, soft tissue liposarcoma, soft tissue neuroblastoma, soft tissue paraganglioma, soft tissue perivascular epitheliod cell tumor, soft tissue sarcoma, soft tissue synovial sarcoma, stomach adenocarcinoma, stomach adenocarcinoma diffuse-type, stomach adenocarcinoma intestinal type, stomach adenocarcinoma intestinal type, stomach leiomyosarcoma, thymus carcinoma, thymus thymoma lymphocytic, thyroid papillary carcinoma, unknown primary adenocarcinoma, unknown primary carcinoma, unknown primary malignant neoplasm, melanoma, unknown primary melanoma, unknown primary sarcomatoid carcinoma, unknown primary squamous cell carcinoma, unknown undifferentiated neuroendocrine carcinoma, unknown primary undifferentiated small cell carcinoma, uterus carcinosarcoma, uterus endometrial adenocarcinoma, uterus endometrial adenocarcinoma endometrioid, uterus endometrial adenocarcinoma papillary serous, and uterus leiomyosarcoma.

Additional examples of cancers (e.g., Trk inhibitor-resistant cancer) include: adrenocortical carcinoma, anal cancer, appendix cancer, atypical teratoid/rhabdoid tumor (e.g., central nervous system atypical teratoid/rhabdoid tumor), B-cell cancer, bile duct cancer, bladder cancer, bone cancer (e.g., osteosarcoma and malignant fibrous histiocytoma), brain cancer (e.g., brain and spinal cord tumor, brain stem glioma, central nervous system embryonal tumors, central nervous system germ cell tumors, craniopharyngioma, and ependymoma), nerve tissue neoplasm, central nervous system neoplasm, breast cancer, bronchial neoplasm, bronchogenic carcinoma, bronchus cancer, cancer of hematological tissues, cancer of the oral cavity or pharynx, carcinoid tumor, cervical cancer, childhood cancers, chordoma, chronic lymphocytic leukemia, chronic myeloproliferative neoplasms, colon cancer, colorectal cancer, cutaneous T-cell lymphoma, ductal carcinoma in situ, embryonal tumor, endometrial cancer, esophageal cancer, esthesioneuroblastoma, extracranial germ cell tumor, extragonadal germ cell tumor, extrahepatic bile duct cancer, eye cancer (e.g., retinoblastoma), fallopian tube cancer, fibrosarcoma, fibrous histiocytoma of bone, gallbladder cancer, thoracic neoplasm, gastric cancer, gastrointestinal carcinoid tumor, germ cell tumor, gestational trophoblastic disease, glioblastoma multiforme, glioma (e.g., lower-grade glioma), head and neck cancer, heart cancer, histiocytosis, hypopharyngeal cancer, inflammatory myofibroblastic tumors, intrahepatic cholangiocarcinoma, islet cell tumor, kidney cancer (e.g., renal cell cancer), Langerhans cell histiocytosis, large cell neuroendocrine cancer, laryngeal cancer, leukemia (e.g., acute lymphoblastic leukemia, acute myeloid leukemia, chronic myelogenous leukemia, and hairy cell leukemia), lip cancer, liver cancer, lung cancer, respiratory tract neoplasm, Burkitt lymphoma, Hodgkin's lymphoma, and primary central nervous system lymphoma), medulloblastoma, mesothelioma, mouth cancer, multiple myeloma, myelodysplastic syndromes, nasal cavity and paranasal sinus cancer, nasopharyngeal cancer, neoplasm (e.g., a melanocystic neoplasm), nephroma, neuroblastoma, non-small cell lung cancer, oral cancer, oropharyngeal cancer, ovarian cancer, pancreatic cancer, paraganglioma, thyroid cancer, parathyroid cancer, pediatric glioma, penile cancer, pharyngeal cancer, pheochromocytoma, pilocytic astrocytoma, pituitary tumor, plasma cell neoplasm, primary peritoneal cancer, prostate cancer, rectum carcinoma, salivary gland cancer, sarcoma (e.g., Ewing sarcoma, rhabdomyosarcoma, uterine sarcoma, and undifferentiated sarcoma), secretory breast carcinoma, Sezary syndrome, skin cancer, small bowel cancer, small cell lung cancer, intestinal neoplasm, small intestine cancer, nevi and melanoma, Spitz nevi, a Spitz tumor, spitzoid melanoma, stomach cancer, squamous cell carcinoma, squamous neck cancer, testicular cancer, throat cancer, thymoma and thymic carcinoma, thyroid carcinoma, urethral cancer, uterine cancer, urinary bladder cancer, vaginal cancer, vulvar cancer, and Wilms tumor.

In some embodiments, the cancer is selected from the group consisting of: non-small cell lung carcinoma, thyroid neoplasms, sarcoma, GIST, malignant peripheral nerve sheath tumors, colorectal neoplasms, salivary gland neoplasms, biliary tract neoplasms, primary brain neoplasm, breast secretory carcinoma, melanoma, glioblastoma, bile duct neoplasms, astrocytoma, head and neck squamous cell carcinoma, pontine glioma, pancreatic neoplasms, ovarian neoplasms, uterine neoplasms, renal cell carcinoma, cholangiocarcinoma, skin carcinoma, bronchogenic carcinoma, bronchial neoplasms, lung neoplasms, respiratory tract neoplasms, thoracic neoplasms, nerve tissue neoplasms, nevi and melanomas, intestinal neoplasm, thyroid cancer, fibrosarcoma, infantile fibrosarcoma, congenital mesoblastic nephroma, and central nervous system neoplasms.

In some embodiments, the cancer is a pediatric cancer. In some embodiments, the pediatric cancer is a mesenchymal cancer. For example, the mesenchymal cancer can be selected from the group consisting of: pediatric nephroma, congenital fibrosarcoma (CFS), pediatric high-grade glioma (HGG), mesenchymal cancers (infant fibrosarcoma (IF), congenital mesoblastic nephroma, congenital infantile fibrosarcoma (CIFS); pilocytic astrocytoma, brain tumors, pediatic acute leukemia, Ph-like acute lymphoblastic leukemia, cellular congenital mesoblastic nephroma (CMN); infantile fibrosarcoma, pediatric high-grade glioma (HGG), diffuse intrinsic pontine gliomas (DIPGs), non-brainstem HGGs (NBS-HGGs), anaplastic large cell lymphoma (ALCL), non-Hodgkin's lymphoma (NHL), pediatric papillary thyroid carcinoma, soft tissue sarcoma, spitzoid melanoma, pediatric hemangiopericytoma-like sarcoma, spindle cell sarcoma, NOS with myo/haemangiopericytic growth pattern, lung cancer, advanced pediatric solid tumors, neuroectodermal-derived tumors, pediatric colorectal cancer, adrenal neuroblastoma, and central nervous system tumors.

In some embodiments, the pediatric cancer is a fibrosarcoma such as infantile fibrosarcoma.

In some embodiments, the pediatric cancer is a glioma. For example, the pediatric cancer is selected from the group consisting of: pediatric high-grade glioma (HGG), diffuse intrinsic pontine gliomas (DIPGs), and on-brainstem HGGs (NBS-HGGs).

Provided herein are methods of treating a subject having a cancer (e.g., any of the cancers described herein) that include identifying a subject in which (i) the cancer in the subject has relapsed during therapy with a first Trk inhibitor; and/or (ii) the cancer in the subject is not responding to therapy with a first Trk inhibitor; and/or (iii) the subject is intolerant to a first Trk inhibitor, and administering to the identified subject a treatment that does not include a first Trk inhibitor (e.g., a first Trk inhibitor such as entrectinib, TPX-0005, PLX7486, or (S)—N-(5-((R)-2-(2,5-difluorophenyl)pyrrolidin-1-yl)pyrazolo[1,5-a]pyrimidin-3-yl)-3-hydroxypyrrolidine-1-carboxamide sulfate) as a monotherapy (e.g., any treatments that do not include a first Trk inhibitor as a monotherapy described herein). For example, the subject can be administered a second Trk inhibitor as a monotherapy or in combination with another anticancer agent or treatment (e.g., the first Trk inhibitor).

Also provided herein are methods of treating a subject having a cancer (e.g., any of the cancers described herein) in which (i) the cancer in the subject has relapsed during therapy with the first Trk inhibitor; and/or (ii) the cancer in the subject is not responding to therapy with the first Trk inhibitor; and/or (iii) the subject is intolerant to the first Trk inhibitor that include administering to the subject a treatment that does not include a first Trk inhibitor (e.g., a first Trk inhibitor such as entrectinib, TPX-0005, PLX7486, or (S)—N-(5-((R)-2-(2,5-difluorophenyl)pyrrolidin-1-yl)pyrazolo[1,5-a]pyrimidin-3-yl)-3-hydroxypyrrolidine-1-carboxamide sulfate) as a monotherapy (e.g., any treatments that do not include a first Trk inhibitor as a monotherapy described herein). For example, the subject can be administered a second Trk inhibitor as a monotherapy or in combination with another anticancer agent or treatment (e.g., the first Trk inhibitor).

Also provided herein are methods of treating a subject that include administering a therapeutically effective amount of a treatment that does not include a first Trk inhibitor as a monotherapy, to a subject having a clinical record that indicates that (i) the cancer in the subject has relapsed during therapy with the first Trk inhibitor; and/or (ii) the cancer in the subject is not responding to therapy with a first Trk inhibitor; and/or (iii) the subject is intolerant to a first Trk inhibitor. For example, the subject can be administered a second Trk inhibitor as a monotherapy or in combination with another anticancer agent or treatment (e.g., the first Trk inhibitor).

Also provided herein are methods of treating a subject having a cancer (e.g., any of the cancers described herein or known in the art) that include: identifying a subject in which (i) the cancer in the subject has relapsed during therapy with a first Trk inhibitor; and/or (ii) the cancer in the subject is not responding to therapy with a first Trk inhibitor; and/or (iii) the subject is intolerant to a first Trk inhibitor; and administering to the identified subject a treatment that includes Compound 1, or a pharmaceutically acceptable salt, amorphous, or polymorph form thereof.

Also provided herein are methods of treating a subject having a cancer (e.g., any of the cancers described herein or known in the art) that include: identifying a subject in which (i) the cancer in the subject has relapsed during therapy with a first Trk inhibitor; and/or (ii) the cancer in the subject is not responding to therapy with a first Trk inhibitor; and/or (iii) the subject is intolerant to a first Trk inhibitor; and administering to the identified subject a treatment that includes Compound 1, or a pharmaceutically acceptable salt, amorphous, or polymorph form thereof, and another anticancer agent (e.g., any one or more of the anticancer agents described herein) or anticancer therapy (e.g., any one or more of the anticancer therapies provided herein).

Also provided herein are methods of treating a subject identified as having a cancer wherein (i) the cancer in the subject has relapsed during therapy with a first Trk inhibitor; and/or (ii) the cancer in the subject is not responding to therapy with a first Trk inhibitor; and/or (iii) the subject is intolerant to a first Trk inhibitor, that include administering to the subject a treatment that includes Compound 1, or a pharmaceutically acceptable salt, amorphous, or polymorph form thereof.

Also provided herein are methods of treating a subject identified as having a cancer and wherein (i) the cancer in the subject has relapsed during during therapy with a first Trk inhibitor; and/or (ii) the cancer in the subject is not responding to therapy with a first Trk inhibitor; and/or (iii) the subject is intolerant to a first Trk inhibitor, that include administering to the subject a treatment that includes Compound 1, or a pharmaceutically acceptable salt, amorphous, or polymorph form thereof, and another anticancer agent (e.g., any one or more of the another anticancer agents described herein) or anticancer therapies (e.g., any one or more of the anticancer therapies described herein).

Also provided herein are methods of treating a subject having a cancer that include administering a therapeutically effective amount of a treatment that includes Compound 1, or a pharmaceutically acceptable salt, amorphous, or polymorph form thereof, to a subject having a clinical record that indicates that (i) the cancer in the subject has relapsed during therapy with a first Trk inhibitor; and/or (ii) the cancer in the subject is not responding to therapy with a first Trk inhibitor; and/or (iii) the subject is intolerant to a first Trk inhibitor.

Also provided herein are methods of treating a subject that include administering a therapeutically effective amount of a treatment that includes Compound 1, or a pharmaceutically acceptable salt, amorphous, or polymorph form thereof, and another anticancer agent (e.g., any one or more of the anticancer agents described herein) or anticancer therapy (e.g., any one or more of the anticancer therapies described herein), to a subject having a clinical record that indicates that (i) the cancer in the subject has relapsed during therapy with a first Trk inhibitor; and/or (ii) the cancer in the subject is not responding to therapy with a first Trk inhibitor; and/or (iii) the subject is intolerant to a first Trk inhibitor.

Also provided herein are methods of treating a subject having a cancer that include (a) administering one or more doses of a first Trk inhibitor to the subject for a period of time; (b) after (a), determining whether (i) the cancer in the subject has relapsed during therapy with the first Trk inhibitor; and/or (ii) the cancer in the subject is not responding to therapy with the first Trk inhibitor; and/or (iii) the subject is intolerant to the first Trk inhibitor; and (c) administering a second Trk inhibitor or a treatment that does not include the Trk inhibitor of step (a) as a monotherapy to a subject in which (i) the cancer in the subject has relapsed during therapy with the first Trk inhibitor; and/or (ii) the cancer in the subject is not responding to therapy with the first Trk inhibitor; and/or (iii) the subject is intolerant to the first Trk inhibitor; or (d) administering additional doses of the first Trk inhibitor to a subject in which (i) the cancer in the subject has not relapsed during therapy with the first Trk inhibitor; and/or (ii) the cancer in the subject is responding to therapy with the first Trk inhibitor; and/or (iii) the subject is not intolerant to the first Trk inhibitor. In some embodiments, the second Trk inhibitor is Compound 1, or a pharmaceutically acceptable salt, amorphous, or polymorph form thereof. In some embodiments the second Trk inhibitor is a pharmaceutical composition comprising a compounding agent as disclosed herein and Compound 1 or a solid form thereof, crystalline form thereof, or solvate or hydrate thereof, or a salt of Compound 1 or solid form thereof, crystalline form thereof, or solvate or hydrate thereof, as described herein. In some embodiments, the cancer is a Trk-associated cancer.

Also provided herein are methods of treating a subject having a cancer that include: (a) determining whether (i) the cancer in the subject has relapsed during therapy with a first Trk inhibitor; and/or (ii) the cancer in the subject is not responding to therapy with a first Trk inhibitor; and/or (iii) the subject is intolerant to a first Trk inhibitor; and (b) administering a second Trk inhibitor or a treatment that does not include the Trk inhibitor of step (a) as a monotherapy to a subject in which (i) the cancer in the subject has relapsed during therapy with the first Trk inhibitor; and/or (ii) the cancer in the subject is not responding to therapy with the first Trk inhibitor; and/or (iii) the subject is intolerant to the first Trk inhibitor; or (c) administering additional doses of the first Trk inhibitor to a subject in which (i) the cancer in the subject has not relapsed during therapy with the first Trk inhibitor; and/or (ii) the cancer in the subject is responding to therapy with the first Trk inhibitor; and/or (iii) the subject is not intolerant to the first Trk inhibitor. In some embodiments, the second Trk inhibitor is Compound 1, or a pharmaceutically acceptable salt, amorphous, or polymorph form thereof. In some embodiments the second Trk inhibitor is a pharmaceutical composition comprising a compounding agent as disclosed herein and Compound 1 or a solid form thereof, crystalline form thereof, or solvate or hydrate thereof, or a salt of Compound 1 or solid form thereof, crystalline form thereof, or solvate or hydrate thereof, as described herein.

In some embodiments, the cancer is a Trk-associated cancer. In some embodiments, the Trk associated cancer exhibits at least one of a NTRK1, NTRK2, and/or NTRK3 fusion. In some embodiments, the at least one NTRK1, NTRK2, and/or NTRK3 fusion results in the expression of one or more of a TrkA fusion protein, and/or a TrkB fusion protein, and/or a TrkC fusion protein, wherein the TrkA fusion protein comprises one or more of the of the fusions selected from the group consisting of: TP53-TrkA, LMNA-TrkA, CD74-TrkA, TFG-TrkA, TPM3-TrkA, NFASC-TrkA, BCAN-TrkA, MPRIP-TrkA, TPR-TrkA, RFWD2-TrkA, IRF2BP2-TrkA, SQSTM1-TrkA, SSBP2-TrkA, RABGAP1L-TrkA, C18ORF8-TrkA, RNF213-TrkA, TBC1D22A-TrkA, C20ORF112-TrkA, DNER-TrkA, ARHGEF2-TrkA, CHTOP-TrkA, PPL-TrkA, PLEKHA6-TrkA, PEAR1-TrkA, MRPL24-TrkA, MDM4-TrkA, LRRC71-TrkA, GRIPAP1-TrkA, TAF-TrkA, EPS15-TrkA, DYNC2H1-TrkA, CEL-TrkA, EPHB2-TrkA, TGF-TrkA, NELL1-TrkA, EPL4-TrkA, CTNND2-TrkA, TCEANC2-TrkA, SCYL3-TrkA, AMOTL2-TrkA, MEF2D-TrkA, L7a-TrkA, ZBTB7B-TrkA, TRIM63-TrkA, DDR2-TrkAl, GON4L-TrkA, PDE4DIP-TrkA, NTRK1-P2RY8, CTRC-TrkA, and VANGL2-TrkA; and/or the TrkB fusion protein comprises one or more of the of the fusions selected from the group consisting of: NACC2-TrkB, QKI-TrkB, AFAP1-TrkB, PAN3-TrkB, SQSTM1-TrkB, TRIM24-TrkB, VCL-TrkB, AGBL4-TrkB, DAB2IP-TrkB, TrkB-TERT, ETV6-TrkB, NOS1AP-TrkB, GKAP1-TrkB, KCTD8-TrkB, TBC1D2-TrkB, VCAN-TrkB, SLMAP-TrkB, TLE4-TrkB, STRN3-TrkB, WNK2-TrkB, TrkB-BEND5, TrkB-TRAF2, Nav1-TrkB, and STRN-TrkB; and/or the TrkC fusion protein comprises one or more of the of the fusions selected from the group consisting of: ETV6-TrkC1, BTBD1-TrkC, LYN-TrkC, RBPMS-TrkC, EML4-TrkC, TrkC-HOMER2, TFG-TrkC, FAT1-TrkC, MYO5A-TrkC, MYH9-TrkC, KANK1-TrkC, SQSTM1-TrkC, UBE2R2-TrkC, HNRNPA2B1-TrkC, VPS18-TrkC, AKAP13-TrkC, TrkC-LOXL2, TrkC-PEAK1, ZNF710-TrkC, TPM4-TrkC, and LMNA-TrkC. In some embodiments, the Trk-associated cancer exhibits one or mutations point mutations/insertions/deletions in one or more of NTRK1, NTRK2, or NTRK3. Non-limiting examples of Trk kinase point mutations/insertions/deletions are described in Tables 3, 4, 6, 7, 9, 10. In some embodiments, the Trk-associated cancer does not exhibit a Trk resistance mutation, e.g., any of the mutations described in Tables 11-13. Also provided herein are methods of treating a subject having a cancer that include (a) detecting a dysregulation of a NTRK gene, a Trk kinase, or the expression or activity or level of any of the same; (b) administering one or more doses of a first Trk inhibitor to the subject for a period of time; (c) after (a) and (b), determining whether (i) the cancer in the subject has relapsed during therapy with the first Trk inhibitor; and/or (ii) the cancer in the subject is not responding to therapy with the first Trk inhibitor; and/or (iii) the subject is intolerant to the first Trk inhibitor; and (d) administering a second Trk inhibitor or a treatment that does not include the Trk inhibitor of step (b) as a monotherapy to a subject in which (i) the cancer in the subject has relapsed during therapy with the first Trk inhibitor; and/or (ii) the cancer in the subject is not responding to therapy with the first Trk inhibitor; and/or (iii) the subject is intolerant to the first Trk inhibitor; or (e) administering additional doses of the first Trk inhibitor to a subject in which (i) the cancer in the subject has not relapsed during therapy with the first Trk inhibitor; and/or (ii) the cancer in the subject is responding to therapy with the first Trk inhibitor; and/or (iii) the subject is not intolerant to the first Trk inhibitor. In some embodiments, the second Trk inhibitor is Compound 1, or a pharmaceutically acceptable salt, amorphous, or polymorph form thereof. In some embodiments the second Trk inhibitor is a pharmaceutical composition comprising a compounding agent as disclosed herein and Compound 1 or a solid form thereof, crystalline form thereof, or solvate or hydrate thereof, or a salt of Compound 1 or solid form thereof, crystalline form thereof, or solvate or hydrate thereof, as described herein.

In some embodiments, step (a) is performed before step (b).

In some embodiments, step (b) is performed before step (a).

In some embodiments, detecting a dysregulation of a NTRK gene, a Trk kinase, or the expression or activity or level of any of the same includes next generation sequencing, immunohistochemistry, fluorescence microscopy, break apart FISH analysis, and PCR-based amplification (e.g., RT-PCR and quantitative real-time RT-PCR).

In some embodiments, the dysregulation of a NTRK gene, a Trk kinase, or the expression or activity or level of any of the same is at least one NTRK1, NTRK2, and/or NTRK3 fusion. In some embodiments, the at least one NTRK1, NTRK2, and/or NTRK3 fusion results in the expression of one or more of a TrkA fusion protein, and/or a TrkB fusion protein, and/or a TrkC fusion protein, wherein the TrkA fusion protein comprises one or more of the of the fusions selected from the group consisting of: TP53-TrkA, LMNA-TrkA, CD74-TrkA, TFG-TrkA, TPM3-TrkA, NFASC-TrkA, BCAN-TrkA, MPRIP-TrkA, TPR-TrkA, RFWD2-TrkA, IRF2BP2-TrkA, SQSTM1-TrkA, SSBP2-TrkA, RABGAP1L-TrkA, C18ORF8-TrkA, RNF213-TrkA, TBC1D22A-TrkA, C20ORF112-TrkA, DNER-TrkA, ARHGEF2-TrkA, CHTOP-TrkA, PPL-TrkA, PLEKHA6-TrkA, PEAR1-TrkA, MRPL24-TrkA, MDM4-TrkA, LRRC71-TrkA, GRIPAP1-TrkA, TAF-TrkA, EPS15-TrkA, DYNC2H1-TrkA, CEL-TrkA, EPHB2-TrkA, TGF-TrkA, NELL1-TrkA, EPL4-TrkA, CTNND2-TrkA, TCEANC2-TrkA, SCYL3-TrkA, AMOTL2-TrkA, MEF2D-TrkA, L7a-TrkA, ZBTB7B-TrkA, TRIM63-TrkA, DDR2-TrkAl, GON4L-TrkA, PDE4DIP-TrkA, NTRK1-P2RY8, CTRC-TrkA, and VANGL2-TrkA; and/or the TrkB fusion protein comprises one or more of the of the fusions selected from the group consisting of: NACC2-TrkB, QKI-TrkB, AFAP1-TrkB, PAN3-TrkB, SQSTM1-TrkB, TRIM24-TrkB, VCL-TrkB, AGBL4-TrkB, DAB2IP-TrkB, TrkB-TERT, ETV6-TrkB, NOS1AP-TrkB, GKAP1-TrkB, KCTD8-TrkB, TBC1D2-TrkB, VCAN-TrkB, SLMAP-TrkB, TLE4-TrkB, STRN3-TrkB, WNK2-TrkB, TrkB-BEND5, TrkB-TRAF2, Nav1-TrkB, and STRN-TrkB; and/or the TrkC fusion protein comprises one or more of the of the fusions selected from the group consisting of: ETV6-TrkC1, BTBD1-TrkC, LYN-TrkC, RBPMS-TrkC, EML4-TrkC, TrkC-HOMER2, TFG-TrkC, FAT1-TrkC, MYO5A-TrkC, MYH9-TrkC, KANK1-TrkC, SQSTM1-TrkC, UBE2R2-TrkC, HNRNPA2B1-TrkC, VPS18-TrkC, AKAP13-TrkC, TrkC-LOXL2, TrkC-PEAK1, ZNF710-TrkC, TPM4-TrkC, and LMNA-TrkC. In some embodiments, the dysregulation of a NTRK gene, a Trk kinase, or the expression or activity or level of any of the same is one or mutations point mutations/insertions/deletions in one or more of NTRK1, NTRK2, or NTRK3. Non-limiting examples of Trk kinase point mutations/insertions/deletions are described in Tables 3, 4, 6, 7, 9, 10. In some embodiments, the Trk-associated cancer does not exhibit a Trk resistance mutation, e.g., any of the mutations described in Tables 11-13.

Also provided herein are methods of treating a subject having a cancer, that include: (a) detecting a dysregulation of a NTRK gene, a Trk kinase, or the expression or activity or level of any of the same; (b) administering one or more doses of a first Trk inhibitor to the subject for a period of time; (c) after (a) and (b), determining whether (i) the cancer in the subject has relapsed during therapy with the first Trk inhibitor; and/or (ii) the cancer in the subject is not responding to therapy with the first Trk inhibitor; and/or (iii) the subject is intolerant to the first Trk inhibitor; and (d) administering a treatment including one or more doses of a second Trk inhibitor to a subject in which (i) the cancer in the subject has relapsed during therapy with the first Trk inhibitor; and/or (ii) the cancer in the subject is not responding to therapy with the first Trk inhibitor; and/or (iii) the subject is intolerant to the first Trk inhibitor; or (e) administering additional doses of the first Trk inhibitor to a subject in which (i) the cancer has not relapsed during therapy with the first Trk inhibitor; and/or (ii) the cancer in the subject is responding to therapy with the first Trk inhibitor; and/or (iii) the subject is not intolerant to the first Trk inhibitor. In some embodiments, the second Trk inhibitor is Compound 1, or a pharmaceutically acceptable salt, amorphous, or polymorph form thereof. In some embodiments the second Trk inhibitor is a pharmaceutical composition comprising a compounding agent as disclosed herein and Compound 1 or a solid form thereof, crystalline form thereof, or solvate or hydrate thereof, or a salt of Compound 1 or solid form thereof, crystalline form thereof, or solvate or hydrate thereof, as described herein.

Also provided herein are methods of treating a subject having a cancer, that include: (a) detecting a dysregulation of a NTRK gene, a Trk kinase, or the expression or activity or level of any of the same; (b) administering one or more doses of a first Trk inhibitor to the subject for a period of time; (c) after (a) and (b), determining whether (i) the cancer in the subject has relapsed during therapy with the first Trk inhibitor; and/or (ii) the cancer in the subject is not responding to therapy with the first Trk inhibitor; and/or (iii) the subject is intolerant to the first Trk inhibitor; and; and (d) administering a treatment including Compound 1, or a pharmaceutically acceptable salt, amorphous, or polymorph form thereof, to a subject in which (i) the cancer in the subject has relapsed during therapy with the first Trk inhibitor; and/or (ii) the cancer in the subject is not responding to therapy with the first Trk inhibitor; and/or (iii) the subject is intolerant to the first Trk inhibitor; or (e) administering additional doses of the first Trk inhibitor to a subject in which (i) the cancer in the subject has not relapsed during therapy with the first Trk inhibitor; and/or (ii) the cancer in the subject is responding to therapy with the first Trk inhibitor; and/or (iii) the subject is not intolerant to the first Trk inhibitor.

Also provided herein are methods of treating a subject having a cancer that include: (a) detecting a dysregulation of a NTRK gene, a Trk kinase, or the expression or activity or level of any of the same; (b) administering one or more doses of a first Trk inhibitor to the subject for a period of time; (c) after (a) and (b), determining whether (i) the cancer in the subject has relapsed during therapy with the first Trk inhibitor; and/or (ii) the cancer in the subject is not responding to therapy with the first Trk inhibitor; and/or (iii) the subject is intolerant to the first Trk inhibitor; and; and (d) administering a treatment including Compound 1, or a pharmaceutically acceptable salt, amorphous, or polymorph form thereof, and another anticancer agent or anticancer therapy to a subject in which (i) the cancer in the subject has relapsed during therapy with the first Trk inhibitor; and/or (ii) the cancer in the subject is not responding to therapy with the first Trk inhibitor; and/or (iii) the subject is intolerant to the first Trk inhibitor; or (e) administering additional doses of the first Trk inhibitor to a subject in which (i) the cancer in the subject has not relapsed during therapy with the first Trk inhibitor; and/or (ii) the cancer in the subject is responding to therapy with the first Trk inhibitor; and/or (iii) the subject is not intolerant to the first Trk inhibitor.

In some embodiments, step (a) is performed before step (b).

In some embodiments, step (b) is performed before step (a).

In some embodiments, detecting a dysregulation of a NTRK gene, a Trk kinase, or the expression or activity or level of any of the same includes next generation sequencing, immunohistochemistry, fluorescence microscopy, break apart FISH analysis, and PCR-based amplification (e.g., RT-PCR and quantitative real-time RT-PCR).

In some embodiments, the dysregulation of a NTRK gene, a Trk kinase, or the expression or activity or level of any of the same is at least one NTRK1, NTRK2, and/or NTRK3 fusion. In some embodiments, the at least one NTRK1, NTRK2, and/or NTRK3 fusion results in the expression of one or more of a TrkA fusion protein, and/or a TrkB fusion protein, and/or a TrkC fusion protein, wherein the TrkA fusion protein comprises one or more of the of the fusions selected from the group consisting of: TP53-TrkA, LMNA-TrkA, CD74-TrkA, TFG-TrkA, TPM3-TrkA, NFASC-TrkA, BCAN-TrkA, MPRIP-TrkA, TPR-TrkA, RFWD2-TrkA, IRF2BP2-TrkA, SQSTM1-TrkA, SSBP2-TrkA, RABGAP1L-TrkA, C18ORF8-TrkA, RNF213-TrkA, TBC1D22A-TrkA, C20ORF112-TrkA, DNER-TrkA, ARHGEF2-TrkA, CHTOP-TrkA, PPL-TrkA, PLEKHA6-TrkA, PEAR1-TrkA, MRPL24-TrkA, MDM4-TrkA, LRRC71-TrkA, GRIPAP1-TrkA, TAF-TrkA, EPS15-TrkA, DYNC2H1-TrkA, CEL-TrkA, EPHB2-TrkA, TGF-TrkA, NELL1-TrkA, EPL4-TrkA, CTNND2-TrkA, TCEANC2-TrkA, SCYL3-TrkA, AMOTL2-TrkA, MEF2D-TrkA, L7a-TrkA, ZBTB7B-TrkA, TRIM63-TrkA, DDR2-TrkAl, GON4L-TrkA, PDE4DIP-TrkA, NTRK1-P2RY8, CTRC-TrkA, and VANGL2-TrkA; and/or the TrkB fusion protein comprises one or more of the of the fusions selected from the group consisting of: NACC2-TrkB, QKI-TrkB, AFAP1-TrkB, PAN3-TrkB, SQSTM1-TrkB, TRIM24-TrkB, VCL-TrkB, AGBL4-TrkB, DAB2IP-TrkB, TrkB-TERT, ETV6-TrkB, NOS1AP-TrkB, GKAP1-TrkB, KCTD8-TrkB, TBC1D2-TrkB, VCAN-TrkB, SLMAP-TrkB, TLE4-TrkB, STRN3-TrkB, WNK2-TrkB, TrkB-BEND5, TrkB-TRAF2, Nav1-TrkB, and STRN-TrkB; and/or the TrkC fusion protein comprises one or more of the of the fusions selected from the group consisting of: ETV6-TrkC1, BTBD1-TrkC, LYN-TrkC, RBPMS-TrkC, EML4-TrkC, TrkC-HOMER2, TFG-TrkC, FAT1-TrkC, MYO5A-TrkC, MYH9-TrkC, KANK1-TrkC, SQSTM1-TrkC, UBE2R2-TrkC, HNRNPA2B1-TrkC, VPS18-TrkC, AKAP13-TrkC, TrkC-LOXL2, TrkC-PEAK1, ZNF710-TrkC, TPM4-TrkC, and LMNA-TrkC. In some embodiments, the dysregulation of a NTRK gene, a Trk kinase, or the expression or activity or level of any of the same is one or mutations point mutations/insertions/deletions in one or more of NTRK1, NTRK2, or NTRK3. Non-limiting examples of Trk kinase point mutations/insertions/deletions are described in Tables 3, 4, 6, 7, 9, 10. In some embodiments, the Trk-associated cancer does not exhibit a Trk resistance mutation, e.g., any of the mutations described in Tables 11-13.

Also provided herein are methods of treating a subject having a cancer that include: (a) determining whether (i) the cancer in the subject has relapsed during therapy with a first Trk inhibitor; and/or (ii) the cancer in the subject is not responding to therapy with a first Trk inhibitor; and/or (iii) the subject is intolerant to a first Trk inhibitor; (b) administering a treatment that includes one or more doses of a second Trk inhibitor to a subject in which (i) the cancer in the subject has relapsed during therapy with the first Trk inhibitor; and/or (ii) the cancer in the subject is not responding to therapy with the first Trk inhibitor; and/or (iii) the subject is intolerant to the first Trk inhibitor; or (c) administering additional doses of the first Trk inhibitor to a subject in which (i) the cancer has not relapsed during therapy with the first Trk inhibitor; and/or (ii) the cancer in the subject is responding to therapy with the first Trk inhibitor; and/or (iii) the subject is not intolerant to the first Trk inhibitor. In some embodiments, the second Trk inhibitor is Compound 1, or a pharmaceutically acceptable salt, amorphous, or polymorph form thereof. In some embodiments the second Trk inhibitor is a pharmaceutical composition comprising a compounding agent as disclosed herein and Compound 1 or a solid form thereof, crystalline form thereof, or solvate or hydrate thereof, or a salt of Compound 1 or solid form thereof, crystalline form thereof, or solvate or hydrate thereof, as described herein.

Also provided herein are methods of treating a subject having a cancer that include: (a) determining whether (i) the cancer in the subject has relapsed during therapy with a first Trk inhibitor; and/or (ii) the cancer in the subject is not responding to therapy with a first Trk inhibitor; and/or (iii) the subject is intolerant to a first Trk inhibitor; (b) administering a treatment that includes Compound 1, or a pharmaceutically acceptable salt, amorphous, or polymorph form thereof, to a subject in which (i) the cancer in the subject has relapsed during therapy with the first Trk inhibitor; and/or (ii) the cancer in the subject is not responding to therapy with the first Trk inhibitor; and/or (iii) the subject is intolerant to the first Trk inhibitor; or (c) administering additional doses of the first Trk inhibitor to a subject in which (i) the cancer has not relapsed during therapy with the first Trk inhibitor; and/or (ii) the cancer in the subject is responding to therapy with the first Trk inhibitor; and/or (iii) the subject is not intolerant to the first Trk inhibitor.

Also provided herein are methods of treating a subject having a cancer, that include: (a) determining whether (i) the cancer in the subject has relapsed during therapy with a first Trk inhibitor; and/or (ii) the cancer in the subject is not responding to therapy with a first Trk inhibitor; and/or (iii) the subject is intolerant to a first Trk inhibitor; (b) administering a treatment that includes Compound 1, or a pharmaceutically acceptable salt, amorphous, or polymorph form thereof, and another anticancer agent or anticancer therapy to a subject in which (i) the cancer in the subject has relapsed during therapy with the first Trk inhibitor; and/or (ii) the cancer in the subject is not responding to therapy with the first Trk inhibitor; and/or (iii) the subject is intolerant to the first Trk inhibitor; or (c) administering additional doses of the first Trk inhibitor to a subject in which (i) the cancer has not relapsed during therapy with the first Trk inhibitor; and/or (ii) the cancer in the subject is responding to therapy with the first Trk inhibitor; and/or (iii) the subject is not intolerant to the first Trk inhibitor.

In some embodiments, the cancer is a Trk-associated cancer. In some embodiments, the Trk associated cancer exhibits at least one of a NTRK1, NTRK2, and/or NTRK3 fusion. In some embodiments, the at least one NTRK1, NTRK2, and/or NTRK3 fusion results in the expression of one or more of a TrkA fusion protein, and/or a TrkB fusion protein, and/or a TrkC fusion protein, wherein the TrkA fusion protein comprises one or more of the of the fusions selected from the group consisting of: TP53-TrkA, LMNA-TrkA, CD74-TrkA, TFG-TrkA, TPM3-TrkA, NFASC-TrkA, BCAN-TrkA, MPRIP-TrkA, TPR-TrkA, RFWD2-TrkA, IRF2BP2-TrkA, SQSTM1-TrkA, SSBP2-TrkA, RABGAP1L-TrkA, C18ORF8-TrkA, RNF213-TrkA, TBC1D22A-TrkA, C20ORF112-TrkA, DNER-TrkA, ARHGEF2-TrkA, CHTOP-TrkA, PPL-TrkA, PLEKHA6-TrkA, PEAR1-TrkA, MRPL24-TrkA, MDM4-TrkA, LRRC71-TrkA, GRIPAP1-TrkA, TAF-TrkA, EPS15-TrkA, DYNC2H1-TrkA, CEL-TrkA, EPHB2-TrkA, TGF-TrkA, NELL1-TrkA, EPL4-TrkA, CTNND2-TrkA, TCEANC2-TrkA, SCYL3-TrkA, AMOTL2-TrkA, MEF2D-TrkA, L7a-TrkA, ZBTB7B-TrkA, TRIM63-TrkA, DDR2-TrkAl, GON4L-TrkA, PDE4DIP-TrkA, NTRK1-P2RY8, CTRC-TrkA, and VANGL2-TrkA; and/or the TrkB fusion protein comprises one or more of the of the fusions selected from the group consisting of: NACC2-TrkB, QKI-TrkB, AFAP1-TrkB, PAN3-TrkB, SQSTM1-TrkB, TRIM24-TrkB, VCL-TrkB, AGBL4-TrkB, DAB2IP-TrkB, TrkB-TERT, ETV6-TrkB, NOS1AP-TrkB, GKAP1-TrkB, KCTD8-TrkB, TBC1D2-TrkB, VCAN-TrkB, SLMAP-TrkB, TLE4-TrkB, STRN3-TrkB, WNK2-TrkB, TrkB-BEND5, TrkB-TRAF2, Nav1-TrkB, and STRN-TrkB; and/or the TrkC fusion protein comprises one or more of the of the fusions selected from the group consisting of: ETV6-TrkC1, BTBD1-TrkC, LYN-TrkC, RBPMS-TrkC, EML4-TrkC, TrkC-HOMER2, TFG-TrkC, FAT1-TrkC, MYO5A-TrkC, MYH9-TrkC, KANK1-TrkC, SQSTM1-TrkC, UBE2R2-TrkC, HNRNPA2B1-TrkC, VPS18-TrkC, AKAP13-TrkC, TrkC-LOXL2, TrkC-PEAK1, ZNF710-TrkC, TPM4-TrkC, and LMNA-TrkC. In some embodiments, the Trk-associated cancer exhibits one or mutations point mutations/insertions/deletions in one or more of NTRK1, NTRK2, or NTRK3. Non-limiting examples of Trk kinase point mutations/insertions/deletions are described in Tables 3, 4, 6, 7, 9, 10. In some embodiments, the Trk-associated cancer does not exhibit a Trk resistance mutation, e.g., any of the mutations described in Tables 11-13.

In some embodiments, the first Trk inhibitor is selected from the group consisting of: entrectinib (N-[5-(3,5-difluoro-benzyl)-1H-indazol-3-yl]-4-(4-methylpiperazin-1-yl)-2-(tetrahydro-pyran-4-ylamino)-benzamide), (S)—N-(5-((R)-2-(2,5-difluorophenyl)pyrrolidin-1-yl)pyrazolo[1,5-a]pyrimidin-3-yl)-3-hydroxypyrrolidine-1-carboxamide sulfate, cabozantinib ((N-(4-((6,7-Dimethoxyquinolin-4-yl)oxy)phenyl)-N′-(4-fluorophenyl)cyclopropane-1,1-dicarboxamide)), dovitinib (4-amino-5-fluoro-3-[6-(4-methylpiperazin-1-yl)-1H-benzimidazol-2-yl]quinolin-2(1H)-one mono 2-hydroxypropanoate hydrate), belizatinib (4-fluoro-N-(6-((4-(2-hydroxypropan-2-yl)piperidin-1-yl)methyl)-1-((1s,4s)-4-(isopropylcarbamoyl)cyclohexyl)-1H-benzo[d]imidazol-2-yl)benzamide), sitravatinib (N-(3-fluoro-4-((2-(5-(((2-methoxyethyl)amino)methyl)pyridin-2-yl)thieno[3,2-b]pyridin-7-yl)oxy)phenyl)-N-(4-fluorophenyl)cyclopropane-1,1-dicarboxamide), PLX7486, altiratinib (N-(4-((2-(cyclopropanecarboxamido)pyridin-4-yl)oxy)-2,5-difluorophenyl)-N-(4-fluorophenyl)cyclopropane-1,1-dicarboxamide), AZD7451 ((S)—N-(1-(5-fluoropyrimidin-2-yl)ethyl)-3-(5-isopropoxy-1H-pyrazol-3-yl)-3H-imidazo[4,5-b]pyridin-5-amine), (6R,15R)-9-fluoro-15-methyl-2,11,16,20,21,24-hexaazapentacyclo[16.5.2.02,6.07,12.021,25]pentacosa-1(24),7,9, 11,18(25),19,22-heptaen-17-one, a (R)-2-phenylpyrrolidine substituted imadazopyridazine, AZD6918, GNF-4256, GTx-186, GNF-5837, AZ623, AG-879, CT327, AR-772, AR-523, AR-786, AR-256, AR-618, AZ-23, CEP-701, CEP-751, PHA-739358, dovitinib, Go 6976, GW441756, MGCD516, ONO-5390556, PHA-848125AC, Regorafenib, Sorafenib, Sunitinib, TSR-011, VM-902A, K252a, a 4-aminopyrazolylpyrimidine, a substituted pyrazolo[1,5-a] pyrimidine compound, BMS-754807, ONO-7579, F17752, ANA-12, ONO-4474, GZ389988, or TPX-0005 ((7S,13R)-11-fluoro-7,13-dimethyl-6,7,13,14-tetrahydro-1,15-ethenopyrazolo[4,3-f][1,4,8,10]benzoxatriazacyclotridecin-4(5H)-one; repotrectinib).

In some embodiments, relapse is one or more of detecting an increase in the number of cancer cells in the subject, an increase in the size of one or more tumors in the subject, an increase in tumor burden, an increase in the rate or extent of metastasis, worsening symptoms, in whole or in part, associated with the cancer, an increase in the extent of disease, and an acceleration of disease progression after a period of improvement. In some embodiments, relapse is progression of the cancer after a period of improvement. In some embodiments, a period of improvement is one or more of a decrease in the number of cancer cells in the subject, a decrease in the size of one or more tumors in the subject, a decrease in tumor burden, a decrease in the rate or extent of metastasis, improving symptoms, in whole or in part, associated with the cancer, a decrease in the extent of disease, and a slowing of disease progression.

In some embodiments, the tumor burden is assessed by RECIST version 1.1.

In some embodiments, the cancer is glioma and the progression of the glioma is assessed by RANO. In some embodiments, the cancer is a primary CNS tumor and the progression of the primary CNS tumor is assessed by RANO.

In some embodiments, a cancer that is not responding to therapy with a first Trk inhibitor is a cancer that is progressing. In some embodiments, progression of a cancer is one or more of an increase in the number of cancer cells in the subject, an increase in the size of one or more tumors in the subject, an increase in tumor burden, an increase in the rate or extent of metastasis, worsening symptoms, in whole or in part, associated with the cancer, an increase in the extent of disease, and an acceleration of disease progression. In some embodiments, the tumor burden is assessed using RECIST version 1.1. In some embodiments, the cancer is glioma and the progression of the glioma is assessed by RANO.

In some embodiments, liquid biopsies can be used to detect the progression of a cancer. In some embodiments, the biological sample to be used in a liquid biopsy can include, blood, plasma, urine, cerebrospinal fluid, saliva, sputum, broncho-alveolar lavage, bile, lymphatic fluid, cyst fluid, stool, ascites, and combinations thereof. In some embodiments, a liquid biopsy can be used to detect circulating tumor cells (CTCs). In some embodiments, a liquid biopsy can be used to detect cell-free DNA. In some embodiments, cell-free DNA detected using a liquid biopsy is circulating tumor DNA (ctDNA) that is derived from tumor cells. Analysis of ctDNA (e.g., using sensitive detection techniques such as, without limitation, next-generation sequencing (NGS), traditional PCR, digital PCR, or microarray analysis) can be used to identify progression of the cancer.

Liquid biopsies can be performed at multiple times during a course of diagnosis, a course of monitoring, and/or a course of therapy to determine one or more clinically relevant parameters including, without limitation, progression of the disease, efficacy of a therapy, or development of resistance mutations after administering a therapy to the subject. For example, a first liquid biopsy can be performed at a first time point and a second liquid biopsy can be performed at a second time point during a course of diagnosis, a course of monitoring, and/or a course of therapy. In some embodiments, the first time point can be a time point prior to diagnosing a subject with a disease (e.g., when the subject is healthy), and the second time point can be a time point after subject has developed the disease (e.g., the second time point can be used to diagnose the subject with the disease). In some embodiments, the first time point can be a time point prior to diagnosing a subject with a disease (e.g., when the subject is healthy), after which the subject is monitored, and the second time point can be a time point after monitoring the subject. In some embodiments, the first time point can be a time point after diagnosing a subject with a disease, after which a therapy is administered to the subject, and the second time point can be a time point after the therapy is administered; in such cases, the second time point can be used to assess the efficacy of the therapy (e.g., if the genetic mutation(s) detected at the first time point are reduced in abundance or are undetectable) or to determine the presence of a resistance mutation that has arisen as a result of the therapy.

In some embodiments provided herein, circulating tumor DNA can be used to monitor the responsiveness of a patient to a particular therapy (e.g., a first Trk inhibitor or a second Trk inhibitor such as Compound 1, or a pharmaceutically acceptable salt, amorphous, or polymorph form thereof). For example, prior to starting a therapy as described herein (e.g., a first Trk inhibitor or a second Trk inhibitor such as Compound 1, or a pharmaceutically acceptable salt, amorphous, or polymorph form thereof), a biological sample can be obtained from the subject and the level of circulating tumor DNA determined in the biological sample. This sample can be considered a base-line sample. The subject can then be administered one or more doses of a therapy as described herein (e e.g., a first Trk inhibitor or a second Trk inhibitor such as Compound 1, or a pharmaceutically acceptable salt, amorphous, or polymorph form thereof) and the levels of circulating tumor DNA can be monitored (e.g., after the first dose, second dose, third dose, etc. or after one week, two weeks, three weeks, four weeks, etc.). If the level of circulating tumor DNA is lower than the baseline sample (e.g., a 1% to about a 99% reduction, a 1% to about a 95% reduction, a 1% to about a 90% reduction, a 1% to about a 85% reduction, a 1% to about a 80% reduction, a 1% to about a 75% reduction, a 1% reduction to about a 70% reduction, a 1% reduction to about a 65% reduction, a 1% reduction to about a 60% reduction, a 1% reduction to about a 55% reduction, a 1% reduction to about a 50% reduction, a 1% reduction to about a 45% reduction, a 1% reduction to about a 40% reduction, a 1% reduction to about a 35% reduction, a 1% reduction to about a 30% reduction, a 1% reduction to about a 25% reduction, a 1% reduction to about a 20% reduction, a 1% reduction to about a 15% reduction, a 1% reduction to about a 10% reduction, a 1% to about a 5% reduction, about a 5% to about a 99% reduction, about a 10% to about a 99% reduction, about a 15% to about a 99% reduction, about a 20% to about a 99% reduction, about a 25% to about a 99% reduction, about a 30% to about a 99% reduction, about a 35% to about a 99% reduction, about a 40% to about a 99% reduction, about a 45% to about a 99% reduction, about a 50% to about a 99% reduction, about a 55% to about a 99% reduction, about a 60% to about a 99% reduction, about a 65% to about a 99% reduction, about a 70% to about a 99% reduction, about a 75% to about a 95% reduction, about a 80% to about a 99% reduction, about a 90% reduction to about a 99% reduction, about a 95% to about a 99% reduction, about a 5% to about a 10% reduction, about a 5% to about a 25% reduction, about a 10% to about a 30% reduction, about a 20% to about a 40% reduction, about a 25% to about a 50% reduction, about a 35% to about a 55% reduction, about a 40% to about a 60% reduction, about a 50% reduction to about a 75% reduction, about a 60% reduction to about 80% reduction, or about a 65% to about a 85% reduction etc.), this is indicative of responsiveness to the therapy. In some embodiments, the level of circulating tumor DNA in a biological sample obtained from the patient (n) is compared to the sample taken just previous (n-1). If the level of circulating tumor DNA in the n sample is lower than the n-1 sample (e.g., a 1% to about a 99% reduction, a 1% to about a 95% reduction, a 1% to about a 90% reduction, a 1% to about a 85% reduction, a 1% to about a 80% reduction, a 1% to about a 75% reduction, a 1% reduction to about a 70% reduction, a 1% reduction to about a 65% reduction, a 1% reduction to about a 60% reduction, a 1% reduction to about a 55% reduction, a 1% reduction to about a 50% reduction, a 1% reduction to about a 45% reduction, a 1% reduction to about a 40% reduction, a 1% reduction to about a 35% reduction, a 1% reduction to about a 30% reduction, a 1% reduction to about a 25% reduction, a 1% reduction to about a 20% reduction, a 1% reduction to about a 15% reduction, a 1% reduction to about a 10% reduction, a 1% to about a 5% reduction, about a 5% to about a 99% reduction, about a 10% to about a 99% reduction, about a 15% to about a 99% reduction, about a 20% to about a 99% reduction, about a 25% to about a 99% reduction, about a 30% to about a 99% reduction, about a 35% to about a 99% reduction, about a 40% to about a 99% reduction, about a 45% to about a 99% reduction, about a 50% to about a 99% reduction, about a 55% to about a 99% reduction, about a 60% to about a 99% reduction, about a 65% to about a 99% reduction, about a 70% to about a 99% reduction, about a 75% to about a 95% reduction, about a 80% to about a 99% reduction, about a 90% reduction to about a 99% reduction, about a 95% to about a 99% reduction, about a 5% to about a 10% reduction, about a 5% to about a 25% reduction, about a 10% to about a 30% reduction, about a 20% to about a 40% reduction, about a 25% to about a 50% reduction, about a 35% to about a 55% reduction, about a 40% to about a 60% reduction, about a 50% reduction to about a 75% reduction, about a 60% reduction to about 80% reduction, or about a 65% to about a 85% reduction, etc.), this is indicative of responsiveness to the therapy. In the case of responsiveness to therapy, the subject can to be administered one or more doses of the therapy and the circulating tumor DNA can be continued to be monitored.

If the level of circulating tumor DNA in the sample is higher than the baseline (e.g., a 1% to about a 99% increase, a 1% to about a 95% increase, a 1% to about a 90% increase, a 1% to about a 85% increase, a 1% to about a 80% increase, a 1% to about a 75% increase, a 1% increase to about a 70% increase, a 1% increase to about a 65% increase, a 1% increase to about a 60% increase, a 1% increase to about a 55% increase, a 1% increase to about a 50% increase, a 1% increase to about a 45% increase, a 1% increase to about a 40% increase, a 1% increase to about a 35% increase, a 1% increase to about a 30% increase, a 1% increase to about a 25% increase, a 1% increase to about a 20% increase, a 1% increase to about a 15% increase, a 1% increase to about a 10% increase, a 1% to about a 5% increase, about a 5% to about a 99% increase, about a 10% to about a 99% increase, about a 15% to about a 99% increase, about a 20% to about a 99% increase, about a 25% to about a 99% increase, about a 30% to about a 99% increase, about a 35% to about a 99% increase, about a 40% to about a 99% increase, about a 45% to about a 99% increase, about a 50% to about a 99% increase, about a 55% to about a 99% increase, about a 60% to about a 99% increase, about a 65% to about a 99% increase, about a 70% to about a 99% increase, about a 75% to about a 95% increase, about a 80% to about a 99% increase, about a 90% increase to about a 99% increase, about a 95% to about a 99% increase, about a 5% to about a 10% increase, about a 5% to about a 25% increase, about a 10% to about a 30% increase, about a 20% to about a 40% increase, about a 25% to about a 50% increase, about a 35% to about a 55% increase, about a 40% to about a 60% increase, about a 50% increase to about a 75% increase, about a 60% increase to about 80% increase, or about a 65% to about a 85% increase, etc.), this can be indicative of progression of the cancer. If the level of circulating tumor DNA in the n sample is higher than the n-1 sample (e.g., a 1% to about a 99% increase, a 1% to about a 95% increase, a 1% to about a 90% increase, a 1% to about a 85% increase, a 1% to about a 80% increase, a 1% to about a 75% increase, a 1% increase to about a 70% increase, a 1% increase to about a 65% increase, a 1% increase to about a 60% increase, a 1% increase to about a 55% increase, a 1% increase to about a 50% increase, a 1% increase to about a 45% increase, a 1% increase to about a 40% increase, a 1% increase to about a 35% increase, a 1% increase to about a 30% increase, a 1% increase to about a 25% increase, a 1% increase to about a 20% increase, a 1% increase to about a 15% increase, a 1% increase to about a 10% increase, a 1% to about a 5% increase, about a 5% to about a 99% increase, about a 10% to about a 99% increase, about a 15% to about a 99% increase, about a 20% to about a 99% increase, about a 25% to about a 99% increase, about a 30% to about a 99% increase, about a 35% to about a 99% increase, about a 40% to about a 99% increase, about a 45% to about a 99% increase, about a 50% to about a 99% increase, about a 55% to about a 99% increase, about a 60% to about a 99% increase, about a 65% to about a 99% increase, about a 70% to about a 99% increase, about a 75% to about a 95% increase, about a 80% to about a 99% increase, about a 90% increase to about a 99% increase, about a 95% to about a 99% increase, about a 5% to about a 10% increase, about a 5% to about a 25% increase, about a 10% to about a 30% increase, about a 20% to about a 40% increase, about a 25% to about a 50% increase, about a 35% to about a 55% increase, about a 40% to about a 60% increase, about a 50% increase to about a 75% increase, about a 60% increase to about 80% increase, or about a 65% to about a 85% increase etc.), this can be indicative of progression of the cancer. When progression of the cancer during therapy with a first Trk inhibitor is suspected, the subject can undergo one or more of imaging, biopsy, surgery, or other diagnostic tests. In some embodiments, when progression of the cancer during therapy with a first Trk inhibitor is suspected, the subject can be administered (either as a monotherapy or in combination with the previous therapy) a second Trk inhibitor, e.g., Compound 1, or a pharmaceutically acceptable salt, amorphous, or polymorph form thereof.

If after a period of improvement, e.g., a period of responsiveness to the therapy as described above, the level of circulating tumor DNA in the sample is higher than the level obtained during the period of improvement (e.g., a 1% to about a 99% increase, a 1% to about a 95% increase, a 1% to about a 90% increase, a 1% to about a 85% increase, a 1% to about a 80% increase, a 1% to about a 75% increase, a 1% increase to about a 70% increase, a 1% increase to about a 65% increase, a 1% increase to about a 60% increase, a 1% increase to about a 55% increase, a 1% increase to about a 50% increase, a 1% increase to about a 45% increase, a 1% increase to about a 40% increase, a 1% increase to about a 35% increase, a 1% increase to about a 30% increase, a 1% increase to about a 25% increase, a 1% increase to about a 20% increase, a 1% increase to about a 15% increase, a 1% increase to about a 10% increase, a 1% to about a 5% increase, about a 5% to about a 99% increase, about a 10% to about a 99% increase, about a 15% to about a 99% increase, about a 20% to about a 99% increase, about a 25% to about a 99% increase, about a 30% to about a 99% increase, about a 35% to about a 99% increase, about a 40% to about a 99% increase, about a 45% to about a 99% increase, about a 50% to about a 99% increase, about a 55% to about a 99% increase, about a 60% to about a 99% increase, about a 65% to about a 99% increase, about a 70% to about a 99% increase, about a 75% to about a 95% increase, about a 80% to about a 99% increase, about a 90% increase to about a 99% increase, about a 95% to about a 99% increase, about a 5% to about a 10% increase, about a 5% to about a 25% increase, about a 10% to about a 30% increase, about a 20% to about a 40% increase, about a 25% to about a 50% increase, about a 35% to about a 55% increase, about a 40% to about a 60% increase, about a 50% increase to about a 75% increase, about a 60% increase to about 80% increase, or about a 65% to about a 85% increase, etc.), this can be indicative of relapse of the cancer. If the level of circulating tumor DNA in the n sample is higher than the n-1 sample (e.g., a 1% to about a 99% increase, a 1% to about a 95% increase, a 1% to about a 90% increase, a 1% to about a 85% increase, a 1% to about a 80% increase, a 1% to about a 75% increase, a 1% increase to about a 70% increase, a 1% increase to about a 65% increase, a 1% increase to about a 60% increase, a 1% increase to about a 55% increase, a 1% increase to about a 50% increase, a 1% increase to about a 45% increase, a 1% increase to about a 40% increase, a 1% increase to about a 35% increase, a 1% increase to about a 30% increase, a 1% increase to about a 25% increase, a 1% increase to about a 20% increase, a 1% increase to about a 15% increase, a 1% increase to about a 10% increase, a 1% to about a 5% increase, about a 5% to about a 99% increase, about a 10% to about a 99% increase, about a 15% to about a 99% increase, about a 20% to about a 99% increase, about a 25% to about a 99% increase, about a 30% to about a 99% increase, about a 35% to about a 99% increase, about a 40% to about a 99% increase, about a 45% to about a 99% increase, about a 50% to about a 99% increase, about a 55% to about a 99% increase, about a 60% to about a 99% increase, about a 65% to about a 99% increase, about a 70% to about a 99% increase, about a 75% to about a 95% increase, about a 80% to about a 99% increase, about a 90% increase to about a 99% increase, about a 95% to about a 99% increase, about a 5% to about a 10% increase, about a 5% to about a 25% increase, about a 10% to about a 30% increase, about a 20% to about a 40% increase, about a 25% to about a 50% increase, about a 35% to about a 55% increase, about a 40% to about a 60% increase, about a 50% increase to about a 75% increase, about a 60% increase to about 80% increase, or about a 65% to about a 85% increase etc.), this can be indicative of relapse of the cancer. When relapse of the cancer during therapy with a first Trk inhibitor is suspected, the subject can undergo one or more of imaging, biopsy, surgery, or other diagnostic tests. In some embodiments, when relapse of the cancer during therapy with a first Trk inhibitor is suspected, the subject can be administered (either as a monotherapy or in combination with the previous therapy) a second Trk inhibitor, e.g., Compound 1, or a pharmaceutically acceptable salt, amorphous, or polymorph form thereof. See, for example, Cancer Discov; 7(12); 1368-70 (2017); and Cancer Discov; 7(12); 1394-403 (2017). In some embodiments, a Trk resistance mutation, e.g., any of the mutations described in Tables 11-13, is not detected.

In some embodiments, the subject that is intolerant to a first Trk inhibitor has had one or more of a severe, disabling, or life-threatening adverse event during therapy with the first Trk inhibitor, an unplanned hospitalization during therapy with the first Trk inhibitor, discontinuation of therapy with the first Trk inhibitor, dose reduction of the first Trk inhibitor, functional decline attributed to therapy with the first Trk inhibitor, and a decrease in performance status.

In some embodiments, the performance status is assessed using the Eastern Cooperative Oncology Group (ECOG) Scale of Performance Status.

In some embodiments, the performance status is assessed using the Karnofsky Performance Status.

In some embodiments, the performance status is assess by the Lansky Performance Score. In some embodiments, the subject is a pediatric patient.

In some embodiments, wherein (i) the cancer in the subject has relapsed during therapy with a first Trk inhibitor; and/or (ii) the cancer in the subject is not responding to therapy with the first Trk inhibitor; and/or (iii) the subject is intolerant to the first Trk inhibitor, a Trk resistance mutation, e.g., any of the mutations described in Tables 11-13, is not detected.

In some embodiments, wherein (i) the cancer in the subject has relapsed during therapy with a first Trk inhibitor; and/or (ii) the cancer in the subject is not responding to therapy with the first Trk inhibitor; and/or (iii) the subject is intolerant to the first Trk inhibitor, a Trk resistance mutation, e.g., any of the mutations described in Tables 11-13, is not detected.

Some examples of these methods further include recording in the subject's clinical record (e.g., a computer readable medium) that the subject should be administered a treatment that does not include the first Trk inhibitor in step (a) as a monotherapy or a different Trk inhibitor in the future.

Further provided herein is a composition for use is in treating a Trk-associated cancer in a subject, the composition comprising a therapeutically effective amount of a pharmaceutical composition as provided herein.

Also provided herein is a composition for use in a method of treating a subject having a cancer, the method comprising: (a) detecting a dysregulation of a NTRK gene, a Trk kinase, or the expression or activity or level of any of the same; (b) administering one or more doses of a first Trk inhibitor to the subject for a period of time; (c) after (a) and (b), determining whether (i) the cancer in the subject has relapsed during therapy with the first Trk inhibitor; and/or (ii) the cancer in the subject is not responding to therapy with the first Trk inhibitor; and/or (iii) the subject is intolerant to the first Trk inhibitor; and (d) administering a treatment including one or more doses of a second Trk inhibitor or a pharmaceutically acceptable salt thereof, to a subject in which (i) the cancer in the subject has relapsed during therapy with the first Trk inhibitor; and/or (ii) the cancer in the subject is not responding to therapy with the first Trk inhibitor; and/or (iii) the subject is intolerant to the first Trk inhibitor; or (e) administering additional doses of the first Trk inhibitor to a subject in which (i) the cancer has not relapsed during therapy with the first Trk inhibitor; and/or (ii) the cancer in the subject is responding to therapy with the first Trk inhibitor; and/or (iii) the subject is not intolerant to the first Trk inhibitor.

Also provided herein is a composition comprising a second Trk inhibitor for use in treating a subject having a cancer, wherein (a) a dysregulation of a NTRK gene, a Trk kinase, or the expression or activity or level of any of the same has been previously detected in the subject; (b) one or more doses of the first Trk inhibitor have been previously administered to the subject for a period of time; (c) a determination is made whether (i) the cancer in the subject has relapsed during therapy with the first Trk inhibitor; and/or (ii) the cancer in the subject is not responding to therapy with the first Trk inhibitor; and/or (iii) the subject is intolerant to the first Trk inhibitor; wherein (d) one or more doses of the second Trk inhibitor or a pharmaceutically acceptable salt thereof are to be administered to a subject in which (i) the cancer in the subject has relapsed during therapy with the first Trk inhibitor; and/or (ii) the cancer in the subject is not responding to therapy with the first Trk inhibitor; and/or (iii) the subject is intolerant to the first Trk inhibitor; or (e) additional doses of the first Trk inhibitor are to be administered to a subject in which (i) the cancer has not relapsed during therapy with the first Trk inhibitor; and/or (ii) the cancer in the subject is responding to therapy with the first Trk inhibitor; and/or (iii) the subject is not intolerant to the first Trk inhibitor.

### Methods of Selecting a Treatment for a Subject Having a Cancer

Also provided herein are methods of selecting a treatment that does not include a first Trk inhibitor (e.g., entrectinib, TPX-0005, PLX7486, or (S)—N-(5-((R)-2-(2,5-difluorophenyl)pyrrolidin-1-yl)pyrazolo[1,5-a]pyrimidin-3-yl)-3-hydroxypyrrolidine-1-carboxamide sulfate) as a monotherapy for a subject having a cancer (e.g., any of the cancers described herein) that include identifying a subject in which (i) the cancer in the subject has relapsed during therapy with the first Trk inhibitor; and/or (ii) the cancer in the subject is not responding to therapy with the first Trk inhibitor; and/or (iii) the subject is intolerant to the first Trk inhibitor, and selecting a treatment that does not include the first Trk inhibitor as a monotherapy (e.g., any of the treatments that do not include the first Trk inhibitor as a monotherapy described herein) for the identified subject.

Also provided herein are methods of selecting a treatment that does not include a first Trk inhibitor (e.g., entrectinib, TPX-0005, PLX7486, or (S)—N-(5-((R)-2-(2,5-difluorophenyl)pyrrolidin-1-yl)pyrazolo[1,5-a]pyrimidin-3-yl)-3-hydroxypyrrolidine-1-carboxamide sulfate) as a monotherapy for a subject having a cancer (e.g., any of the treatments that do not include a Trk inhibitor as a monotherapy described herein) that include selecting a treatment that does not include the first Trk inhibitor as a monotherapy (e.g., any of the treatments that do not include the first Trk inhibitor as a monotherapy described herein) for a subject identified as having a cancer and wherein (i) the cancer in the subject has relapsed during therapy with the first Trk inhibitor; and/or (ii) the cancer in the subject is not responding to therapy with the first Trk inhibitor; and/or (iii) the subject is intolerant to the first Trk inhibitor.

Some of these methods include selecting a different Trk inhibitor (e.g., a second Trk inhibitor) or a treatment that does not include the first Trk inhibitor of step (a) as a monotherapy to a subject in which (i) the cancer in the subject has relapsed during therapy with the first Trk inhibitor; and/or (ii) the cancer in the subject is not responding to therapy with the first Trk inhibitor; and/or (iii) the subject is intolerant to the first Trk inhibitor. In some embodiments, the second Trk inhibitor is Compound 1, or a pharmaceutically acceptable salt, amorphous, or polymorph form thereof. In some embodiments the second Trk inhibitor is a pharmaceutical composition comprising a compounding agent as disclosed herein and Compound 1 or a solid form thereof, crystalline form thereof, or solvate or hydrate thereof, or a salt of Compound 1 or solid form thereof, crystalline form thereof, or solvate or hydrate thereof, as described herein.

Also provided herein are methods of selecting a treatment for a subject having a cancer (e.g., any of the cancers described herein or known in the art) that include: identifying a subject in which (i) the cancer in the subject has relapsed during therapy with the first Trk inhibitor; and/or (ii) the cancer in the subject is not responding to therapy with the first Trk inhibitor; and/or (iii) the subject is intolerant to the first Trk inhibitor; and selecting a treatment that includes Compound 1, or a pharmaceutically acceptable salt, amorphous, or polymorph form thereof, for the identified subject.

Also provided herein are methods of selecting a treatment for a subject having a cancer (e.g., any of the cancers described herein or known in the art) that include: identifying a subject in which (i) the cancer in the subject has relapsed during therapy with the first Trk inhibitor; and/or (ii) the cancer in the subject is not responding to therapy with the first Trk inhibitor; and/or (iii) the subject is intolerant to the first Trk inhibitor; and selecting a treatment that includes Compound 1, or a pharmaceutically acceptable salt, amorphous, or polymorph form thereof, and another anticancer agent (e.g., any one or more of the anticancer agents described herein or known in the art) or anticancer therapy (e.g., any one or more of the anticancer therapies described herein or known in the art) for the identified subject.

Also provided herein are methods of selecting a treatment for a subject having a cancer that include: selecting a treatment that includes Compound 1, or a pharmaceutically acceptable salt, amorphous, or polymorph form thereof, for a subject identified as having a cancer and wherein (i) the cancer in the subject has relapsed during therapy with the first Trk inhibitor; and/or (ii) the cancer in the subject is not responding to therapy with the first Trk inhibitor; and/or (iii) the subject is intolerant to the first Trk inhibitor.

Also provided herein are methods of selecting a treatment for a subject having a cancer that include: selecting a treatment that includes Compound 1, or a pharmaceutically acceptable salt, amorphous, or polymorph form thereof, and another anticancer agent (e.g., any one or more of the anticancer agents described herein or known in the art) or anticancer therapy (e.g., any one or more of the anticancer therapies described herein or known in the art) for a subject and wherein (i) the cancer in the subject has relapsed during therapy with the first Trk inhibitor; and/or (ii) the cancer in the subject is not responding to therapy with the first Trk inhibitor; and/or (iii) the subject is intolerant to the first Trk inhibitor.

In some embodiments, the cancer is a Trk-associated cancer. In some embodiments, the Trk-associated cancer exhibits at least one of a NTRK1, NTRK2, and/or NTRK3 fusion. In some embodiments, the at least one NTRK1, NTRK2, and/or NTRK3 fusion results in the expression of one or more of a TrkA fusion protein, and/or a TrkB fusion protein, and/or a TrkC fusion protein, e.g., one or more of the Trk fusions described in Tables 2, 5, and 8. In some embodiments, the Trk-associated cancer exhibits one or mutations point mutations/insertions/deletions in one or more of NTRK1, NTRK2, or NTRK3. Non-limiting examples of Trk kinase point mutations/insertions/deletions are described in Tables 3, 4, 6, 7, 9, and 10. In some embodiments, the Trk-associated cancer does not exhibit a Trk resistance mutation, e.g., any of the mutations described in Tables 11-13.

Also provided herein are methods of selecting a treatment for a subject having a cancer that include: (a) detecting a dysregulation of a NTRK gene, a Trk kinase, or the expression or activity or level of any of the same; (b) administering one or more doses of a first Trk inhibitor (e.g., entrectinib, TPX-0005, PLX7486, or (S)—N-(5-((R)-2-(2,5-difluorophenyl)pyrrolidin-1-yl)pyrazolo[1,5-a]pyrimidin-3-yl)-3-hydroxypyrrolidine-1-carboxamide sulfate) to the subject for a period of time; (c) after (a) and (b), determining whether (i) the cancer in the subject has relapsed during therapy with the first Trk inhibitor; and/or (ii) the cancer in the subject is not responding to therapy with the first Trk inhibitor; and/or (iii) the subject is intolerant to the first Trk inhibitor; and (d) selecting a second Trk inhibitor or a treatment that does not include the first Trk inhibitor of step (b) (e.g., (S)—N-(5-((R)-2-(2,5-difluorophenyl)pyrrolidin-1-yl)pyrazolo[1,5-a]pyrimidin-3-yl)-3-hydroxypyrrolidine-1-carboxamide sulfate) as a monotherapy for a subject in which (i) the cancer in the subject has relapsed during therapy with the first Trk inhibitor; and/or (ii) the cancer in the subject is not responding to therapy with the first Trk inhibitor; and/or (iii) the subject is intolerant to the first Trk inhibitor; or (e) selecting additional doses of the first Trk inhibitor of step (b) (e.g., (S)—N-(5-((R)-2-(2,5-difluorophenyl)pyrrolidin-1-yl)pyrazolo[1,5-a]pyrimidin-3-yl)-3-hydroxypyrrolidine-1-carboxamide sulfate) for a subject in which (i) the cancer in the subject has not relapsed during therapy with the first Trk inhibitor; and/or (ii) the cancer in the subject is responding to therapy with the first Trk inhibitor; and/or (iii) the subject is not intolerant to the first Trk inhibitor.

In some embodiments, step (a) is performed before step (b).

In some embodiments, step (b) is performed before step (a).

In some embodiments, detecting a dysregulation of a NTRK gene, a Trk kinase, or the expression or activity or level of any of the same includes next generation sequencing, immunohistochemistry, fluorescence microscopy, break apart FISH analysis, and PCR-based amplification (e.g., RT-PCR and quantitative real-time RT-PCR).

In some embodiments, the dysregulation of a NTRK gene, a Trk kinase, or the expression or activity or level of any of the same is at least one NTRK1, NTRK2, and/or NTRK3 fusion. In some embodiments, the at least one NTRK1, NTRK2, and/or NTRK3 fusion results in the expression of one or more of a TrkA fusion protein, and/or a TrkB fusion protein, and/or a TrkC fusion protein, e.g., one or more of the Trk fusions described in Tables 2, 5, and 8. In some embodiments, the at least one NTRK1, NTRK2, and/or NTRK3 fusion results in the expression of one or more of a TrkA fusion protein, and/or a TrkB fusion protein, and/or a TrkC fusion protein, wherein the TrkA fusion protein comprises one or more of the of the fusions selected from the group consisting of: TP53-TrkA, LMNA-TrkA, CD74-TrkA, TFG-TrkA, TPM3-TrkA, NFASC-TrkA, BCAN-TrkA, MPRIP-TrkA, TPR-TrkA, RFWD2-TrkA, IRF2BP2-TrkA, SQSTM1-TrkA, SSBP2-TrkA, RABGAP1L-TrkA, C18ORF8-TrkA, RNF213-TrkA, TBC1D22A-TrkA, C20ORF112-TrkA, DNER-TrkA, ARHGEF2-TrkA, CHTOP-TrkA, PPL-TrkA, PLEKHA6-TrkA, PEAR1-TrkA, MRPL24-TrkA, MDM4-TrkA, LRRC71-TrkA, GRIPAP1-TrkA, TAF-TrkA, EPS15-TrkA, DYNC2H1-TrkA, CEL-TrkA, EPHB2-TrkA, TGF-TrkA, NELL1-TrkA, EPL4-TrkA, CTNND2-TrkA, TCEANC2-TrkA, SCYL3-TrkA, AMOTL2-TrkA, MEF2D-TrkA, L7a-TrkA, ZBTB7B-TrkA, TRIM63-TrkA, DDR2-TrkAl, GON4L-TrkA, PDE4DIP-TrkA, NTRK1-P2RY8, CTRC-TrkA, and VANGL2-TrkA; and/or the TrkB fusion protein comprises one or more of the of the fusions selected from the group consisting of: NACC2-TrkB, QKI-TrkB, AFAP1-TrkB, PAN3-TrkB, SQSTM1-TrkB, TRIM24-TrkB, VCL-TrkB, AGBL4-TrkB, DAB2IP-TrkB, TrkB-TERT, ETV6-TrkB, NOS1AP-TrkB, GKAP1-TrkB, KCTD8-TrkB, TBC1D2-TrkB, VCAN-TrkB, SLMAP-TrkB, TLE4-TrkB, STRN3-TrkB, WNK2-TrkB, TrkB-BEND5, TrkB-TRAF2, Nav1-TrkB, and STRN-TrkB; and/or the TrkC fusion protein comprises one or more of the of the fusions selected from the group consisting of: ETV6-TrkC1, BTBD1-TrkC, LYN-TrkC, RBPMS-TrkC, EML4-TrkC, TrkC-HOMER2, TFG-TrkC, FAT1-TrkC, MYO5A-TrkC, MYH9-TrkC, KANK1-TrkC, SQSTM1-TrkC, UBE2R2-TrkC, HNRNPA2B1-TrkC, VPS18-TrkC, AKAP13-TrkC, TrkC-LOXL2, TrkC-PEAK1, ZNF710-TrkC, TPM4-TrkC, and LMNA-TrkC. In some embodiments, the dysregulation of a NTRK gene, a Trk kinase, or the expression or activity or level of any of the same is one or mutations point mutations/insertions/deletions in one or more of NTRK1, NTRK2, or NTRK3. Non-limiting examples of Trk kinase point mutations/insertions/deletions are described in Tables 3, 4, 6, 7, 9, 10. In some embodiments, the Trk-associated cancer does not exhibit a Trk resistance mutation, e.g., any of the mutations described in Tables 11-13.

Also provided herein are methods of selecting a treatment for a subject having a cancer that include: (a) determining whether for a subject having a cancer and previously administered one or more doses of a first Trk inhibitor (e.g., entrectinib, TPX-0005, PLX7486, or (S)—N-(5-((R)-2-(2,5-difluorophenyl)pyrrolidin-1-yl)pyrazolo[1,5-a]pyrimidin-3-yl)-3-hydroxypyrrolidine-1-carboxamide sulfate), (i) the cancer in the subject has relapsed during therapy with the first Trk inhibitor; and/or (ii) the cancer in the subject is not responding to therapy with the first Trk inhibitor; and/or (iii) the subject is intolerant to the first Trk inhibitor; (b) selecting a second Trk inhibitor or a treatment that does not include the first Trk inhibitor of step (a) as a monotherapy for a subject in which (i) the cancer in the subject has relapsed during therapy with the first Trk inhibitor; and/or (ii) the cancer in the subject is not responding to therapy with the first Trk inhibitor; and/or (iii) the subject is intolerant to the first Trk inhibitor; or (c) selecting additional doses of the first Trk inhibitor of step (a) for a subject in which (i) the cancer has not relapsed during therapy with the first Trk inhibitor; and/or (ii) the cancer in the subject is responding to therapy with the first Trk inhibitor; and/or (iii) the subject is not intolerant to the first Trk inhibitor. In some embodiments, the second Trk inhibitor is Compound 1, or a pharmaceutically acceptable salt, amorphous, or polymorph form thereof. In some embodiments the second Trk inhibitor is a pharmaceutical composition comprising a compounding agent as disclosed herein and Compound 1 or a solid form thereof, crystalline form thereof, or solvate or hydrate thereof, or a salt of Compound 1 or solid form thereof, crystalline form thereof, or solvate or hydrate thereof, as described herein. In some embodiments, the cancer is a Trk-associated cancer. In some embodiments, the Trk associated cancer exhibits at least one of a NTRK1, NTRK2, and/or NTRK3 fusion. In some embodiments, the at least one NTRK1, NTRK2, and/or NTRK3 fusion results in the expression of one or more of a TrkA fusion protein, and/or a TrkB fusion protein, and/or a TrkC fusion protein, e.g., one or more of the Trk fusions described in Tables 2, 5, and 8. In some embodiments, the Trk-associated cancer exhibits one or mutations point mutations/insertions/deletions in one or more of NTRK1, NTRK2, or NTRK3. Non-limiting examples of Trk kinase point mutations/insertions/deletions are described in Tables 3, 4, 6, 7, 9, 10. In some embodiments, the Trk-associated cancer does not exhibit a Trk resistance mutation, e.g., any of the mutations described in Tables 11-13.

Also provided herein are methods of selecting a treatment for a subject having a cancer, that include: (a) detecting a dysregulation of a NTRK gene, a Trk kinase, or the expression or activity or level of any of the same; (b) administering one or more doses of a first Trk inhibitor to the subject for a period of time; (c) after (a) and (b), determining whether (i) the cancer in the subject has relapsed during therapy with the first Trk inhibitor; and/or (ii) the cancer in the subject is not responding to therapy with the first Trk inhibitor; and/or (iii) the subject is intolerant to the first Trk inhibitor; and (d) selecting a treatment including one or more doses of a second Trk inhibitor for a subject in which (i) the cancer in the subject has relapsed during therapy with the first Trk inhibitor; and/or (ii) the cancer in the subject is not responding to therapy with the first Trk inhibitor; and/or (iii) the subject is intolerant to the first Trk inhibitor; or (e) selecting additional doses of the first Trk inhibitor for a subject in which (i) the cancer has not relapsed during therapy with the first Trk inhibitor; and/or (ii) the cancer in the subject is responding to therapy with the first Trk inhibitor; and/or (iii) the subject is not intolerant to the first Trk inhibitor. In some embodiments, the second Trk inhibitor is Compound 1, or a pharmaceutically acceptable salt, amorphous, or polymorph form thereof. In some embodiments the second Trk inhibitor is a pharmaceutical composition comprising a compounding agent as disclosed herein and Compound 1 or a solid form thereof, crystalline form thereof, or solvate or hydrate thereof, or a salt of Compound 1 or solid form thereof, crystalline form thereof, or solvate or hydrate thereof, as described herein.

Also provided herein are methods of selecting a treatment for a subject having a cancer, that include: (a) detecting a dysregulation of a NTRK gene, a Trk kinase, or the expression or activity or level of any of the same; (b) administering one or more doses of a first Trk inhibitor to the subject for a period of time; (c) after (a) and (b), determining whether (i) the cancer in the subject has relapsed during therapy with the first Trk inhibitor; and/or (ii) the cancer in the subject is not responding to therapy with the first Trk inhibitor; and/or (iii) the subject is intolerant to the first Trk inhibitor; and (d) selecting a treatment including Compound 1, or a pharmaceutically acceptable salt, amorphous, or polymorph form thereof, for a subject in which (i) the cancer in the subject has relapsed during therapy with the first Trk inhibitor; and/or (ii) the cancer in the subject is not responding to therapy with the first Trk inhibitor; and/or (iii) the subject is intolerant to the first Trk inhibitor; or (e) selecting additional doses of the first Trk inhibitor for a subject in which (i) the cancer has not relapsed during therapy with the first Trk inhibitor; and/or (ii) the cancer in the subject is responding to therapy with the first Trk inhibitor; and/or (iii) the subject is not intolerant to the first Trk inhibitor.

Also provided herein are methods of selecting a treatment for a subject having a cancer, that include: (a) detecting a dysregulation of a NTRK gene, a Trk kinase, or the expression or activity or level of any of the same; (b) administering one or more doses of a first Trk inhibitor to the subject for a period of time; (c) after (a) and (b), determining whether (i) the cancer in the subject has relapsed during therapy with the first Trk inhibitor; and/or (ii) the cancer in the subject is not responding to therapy with the first Trk inhibitor; and/or (iii) the subject is intolerant to the first Trk inhibitor; and (d) selecting a treatment including Compound 1, or a pharmaceutically acceptable salt, amorphous, or polymorph form thereof, and another anticancer agent or anticancer therapy for a subject in which (i) the cancer in the subject has relapsed during therapy with the first Trk inhibitor; and/or (ii) the cancer in the subject is not responding to therapy with the first Trk inhibitor; and/or (iii) the subject is intolerant to the first Trk inhibitor; or (d) selecting additional doses of the first Trk inhibitor for a subject in which (i) the cancer has not relapsed during therapy with the first Trk inhibitor; and/or (ii) the cancer in the subject is responding to therapy with the first Trk inhibitor; and/or (iii) the subject is not intolerant to the first Trk inhibitor.

In some embodiments, step (a) is performed before step (b).

In some embodiments, step (b) is performed before step (a).

In some embodiments, detecting a dysregulation of a NTRK gene, a Trk kinase, or the expression or activity or level of any of the same includes next generation sequencing, immunohistochemistry, fluorescence microscopy, break apart FISH analysis, and PCR-based amplification (e.g., RT-PCR and quantitative real-time RT-PCR).

In some embodiments, a dysregulation of a NTRK gene, a Trk kinase, or the expression or activity or level of any of the same is at least one NTRK1, NTRK2, and/or NTRK3 fusion. In some embodiments, the at least one NTRK1, NTRK2, and/or NTRK3 fusion results in the expression of one or more of a TrkA fusion protein, and/or a TrkB fusion protein, and/or a TrkC fusion protein, e.g., one or more of the Trk fusions described in Tables 2, 5, and 8. In some embodiments, the at least one NTRK1, NTRK2, and/or NTRK3 fusion results in the expression of one or more of a TrkA fusion protein, and/or a TrkB fusion protein, and/or a TrkC fusion protein, wherein the TrkA fusion protein comprises one or more of the of the fusions selected from the group consisting of: TP53-TrkA, LMNA-TrkA, CD74-TrkA, TFG-TrkA, TPM3-TrkA, NFASC-TrkA, BCAN-TrkA, MPRIP-TrkA, TPR-TrkA, RFWD2-TrkA, IRF2BP2-TrkA, SQSTM1-TrkA, SSBP2-TrkA, RABGAP1L-TrkA, C18ORF8-TrkA, RNF213-TrkA, TBC1D22A-TrkA, C20ORF112-TrkA, DNER-TrkA, ARHGEF2-TrkA, CHTOP-TrkA, PPL-TrkA, PLEKHA6-TrkA, PEAR1-TrkA, MRPL24-TrkA, MDM4-TrkA, LRRC71-TrkA, GRIPAP1-TrkA, TAF-TrkA, EPS15-TrkA, DYNC2H1-TrkA, CEL-TrkA, EPHB2-TrkA, TGF-TrkA, NELL1-TrkA, EPL4-TrkA, CTNND2-TrkA, TCEANC2-TrkA, SCYL3-TrkA, AMOTL2-TrkA, MEF2D-TrkA, L7a-TrkA, ZBTB7B-TrkA, TRIM63-TrkA, DDR2-TrkAl, GON4L-TrkA, PDE4DIP-TrkA, NTRK1-P2RY8, CTRC-TrkA, and VANGL2-TrkA; and/or the TrkB fusion protein comprises one or more of the of the fusions selected from the group consisting of: NACC2-TrkB, QKI-TrkB, AFAP1-TrkB, PAN3-TrkB, SQSTM1-TrkB, TRIM24-TrkB, VCL-TrkB, AGBL4-TrkB, DAB2IP-TrkB, TrkB-TERT, ETV6-TrkB, NOS1AP-TrkB, GKAP1-TrkB, KCTD8-TrkB, TBC1D2-TrkB, VCAN-TrkB, SLMAP-TrkB, TLE4-TrkB, STRN3-TrkB, WNK2-TrkB, TrkB-BEND5, TrkB-TRAF2, Nav1-TrkB, and STRN-TrkB; and/or the TrkC fusion protein comprises one or more of the of the fusions selected from the group consisting of: ETV6-TrkC1, BTBD1-TrkC, LYN-TrkC, RBPMS-TrkC, EML4-TrkC, TrkC-HOMER2, TFG-TrkC, FAT1-TrkC, MYO5A-TrkC, MYH9-TrkC, KANK1-TrkC, SQSTM1-TrkC, UBE2R2-TrkC, HNRNPA2B1-TrkC, VPS18-TrkC, AKAP13-TrkC, TrkC-LOXL2, TrkC-PEAK1, ZNF710-TrkC, TPM4-TrkC, and LMNA-TrkC. In some embodiments, the dysregulation of a NTRK gene, a Trk kinase, or the expression or activity or level of any of the same is one or mutations point mutations/insertions/deletions in one or more of NTRK1, NTRK2, or NTRK3. Non-limiting examples of Trk kinase point mutations/insertions/deletions are described in Tables 3, 4, 6, 7, 9, 10. In some embodiments, the Trk-associated cancer does not exhibit a Trk resistance mutation, e.g., any of the mutations described in Tables 11-13.

Also provided herein are methods of selecting a treatment for a subject having a cancer, that include: (a) determining whether (i) the cancer in the subject has relapsed during therapy with a first Trk inhibitor; and/or (ii) the cancer in the subject is not responding to therapy with a first Trk inhibitor; and/or (iii) the subject is intolerant to a first Trk inhibitor; (b) selecting a treatment that includes one or more doses of a second Trk inhibitor to a subject in which (i) the cancer in the subject has relapsed during therapy with the first Trk inhibitor; and/or (ii) the cancer in the subject is not responding to therapy with the first Trk inhibitor; and/or (iii) the subject is intolerant to the first Trk inhibitor; or (c) selecting additional doses of the first Trk inhibitor to a subject in which (i) the cancer has not relapsed during therapy with the first Trk inhibitor; and/or (ii) the cancer in the subject is responding to therapy with the first Trk inhibitor; and/or (iii) the subject is not intolerant to the first Trk inhibitor. In some embodiments, the second Trk inhibitor is Compound 1, or a pharmaceutically acceptable salt, amorphous, or polymorph form thereof. In some embodiments the second Trk inhibitor is a pharmaceutical composition comprising a compounding agent as disclosed herein and Compound 1 or a solid form thereof, crystalline form thereof, or solvate or hydrate thereof, or a salt of Compound 1 or solid form thereof, crystalline form thereof, or solvate or hydrate thereof, as described herein.

Also provided herein are methods of selecting a treatment for a subject having a cancer, that include: (a) determining whether (i) the cancer in the subject has relapsed during therapy with a first Trk inhibitor; and/or (ii) the cancer in the subject is not responding to therapy with a first Trk inhibitor; and/or (iii) the subject is intolerant to a first Trk inhibitor; (b) selecting a treatment that includes Compound 1, or a pharmaceutically acceptable salt, amorphous, or polymorph form thereof, to a subject in which (i) the cancer in the subject has relapsed during therapy with the first Trk inhibitor; and/or (ii) the cancer in the subject is not responding to therapy with the first Trk inhibitor; and/or (iii) the subject is intolerant to the first Trk inhibitor; or (c) selecting additional doses of the first Trk inhibitor to a subject in which (i) the cancer has not relapsed during therapy with the first Trk inhibitor; and/or (ii) the cancer in the subject is responding to therapy with the first Trk inhibitor; and/or (iii) the subject is not intolerant to the first Trk inhibitor.

Also provided herein are methods of selecting a treatment for a subject having a cancer, that include: (a) determining whether (i) the cancer in the subject has relapsed during therapy with a first Trk inhibitor; and/or (ii) the cancer in the subject is not responding to therapy with a first Trk inhibitor; and/or (iii) the subject is intolerant to a first Trk inhibitor; (b) selecting a treatment that includes Compound 1, or a pharmaceutically acceptable salt, amorphous, or polymorph form thereof, and an another anticancer agent or anticancer therapy to a subject in which (i) the cancer in the subject has relapsed during during therapy with the first Trk inhibitor; and/or (ii) the cancer in the subject is not responding to therapy with the first Trk inhibitor; and/or (iii) the subject is intolerant to the first Trk inhibitor; or (c) selecting additional doses of the first Trk inhibitor to a subject in which (i) the cancer has not relapsed during therapy with the first Trk inhibitor; and/or (ii) the cancer in the subject is responding to therapy with the first Trk inhibitor; and/or (iii) the subject is not intolerant to the first Trk inhibitor.

In some embodiments, the cancer is a Trk-associated cancer. In some embodiments, the Trk-associated cancer exhibits at least one of a NTRK1, NTRK2, and/or NTRK3 fusion. In some embodiments, the at least one NTRK1, NTRK2, and/or NTRK3 fusion results in the expression of one or more of a TrkA fusion protein, and/or a TrkB fusion protein, and/or a TrkC fusion protein, e.g., one or more of the Trk fusions described in Tables 2, 5, and 8. In some embodiments, the Trk-associated cancer exhibits one or mutations point mutations/insertions/deletions in one or more of NTRK1, NTRK2, or NTRK3. Non-limiting examples of Trk kinase point mutations/insertions/deletions are described in Tables 3, 4, 6, 7, 9, and 10. In some embodiments, the Trk-associated cancer does not exhibit a Trk resistance mutation, e.g., any of the mutations described in Tables 11-13.

In some embodiments, the first Trk inhibitor is selected from the group consisting of: entrectinib (N-[5-(3,5-difluoro-benzyl)-1H-indazol-3-yl]-4-(4-methylpiperazin-1-yl)-2-(tetrahydro-pyran-4-ylamino)-benzamide), (S)—N-(5-((R)-2-(2,5-difluorophenyl)pyrrolidin-1-yl)pyrazolo[1,5-a]pyrimidin-3-yl)-3-hydroxypyrrolidine-1-carboxamide sulfate, cabozantinib ((N-(4-((6,7-Dimethoxyquinolin-4-yl)oxy)phenyl)-N′-(4-fluorophenyl)cyclopropane-1,1-dicarboxamide)), dovitinib (4-amino-5-fluoro-3-[6-(4-methylpiperazin-1-yl)-1H-benzimidazol-2-yl]quinolin-2(1H)-one mono 2-hydroxypropanoate hydrate), belizatinib (4-fluoro-N-(6-((4-(2-hydroxypropan-2-yl)piperidin-1-yl)methyl)-1-((1s,4s)-4-(isopropylcarbamoyl)cyclohexyl)-1H-benzo[d]imidazol-2-yl)benzamide), sitravatinib (N-(3-fluoro-4-((2-(5-(((2-methoxyethyl)amino)methyl)pyridin-2-yl)thieno[3,2-b]pyridin-7-yl)oxy)phenyl)-N-(4-fluorophenyl)cyclopropane-1,1-dicarboxamide), PLX7486, altiratinib (N-(4-((2-(cyclopropanecarboxamido)pyridin-4-yl)oxy)-2,5-difluorophenyl)-N-(4-fluorophenyl)cyclopropane-1,1-dicarboxamide), AZD7451 ((S)—N-(1-(5-fluoropyrimidin-2-yl)ethyl)-3-(5-isopropoxy-1H-pyrazol-3-yl)-3H-imidazo[4,5-b]pyridin-5-amine), (6R,15R)-9-fluoro-15-methyl-2,11,16,20,21,24-hexaazapentacyclo[16.5.2.02,6.07,12.021,25]pentacosa-1(24),7,9, 11,18(25),19,22-heptaen-17-one, a (R)-2-phenylpyrrolidine substituted imadazopyridazine, AZD6918, GNF-4256, GTx-186, GNF-5837, AZ623, AG-879, CT327, AR-772, AR-523, AR-786, AR-256, AR-618, AZ-23, CEP-701, CEP-751, PHA-739358, dovitinib, Go 6976, GW441756, MGCD516, ONO-5390556, PHA-848125AC, Regorafenib, Sorafenib, Sunitinib, TSR-011, VM-902A, K252a, a 4-aminopyrazolylpyrimidine, a substituted pyrazolo[1,5-a] pyrimidine compound, BMS-754807, ONO-7579, F17752, ANA-12, ONO-4474, GZ389988, or TPX-0005 ((7S,13R)-11-fluoro-7,13-dimethyl-6,7,13,14-tetrahydro-1,15-ethenopyrazolo[4,3-f][1,4,8,10]benzoxatriazacyclotridecin-4(5H)-one; repotrectinib).

In some embodiments, relapse is one or more of detecting an increase in the number of cancer cells in the subject, an increase in the size of one or more tumors in the subject, an increase in tumor burden, an increase in the rate or extent of metastasis, worsening symptoms, in whole or in part, associated with the cancer, an increase in the extent of disease, and an acceleration of disease progression after a period of improvement. In some embodiments, relapse is progression of the cancer after a period of improvement. In some embodiments, a period of improvement is one or more of a decrease in the number of cancer cells in the subject, a decrease in the size of one or more tumors in the subject, a decrease in tumor burden, a decrease in the rate or extent of metastasis, improving symptoms, in whole or in part, associated with the cancer, a decrease in the extent of disease, and a slowing of disease progression.

In some embodiments, the tumor burden is assessed by RECIST version 1.1.

In some embodiments, the cancer is glioma and the progression of the glioma is assessed by RANO.

In some embodiments, a cancer that is not responding to therapy with a first Trk inhibitor is a cancer that is progressing. In some embodiments, progression of a cancer is one or more of an increase in the number of cancer cells in the subject, an increase in the size of one or more tumors in the subject, an increase in tumor burden, an increase in the rate or extent of metastasis, worsening symptoms, in whole or in part, associated with the cancer, an increase in the extent of disease, and an acceleration of disease progression. In some embodiments, the tumor burden is assessed using RECIST version 1.1. In some embodiments, the cancer is glioma and the progression of the glioma is assessed by RANO.

In some embodiments, circulating tumor DNA is used to monitor the responsiveness to therapy with a first Trk inhibitor. In some embodiments, an increase in the level ctDNA compared to the baseline is indicative of progression of the cancer, as described herein. In some embodiments, an increase in the level of ctDNA compared to the level during a period of improvement is indicative of relapse of the cancer.

In some embodiments, the subject that is intolerant to a first Trk inhibitor has had one or more of a severe, disabling, or life-threatening adverse event during therapy with the first Trk inhibitor, an unplanned hospitalization during therapy with the first Trk inhibitor, discontinuation of therapy with the first Trk inhibitor, dose reduction of the first Trk inhibitor, functional decline attributed to therapy with the first Trk inhibitor, and a decrease in performance status.

In some embodiments, the performance status is assessed using the Eastern Cooperative Oncology Group (ECOG) Scale of Performance Status.

In some embodiments, the performance status is assessed using the Karnofsky Performance Status.

In some embodiments, the performance status is assess by the Lansky Performance Score. In some embodiments, the subject is a pediatric patient. In some embodiments, wherein (i) the cancer in the subject has relapsed during therapy with a first Trk inhibitor; and/or (ii) the cancer in the subject is not responding to therapy with the first Trk inhibitor; and/or (iii) the subject is intolerant to the first Trk inhibitor, a Trk resistance mutation, e.g., any of the mutations described in Tables 11-13, is not detected.

Some examples of these methods further include administering the selected treatment to the identified subject. In some examples, the selected treatment is self-administered. In other examples, the selected treatment is administered by a medical professional (e.g., any of the medical professionals described herein). Some examples of these methods further include recording the selected treatment in the identified subject's clinical record (e.g., a computer readable medium).

Some examples of these methods further include administering the selected treatment to the identified subject. In some examples, the selected treatment is self-administered. In other examples, the selected treatment is administered by a medical professional (e.g., any of the medical professionals described herein). Some examples of these methods further include recording the selected treatment in the identified subject's clinical record (e.g., a computer readable medium).

Further provided herein is a composition for use in a method of selecting a treatment for a subject having a cancer, the method comprises: (a) determining whether, for a subject having a cancer and previously administered one or more doses of a first Trk inhibitor, (i) the cancer in the subject has relapsed during therapy with a first Trk inhibitor; and/or (ii) the cancer in the subject is not responding to therapy with a first Trk inhibitor; and/or (iii) the subject is intolerant to a first Trk inhibitor; (b) selecting a treatment that includes Compound 1, or a pharmaceutically acceptable salt, amorphous, or polymorph form thereof, and an another anticancer agent or anticancer therapy to a subject in which (i) the cancer in the subject has relapsed during therapy with a first Trk inhibitor; and/or (ii) the cancer in the subject is not responding to therapy with the first Trk inhibitor; and/or (iii) the subject is intolerant to the first Trk inhibitor; or (c) selecting additional doses of the first Trk inhibitor to a subject in which (i) the cancer has not relapsed during therapy with a first Trk inhibitor; and/or (ii) the cancer in the subject is responding to therapy with the first Trk inhibitor; and/or (iii) the subject is not intolerant to the first Trk inhibitor.

### Methods of Selecting a Subject Having a Cancer for Treatment

Also provided herein are methods of selecting a subject having a cancer for a treatment that does not include a first Trk inhibitor (e.g., entrectinib, TPX-0005, PLX7486, or (S)—N-(5-((R)-2-(2,5-difluorophenyl)pyrrolidin-1-yl)pyrazolo[1,5-a]pyrimidin-3-yl)-3-hydroxypyrrolidine-1-carboxamide sulfate) as a monotherapy that include identifying a subject in which (i) the cancer in the subject has relapsed during therapy with a first Trk inhibitor; and/or (ii) the cancer in the subject is not responding to therapy with a first Trk inhibitor; and/or (iii) the subject is intolerant to a first Trk inhibitor, and selecting the identified subject for a treatment that does not include the first Trk inhibitor as a monotherapy (e.g., any of the treatments that do not include a Trk inhibitor as a monotherapy described herein). For example, the subject can be administered a second Trk inhibitor as a monotherapy or in combination with another anticancer agent or treatment (e.g., the first Trk inhibitor).

Also provided herein are methods of selecting a subject having a cancer for a treatment that does not include a first Trk inhibitor (e.g., entrectinib, TPX-0005, PLX7486, or (S)—N-(5-((R)-2-(2,5-difluorophenyl)pyrrolidin-1-yl)pyrazolo[1,5-a]pyrimidin-3-yl)-3-hydroxypyrrolidine-1-carboxamide sulfate) as a monotherapy that include selecting a subject having a cancer (e.g., any of the cancers described herein) and identified as a subject in which (i) the cancer in the subject has relapsed during therapy with a first Trk inhibitor; and/or (ii) the cancer in the subject is not responding to therapy with a first Trk inhibitor; and/or (iii) the subject is intolerant to a first Trk inhibitor, for a treatment that does not include the first Trk inhibitor as a monotherapy (e.g., any of the treatments that do not include a Trk inhibitor as a monotherapy described herein). For example, the subject can be administered a second Trk inhibitor as a monotherapy or in combination with another anticancer agent or treatment (e.g., the first Trk inhibitor).

In some examples, the treatment that does not include a first Trk inhibitor as a monotherapy includes one or more of: surgery (e.g., open surgery or minimally invasive surgery), radiation therapy (e.g., external beam radiation therapy or internal radiation therapy), chemotherapy (e.g., an alkylating agent, antimetabolites, anti-microtubule agents, topoisomerase inhibitors, and cytotoxic antibiotics), immunotherapy (e.g., adoptive cell transfer, a cytokine, a cancer vaccine, and Bacillus Calmette-Guérom), hormone therapy (e.g., a drug that blocks estrogen, a drug that lowers estrogen levels, a progesterone-like drug, or an anti-androgen drug), small molecule drugs targeting other kinases in a Trk-signaling pathway, recombinant antibodies (e.g., any of exemplary recombinant antibodies described herein, e.g., anti-NGF antibodies), and stem cell transplant. In some examples, the treatment that does not include a first Trk inhibitor as a monotherapy can be, e.g., a treatment that includes (i) one or more of surgery, radiation therapy, chemotherapy, immunotherapy, hormone therapy, small molecule drugs targeting other kinases in a Trk-signaling pathway, recombinant antibodies, and stem cell transplant, and (ii) one or more Trk inhibitors (e.g., a second Trk inhibitor). In some embodiments, the treatment that does not include a first Trk inhibitor as a monotherapy can be, e.g., a treatment that includes two or more Trk inhibitors (e.g., any of the Trk inhibitors described herein). In some embodiments, the treatment that does not include a first Trk inhibitor includes a second Trk inhibitor. In some embodiments, the second Trk inhibitor is Compound 1, or a pharmaceutically acceptable salt, amorphous, or polymorph form thereof. In some embodiments the second Trk inhibitor is a pharmaceutical composition comprising a compounding agent as disclosed herein and Compound 1 or a solid form thereof, crystalline form thereof, or solvate or hydrate thereof, or a salt of Compound 1 or solid form thereof, crystalline form thereof, or solvate or hydrate thereof, as described herein. Additional examples of treatments that do not include a first Trk inhibitor as a monotherapy, and doses and routes of administration of the same, are described herein or known in the art.

Some examples of these methods further include administering a treatment that does not include a first Trk inhibitor as a monotherapy (e.g., using any of the treatments that do not include a first Trk inhibitor as a monotherapy, any of the routes of administration, any of the doses, and/or any of the frequencies of administration described herein) to the selected subject. In some examples, the treatment that does not include a first Trk inhibitor as a monotherapy is self-administered. In other examples, the treatment that does not include a first Trk inhibitor as a monotherapy is administered to the selected subject by a medical professional. In some examples, the selected subject is hospitalized. In other examples, the subject is administered the treatment that does not include a first Trk inhibitor as a monotherapy, on an outpatient basis. Some methods further include recording in the subject's clinical record (e.g., a computer readable medium) that the subject is selected for a treatment that does not include a first Trk inhibitor as a monotherapy.

Also provided herein are methods of selecting a subject having a cancer for a treatment that includes Compound 1, or a pharmaceutically acceptable salt, amorphous, or polymorph form thereof, that include: identifying a subject in which (i) the cancer in the subject has relapsed during therapy with a first Trk inhibitor; and/or (ii) the cancer in the subject is not responding to treatment with a first Trk inhibitor; and/or (iii) the subject is intolerant to a first Trk inhibitor; and selecting the identified subject for a treatment that includes Compound 1, or a pharmaceutically acceptable salt, amorphous, or polymorph form thereof.

Also provided herein are methods of selecting a subject having a cancer for a treatment that includes Compound 1, or a pharmaceutically acceptable salt, amorphous, or polymorph form thereof, and another anticancer agent (e.g., any one or more of the another anticancer agents described herein or known in the art) or another anticancer therapy that include: in which (i) the cancer in the subject has relapsed during therapy with a first Trk inhibitor; and/or (ii) the cancer in the subject is not responding to treatment with a first Trk inhibitor; and/or (iii) the subject is intolerant to a first Trk inhibitor; and selecting the identified subject for a treatment that includes Compound 1, or a pharmaceutically acceptable salt, amorphous, or polymorph form thereof, and another anticancer agent or anticancer therapy.

Also provided herein are methods of selecting a subject having a cancer for a treatment that includes Compound 1, or a pharmaceutically acceptable salt, amorphous, or polymorph form thereof, that include: identifying a subject in which (i) the cancer in the subject has relapsed during therapy with a first Trk inhibitor; and/or (ii) the cancer in the subject is not responding to treatment with a first Trk inhibitor; and/or (iii) the subject is intolerant to a first Trk inhibitor; and selecting the identified subject for a treatment that includes Compound 1, or a pharmaceutically acceptable salt, amorphous, or polymorph form thereof.

Also provided herein are methods of selecting a subject having a cancer for a treatment that includes Compound 1, or a pharmaceutically acceptable salt, amorphous, or polymorph form thereof, and another anticancer agent or anticancer therapy that include: identifying a subject in which (i) the cancer in the subject has relapsed during therapy with a first Trk inhibitor; and/or (ii) the cancer in the subject is not responding to treatment with a first Trk inhibitor; and/or (iii) the subject is intolerant to a first Trk inhibitor; and selecting the identified subject for a treatment that includes Compound 1, or a pharmaceutically acceptable salt, amorphous, or polymorph form thereof, and another anticancer agent or anticancer therapy.

Some examples of these methods further include administering a treatment that includes Compound 1, or a pharmaceutically acceptable salt, amorphous, or polymorph form thereof, and another anticancer agent or anticancer therapy to the selected subject.

In some embodiments, the cancer is a Trk-associated cancer. In some embodiments, the Trk-associated cancer exhibits at least one of a NTRK1, NTRK2, and/or NTRK3 fusion. In some embodiments, the at least one NTRK1, NTRK2, and/or NTRK3 fusion results in the expression of one or more of a TrkA fusion protein, and/or a TrkB fusion protein, and/or a TrkC fusion protein, e.g., one or more of the Trk fusions described in Tables 2, 5, and 8. In some embodiments, the Trk-associated cancer exhibits one or mutations point mutations/insertions/deletions in one or more of NTRK1, NTRK2, or NTRK3. Non-limiting examples of Trk kinase point mutations/insertions/deletions are described in Tables 3, 4, 6, 7, 9, and 10. In some embodiments, the Trk-associated cancer does not exhibit a Trk resistance mutation, e.g., any of the mutations described in Tables 11-13.

In some embodiments, the first Trk inhibitor is selected from the group consisting of: entrectinib (N-[5-(3,5-difluoro-benzyl)-1H-indazol-3-yl]-4-(4-methylpiperazin-1-yl)-2-(tetrahydro-pyran-4-ylamino)-benzamide), (S)—N-(5-((R)-2-(2,5-difluorophenyl)pyrrolidin-1-yl)pyrazolo[1,5-a]pyrimidin-3-yl)-3-hydroxypyrrolidine-1-carboxamide sulfate, cabozantinib ((N-(4-((6,7-Dimethoxyquinolin-4-yl)oxy)phenyl)-N′-(4-fluorophenyl)cyclopropane-1,1-dicarboxamide)), dovitinib (4-amino-5-fluoro-3-[6-(4-methylpiperazin-1-yl)-1H-benzimidazol-2-yl]quinolin-2(1H)-one mono 2-hydroxypropanoate hydrate), belizatinib (4-fluoro-N-(6-((4-(2-hydroxypropan-2-yl)piperidin-1-yl)methyl)-1-((1s,4s)-4-(isopropylcarbamoyl)cyclohexyl)-1H-benzo[d]imidazol-2-yl)benzamide), sitravatinib (N-(3-fluoro-4-((2-(5-(((2-methoxyethyl)amino)methyl)pyridin-2-yl)thieno[3,2-b]pyridin-7-yl)oxy)phenyl)-N-(4-fluorophenyl)cyclopropane-1,1-dicarboxamide), PLX7486, altiratinib (N-(4-((2-(cyclopropanecarboxamido)pyridin-4-yl)oxy)-2,5-difluorophenyl)-N-(4-fluorophenyl)cyclopropane-1,1-dicarboxamide), AZD7451 ((S)—N-(1-(5-fluoropyrimidin-2-yl)ethyl)-3-(5-isopropoxy-1H-pyrazol-3-yl)-3H-imidazo[4,5-b]pyridin-5-amine), (6R,15R)-9-fluoro-15-methyl-2,11,16,20,21,24-hexaazapentacyclo[16.5.2.02,6.07,12.021,25]pentacosa-1(24),7,9, 11,18(25),19,22-heptaen-17-one, a (R)-2-phenylpyrrolidine substituted imadazopyridazine, AZD6918, GNF-4256, GTx-186, GNF-5837, AZ623, AG-879, CT327, AR-772, AR-523, AR-786, AR-256, AR-618, AZ-23, CEP-701, CEP-751, PHA-739358, dovitinib, Go 6976, GW441756, MGCD516, ONO-5390556, PHA-848125AC, Regorafenib, Sorafenib, Sunitinib, TSR-011, VM-902A, K252a, a 4-aminopyrazolylpyrimidine, a substituted pyrazolo[1,5-a] pyrimidine compound, BMS-754807, ONO-7579, F17752, ANA-12, ONO-4474, GZ389988, or TPX-0005 ((7S,13R)-11-fluoro-7,13-dimethyl-6,7,13,14-tetrahydro-1,15-ethenopyrazolo[4,3-f][1,4,8,10]benzoxatriazacyclotridecin-4(5H)-one; repotrectinib).

In some embodiments, relapse is one or more of detecting an increase in the number of cancer cells in the subject, an increase in the size of one or more tumors in the subject, an increase in tumor burden, an increase in the rate or extent of metastasis, worsening symptoms, in whole or in part, associated with the cancer, an increase in the extent of disease, and an acceleration of disease progression after a period of improvement. In some embodiments, relapse is progression of the cancer after a period of improvement. In some embodiments, a period of improvement is one or more of a decrease in the number of cancer cells in the subject, a decrease in the size of one or more tumors in the subject, a decrease in tumor burden, a decrease in the rate or extent of metastasis, improving symptoms, in whole or in part, associated with the cancer, a decrease in the extent of disease, and a slowing of disease progression.

In some embodiments, the tumor burden is assessed by RECIST version 1.1.

In some embodiments, the cancer is glioma and the progression of the glioma is assessed by RANO.

In some embodiments, a cancer that is not responding to therapy with a first Trk inhibitor is a cancer that is progressing. In some embodiments, progression of a cancer is one or more of an increase in the number of cancer cells in the subject, an increase in the size of one or more tumors in the subject, an increase in tumor burden, an increase in the rate or extent of metastasis, worsening symptoms, in whole or in part, associated with the cancer, an increase in the extent of disease, and an acceleration of disease progression. In some embodiments, the tumor burden is assessed using RECIST version 1.1. In some embodiments, the cancer is glioma and the progression of the glioma is assessed by RANO.

In some embodiments, circulating tumor DNA is used to monitor the responsiveness to therapy with a first Trk inhibitor. In some embodiments, an increase in the level ctDNA compared to the baseline is indicative of progression of the cancer, as described herein. In some embodiments, an increase in the level of ctDNA compared to the level during a period of improvement is indicative of relapse of the cancer.

In some embodiments, the subject that is intolerant to a first Trk inhibitor has had one or more of a severe, disabling, or life-threatening adverse event during therapy with the first Trk inhibitor, an unplanned hospitalization during therapy with the first Trk inhibitor, discontinuation of therapy with the first Trk inhibitor, dose reduction of the first Trk inhibitor, functional decline attributed to treatment with the first Trk inhibitor, and a decrease in performance status.

In some embodiments, the performance status is assessed using the Eastern Cooperative Oncology Group (ECOG) Scale of Performance Status.

In some embodiments, the performance status is assessed using the Karnofsky Performance Status.

In some embodiments, the performance status is assess by the Lansky Performance Score. In some embodiments, the subject is a pediatric patient. In some embodiments, wherein (i) the cancer in the subject has relapsed during therapy with a first Trk inhibitor; and/or (ii) the cancer in the subject is not responding to therapy with the first Trk inhibitor; and/or (iii) the subject is intolerant to the first Trk inhibitor, a Trk resistance mutation, e.g., any of the mutations described in Tables 11-13, is not detected.

Methods of Determining the Likelihood that a Subject Having a Cancer Will have a Positive Response to a Therapy with a Trk Inhibitor as a Monotherapy

Also provided herein are methods of determining the likelihood that a subject having a cancer (e.g., any of the cancers described herein) will have a positive response to a therapy with a first Trk inhibitor (e.g., entrectinib, TPX-0005, PLX7486, or (S)—N-(5-((R)-2-(2,5-difluorophenyl)pyrrolidin-1-yl)pyrazolo[1,5-a]pyrimidin-3-yl)-3-hydroxypyrrolidine-1-carboxamide sulfate) as a monotherapy that include determining whether (i) the cancer in the subject has relapsed during therapy with a first Trk inhibitor; and/or (ii) the cancer in the subject is not responding to therapy with the first Trk inhibitor; and/or (iii) the subject is intolerant to the first Trk inhibitor, has a decreased likelihood of having a positive response to a therapy with a first Trk inhibitor as a monotherapy (e.g., as compared to a subject in which (i) the cancer has not relapsed during therapy with the first Trk inhibitor; and/or (ii) the cancer in the subject is responding to therapy with the first Trk inhibitor; and/or (iii) the subject is not intolerant to the first Trk inhibitor).

Also provided herein are methods of determining the likelihood that a subject having cancer (e.g., any of the cancers described herein) will have a positive response to a therapy with a first Trk inhibitor (e.g., entrectinib, TPX-0005, PLX7486, or (S)—N-(5-((R)-2-(2,5-difluorophenyl)pyrrolidin-1-yl)pyrazolo[1,5-a]pyrimidin-3-yl)-3-hydroxypyrrolidine-1-carboxamide sulfate) as a monotherapy that include determining that a subject in which (i) the cancer in the subject has relapsed during therapy with a first Trk inhibitor; and/or (ii) the cancer in the subject is not responding to therapy with the first Trk inhibitor; and/or (iii) the subject is intolerant to the first Trk inhibitor, has a decreased likelihood of having a positive response to therapy with the first Trk inhibitor as a monotherapy (e.g., as compared to a subject in which (i) the cancer has not relapsed during therapy with the first Trk inhibitor; and/or (ii) the cancer in the subject is responding to therapy with the first Trk inhibitor; and/or (iii) the subject is not intolerant to the first Trk inhibitor).

Some examples of these methods include administering a treatment that does not include a first Trk inhibitor as a monotherapy (e.g., any of the treatments that do not include a first Trk inhibitor as a monotherapy described herein) to a subject determined to have a decreased likelihood of having a positive response to therapy with a first Trk inhibitor as a monotherapy.

In some examples, the treatment that does not include a first Trk inhibitor as a monotherapy includes one or more of: surgery (e.g., open surgery or minimally invasive surgery), radiation therapy (e.g., external beam radiation therapy or internal radiation therapy), chemotherapy (e.g., an alkylating agent, antimetabolites, anti-microtubule agents, topoisomerase inhibitors, and cytotoxic antibiotics), immunotherapy (e.g., adoptive cell transfer, a cytokine, a cancer vaccine, and Bacillus Calmette-Guérom), hormone therapy (e.g., a drug that blocks estrogen, a drug that lowers estrogen levels, a progesterone-like drug, or an anti-androgen drug), small molecule drugs targeting other kinases in a Trk-signaling pathway, recombinant antibodies (e.g., any of exemplary recombinant antibodies described herein, e.g., anti-NGF antibodies), and stem cell transplant. In some examples, the treatment that does not include a first Trk inhibitor as a monotherapy can be, e.g., a treatment that includes (i) one or more of surgery, radiation therapy, chemotherapy, immunotherapy, hormone therapy, small molecule drugs targeting other kinases in a Trk-signaling pathway, recombinant antibodies, and stem cell transplant, and (ii) one or more Trk inhibitors (e.g., a second Trk inhibitor or any of the Trk inhibitors described herein). In some embodiments, the treatment that does not include a first Trk inhibitor includes a second Trk inhibitor. In some embodiments, the second Trk inhibitor is Compound 1, or a pharmaceutically acceptable salt, amorphous, or polymorph form thereof. In some embodiments the second Trk inhibitor is a pharmaceutical composition comprising a compounding agent as disclosed herein and Compound 1 or a solid form thereof, crystalline form thereof, or solvate or hydrate thereof, or a salt of Compound 1 or solid form thereof, crystalline form thereof, or solvate or hydrate thereof, as described herein. In some embodiments, the treatment that does not include a first Trk inhibitor as a monotherapy can be, e.g., a treatment that includes two or more Trk inhibitors (e.g., any of the Trk inhibitors described herein). Additional examples of treatments that do not include a first Trk inhibitor as a monotherapy, and doses and routes of administration of the same, are described herein or known in the art.

In some examples, the treatment that does not include a first Trk inhibitor as a monotherapy is self-administered. In other examples, the treatment that does not include a first Trk inhibitor as a monotherapy is administered to the subject by a medical professional. In some examples, the subject is hospitalized. In other examples, the subject is administered the treatment that does not include a first Trk inhibitor as a monotherapy, on an outpatient basis. Some methods further include recording in the subject's clinical record (e.g., a computer readable medium) that the subject has a decreased likelihood of having a positive response to treatment with a first Trk inhibitor as a monotherapy.

Also provided herein are methods of determining the likelihood that a subject having a cancer will have a positive response to therapy that includes Compound 1, or a pharmaceutically acceptable salt, amorphous, or polymorph form thereof, that include: determining whether (i) the cancer in the subject has relapsed during therapy with a first Trk inhibitor; and/or (ii) the cancer in the subject is not responding to therapy with the first Trk inhibitor; and/or (iii) the subject is intolerant to the first Trk inhibitor, has an increased likelihood of having a positive response to treatment that includes Compound 1, or a pharmaceutically acceptable salt, amorphous, or polymorph form thereof.

Also provided herein are methods of determining the likelihood that a subject having cancer will have a positive response to treatment that includes Compound 1, or a pharmaceutically acceptable salt, amorphous, or polymorph form thereof, that include: determining that a subject in which (i) the cancer in the subject has relapsed during therapy with a first Trk inhibitor; and/or (ii) the cancer in the subject is not responding to therapy with the first Trk inhibitor; and/or (iii) the subject is intolerant to the first Trk inhibitor, has an increased likelihood of having a positive response to treatment including Compound 1, or a pharmaceutically acceptable salt, amorphous, or polymorph form thereof.

In some embodiments, the cancer is a Trk-associated cancer. In some embodiments, the Trk-associated cancer exhibits at least one of a NTRK1, NTRK2, and/or NTRK3 fusion. In some embodiments, the at least one NTRK1, NTRK2, and/or NTRK3 fusion results in the expression of one or more of a TrkA fusion protein, and/or a TrkB fusion protein, and/or a TrkC fusion protein, e.g., one or more of the Trk fusions described in Tables 2, 5, and 8. In some embodiments, the at least one NTRK1, NTRK2, and/or NTRK3 fusion results in the expression of one or more of a TrkA fusion protein, and/or a TrkB fusion protein, and/or a TrkC fusion protein, wherein the TrkA fusion protein comprises one or more of the of the fusions selected from the group consisting of: TP53-TrkA, LMNA-TrkA, CD74-TrkA, TFG-TrkA, TPM3-TrkA, NFASC-TrkA, BCAN-TrkA, MPRIP-TrkA, TPR-TrkA, RFWD2-TrkA, IRF2BP2-TrkA, SQSTM1-TrkA, SSBP2-TrkA, RABGAP1L-TrkA, C18ORF8-TrkA, RNF213-TrkA, TBC1D22A-TrkA, C20ORF112-TrkA, DNER-TrkA, ARHGEF2-TrkA, CHTOP-TrkA, PPL-TrkA, PLEKHA6-TrkA, PEAR1-TrkA, MRPL24-TrkA, MDM4-TrkA, LRRC71-TrkA, GRIPAP1-TrkA, TAF-TrkA, EPS15-TrkA, DYNC2H1-TrkA, CEL-TrkA, EPHB2-TrkA, TGF-TrkA, NELL1-TrkA, EPL4-TrkA, CTNND2-TrkA, TCEANC2-TrkA, SCYL3-TrkA, AMOTL2-TrkA, MEF2D-TrkA, L7a-TrkA, ZBTB7B-TrkA, TRIM63-TrkA, DDR2-TrkAl, GON4L-TrkA, PDE4DIP-TrkA, NTRK1-P2RY8, CTRC-TrkA, and VANGL2-TrkA; and/or the TrkB fusion protein comprises one or more of the of the fusions selected from the group consisting of: NACC2-TrkB, QKI-TrkB, AFAP1-TrkB, PAN3-TrkB, SQSTM1-TrkB, TRIM24-TrkB, VCL-TrkB, AGBL4-TrkB, DAB2IP-TrkB, TrkB-TERT, ETV6-TrkB, NOS1AP-TrkB, GKAP1-TrkB, KCTD8-TrkB, TBC1D2-TrkB, VCAN-TrkB, SLMAP-TrkB, TLE4-TrkB, STRN3-TrkB, WNK2-TrkB, TrkB-BEND5, TrkB-TRAF2, Nav1-TrkB, and STRN-TrkB; and/or the TrkC fusion protein comprises one or more of the of the fusions selected from the group consisting of: ETV6-TrkC1, BTBD1-TrkC, LYN-TrkC, RBPMS-TrkC, EML4-TrkC, TrkC-HOMER2, TFG-TrkC, FAT1-TrkC, MYO5A-TrkC, MYH9-TrkC, KANK1-TrkC, SQSTM1-TrkC, UBE2R2-TrkC, HNRNPA2B1-TrkC, VPS18-TrkC, AKAP13-TrkC, TrkC-LOXL2, TrkC-PEAK1, ZNF710-TrkC, TPM4-TrkC, and LMNA-TrkC. In some embodiments, the Trk-associated cancer exhibits one or mutations point mutations/insertions/deletions in one or more of NTRK1, NTRK2, or NTRK3. Non-limiting examples of Trk kinase point mutations/insertions/deletions are described in Tables 3, 4, 6, 7, 9, and 10. In some embodiments, the Trk-associated cancer does not exhibit a Trk resistance mutation, e.g., any of the mutations described in Tables 11-13.

In some embodiments, the first Trk inhibitor is selected from the group consisting of: entrectinib (N-[5-(3,5-difluoro-benzyl)-1H-indazol-3-yl]-4-(4-methylpiperazin-1-yl)-2-(tetrahydro-pyran-4-ylamino)-benzamide), (S)—N-(5-((R)-2-(2,5-difluorophenyl)pyrrolidin-1-yl)pyrazolo[1,5-a]pyrimidin-3-yl)-3-hydroxypyrrolidine-1-carboxamide sulfate, cabozantinib ((N-(4-((6,7-Dimethoxyquinolin-4-yl)oxy)phenyl)-N′-(4-fluorophenyl)cyclopropane-1,1-dicarboxamide)), dovitinib (4-amino-5-fluoro-3-[6-(4-methylpiperazin-1-yl)-1H-benzimidazol-2-yl]quinolin-2(1H)-one mono 2-hydroxypropanoate hydrate), belizatinib (4-fluoro-N-(6-((4-(2-hydroxypropan-2-yl)piperidin-1-yl)methyl)-1-((1s,4s)-4-(isopropylcarbamoyl)cyclohexyl)-1H-benzo[d]imidazol-2-yl)benzamide), sitravatinib (N-(3-fluoro-4-((2-(5-(((2-methoxyethyl)amino)methyl)pyridin-2-yl)thieno[3,2-b]pyridin-7-yl)oxy)phenyl)-N-(4-fluorophenyl)cyclopropane-1,1-dicarboxamide), PLX7486, altiratinib (N-(4-((2-(cyclopropanecarboxamido)pyridin-4-yl)oxy)-2,5-difluorophenyl)-N-(4-fluorophenyl)cyclopropane-1,1-dicarboxamide), AZD7451 ((S)—N-(1-(5-fluoropyrimidin-2-yl)ethyl)-3-(5-isopropoxy-1H-pyrazol-3-yl)-3H-imidazo[4,5-b]pyridin-5-amine), (6R,15R)-9-fluoro-15-methyl-2,11,16,20,21,24-hexaazapentacyclo[16.5.2.02,6.07,12.021,25]pentacosa-1(24),7,9, 11,18(25),19,22-heptaen-17-one, a (R)-2-phenylpyrrolidine substituted imadazopyridazine, AZD6918, GNF-4256, GTx-186, GNF-5837, AZ623, AG-879, CT327, AR-772, AR-523, AR-786, AR-256, AR-618, AZ-23, CEP-701, CEP-751, PHA-739358, dovitinib, Go 6976, GW441756, MGCD516, ONO-5390556, PHA-848125AC, Regorafenib, Sorafenib, Sunitinib, TSR-011, VM-902A, K252a, a 4-aminopyrazolylpyrimidine, a substituted pyrazolo[1,5-a] pyrimidine compound, BMS-754807, ONO-7579, F17752, ANA-12, ONO-4474, GZ389988, or TPX-0005 ((7S,13R)-11-fluoro-7,13-dimethyl-6,7,13,14-tetrahydro-1,15-ethenopyrazolo[4,3-f][1,4,8,10]benzoxatriazacyclotridecin-4(5H)-one; repotrectinib).

In some embodiments, relapse is one or more of detecting an increase in the number of cancer cells in the subject, an increase in the size of one or more tumors in the subject, an increase in tumor burden, an increase in the rate or extent of metastasis, worsening symptoms, in whole or in part, associated with the cancer, an increase in the extent of disease, and an acceleration of disease progression after a period of improvement. In some embodiments, relapse is progression of the cancer after a period of improvement. In some embodiments, a period of improvement is one or more of a decrease in the number of cancer cells in the subject, a decrease in the size of one or more tumors in the subject, a decrease in tumor burden, a decrease in the rate or extent of metastasis, improving symptoms, in whole or in part, associated with the cancer, a decrease in the extent of disease, and a slowing of disease progression.

In some embodiments, the tumor burden is assessed by RECIST version 1.1.

In some embodiments, the cancer is glioma and the progression of the glioma is assessed by RANO.

In some embodiments, a cancer that is not responding to therapy with a first Trk inhibitor is a cancer that is progressing. In some embodiments, progression of a cancer is one or more of an increase in the number of cancer cells in the subject, an increase in the size of one or more tumors in the subject, an increase in tumor burden, an increase in the rate or extent of metastasis, worsening symptoms, in whole or in part, associated with the cancer, an increase in the extent of disease, and an acceleration of disease progression. In some embodiments, the tumor burden is assessed using RECIST version 1.1. In some embodiments, the cancer is glioma and the progression of the glioma is assessed by RANO.

In some embodiments, circulating tumor DNA is used to monitor the responsiveness to therapy with a first Trk inhibitor. In some embodiments, an increase in the level ctDNA compared to the baseline is indicative of progression of the cancer, as described herein. In some embodiments, an increase in the level of ctDNA compared to the level during a period of improvement is indicative of relapse of the cancer.

In some embodiments, the subject that is intolerant to a first Trk inhibitor has had one or more of a severe, disabling, or life-threatening adverse event during therapy with the first Trk inhibitor, an unplanned hospitalization during therapy with the first Trk inhibitor, discontinuation of therapy with the first Trk inhibitor, dose reduction of the first Trk inhibitor, functional decline attributed to therapy with the first Trk inhibitor, and a decrease in performance status.

In some embodiments, the performance status is assessed using the Eastern Cooperative Oncology Group (ECOG) Scale of Performance Status.

In some embodiments, the performance status is assessed using the Karnofsky Performance Status.

In some embodiments, the performance status is assess by the Lansky Performance Score. In some embodiments, the subject is a pediatric patient. In some embodiments, wherein (i) the cancer in the subject has relapsed during therapy with a first Trk inhibitor; and/or (ii) the cancer in the subject is not responding to therapy with the first Trk inhibitor; and/or (iii) the subject is intolerant to the first Trk inhibitor, a Trk resistance mutation, e.g., any of the mutations described in Tables 11-13, is not detected.

Methods of Predicting the Efficacy of Therapy with a Trk Inhibitor as a Monotherapy in a Subject Having Cancer

Also provided herein are methods of predicting the efficacy of therapy with a first Trk inhibitor (e.g., entrectinib, TPX-0005, PLX7486, or (S)—N-(5-((R)-2-(2,5-difluorophenyl)pyrrolidin-1-yl)pyrazolo[1,5-a]pyrimidin-3-yl)-3-hydroxypyrrolidine-1-carboxamide sulfate) as a monotherapy in a subject having cancer (e.g., any of the cancers described herein) that include determining whether (i) the cancer in the subject has relapsed during therapy with a first Trk inhibitor; and/or (ii) the cancer in the subject is not responding to therapy with a first Trk inhibitor; and/or (iii) the subject is intolerant to a first Trk inhibitor, and determining that a therapy with a first Trk inhibitor as a monotherapy is less likely to be effective in a subject in which (i) the cancer in the subject has relapsed during therapy with the first Trk inhibitor; and/or (ii) the cancer in the subject is not responding to therapy with the first Trk inhibitor; and/or (iii) the subject is intolerant to the first Trk inhibitor (e.g., as compared to a subject in which (i) the cancer has not relapsed during therapy with the first Trk inhibitor; and/or (ii) the cancer in the subject is responding to therapy with the first Trk inhibitor; and/or (iii) the subject is not intolerant to the first Trk inhibitor).

Also provided herein are methods of predicting the efficacy of a therapy with a first Trk inhibitor (e.g., entrectinib, TPX-0005, PLX7486, or (S)—N-(5-((R)-2-(2,5-difluorophenyl)pyrrolidin-1-yl)pyrazolo[1,5-a]pyrimidin-3-yl)-3-hydroxypyrrolidine-1-carboxamide sulfate) as a monotherapy in a subject having a cancer (e.g., any of the cancers described herein) that include determining that therapy with a first Trk inhibitor as a monotherapy is less likely to be effective in a subject in which (i) the cancer in the subject has relapsed during therapy with a first Trk inhibitor; and/or (ii) the cancer in the subject is not responding to therapy with a first Trk inhibitor; and/or (iii) the subject is intolerant to a first Trk inhibitor (e.g., as compared to a subject in which (i) the cancer has not relapsed during therapy with the first Trk inhibitor; and/or (ii) the cancer in the subject is responding to therapy with the first Trk inhibitor; and/or (iii) the subject is not intolerant to the first Trk inhibitor).

Some methods further include recording in the subject's clinical record (e.g., a computer readable medium) the predicted efficacy of a therapy with a first Trk inhibitor as a monotherapy, in the subject having a cancer. Some examples of these methods further include selecting a treatment that does not include a first Trk inhibitor as a monotherapy for the subject. Some examples further include administering the selected treatment to the subject (e.g., using any of the treatments that do not include a first Trk inhibitor as a monotherapy, any of the routes of administration, any of the doses, and/or any of the frequencies of administration described herein).

In some examples, the treatment that does not include a first Trk inhibitor as a monotherapy includes one or more of: surgery (e.g., open surgery or minimally invasive surgery), radiation therapy (e.g., external beam radiation therapy or internal radiation therapy), chemotherapy (e.g., an alkylating agent, antimetabolites, anti-microtubule agents, topoisomerase inhibitors, and cytotoxic antibiotics), immunotherapy (e.g., adoptive cell transfer, a cytokine, a cancer vaccine, and Bacillus Calmette-Guérom), hormone therapy (e.g., a drug that blocks estrogen, a drug that lowers estrogen levels, a progesterone-like drug, or an anti-androgen drug), small molecule drugs targeting other kinases in a Trk-signaling pathway, recombinant antibodies (e.g., any of exemplary recombinant antibodies described herein, e.g., anti-NGF antibodies), and stem cell transplant. In some examples, the treatment that does not include a first Trk inhibitor as a monotherapy can be, e.g., a treatment that includes (i) one or more of surgery, radiation therapy, chemotherapy, immunotherapy, hormone therapy, small molecule drugs targeting other kinases in a Trk-signaling pathway, recombinant antibodies, and stem cell transplant, and (ii) one or more Trk inhibitors (e.g., a second Trk inhibitor or any of the Trk inhibitors described herein). In some embodiments, the treatment that does not include a first Trk inhibitor as a monotherapy can be, e.g., a treatment that includes two or more Trk inhibitors (e.g., any of the Trk inhibitors described herein). Additional examples of treatments that do not include a first Trk inhibitor as a monotherapy, and doses and routes of administration of the same, are described herein or known in the art.

In some examples, the treatment that does not include a first Trk inhibitor as a monotherapy is self-administered. In other examples, the treatment that does not include a first Trk inhibitor as a monotherapy is administered to the subject by a medical professional. In some examples, the subject is hospitalized. In other examples, the subject is administered the treatment that does not include a first Trk inhibitor as a monotherapy, on an outpatient basis.

Also provided herein are methods of predicting the efficacy of therapy with a therapy including Compound 1, or a pharmaceutically acceptable salt, amorphous, or polymorph form thereof, in a subject having cancer, that include: determining whether (i) the cancer in the subject has relapsed during therapy with a first Trk inhibitor; and/or (ii) the cancer in the subject is not responding to therapy with a first Trk inhibitor; and/or (iii) the subject is intolerant to a first Trk inhibitor; and determining that therapy including Compound 1, or a pharmaceutically acceptable salt, amorphous, or polymorph form thereof, is more likely to be more effective than the first Trk inhibitor in the subject in which (i) the cancer in the subject has relapsed during therapy with a first Trk inhibitor; and/or (ii) the cancer in the subject is not responding to therapy with a first Trk inhibitor; and/or (iii) the subject is intolerant to a first Trk inhibitor.

Also provided herein are methods of predicting the efficacy of therapy including Compound 1, or a pharmaceutically acceptable salt, amorphous, or polymorph form thereof, in a subject having cancer, that include: determining that therapy including Compound 1, or a pharmaceutically acceptable salt, amorphous, or polymorph form thereof, is more likely to be more effective than a first Trk inhibitor in a subject in which (i) the cancer in the subject has relapsed during therapy with a first Trk inhibitor; and/or (ii) the cancer in the subject is not responding to therapy with a first Trk inhibitor; and/or (iii) the subject is intolerant to a first Trk inhibitor.

In some embodiments, the cancer is a Trk-associated cancer. In some embodiments, the Trk-associated cancer exhibits at least one of a NTRK1, NTRK2, and/or NTRK3 fusion. In some embodiments, the at least one NTRK1, NTRK2, and/or NTRK3 fusion results in the expression of one or more of a TrkA fusion protein, and/or a TrkB fusion protein, and/or a TrkC fusion protein, e.g., one or more of the Trk fusions described in Tables 2, 5, and 8. In some embodiments, the at least one NTRK1, NTRK2, and/or NTRK3 fusion results in the expression of one or more of a TrkA fusion protein, and/or a TrkB fusion protein, and/or a TrkC fusion protein, wherein the TrkA fusion protein comprises one or more of the of the fusions selected from the group consisting of: TP53-TrkA, LMNA-TrkA, CD74-TrkA, TFG-TrkA, TPM3-TrkA, NFASC-TrkA, BCAN-TrkA, MPRIP-TrkA, TPR-TrkA, RFWD2-TrkA, IRF2BP2-TrkA, SQSTM1-TrkA, SSBP2-TrkA, RABGAP1L-TrkA, C18ORF8-TrkA, RNF213-TrkA, TBC1D22A-TrkA, C20ORF112-TrkA, DNER-TrkA, ARHGEF2-TrkA, CHTOP-TrkA, PPL-TrkA, PLEKHA6-TrkA, PEAR1-TrkA, MRPL24-TrkA, MDM4-TrkA, LRRC71-TrkA, GRIPAP1-TrkA, TAF-TrkA, EPS15-TrkA, DYNC2H1-TrkA, CEL-TrkA, EPHB2-TrkA, TGF-TrkA, NELL1-TrkA, EPL4-TrkA, CTNND2-TrkA, TCEANC2-TrkA, SCYL3-TrkA, AMOTL2-TrkA, MEF2D-TrkA, L7a-TrkA, ZBTB7B-TrkA, TRIM63-TrkA, DDR2-TrkAl, GON4L-TrkA, PDE4DIP-TrkA, NTRK1-P2RY8, CTRC-TrkA, and VANGL2-TrkA; and/or the TrkB fusion protein comprises one or more of the of the fusions selected from the group consisting of: NACC2-TrkB, QKI-TrkB, AFAP1-TrkB, PAN3-TrkB, SQSTM1-TrkB, TRIM24-TrkB, VCL-TrkB, AGBL4-TrkB, DAB2IP-TrkB, TrkB-TERT, ETV6-TrkB, NOS1AP-TrkB, GKAP1-TrkB, KCTD8-TrkB, TBC1D2-TrkB, VCAN-TrkB, SLMAP-TrkB, TLE4-TrkB, STRN3-TrkB, WNK2-TrkB, TrkB-BEND5, TrkB-TRAF2, Nav1-TrkB, and STRN-TrkB; and/or the TrkC fusion protein comprises one or more of the of the fusions selected from the group consisting of: ETV6-TrkC1, BTBD1-TrkC, LYN-TrkC, RBPMS-TrkC, EML4-TrkC, TrkC-HOMER2, TFG-TrkC, FAT1-TrkC, MYO5A-TrkC, MYH9-TrkC, KANK1-TrkC, SQSTM1-TrkC, UBE2R2-TrkC, HNRNPA2B1-TrkC, VPS18-TrkC, AKAP13-TrkC, TrkC-LOXL2, TrkC-PEAK1, ZNF710-TrkC, TPM4-TrkC, and LMNA-TrkC. In some embodiments, the Trk-associated cancer exhibits one or mutations point mutations/insertions/deletions in one or more of NTRK1, NTRK2, or NTRK3. Non-limiting examples of Trk kinase point mutations/insertions/deletions are described in Tables 3, 4, 6, 7, 9, and 10. In some embodiments, the Trk-associated cancer does not exhibit a Trk resistance mutation, e.g., any of the mutations described in Tables 11-13.

In some embodiments, the first Trk inhibitor is selected from the group consisting of: entrectinib (N-[5-(3,5-difluoro-benzyl)-1H-indazol-3-yl]-4-(4-methylpiperazin-1-yl)-2-(tetrahydro-pyran-4-ylamino)-benzamide), (S)—N-(5-((R)-2-(2,5-difluorophenyl)pyrrolidin-1-yl)pyrazolo[, 5-a]pyrimidin-3-yl)-3-hydroxypyrrolidine-1-carboxamide sulfate, cabozantinib ((N-(4-((6,7-Dimethoxyquinolin-4-yl)oxy)phenyl)-N′-(4-fluorophenyl)cyclopropane-1,1-dicarboxamide)), dovitinib (4-amino-5-fluoro-3-[6-(4-methylpiperazin-1-yl)-1H-benzimidazol-2-yl]quinolin-2(1H)-one mono 2-hydroxypropanoate hydrate), belizatinib (4-fluoro-N-(6-((4-(2-hydroxypropan-2-yl)piperidin-1-yl)methyl)-1-((1s,4s)-4-(isopropylcarbamoyl)cyclohexyl)-1H-benzo[d]imidazol-2-yl)benzamide), sitravatinib (N-(3-fluoro-4-((2-(5-(((2-methoxyethyl)amino)methyl)pyridin-2-yl)thieno[3,2-b]pyridin-7-yl)oxy)phenyl)-N-(4-fluorophenyl)cyclopropane-1,1-dicarboxamide), PLX7486, altiratinib (N-(4-((2-(cyclopropanecarboxamido)pyridin-4-yl)oxy)-2,5-difluorophenyl)-N-(4-fluorophenyl)cyclopropane-1,1-dicarboxamide), AZD7451 ((S)—N-(1-(5-fluoropyrimidin-2-yl)ethyl)-3-(5-isopropoxy-1H-pyrazol-3-yl)-3H-imidazo[4,5-b]pyridin-5-amine), (6R,15R)-9-fluoro-15-methyl-2,11,16,20,21,24-hexaazapentacyclo[16.5.2.02,6.07,12.021,25]pentacosa-1(24),7,9, 11,18(25),19,22-heptaen-17-one, a (R)-2-phenylpyrrolidine substituted imadazopyridazine, AZD6918, GNF-4256, GTx-186, GNF-5837, AZ623, AG-879, CT327, AR-772, AR-523, AR-786, AR-256, AR-618, AZ-23, CEP-701, CEP-751, PHA-739358, dovitinib, Go 6976, GW441756, MGCD516, ONO-5390556, PHA-848125AC, Regorafenib, Sorafenib, Sunitinib, TSR-011, VM-902A, K252a, a 4-aminopyrazolylpyrimidine, a substituted pyrazolo[1,5-a] pyrimidine compound, BMS-754807, ONO-7579, F17752, ANA-12, ONO-4474, GZ389988, or TPX-0005 ((7S,13R)-11-fluoro-7,13-dimethyl-6,7,13,14-tetrahydro-1,15-ethenopyrazolo[4,3-f][1,4,8,10]benzoxatriazacyclotridecin-4(5H)-one; repotrectinib).

In some embodiments, relapse is one or more of detecting an increase in the number of cancer cells in the subject, an increase in the size of one or more tumors in the subject, an increase in tumor burden, an increase in the rate or extent of metastasis, worsening symptoms, in whole or in part, associated with the cancer, an increase in the extent of disease, and an acceleration of disease progression after a period of improvement. In some embodiments, relapse is progression of the cancer after a period of improvement. In some embodiments, a period of improvement is one or more of a decrease in the number of cancer cells in the subject, a decrease in the size of one or more tumors in the subject, a decrease in tumor burden, a decrease in the rate or extent of metastasis, improving symptoms, in whole or in part, associated with the cancer, a decrease in the extent of disease, and a slowing of disease progression.

In some embodiments, the tumor burden is assessed by RECIST version 1.1.

In some embodiments, the cancer is glioma and the progression of the glioma is assessed by RANO.

In some embodiments, a cancer that is not responding to therapy with a first Trk inhibitor is a cancer that is progressing. In some embodiments, progression of a cancer is one or more of an increase in the number of cancer cells in the subject, an increase in the size of one or more tumors in the subject, an increase in tumor burden, an increase in the rate or extent of metastasis, worsening symptoms, in whole or in part, associated with the cancer, an increase in the extent of disease, and an acceleration of disease progression.

In some embodiments, the tumor burden is assessed using RECIST version 1.1.

In some embodiments, the cancer is glioma and the progression of the glioma is assessed by RANO.

In some embodiments, circulating tumor DNA is used to monitor the responsiveness to therapy with a first Trk inhibitor. In some embodiments, an increase in the level ctDNA compared to the baseline is indicative of progression of the cancer, as described herein. In some embodiments, an increase in the level of ctDNA compared to the level during a period of improvement is indicative of relapse of the cancer.

In some embodiments, the subject that is intolerant to a first Trk inhibitor has had one or more of a severe, disabling, or life-threatening adverse event during therapy with the first Trk inhibitor, an unplanned hospitalization during therapy with the first Trk inhibitor, discontinuation of therapy with the first Trk inhibitor, dose reduction of the first Trk inhibitor, functional decline attributed to therapy with the first Trk inhibitor, and a decrease in performance status.

In some embodiments, the performance status is assessed using the Eastern Cooperative Oncology Group (ECOG) Scale of Performance Status.

In some embodiments, the performance status is assessed using the Karnofsky Performance Status.

In some embodiments, the performance status is assess by the Lansky Performance Score. In some embodiments, the subject is a pediatric patient. In some embodiments, wherein (i) the cancer in the subject has relapsed during therapy with a first Trk inhibitor; and/or (ii) the cancer in the subject is not responding to therapy with the first Trk inhibitor; and/or (iii) the subject is intolerant to the first Trk inhibitor, a Trk resistance mutation, e.g., any of the mutations described in Tables 11-13, is not detected.

### Methods of Selecting a Subject Having a Cancer for Participation in a Clinical Study

Also provided herein are methods of selecting a subject having a cancer for participation in a clinical study that includes administration of treatment for a cancer that include (a) determining whether (i) the cancer in the subject has relapsed during therapy with a first Trk inhibitor; and/or (ii) the cancer in the subject is not responding to therapy with a first Trk inhibitor; and/or (iii) the subject is intolerant to a first Trk inhibitor; and (b) selecting a subject in which (i) the cancer in the subject has relapsed during therapy with a first Trk inhibitor; and/or (ii) the cancer in the subject is not responding to therapy with a first Trk inhibitor; and/or (iii) the subject is intolerant to a first Trk inhibitor, for participation in a clinical study that includes administration of a therapy for a cancer.

Also provided herein are methods of selecting a subject having a cancer for participation in a clinical study that includes administration of a second Trk inhibitor that include (a) determining whether (i) the cancer in the subject has relapsed during therapy with a first Trk inhibitor; and/or (ii) the cancer in the subject is not responding to therapy with a first Trk inhibitor; and/or (iii) the subject is intolerant to a first Trk inhibitor; and (b) selecting a subject in which (i) the cancer in the subject has relapsed during therapy with a first Trk inhibitor; and/or (ii) the cancer in the subject is not responding to therapy with a first Trk inhibitor; and/or (iii) the subject is intolerant to a first Trk inhibitor, for participation in a clinical study that includes administration of a second Trk inhibitor. In some embodiments, the second Trk inhibitor is Compound 1, or a pharmaceutically acceptable salt, amorphous, or polymorph form thereof. In some embodiments the second Trk inhibitor is a pharmaceutical composition comprising a compounding agent as disclosed herein and Compound 1 or a solid form thereof, crystalline form thereof, or solvate or hydrate thereof, or a salt of Compound 1 or solid form thereof, crystalline form thereof, or solvate or hydrate thereof, as described herein.

The cancer can be any of the exemplary cancers described herein. In some embodiments, the subject has previously been identified or diagnosed as having a cancer. In some examples, the subject has previously been administered a therapy for cancer, and the therapy for cancer has been unsuccessful (e.g., high toxicity in the subject or no positive response to the previously administered therapy for cancer).

In some embodiments, the cancer is a Trk-associated cancer. In some embodiments, the Trk-associated cancer exhibits at least one of a NTRK1, NTRK2, and/or NTRK3 fusion. In some embodiments, the at least one NTRK1, NTRK2, and/or NTRK3 fusion results in the expression of one or more of a TrkA fusion protein, and/or a TrkB fusion protein, and/or a TrkC fusion protein, e.g., one or more of the Trk fusions described in Tables 2, 5, and 8. In some embodiments, the Trk-associated cancer exhibits one or mutations point mutations/insertions/deletions in one or more of NTRK1, NTRK2, or NTRK3. Non-limiting examples of Trk kinase point mutations/insertions/deletions are described in Tables 3, 4, 6, 7, 9, and 10. In some embodiments, the Trk-associated cancer does not exhibit a Trk resistance mutation, e.g., any of the mutations described in Tables 11-13.

In some embodiments, the first Trk inhibitor is selected from the group consisting of: entrectinib (N-[5-(3,5-difluoro-benzyl)-1H-indazol-3-yl]-4-(4-methylpiperazin-1-yl)-2-(tetrahydro-pyran-4-ylamino)-benzamide), (S)—N-(5-((R)-2-(2,5-difluorophenyl)pyrrolidin-1-yl)pyrazolo[1,5-a]pyrimidin-3-yl)-3-hydroxypyrrolidine-1-carboxamide sulfate, cabozantinib ((N-(4-((6,7-Dimethoxyquinolin-4-yl)oxy)phenyl)-N′-(4-fluorophenyl)cyclopropane-1,1-dicarboxamide)), dovitinib (4-amino-5-fluoro-3-[6-(4-methylpiperazin-1-yl)-1H-benzimidazol-2-yl]quinolin-2(1H)-one mono 2-hydroxypropanoate hydrate), belizatinib (4-fluoro-N-(6-((4-(2-hydroxypropan-2-yl)piperidin-1-yl)methyl)-1-((1s,4s)-4-(isopropylcarbamoyl)cyclohexyl)-1H-benzo[d]imidazol-2-yl)benzamide), sitravatinib (N-(3-fluoro-4-((2-(5-(((2-methoxyethyl)amino)methyl)pyridin-2-yl)thieno[3,2-b]pyridin-7-yl)oxy)phenyl)-N-(4-fluorophenyl)cyclopropane-1,1-dicarboxamide), PLX7486, altiratinib (N-(4-((2-(cyclopropanecarboxamido)pyridin-4-yl)oxy)-2,5-difluorophenyl)-N-(4-fluorophenyl)cyclopropane-1,1-dicarboxamide), AZD7451 ((S)—N-(1-(5-fluoropyrimidin-2-yl)ethyl)-3-(5-isopropoxy-1H-pyrazol-3-yl)-3H-imidazo[4,5-b]pyridin-5-amine), (6R,15R)-9-fluoro-15-methyl-2,11,16,20,21,24-hexaazapentacyclo[16.5.2.02,6.07,12.021,25]pentacosa-1(24),7,9, 11,18(25),19,22-heptaen-17-one, a (R)-2-phenylpyrrolidine substituted imadazopyridazine, AZD6918, GNF-4256, GTx-186, GNF-5837, AZ623, AG-879, CT327, AR-772, AR-523, AR-786, AR-256, AR-618, AZ-23, CEP-701, CEP-751, PHA-739358, dovitinib, Go 6976, GW441756, MGCD516, ONO-5390556, PHA-848125AC, Regorafenib, Sorafenib, Sunitinib, TSR-011, VM-902A, K252a, a 4-aminopyrazolylpyrimidine, a substituted pyrazolo[1,5-a] pyrimidine compound, BMS-754807, ONO-7579, F17752, ANA-12, ONO-4474, GZ389988, or TPX-0005 ((7S,13R)-11-fluoro-7,13-dimethyl-6,7,13,14-tetrahydro-1,15-ethenopyrazolo[4,3-f][1,4,8,10]benzoxatriazacyclotridecin-4(5H)-one; repotrectinib).

In some embodiments, relapse is one or more of detecting an increase in the number of cancer cells in the subject, an increase in the size of one or more tumors in the subject, an increase in tumor burden, an increase in the rate or extent of metastasis, worsening symptoms, in whole or in part, associated with the cancer, an increase in the extent of disease, and an acceleration of disease progression after a period of improvement. In some embodiments, relapse is progression of the cancer after a period of improvement. In some embodiments, a period of improvement is one or more of a decrease in the number of cancer cells in the subject, a decrease in the size of one or more tumors in the subject, a decrease in tumor burden, a decrease in the rate or extent of metastasis, improving symptoms, in whole or in part, associated with the cancer, a decrease in the extent of disease, and a slowing of disease progression.

In some embodiments, the tumor burden is assessed by RECIST version 1.1.

In some embodiments, the cancer is glioma and the progression of the glioma is assessed by RANO.

In some embodiments, circulating tumor DNA is used to monitor the responsiveness to therapy with a first Trk inhibitor. In some embodiments, an increase in the level ctDNA compared to the baseline is indicative of progression of the cancer, as described herein. In some embodiments, an increase in the level of ctDNA compared to the level during a period of improvement is indicative of relapse of the cancer.

In some embodiments, a cancer that is not responding to therapy with a first Trk inhibitor is a cancer that is progressing. In some embodiments, progression of a cancer is one or more of an increase in the number of cancer cells in the subject, an increase in the size of one or more tumors in the subject, an increase in tumor burden, an increase in the rate or extent of metastasis, worsening symptoms, in whole or in part, associated with the cancer, an increase in the extent of disease, and an acceleration of disease progression.

In some embodiments, the tumor burden is assessed using RECIST version 1.1.

In some embodiments, the cancer is glioma and the progression of the glioma is assessed by RANO.

In some embodiments, circulating tumor DNA is used to monitor the responsiveness to therapy with a first Trk inhibitor. In some embodiments, an increase in the level ctDNA compared to the baseline is indicative of progression of the cancer, as described herein. In some embodiments, an increase in the level of ctDNA compared to the level during a period of improvement is indicative of relapse of the cancer.

In some embodiments, the subject that is intolerant to a first Trk inhibitor has had one or more of a severe, disabling, or life-threatening adverse event during therapy with the first Trk inhibitor, an unplanned hospitalization during therapy with the first Trk inhibitor, discontinuation of therapy with the first Trk inhibitor, dose reduction of the first Trk inhibitor, functional decline attributed to therapy with the first Trk inhibitor, and a decrease in performance status.

In some embodiments, the performance status is assessed using the Eastern Cooperative Oncology Group (ECOG) Scale of Performance Status.

In some embodiments, the performance status is assessed using the Karnofsky Performance Status.

In some embodiments, the performance status is assess by the Lansky Performance Score. In some embodiments, the subject is a pediatric patient. In some embodiments, wherein (i) the cancer in the subject has relapsed during therapy with a first Trk inhibitor; and/or (ii) the cancer in the subject is not responding to therapy with the first Trk inhibitor; and/or (iii) the subject is intolerant to the first Trk inhibitor, a Trk resistance mutation, e.g., any of the mutations described in Tables 11-13, is not detected.

Also provided herein are methods of treating a subject having a cancer (e.g., any of the cancers described herein) that include identifying a subject having a cancer cell that has at least one point mutation in a NTRK gene that results in the expression of a Trk protein including a mutation at one or more of the amino acid positions shown in Tables 4, 4a, 7, or 10 and administering to the identified subject a pharmaceutical composition comprising a compounding agent as disclosed herein and Compound 1 or a solid form thereof, crystalline form thereof, or solvate or hydrate thereof, or a salt of Compound 1 or solid form thereof, crystalline form thereof, or solvate or hydrate thereof, as described herein.

Also provided herein are methods of treating a subject that include administering a therapeutically effective amount of a pharmaceutical composition comprising a compounding agent as disclosed herein and Compound 1 or a solid form thereof, crystalline form thereof, or solvate or hydrate thereof, or a salt of Compound 1 or solid form thereof, crystalline form thereof, or solvate or hydrate thereof, as described herein, to a subject having a clinical record that indicates that the subject has a cancer cell that has at least one point mutation in a NTRK gene that results in the expression of a Trk protein including a mutation at one or more amino acid positions (e.g., a mutation at one or more of the amino acid positions shown in Tables 4, 4a, 7, or 10).

Also provided herein are methods of treating a subject having a cancer (e.g., any of the cancers described herein or known in the art) that include: identifying a subject having a cancer cell that has at least one point mutation in a NTRK gene that results in the expression of a Trk protein including a mutation at one or more amino acid positions (e.g., a mutation at one or more of the amino acid positions shown in Tables 4, 4a, 7, or 10); and administering to the identified subject a pharmaceutical composition comprising a compounding agent as disclosed herein and Compound 1 or a solid form thereof, crystalline form thereof, or solvate or hydrate thereof, or a salt of Compound 1 or solid form thereof, crystalline form thereof, or solvate or hydrate thereof, as described herein.

Also provided herein are methods of treating a subject having a cancer (e.g., any of the cancers described herein or known in the art) that include: identifying a subject having a cancer cell that has at least one point mutation in a NTRK gene that results in the expression of a Trk protein including a mutation at one or more amino acid positions (e.g., a mutation at one or more of the amino acid positions shown in Tables 4, 4a, 7, or 10); and administering to the identified subject a pharmaceutical composition comprising a compounding agent as disclosed herein and Compound 1 or a solid form thereof, crystalline form thereof, or solvate or hydrate thereof, or a salt of Compound 1 or solid form thereof, crystalline form thereof, or solvate or hydrate thereof, as described herein, and another anticancer agent (e.g., any one or more of the anticancer agents described herein) or anticancer therapy (e.g., any one or more of the anticancer therapies provided herein.

Also provided herein are methods of treating a subject that include administering a therapeutically effective amount of a pharmaceutical composition comprising a compounding agent as disclosed herein and Compound 1 or a solid form thereof, crystalline form thereof, or solvate or hydrate thereof, or a salt of Compound 1 or solid form thereof, crystalline form thereof, or solvate or hydrate thereof, as described herein, to a subject having a clinical record that indicates that the subject has a cancer cell that has at least one point mutation in a NTRK gene that results in the expression of a Trk protein including a mutation at one or more amino acid positions (e.g., a mutation at one or more of the amino acid positions shown in Tables 4, 4a, 7, or 10).

Also provided herein are methods of treating a subject that include administering a therapeutically effective amount of a pharmaceutical composition comprising a compounding agent as disclosed herein and Compound 1 or a solid form thereof, crystalline form thereof, or solvate or hydrate thereof, or a salt of Compound 1 or solid form thereof, crystalline form thereof, or solvate or hydrate thereof, as described herein, and another anticancer agent (e.g., any one or more of the anticancer agents described herein) or anticancer therapies (e.g., any one or more of the anticancer therapies described herein), to a subject having a clinical record that indicates that the subject has a cancer cell that has at least one point mutation in a NTRK gene that results in the expression of a Trk protein including a mutation at one or more amino acid positions (e.g., a mutation at one or more of the amino acid positions shown in Tables 4, 4a, 7, or 10).

In some embodiments, the cancer is a Trk inhibitor-resistant cancer. In some embodiments, a Trk inhibitor-resistant cancer can be resistant to therapy with (S)—N-(5-((R)-2-(2,5-difluorophenyl)pyrrolidin-1-yl)pyrazolo[1,5-a]pyrimidin-3-yl)-3-hydroxypyrrolidine-1-carboxamide sulfate (or a polymorph thereof), but the Trk inhibitor-resistant cancer is still sensitive to a treatment including (6R,15R)-9-fluoro-15-methyl-2,11,16,20,21,24-hexaazapentacyclo[16.5.2.0^{2,6}.0^{7,12}.0^{21,25}]pentacosa-1(24),7,9, 11,18(25),19,22-heptaen-17-one or a pharmaceutically acceptable salt thereof. In some embodiments, a Trk inhibitor-resistant cancer can be resistant to therapy with entrectinib, but the Trk inhibitor-resistant cancer is still sensitive to a treatment including (6R,15R)-9-fluoro-15-methyl-2,11,16,20,21,24-hexaazapentacyclo[16.5.2.0^{2,6}.0^{7,12}.0^{21,25}]pentacosa-1(24),7,9,11,18(25),19,22-heptaen-17-one or pharmaceutically acceptable salt thereof.

A Trk inhibitor-resistant cancer cell can have, e.g., an increased rate of growth in the presence of at least one Trk inhibitor (e.g., any of the Trk inhibitors described herein or known in the art) as compared to the rate of growth of a control cell from a control subject having the same type of cancer and not having one or more of the point mutations in a NTRK1 gene described herein or one or more of the point mutations in a NTRK2 gene described herein or a point mutation in a NTRK3 gene described herein, when it is contacted with the at least one Trk inhibitor (e.g., a first Trk inhibitor). One of skill in the art will appreciate that the Trk inhibitor-resistant cancer cell and the control cell are contacted with the same concentration of the at least one Trk inhibitor.

A Trk inhibitor-resistant cancer in a subject can have, e.g., an increased rate of growth of a solid tumor when the subject is treated with at least one Trk inhibitor (e.g., a first Trk inhibitor) as compared to the rate of growth of a control solid tumor in a control subject treated with the at least one Trk inhibitor and having the same type of cancer and not having one or more of the point mutations in a NTRK1 gene described herein or one or more of the point mutations in a NTRK2 gene described herein or a point mutation in a NTRK3 gene described herein). One of skill in the art will appreciate that the subject and the control subject are administered the same concentration of the at least one Trk inhibitor.

Trk inhibitor-resistant cancer in a subject can have, e.g., a decreased rate of apoptosis in a solid tumor when the subject is treated with at least one Trk inhibitor (e.g., any of the Trk inhibitors described herein or known in the art) as compared to the rate of apoptosis of a control solid tumor in a control subject treated with the at least one Trk inhibitor and having the same type of cancer and not having one or more of the point mutations in a NTRK1 gene described herein or one or more of the point mutations in a NTRK2 gene described herein or one or more point mutations in a NTRK3 gene described herein). One of skill in the art will appreciate that the subject and the control subject are administered the same concentration of the at least one Trk inhibitor.

### Other Methods of Treatment

Certain compounds which are inhibitors of TrkA and/or TrkB may be useful in the treatment of multiple types of pain including inflammatory pain, neuropathic pain, and pain associated with cancer, surgery, and bone fracture.

In one embodiment, a pharmaceutical composition comprising a compounding agent as disclosed herein and Compound 1 or its solid forms, crystalline forms, solvates or hydrates, or the salts of Compound 1 or their solid forms, crystalline forms, solvates and hydrates as described herein, is useful for treating pain, including chronic and acute pain, in a mammal.

Pharmaceutical compositions comprising a compounding agent as disclosed herein and Compound 1 or its solid forms, crystalline forms, solvates or hydrates, or the salts of Compound 1 or their solid forms, crystalline forms, solvates and hydrates as described herein, are also useful for treating inflammation in a mammal.

Pharmaceutical compositions comprising a compounding agent as disclosed herein and Compound 1 or its solid forms, crystalline forms, solvates or hydrates, or the salts of Compound 1 or their solid forms, crystalline forms, solvates and hydrates as described herein, are also useful for treating certain infectious diseases in a mammal, such as Trypanosoma cruzi infection.

Pharmaceutical compositions comprising a compounding agent as disclosed herein and Compound 1 or its solid forms, crystalline forms, solvates or hydrates, or the salts of Compound 1 or their solid forms, crystalline forms, solvates and hydrates as described herein, may also be used to treat neurodegenerative diseases in a mammal. Examples of neurodegenerative disease include demyelination and dysmyelination. Additional examples of neurodegenerative diseases include multiple sclerosis, Parkinson's disease and Alzheimer's disease.

In addition, pharmaceutical compositions comprising a compounding agent as disclosed herein and Compound 1 or its solid forms, crystalline forms, solvates or hydrates, or the salts of Compound 1 or their solid forms, crystalline forms, solvates and hydrates as described herein, may also be used to treat interstitial cystitis (IC), painful bladder syndrome (PBS), urinary incontinence, asthma, anorexia, atopic dermatitis, and psoriasis in a subject (e.g., a mammal such as a human).

Accordingly, another embodiment of the present application provides a method of treating or preventing pain in a subject (e.g., mammal), comprising administering to said mammal a pharmaceutical composition comprising a compounding agent as disclosed herein and Compound 1 or a solid form thereof, crystalline form thereof, or solvate or hydrate thereof, or a salt of Compound 1 or solid form thereof, crystalline form thereof, or solvate or hydrate thereof, as described herein, in an amount effective to treat or prevent said pain. In one embodiment, the pain is chronic pain. In one embodiment, the pain is acute pain. In one embodiment, the pain is inflammatory pain. In one embodiment, the pain is neuropathic pain. In one embodiment, the pain is pain associated with cancer. In one embodiment, the pain is pain associated with surgery. In one embodiment, the pain is pain associated with bone fracture. In one embodiment, the method comprises a method of treating said pain in a mammal. In one embodiment, the method comprises a method of preventing said pain in a mammal.

Another embodiment of the present disclosure provides a method of treating or preventing inflammation in a subject (e.g., mammal), comprising administering to said mammal a pharmaceutical composition comprising a compounding agent as disclosed herein and Compound 1 or a solid form thereof, crystalline form thereof, or solvate or hydrate thereof, or a salt of Compound 1 or solid form thereof, crystalline form thereof, or solvate or hydrate thereof, as described herein, in an amount effective to treat or prevent the inflammation. In one embodiment, the method comprises treating the inflammation in a subject. In one embodiment, the method comprises preventing the inflammation in a subject.

Another embodiment of the present application provides a method of treating or preventing a neurodegenerative disease in a mammal, comprising administering to said mammal a pharmaceutical composition comprising a compounding agent as disclosed herein and Compound 1 or a solid form thereof, crystalline form thereof, or solvate or hydrate thereof, or a salt of Compound 1 or solid form thereof, crystalline form thereof, or solvate or hydrate thereof, as described herein, in an amount effective to treat or prevent said neurodegenerative disease. In one embodiment, the neurodegenerative disease is demyelination. In one embodiment, the neurodegenerative disease is dysmyelination. In one embodiment, the neurodegenerative disease is multiple sclerosis. In one embodiment, the neurodegenerative disease is Parkinson's disease. In one embodiment, the neurodegenerative disease is Alzheimer's disease.

Another embodiment of the present disclosure provides a method of treating or preventing an infectious disease in a subject, comprising administering to the subject a pharmaceutical composition comprising a compounding agent as disclosed herein and Compound 1 or a solid form thereof, crystalline form thereof, or solvate or hydrate thereof, or a salt of Compound 1 or solid form thereof, crystalline form thereof, or solvate or hydrate thereof, as described herein, in an amount effective to treat or prevent said infectious disease. In one embodiment, the infectious disease is Trypanosoma cruzi infection. In one embodiment, the method comprises treating the neurodegenerative disease in a subject. In one embodiment, the method comprises preventing the neurodegenerative disease in a subject.

### Dosages

The pharmaceutical compositions herein can contain an amount of the active ingredient necessary to deliver an effective dose as described above.

The pharmaceutical compositions herein contain, per unit dosage unit, e.g., suspension, solution, and the like, of from about 0.1-1000 mg or any range therein, and may be given at a dosage of from about 0.01-300 mg/kg/day, or any range therein, preferably from about 0.5-50 mg/kg/day, or any range therein. In some embodiments, the pharmaceutical compositions provided herein contain, per unit dosage unit, about 25 mg to about 500 mg of Compound 1 or any one of crystalline forms, solid forms, solvates, hydrates or salts described herein (for example, about 25 mg to about 400 mg, about 25 mg to about 300 mg, about 25 mg to about 250 mg, about 25 mg to about 200 mg, about 25 mg to about 150 mg, about 25 mg to about 100 mg, about 25 mg to about 75 mg, about 25 mg to a about 50 mg, about 50 mg to about 500 mg, about 100 mg to about 500 mg, about 150 mg to about 500 mg, about 200 mg to about 500 mg, about 250 mg to about 500 mg, about 300 mg to about 500 mg, about 400 mg to about 500 mg, about 50 to about 200 mg, about 100 to about 250 mg, about 50 to about 150 mg). In some embodiments, the pharmaceutical compositions provided herein contain, per unit dosage unit, about 25 mg, about 50 mg, about 100 mg, about 150 mg, about 200 mg, about 250 mg, about 300 mg, about 400 mg, or about 500 mg of Compound 1 or any one of crystalline forms, solid forms, solvates, hydrates or salts described herein. The dosages, however, can be varied depending upon the requirement of the patient, the severity of the condition being treated, and/or (if applicable) the crystalline form, solid form, solvate, hydrate or salt being employed. In some embodiments, the dosages are administered once daily (QD) or twice daily (BID).

Preferably, these compositions are in unit dosage forms, such as sterile solutions or suspensions for oral administration. Thus, in some embodiments, provided herein is a pharmaceutical composition comprising Compound 1 and a compounding agent as disclosed herein, wherein the pharmaceutical composition is a suspension. In some embodiments, provided herein is a pharmaceutical composition comprising Compound 1 and a compounding agent as disclosed herein, wherein the pharmaceutical composition is a solution.

The liquid forms in which the compositions provided herein can be incorporated for administration orally or by injection include, for example, aqueous solutions, cyclodextrins, suitably flavored syrups, aqueous or oil suspensions, and flavored emulsions with edible oils, such as cottonseed oil, sesame oil, coconut oil, or peanut oil, as well as elixirs and similar pharmaceutical vehicles. Suitable dispersing or suspending agents for aqueous suspensions include synthetic and natural gums, such as tragacanth, acacia, alginate, dextran, sodium carboxymethylcellulose, methylcellulose, polyvinyl-pyrrolidone, and gelatin. For parenteral administration, sterile suspensions and solutions are desired. Isotonic preparations which generally contain suitable preservatives are employed when intravenous administration is desired.

Pharmaceutical compositions comprising a compounding agent as disclosed herein and Compound 1 or a solid form thereof, crystalline form thereof, or solvate or hydrate thereof, or a salt of Compound 1 or solid form thereof, crystalline form thereof, or solvate or hydrate thereof, as described herein can be administered according to dosage regimens established in the art whenever treatment of, e.g., cancer, pain, inflammation, neurodegenerative disease or Trypanosoma cruzi infection is required. For example, in some embodiments provided herein is a method of treating a disease or disorder selected from pain, cancer, inflammation, neurodegenerative disease and Trypanosoma cruzi infection in a subject, the method comprising administering to the subject in need thereof a therapeutically effective amount of the crystalline form of Compound 1. For example, in some embodiments provided herein is a method of treating a disease or disorder selected from pain, cancer, inflammation, neurodegenerative disease and Trypanosoma cruzi infection in a subject, the method comprising administering to the subject in need thereof a therapeutically effective amount of Compound 1 besylate. For example, in some embodiments provided herein is a method of treating a disease or disorder selected from pain, cancer, inflammation, neurodegenerative disease and Trypanosoma cruzi infection in a subject, the method comprising administering to the subject in need thereof a therapeutically effective amount of Compound 1 citrate.

The daily dosage of Compound 1 or any one of crystalline forms, solid forms, solvates, hydrates or salts in a pharmaceutical composition comprising a compounding agent as disclosed herein can be varied over a wide range from 1.0 to 10,000 mg per adult human per day, or higher, or any range therein. For oral administration, the compositions are preferably provided in an amount of 0.01, 0.05, 0.1, 0.5, 1.0, 2.5, 5.0, 10.0, 15.0, 25.0, 50.0, 100, 150, 200, 250 or 500 milligrams of the active ingredient for the symptomatic adjustment of the dosage to the patient to be treated. An effective amount of the drug is ordinarily supplied at a dosage level of from about 0.1 mg/kg to about 1000 mg/kg of body weight per day, or any range therein. Preferably, the range is from about 0.5 to about 500 mg/kg of body weight per day, or any range therein. More preferably, from about 1.0 to about 250 mg/kg of body weight per day, or any range therein. More preferably, from about 0.1 to about 100 mg/kg of body weight per day, or any range therein. In an example, the range can be from about 0.1 to about 50.0 mg/kg of body weight per day, or any amount or range therein. In another example, the range can be from about 0.1 to about 15.0 mg/kg of body weight per day, or any range therein. In yet another example, the range can be from about 0.5 to about 7.5 mg/kg of body weight per day, or any amount to range therein. Any one of crystalline forms, solid forms, solvates, hydrates or salts described herein can be administered on a regimen of 1 to 4 times per day or in a single daily dose. Optimal dosages to be administered can be readily determined by those skilled in the art, and can vary with the mode of administration, the strength of the preparation, the mode of administration, and the advancement of the disease condition. In addition, factors associated with the particular patient being treated, including patient age, weight, diet, and time of administration, can result in the need to adjust dosages.

### EXAMPLES

### Materials and Methods

Compound 1 Free Base

The compound 1 free base was obtained using methods and procedures similar to those described in U.S. provisional application No. 62/524,801, each of which is incorporated by reference herein in its entirety. In accordance with U.S. provisional application No. 62/524,801, compound 1 was obtained as follows:

### Example A. Preparation of Compound 1

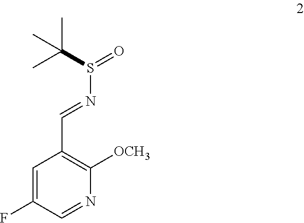 [SMILES: COc1ncc(F)cc1/C=N/[S@](=O)C(C)(C)C]

### (R,E)-N-((5-fluoro-2-methoxypyridin-3-yl) methylene)-2-methylpropane-2-sulfinamide (2)

A flask (equipped with a nitrogen inlet, overhead stirring, and thermocouple) was charged with DCM (3 L, 10 vol). The mixture was agitated, and the mixture was deoxygenated with subsurface nitrogen for 1 h. Next 5-fluoro-2-methoxynicotinaldehyde (1) (300 g, 1934 mmol) and (R)-2-methylpropane-2-sulfinamide (246 g, 2031 mmol) were charged. The Cs₂CO₃ (441 g, 1354 mmol) was charged in portions, with agitation, over several minutes. The reaction was agitated overnight at ambient temperature under nitrogen. The reaction was sampled and analyzed by HPLC for reaction completion. A 15 wt % solution of the citric acid (in water) was prepared (using 1.5 eq of citric acid based on the Cs₂CO₃ input). This solution was charged into the reactor with the reaction mixture, using an addition funnel. The charge was done in portions. The biphasic mixture was transferred to a separatory funnel, and the lower DCM layer was removed. The upper aqueous layer was removed and discarded. The DCM layer was transferred back into the separatory funnel, and washed with saturated brine (2 L). Again, the lower DCM layer was removed, and the upper aqueous layer was discarded. The DCM layer was concentrated under vacuum (rotovap) to give the desired product.

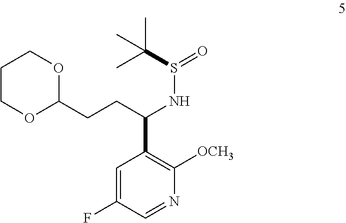

### (S)—N—((S)-3-(1,3-dioxan-2-yl)-1-(5-fluoro-2-methoxypyridin-3-yl)propyl)-2-methylpropane-2-sulfinamide (5)

A flask (equipped with a nitrogen inlet, overhead stirring, reflux condenser, thermocouple, and addition funnel) was charged with Mg turnings (565 g, 23.2 moles) followed by THF (24 L, 8 vol) under nitrogen. This mixture was agitated and warmed to ˜30° C. When the internal temperature was 29.9° C., DIBAL (31.2 mL, 0.004 eq.) was added. A separate flask was charged with 2-(2-bromoethyl)-1,3-dioxane (4531 g, 23.2 moles) and THF (15.9 L, 5.3 vol). The mixture was agitated at ambient temperature to dissolve. The reaction flask with the Mg/Dibal-H mixture was slowly charged with the 2-(2-bromoethyl)-1,3-dioxane (3)/THF solution via an addition funnel. The charge was made in portions over ˜5 h. The bromide solution was added so that the internal temperature did not rise above 50° C. The reaction mixture was then held for 45 minutes. After the 45-minute hold, the active Grignard mixture was cooled to −30 to −40° C. (dry ice/acetonitrile). A separate flask was charged with the (R,E)-N-((5-fluoro-2-methoxypyridin-3-yl)methylene)-2-methylpropane-2-sulfinamide (3000 g, 11.6 moles), followed by THF (5.1 L, 1.7 vol). Using an addition funnel, the starting material solution was portion-wise transferred at ambient temperature into the Grignard mixture over ˜2 h and the internal temp was kept at −37.3 to −28.9° C. The reaction mixture was agitated at low temperature and analyzed by HPLC for reaction completion. To quench the reaction, a 15 wt % solution of citric acid (˜11 vol) was charged into a round bottom flask and cooled with an ice bath to ˜10° C. The reaction mixture was transferred into the citric acid solution in portions. When the transfer was complete, the mixture was allowed to stir for ˜15 minutes. MTBE (9 L, 3 vol) was charged into the mixture and then the entire mixture was transferred to a separatory funnel. The reaction flask was rinsed with MTBE (3 L, 1 vol) and transferred to the separatory funnel. The biphasic mixture was agitated for 5 minutes and then the phases were allowed to settle. The layers were separated, and the bottom aqueous layer was back extracted with additional MTBE (16 L, ˜5 vol). After mixing and settling, the layers were separated. The MTBE layers were combined and washed with sat. brine (15 L, 5 vol). After mixing and settling, the aqueous layer was discarded. The MTBE layer was concentrated under vacuum. MTBE (6 L, 2 vol) was charged, and the product was dissolved with agitation at ambient temperature. To the MTBE solution, heptane (30 L, 10 vol) was charged over ˜1 h. The slurry was allowed to agitate at ambient temperature overnight, and then filtered through polypropylene filter cloth. The cake was rinsed with heptane (9 L, 3 vol), and the wet solid 5 was dried in trays under vacuum at ˜50° C. to constant weight.

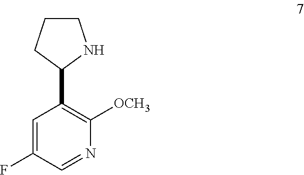

### (R)-5-fluoro-2-methoxy-3-(pyrrolidin-2-yl)pyridine (7)

A flask (equipped with mechanical stirring, N2 inlet, condenser and J-Kem) was charged with 5 (1993 g, 5322 mmol) 2,2,2-trifluoroacetic acid (7971 mL) and water (1918 mL). The reaction was sampled to monitor completion of the deprotection by HPLC. After the reaction was judged to be complete, the reaction was charged with triethylsilane (2550 mL, 16.0 moles) via addition funnel over ˜1 h. The reaction mixture was stirred at ambient temperature overnight and the solvent was removed under vacuum with heating to 45-50° C. The resulting product was added to a 100 L separatory funnel and was diluted with MTBE (15 L) and water (15 L). The layers were agitated and the separated layers were dropped into tared carboys (Aq 1 and MTBE 1). The MTBE layer was added back to the separatory funnel and was back extracted with 6000 mL 1 M HCl. After mixing, the separated layers were dropped into tared carboys (Aq2 and MTBE 2). The aqueous layers were combined in the separatory funnel. To the aqueous layer was added DCM (16 L). To the mixture was added 50 wt % NaOH (˜900 mL) to reach pH≥12. After mixing, the organic layer was dropped into a tared flask (DCM 1). The aqueous layer was extracted with DCM (16 L) and the organic layer was dropped into a tared flask. The aqueous layer was extracted a third time with DCM (8 L). The organic layer was dropped into a tared flask (DCM 3). The combined organic layers were transferred to the separatory funnel and washed with sat. brine (9 L). The layers were separated and the organic layers were dropped and then the solvent was removed under vacuum to isolated the product.

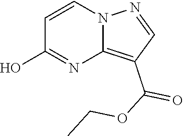

### Ethyl 5-hydroxypyrazolo[1,5-a]pyrimidine-3-carboxylate

To a reactor was charged K₃PO₄ (4104 g granular, 19.3 moles), ethyl 3-amino-1H-pyrazole-4-carboxylate (2000 g, 12.9 moles), and DMF (18.8 kg) and the mixture was agitated. After 20 min, (E)-ethyl 3-ethoxyacrylate, (2230 g, 15.5 moles) was added and the mixture was heated to 110-115° C. internal temperature (IT). After the reaction was judged to be complete based on consumption of starting material, heating was ceased. The mixture was allowed to stir and cool overnight. Aqueous hydrochloric acid (3 M, 13 L) was added over ˜2 h. DI water (6 L) was added and the mixture was allowed to stir overnight. The mixture was filtered through polypropylene filter cloth (PPFC) and the residue was washed with water (3×5 vol, 3×10 L). The solid was placed in trays and oven dried under vacuum at 55° C. for 3 days and then 45° C. for 4 days to constant weight of (2553 g 95.6%).

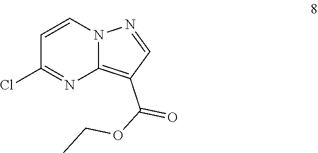

### Ethyl 5-chloropyrazolo[1,5-a]pyrimidine-3-carboxylate (8)

To a flask, under nitrogen, outfitted with mechanical stirring, J-Kem temperature probe, and condenser was added ethyl 5-hydroxypyrazolo[1,5-a]pyrimidine-3-carboxylate (2319 g, 11.2 moles), acetonitrile (9200 mL), and phosphoryl trichloride (1230 mL, 13.4 mmol). The reaction mixture was heated to ˜74° C. (IT) until it was judged complete by HPLC. The reaction was cooled to ˜30° C. While cooling, a separate flask was outfitted with mechanical stirring and a J-Kem temperature probe. Water (37 L) was added to this and the water was cooled to below 15° C. The reaction mixture was added portion-wise producing a mixture. The chlorination reactor was rinsed with 4:1 water/MeCN (2 L) and the rinse was added to the mixture. To the mixture was added MeCN (1 L). The transfer line was rinsed with 4:1 water/MeCN (2 L), and the rinse was added to the mixture. The mixture was cooled back to below 20° C. and a solution of tribasic phosphate (2312 g, 10.9 mol) in water (4.0 L) was added portion-wise at a rate to keep the IT below 25° C. The slurry was stirred at ambient temperature overnight. The slurry was filtered (PPFC) and rinsed with 4:1 water/MeCN (6 L). The cake was rinsed a second time with water (7.0 L). The solid was placed in trays and dried in a vacuum oven at 50° C. for 36-72 h to give 8.

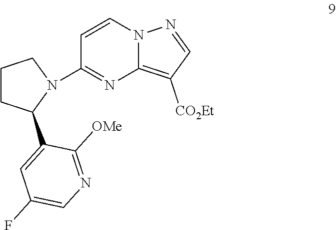 [SMILES: CCOC(=O)c1cnn2ccc(N3CCC[C@@H]3c3cc(F)cnc3OC)nc12]

### Ethyl (R)-5-(2-(5-fluoro-2-methoxypyridin-3-yl)pyrrolidin-1-yl)pyrazolo[1,5-a]pyrimidine-3-carboxylate (9)

Combined triethylamine (1187 mL, 8518 mmol), (R)-5-fluoro-2-methoxy-3-(pyrrolidin-2-yl) pyridine (7) (889 g, 4259 mmol) in EtOH (200 proof, 5 mL/g, 4.4 L) and then ethyl 5-chloropyrazolo[1,5-a]pyrimidine-3-carboxylate (8)(1001 g, 4259 mmol) were added. The reaction was stirred overnight at ambient temp (19 h). The next day, water (10 mL/g, 8.9 L) was added and after stirring at room temperature for 2 h it was filtered through polypropylene filter cloth (PPFC), 23° C. and washed with 2:1 water:EtOH (2×1.8 L) then heptane (1.8 L). The product was placed in trays and dried under vacuum (with N2 bleed) at 55° C. to give 9.

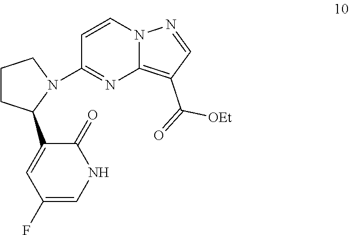 [SMILES: CCOC(=O)c1cnn2ccc(N3CCC[C@@H]3c3cc(F)c[nH]c3=O)nc12]

### Ethyl (R)-5-(2-(5-fluoro-2-oxo-1,2-dihydropyridin-3-yl)pyrrolidin-1-yl)pyrazolo[1,5-a]pyrimidine-3-carboxylate (10)

A solution of 4 M HCl in dioxane (1.0 L) was added to a flask containing (R)-ethyl 5-(2-(5-fluoro-2-methoxypyridin-3-yl) pyrrolidin-1-yl)pyrazolo[1,5-a]pyrimidine-3-carboxylate (9) (2500 g, 6486.9 mmol). The mixture was heated to 60° C. with an outlet at the top of the condenser (not under nitrogen). Once complete by HPLC, it was put under nitrogen and allowed to cool to room temperature with stirring overnight. The next day 20% K₃PO₄ (aq) (19 L, 7.5 mL/g—made by diluting 3800 g of K₃PO₄ to 19 kg total with water), was added. Once the temp was <35° C., EtOAc (12.5 L, 5 mL/g) was added and stirring continued for another 30 min. The mixture was pumped into a separatory vessel, and the aqueous layer dropped. The organic layer was concentrated under vacuum (rotovap) and the product was dried on vacuum pump at ambient temp to give 10.

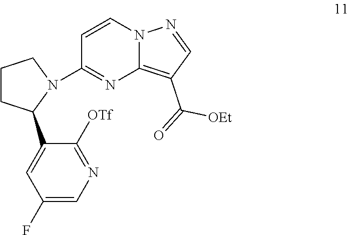 [SMILES: CCOC(=O)c1cnn2ccc(N3CCC[C@@H]3c3cc(F)cnc3OS(=O)(=O)C(F)(F)F)nc12]

### Ethyl (R)-5-(2-(5-fluoro-2-(((trifluoromethyl) sulfonyl)oxy)pyridin-3-yl) pyrrolidin-1-yl) pyrazolo[1,5-a]pyrimidine-3-carboxylate (11)

To a DCM solution of 10 was added triethylamine (1467 mL, 105.2 mol) and the mixture cooled to <5° C. Trifluoromethanesulfonic anhydride (1930 g, 684.0 mol) was added in portions maintaining temp <15° C. After 1 h reaction time sat. NaHCO₃(5 mL/g, 11 L) was added. The mixture was stirred for 1 h and was then transferred to a separatory vessel with DCM and the layers were separated. The organic layer was washed with NaHCO₃(11 L). The organic layer was concentrated to minimum volume and solvent-swapped to MeOH (target MeOH volume about 10 L). The MeOH solution was added to a flask containing 1:1 MeOH:water (20 L), the suspension was stirred at room temperature for 2 h, filtered, and washed with 1:2 water:MeOH (2×2 mL/g). The solid was oven dried under vacuum at 55° C. until constant weight, to give 11.

N-Phenyl-bis(trifluoromethanesulfonimide) may be used instead of Trifluoromethanesulfonic anhydride to provide 11.

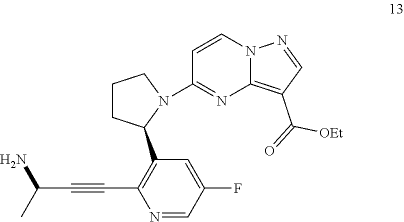 [SMILES: CCOC(=O)c1cnn2ccc(N3CCC[C@@H]3c3cc(F)cnc3C#C[C@@H](C)N)nc12]

### Ethyl 5-((R)-2-(2-((R)-3-aminobut-1-yn-1-yl)-5-fluoropyridin-3-yl)pyrrolidin-1-yl)pyrazolo[1,5-a]pyrimidine-3-carboxylate (13)

Toluene (16 L) was deoxygenated by N2 bubbling for ˜2 h. To a separate flask equipped with a heat source and reflux condenser were charged (R)-ethyl 5-(2-(5-fluoro-2-(((trifluoromethyl)sulfonyl)oxy)pyridin-3-yl)pyrrolidin-1-yl)pyrazolo[1,5-a]pyrimidine-3-carboxylate 11 (1440 g, 2860 mmol), copper(I) iodide (105 g, 551.3 mmol), Pd catalyst (398 g, 567.0 mmol), and the deoxygenated toluene. Diisopropylamine (810 ml, 5779 mmol) was added and the mixture was heated to 60° C. After ˜1 h, the reaction temp was 60° C. and commercially available (R)-tert-butyl but-3-yn-2-ylcarbamate (12) (728 g, 4302 mmol) was added in three portions. After ˜1 h, the mixture was cooled with an ice/water bath and then water (14 L) was added. When the reaction temp reaches ˜35° C., it was filtered (PPFC) and washed with water (2×3.5 L). The filtrate was transferred to a separatory vessel and the aqueous layer was washed with toluene (2×3.5 L). The aqueous layer was transferred to a separate flask and added DCM (14 L) was added. The mixture was cooled to <15° C., then sat. NH₄OH (2.4 L) was added. The solution was transferred to a separatory vessel and then washed with DCM (7 L). The DCM layers were allowed to stand at ambient temperature overnight and then they were combined and washed with brine (7 L). The organic layer was then concentrated, MeOH (5 L) was added and the mixture was concentrated to give 13.

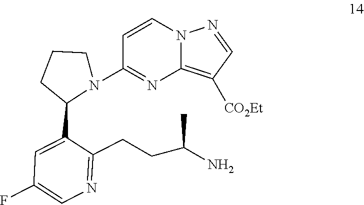 [SMILES: CCOC(=O)c1cnn2ccc(N3CCC[C@@H]3c3cc(F)cnc3CC[C@@H](C)N)nc12]

### Ethyl 5-((R)-2-(2-((R)-3-aminobutyl)-5-fluoropyridin-3-yl) pyrrolidin-1-yl) pyrazolo[1,5-a] pyrimidine-3-carboxylate (14)

Palladium on carbon (235 g, 104 mmol, 4.7 wt %), a 1285 g methanolic solution of ethyl 5-((R)-2-(2-((R)-3-aminobut-1-yn-1-yl)-5-fluoropyridin-3-yl)pyrrolidin-1-yl)pyrazolo[1,5-a]pyrimidine-3-carboxylate (13) (472 g, 1117 mmol) and MeOH (2.5 L˜4 L total volume) were charged into a 8 L Parr reactor. The mixture was stirred at 50 psi H2 until it was judged complete. The hydrogen atmosphere was replaced with nitrogen and the reaction mixture was allowed to stand overnight. The next day it was filtered through GF/F filter paper. The solution was concentrated to give 14.

Alternatively, 14 was also prepared as follows: Ethyl 5-((R)-2-(2-((R)-3-aminobut-1-yn-1-yl)-5-fluoropyridin-3-yl)pyrrolidin-1-yl)pyrazolo[1,5-a]pyrimidine-3-carboxylate (13) (˜102 g, 2414 mmol) and methanol (˜1.1 L total volume) were charged into a reactor. 10% Pd on Carbon (2 mol %) was added followed by Formic Acid (111 g, 10 eq). The reactor was inverted and held at 25° C. for 22.5 hours. The head space of the reactor was purged with nitrogen to remove any remaining hydrogen gas that may have been generated during the reaction and then additional 10% Pd on Carbon (2 mol %) was added. The batch was then held an additional three days. Before proceeding to the next step, the head space of the reactor was purged with nitrogen to remove any remaining hydrogen gas.

The solution was then filtered to separate the Pd on Carbon out of the solution. The reactor and filter were rinsed with methanol (3×240 mL). The filtrate was charged back to a clean reactor and concentrated to a minimum stir volume. 2-Methyltetrahydrofuran (610 mL) was then added into the reactor. The solution was concentrated again and additional 2-methyltetrahydrofuran was added to give about 1.2 L total. The 2-methyltetrahydrofuran mixture was extracted with 0.5 M hydrochloric acid solution (2×610 mL).

The aqueous layer was charged back to the reactor and methylene chloride (610 mL) was added. The solution was cooled to 0° C. and then neutralized with a 40% sodium hydroxide solution to pH 12-13. The batch temperature was adjusted to 5° C. The solution was mixed and then the phases were split. The lower, organic phase was removed and then additional methylene chloride (610 mL) was added. The solution was mixed and then the phases split again. The organic layers were added back into a clean reactor and solvent-swapped into isopropyl alcohol for use directly in the next step.

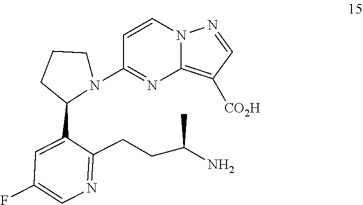 [SMILES: C[C@@H](N)CCc1ncc(F)cc1[C@H]1CCCN1c1ccn2ncc(C(=O)O)c2n1]

### 5-((R)-2-(2-((R)-3-aminobutyl)-5-fluoropyridin-3-yl) pyrrolidin-1-yl)pyrazolo[1,5-a]pyrimidine-3-carboxylic acid (15)

A methanol solution of 14 (861 g, 2019 mmol) was combined with IPA (4 L) and then concentrated to 2.2 kg under vacuum. The concentrate was transferred to a reactor (with reflux condenser) with further dilution in IPA (10 L). The mixture was heated to 75° C. (IT). Sodium hydroxide (184 mL, 2631 mmol) was added and the reaction continues until it was judged complete by HPLC. The heat was removed and the mixture was allowed to cool to ambient temp overnight. Concentrated hydrochloric acid (214 mL, 2632 mmol) was added. The mixture was concentrated under vacuum with external heating to 45° C. to ˜5 mL/g. Heptane (12 L) was added and the suspension was allowed to cool to ambient temp and then stirred for ˜1 h. The suspension was filtered (PPFC) and washed with 3:1 heptane:IPA (2×1600 mL). The wet cake was placed in trays and dried under vacuum at 55° C. to constant weight to give 15.

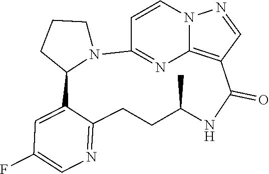 [SMILES: C[C@@H]1CCc2ncc(F)cc2[C@H]2CCCN2c2ccn3ncc(c3n2)C(=O)N1]

### (13E,14E,22R,6R)-35-fluoro-6-methyl-7-aza-1(5,3)-pyrazolo[1,5-a] pyrimidina-3(3,2)-pyridina-2(1,2)-pyrrolidinacyclooctaphan-8-one (Compound 1)

To a flask containing EDCI (157 g, 819 mmol) and DMAP (133 g, 1091 mmol) in DCM (50 mL/g, 125 mL) was added 5-((R)-2-(2-((R)-3-aminobutyl)-5-fluoropyridin-3-yl)pyrrolidin-1-yl)pyrazolo[1,5-a]pyrimidine-3-carboxylic acid (15) (302 g, 546 mmol) in 8 portions (37.8 g each). The portions were added ˜60 min apart. The reaction mixture was stirred overnight at ambient temperature. The mixture was transferred to a separatory funnel with minimal DCM and washed with sat. NaHCO₃(2×3 L), and 0.25 M citric acid (2×3 L, pH 5.5). The combined aqueous layers were washed with DCM (3 L, 10 mL/g) and then concentrated under vacuum (rotovap). The concentrate was dissolved in 3% MeOH in DCM and loaded onto a flash column (3 kg, SiO₂) and eluted with 3% MeOH in DCM (40 L total). The fractions containing the product were concentrated to give Compound 1 Combined lots of solid Compound 1 were triturated in IPAc (2.5 L, ca. 5 mL/g) at room temperature for 2 h. The mixture was heated to 40-45° C. for 10 minutes, then triturated at room temperature. The suspension was filtered and washed with IPAc (2×250 mL, ca. 2×0.5 mL/g) to give, after oven drying at 55° C., Compound 1.

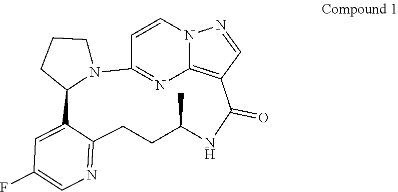 [SMILES: C[C@@H]1CCc2ncc(F)cc2[C@H]2CCCN2c2ccn3ncc(c3n2)C(=O)N1]

### (13E,14E,22R,6R)-35-fluoro-6-methyl-7-aza-1(5,3)-pyrazolo[1,5-a]pyrimidina-3(3,2)-pyridina-2(1,2)-pyrrolidinacyclooctaphan-8-one (Compound 1) (Alternative Preparation)

To a flask containing EDCI (1091 g, 5.7 mol, 1.7 eq) and DMAP (941 g, 7.71 mol, 2.3 eq) in DCM (38 L) were added the amino-acid 15 [5-((R)-2-(2-((R)-3-aminobutyl)-5-fluoropyridin-3-yl)pyrrolidin-1-yl)pyrazolo[1,5-a]pyrimidine-3-carboxylic acid] (1900 g, 3.35 mol) in 6 portions (added at least one hour apart), and the reaction was stirred at room temperature overnight. Once the reaction was complete it was transferred to a separatory funnel and washed with sat'd NaHCO₃ solution (2×19 L). The DCM layer was then washed with 0.25 M citric acid (38 L). The combined, citric acid aqueous layers were back-extracted with DCM (19 L), and the organic phases were added back to the 100 L round-bottomed flask. Charcoal (2.01 kg) and silica gel (2.01 kg) were added, and the suspension stirred at room temperature overnight. The next day, the suspension was filtered, and the charcoal cake was washed with DCM (3×19 L). The DCM filtrates were filtered a second time. The pale yellow solution was concentrated to minimum volume. Isopropyl acetate (28.5 L) was added and concentrated to 10 to 20 L. The suspension was heated overnight at 75° C., and the mixture was allowed to cool to room temperature. The solids were collected by filtration and washed with isopropyl acetate (2×1.9 L). The crude product was transferred to trays and dried in a vacuum oven 55° C. until constant mass was achieved.

To a flask was charged [(1³E,1⁴E,2²R,6R)-3⁵-fluoro-6-methyl-7-aza-1(5,3)-pyrazolo[1,5-a]pyrimidina-3 (3,2)-pyridina-2(1,2)-pyrrolidinacyclooctaphan-8-one]followed by 2-butanone (6.3 L). The slurry was agitated at 75° C. for 2 days and then the product was collected by filtration, and the product cake was washed with 2-butanone (2×950 mL g). The product was transferred to trays and dried in a vacuum oven at 55° C. until constant mass was achieved to provide Compound 1.

The average purity of Compound 1 was 98.8% as determined by HPLC-UV. The structure of Compound 1 was confirmed using ¹H NMR.

### General Methods for Preparation and Characterization of Compound 1 Salts

Approximately 20 mg Compound 1 was weighed into 2 mL vials. Acid counterions were weighed into separate vials and stock solutions prepared for the liquid counterions (1.05 eq.). Table 15 shows acid weights and volumes.

TABLE 15

| | | | | | Neat Addition | |
|---|---|---|---|---|---|---|
| | | | | | Amounts | |
| | | | | | By | By |
| Acid | | pKa | | | Weight | Volume |
| No. | Acid | 1 | 2 | 3 | (mg) | (μL) |
| 1 | Hydrochloric acid 37 wt. | −6.10 | | | 5.45 | 4.6 |
| | % (12M) | | | | | |
| 2 | Sulfuric acid | −3.00 | 1.92 | | 5.71 | 3.1 |
| 3 | 1-2-Ethane disulfonic | −2.10 | −1.50 | | 12.94 | |
| | acid | | | | | |
| 4 | p-Toluene sulfonic acid | −1.34 | | | 10.84 | |
| 5 | Methane sulfonic acid | −1.20 | | | 5.31 | 3.6 |
| 6 | Naphthalene-2-sulfonic | 0.17 | | | 14.14 | |
| | acid | | | | | |
| 7 | Benzene sulfonic acid | 0.70 | | | 8.92 | |
| 8 | Oxalic acid | 1.27 | 4.27 | | 5.08 | |
| 9 | 2-Hydroxy | 1.66 | | | 8.19 | |
| | ethanesulfonic acid | | | | | |
| 10 | L-Aspartic acid | 1.88 | 3.65 | | 7.36 | |
| 11 | Maleic acid | 1.92 | 6.23 | | 6.48 | |
| 12 | Phosphoric acid | 1.96 | 7.12 | 12.32 | 5.42 | |
| 13 | Ethane sulfonic acid | −2.05 | | | 6.41 | 4.7 |
| 14 | L-Glutamic acid | 2.19 | 4.25 | | 8.13 | |
| 15 | L-Tartaric acid | 3.02 | 4.36 | | 8.34 | |
| 16 | Fumaric acid | 3.03 | 4.38 | | 6.48 | |
| 17 | Citric acid | 3.13 | 4.76 | 6.40 | 10.67 | |
| 18 | D-Glucuronic acid | 3.18 | | | 10.73 | |
| 19 | L-Malic acid | 3.46 | 5.10 | | 7.49 | |
| 20 | Hippuric acid | 3.55 | | | 10.1 | |
| 21 | D-Gluconic acid (50% | 3.76 | | | 21.68 | 17.6 |
| | in water) | | | | | |
| 22 | DL-Lactic acid (85% | 3.86 | | | 5.86 | 4.8 |
| | aq. solution) | | | | | |
| 23 | L-Ascorbic acid | 4.17 | 11.57 | | 9.73 | |
| 24 | Benzoic acid | 4.19 | | | 6.82 | |
| 25 | Succinic acid | 4.21 | 5.64 | | 6.59 | |

Preparation of samples of salts of these acids and the Compound 1 in selective solvents (acetone, ethanol, methanol, 2-propanol, TBME and THF) is described in the Examples. In the Examples 8-32, solids observed post-temperature cycling were collected and analyzed by XRPD. Samples in which solid was not observed had anti-solvent additions made to saturated solutions and the resultant solids were analyzed by XRPD.

Anti-Solvent Additions

Approximately 1 mL of anti-solvent (heptane or TBME depending on miscibility) was added dropwise to saturated salt solutions of Compound 1 free base. Any resulting solid was analyzed by XRPD.

Salt Stability Studies

Recovered salts were placed in an oven at 40° C./75% RH for 1 week, and the resultant materials were analyzed by XRPD to determine any changes to form or crystallinity.

Thermodynamic Solubility

Thermodynamic solubility studies were carried out as follows: 10 mg of prepared salts were suspended in pH 1, 4.5, 6.8 and un-buffered water (300 μL). The pH of the slurries was measured and adjusted accordingly using either 0.2M HCl solution or 0.2M sodium hydroxide solution. The slurries were agitated for 24 hours at ambient temperature using an incubator shaker. The resulting slurries were filtered, any solids recovered were analyzed by XRPD and filtrate pH measured and submitted for UPLC analysis. pH 1.0 Buffer: 67 mL 0.1M hydrochloric acid solution was added to 12.5 mL 0.2M potassium chloride solution and diluted to 100 mL using de-ionized water and adjusted accordingly. pH 4.5 Buffer: 7.0 mL 0.2M sodium hydroxide solution was added to 25 mL 0.2 potassium hydrogen phthalate solution and diluted to 100 mL using de-ionized water and adjusted accordingly. pH 6.8 Buffer: 11.2 mL 0.2M sodium hydroxide solution was added to 25 mL 0.2M potassium phosphate mono-basic and diluted to 100 mL using de-ionized water and adjusted accordingly.

Salt Disproportionation Studies

Salt disproportionation studies were carried out as follows: 20 mg of prepared salts were weighed into a vial and 0.5 mL of deionized water was added. The samples were then agitated for 24 h at ambient temperature. The pH of the samples was taken pre- and post-agitation. Any solids recovered were submitted for XRPD analysis to determine any changes to form.

Hydration Screen

Hydration screen was carried out as follows: 10 mg of prepared salts were suspended in several acetone/water mixtures of various water activities (low: aw=0.281, medium: aw=0.776 and high: aw=0.919) and agitated at ambient temperatures for 24 hr. Any recovered solids were submitted for XRPD analysis to determine any changes to form.

### Analytical Methods

X-Ray Powder Diffraction (XRPD)

XRPD analysis was carried out on a Panalytical X'pert pro, scanning the samples between 3 and 35° 2θ. The material was gently ground and loaded onto a multi-well plate with Kapton or mylar polymer film to support the sample. The multi well plate was then loaded into a Panalytical diffractometer running in transmission mode, using Cu K radiation, and analyzed. The experimental conditions are shown in Table 16.

TABLE 16

| Raw Data Origin: | XRD measurement |
|---|---|
| Scan Axis: | Gonio |
| Start Position [°2θ]: | 3.0066 |
| End Position [°2θ]: | 34.9866 |
| Step Size [°2θ]: | 0.0130 |
| Scan Step Time [s]: | 18.8700 |
| Scan Type: | Continuous |
| PSD Mode: | Scanning |
| PSD Length [°2θ]: | 3.35 |
| Offset [°2θ]: | 0.0000 |
| Divergence Slit Type: | Fixed |
| Divergence Slit Size [°]: | 1.0000 |
| Measurement Temperature | 25.00 |
| Anode Material: | Cu |
| K-Alpha1 [Å]: | 1.54060 |
| K-Alpha2 [Å]: | 1.54443 |
| K-Beta [Å]: | 1.39225 |
| K-A2/K-A1 Ratio: | 0.50000 |
| Generator Settings: | 40 mA, 40 kV |
| Goniometer Radius [mm]: | 240.00 |
| Dist. Focus-Diverg. Slit [mm]: | 91.00 |
| Incident Beam Monochromator: | No |
| Spinning: | No |

Single Crystal X-Ray Analysis (SXRD)

SXRD analysis was conducted on a Agilent Technologies (Dual Source) SuperNova diffractometer using monochromated Cu Kα (λ=1.54184 Å) radiation. The diffractometer was fitted with an Oxford Cryosystems low temperature device to enable data collection to be performed at 120(1) K and the crystal encased in a protective layer of Paratone oil. The data collected were corrected for absorption effects based on Gaussian integration over a multifaceted crystal model, implemented as a part of the CrysAlisPro software package (Agilent Technologies, 2014).

The structure was solved by direct methods (SHELXS97) (Sheldrick, G. M. Acta Cryst. Sect. A 2008, 64, 112) and developed by full least squares refinement on F2 (SHELXL97) interfaced via the OLEX2 software package. Images were produced using OLEX2 (Dolomanov, O. V. et al. J Appl. Cryst. 2009, 42, 339-341).

Polarized Light Microscopy (PLM)

The presence of crystallinity (birefringence) was determined using an Olympus BX50 polarizing microscope, equipped with a Motic camera and image capture software (Motic Images Plus 2.0). All images were recorded using the 20× objective, unless otherwise stated.

Thermogravimetric Analysis (TGA)/Differential Thermal Analysis (DTA)

Approximately, 5 mg of material was weighed into an open aluminum pan and loaded into a simultaneous thermogravimetric/differential thermal analyzer (TG/DTA) and held at room temperature. The sample was then heated at a rate of 10° C./min from 20° C. to 400° C. during which time the change in sample weight was recorded along with any differential thermal events (DTA). Nitrogen was used as the purge gas, at a flow rate of 300 cm³/min.

Differential Scanning Calorimetry (DSC)

Approximately, 5 mg of material was weighed into an aluminum DSC pan and sealed non-hermetically with a pierced aluminum lid. The sample pan was then loaded into a Seiko DSC6200 (equipped with a cooler) cooled and held at 20° C. Once a stable heat-flow response was obtained, the sample and reference were heated to 350° C. at scan rate of 10° C./min and the resulting heat flow response monitored.

Infrared Spectroscopy (IR)

Infrared spectroscopy was carried out on a Bruker ALPHA P spectrometer. Sufficient material was placed onto the center of the plate of the spectrometer and the spectra were obtained using parameters indicated in Table 17:

TABLE 17

| | |
|---|---|
| Resolution: | 4 cm⁻¹ |
| Background Scan Time: | 16 scans |
| Sample Scan Time: | 16 scans |
| Data Collection: | 4000 to 400 cm⁻¹ |
| Result Spectrum: | Transmittance |

Nuclear Magnetic Resonance (NMR)

NMR experiments were performed on a Bruker AVIIIHD spectrometer equipped with a DCH cryoprobe operating at 500.12 MHz for ¹H channel. Experiments were performed in deuterated DMSO and each sample was prepared to about 10 mM concentration.

Dynamic Vapor Sorption (DVS)

Approximately, 10 mg of sample was placed into a mesh vapor sorption balance pan and loaded into a DVS-1 dynamic vapor sorption balance by Surface Measurement Systems. The sample was subjected to a ramping profile from 40-90% relative humidity (RH) at 10% increments, maintaining the sample at each step until a stable weight had been achieved (99.5% step completion). After completion of the sorption cycle, the sample was dried using the same procedure to 0% RH and then a second sorption cycle back to 40% RH. The weight change during the sorption/desorption cycles were plotted, allowing for the hygroscopic nature of the sample to be determined. XRPD analysis was then carried out on any solid retained.

Gravimetric Vapor Sorption (GVS)

Approximately 10-20 mg of sample was placed into a mesh vapor sorption balance pan and loaded into an IGASorp Moisture Sorption Analyzer balance by Hiden Analytical. The sample was subjected to a ramping profile from 40-90% relative humidity (RH) at 10% increments, maintaining the sample at each step until a stable weight had been achieved (98% step completion). After completion of the sorption cycle, the sample was dried using the same procedure to 0% RH, and finally taken back to the starting point of 40% RH. The weight change during the sorption/desorption cycles were plotted, allowing for the hygroscopic nature of the sample to be determined.

High Performance Liquid Chromatography-Ultraviolet Detection (HPLC-UV)

HPLC experiments were performed on Agilent 1100 HPLC instrument with diode array detector (DAD) using parameters indicated in Table 18:

TABLE 18

| | |
|---|---|
| Column: | ACE3 C181-PFP 50 × 4.6 × 3 μm |
| Column Temperature: | 45.0° C. |
| Autosampler Temperature: | Ambient |
| UV wavelength: | 265 nm |
| Injection Volume: | 2.00 μL |
| Flow Rate: | 2 mL/min |
| Mobile Phase A: | 95.0% (0.1% TFA/DI-H₂O) |
| Mobile Phase B: | 5.0% (0.1% TFA/MeCN) |

Gradient program is shown in Table 19:

TABLE 19

| Time (minutes) | Solvent B [%] |
|---|---|
| 0.00 | 5.0 |
| 2.50 | 60.0 |
| 3.20 | 80.0 |
| 3.21 | 5.0 |
| 5.50 | 5.0 |

### Example 1—Solubility of Compound 1 Free Base

A solid Compound 1 was obtained as follows. A 53 mL of solution containing about 330 mg of Compound 1 in warm 1,4-dioxane was divided between 33, 2 mL glass vials (1.5 mL in each). The solutions were frozen and freeze-dried by lyophilization overnight. The resulting material was then analyzed by XRPD to confirm mostly amorphous material.

Approximately 10 mg of amorphous Compound 1 was produced in 32×2 mL glass vials from freeze drying and 100 μL of the appropriate solvent system was added to the appropriate vial. Between each addition, the mixture was checked for dissolution and if no dissolution was apparent, the mixture was heated to about 40° C. and checked again. This procedure was continued until dissolution was observed or until 2 mL had been added (to the compound concentration of <5 mg/mL). The results of the solubility measurements are shown in Table 20.

TABLE 20

| Solvent | Approx. Solubility mg/mL |
|---|---|
| Acetone | 11.1 |
| Acetonitrile | 12.5 |
| Anisole | 11.1 |
| 1-Butanol | 17 |
| 2-Butanone | 11.1 |
| TBME | <5 |
| Cyclohexane | <5 |
| Cyclopentylmethyl ether | <5 |
| Dichloromethane | >100 |
| Diisopropyl ether | <5 |
| N,N-Dimethylacetamide | >100 |
| 1,2-Dimethoxyethane | 8.3 |
| Diglyme (bis(2-methoxyethyl ether) | 8.3 |
| 1,4-Dioxane | 8.3 |
| Dimethylformamide | >100 |
| Dimethylsulfoxide | 50 |
| Ethanol | 20 |
| Ethyl acetate | <5 |
| 2-Ethoxy ethanol | 50 |
| Heptane | <5 |
| Isobutyl acetate | <5 |
| Isopropyl acetate | <5 |
| Methanol | 50 |
| Methylisobutyl ketone | 6.25 |
| 2-Methyl THF | <5 |
| N-Methylpyrrolidone | >100 |
| 2-Propanol | 14.3 |
| 1-Propanol | >100 |
| Tetrahydrofuran | 20 |
| Toluene | <5 |
| TBME:Heptane (60:40 v/v) | <5 |
| Water | <5 |

Compound 1 showed low solubility in non-polar solvents such as toluene and 1,4-dioxane, medium solubility in polar aprotic solvents such as acetone, ethyl acetate and acetonitrile and high solubility in polar solvents such as DMSO, DMF and protic solvents such as methanol. In the remainder of cases and where “<” is present, solid was still present after the maximum volume of 2 mL was added. XRPD analysis of the recovered solids from the solvent solubility study returned the same crystalline form of free base in all cases (Form I), however, showing varying degrees of crystallinity and peak intensity (preferred orientation may have an effect on crystallinity of a sample). Insufficient solids were recovered from anisole, 1-butanol, diglyme, 2-ethoxy ethanol, MIBK and N-methylpyrrolidone.

### Example 2—Preparation of Crystalline Compound 1 (Form I)

Solid Compound 1 was obtained as follows. A 212 mL of solution containing about 1.04 g of Compound 1 in warm 1,4-dioxane was divided between 26, 20 mL glass vials (approximately 8 mL in each). The solutions were frozen and freeze-dried by lyophilization overnight. The resulting material was then analyzed by XRPD to confirm mostly amorphous material.

The 25 vials each containing approximately 40 mg of amorphous freeze-dried Compound 1 were used. A solvent was added to each vial and Compound 1 was suspended in the solvent. The following 25 solvents were used: acetone, acetonitrile, anisole, 1-butanol, 2-butanone, TBME, cyclohexane, cyclopentylmethyl ether, 1,2-dimethoxyethane, 1,4-dioxane, ethanol, ethyl acetate, 2-ethoxy ethanol, heptane, isobutyl acetate, isopropyl acetate, methanol, methylisobutyl ketone, 2-methyl THF, 2-propanol, 1-propanol, tetrahydrofuran (THF), toluene, TBME:heptane (60:40 v/v), and water. The crystallization conditions consisted of maturation cycles, evaporation, cooling and anti-solvent addition techniques.

Temperature Cycling

Each of the 25 vials was temperature cycled between ambient temperature and 40° C. in 4 hour cycles over 72 h. The resulting solids were isolated by centrifugation and analyzed by XRPD and PLM. Solids recovered from temperature cycling and analyzed by XRPD appeared to be the same as the input material (Form I) with varying degrees in crystallinity. No residual solid material was recovered from anisole, 1-butanol, 2-butanone, 2-ethoxy ethanol, 2-methyl THF, 1-propanol and THF.

A filtered saturated solution of Compound 1 in a specified solvent was divided into five vials and used to prepare crystalline forms of the compound according to the procedures described below:

Crash Cool (2° C.)

Saturated solutions of Compound 1 were stored at 2° C. for 24-72h. At this time any material recovered was analyzed by XRPD. The crash cooling experiments at 2° C. recovered insufficient solids from all solvents for XRPD analysis except from 2-propanol which returned Compound 1 (Form I).

Crash Cool (−18° C.)

Saturated solutions of Compound 1 were stored at −18° C. for 24-72h. At this time any material recovered was analyzed by XRPD. The crash cooling experiments at −18° C. recovered insufficient solids from all solvents for XRPD analysis except 1-butanol, ethanol, 2-propanol and 1-propanol. From the solids that were analyzed by XRPD analysis, all returned Compound 1 (Form I) with varying degrees of crystallinity.

Anti-Solvent Addition at Ambient Temperature

Approximately 1 mL of anti-solvent (heptane or TBME depending on miscibility) was added dropwise to saturated solutions of Compound 1 free base. Any resulting solid was analyzed by XRPD. The anti-solvent addition at ambient temperature experiments recovered insufficient solids from all solvents for XRPD analysis except acetone, acetonitrile, 2-butanone, 1,2-dimethoxyethane, 1,4-dioxane and ethanol. From the solids that were analyzed by XRPD analysis, all returned Compound 1 (Form I) with varying degrees of crystallinity.

Anti-Solvent Addition at 2° C.

Approximately 1 mL of anti-solvent (heptane or TBME depending on miscibility) was added dropwise to saturated solutions of Compound 1 free base. Any resulting solid was analyzed by XRPD. The anti-solvent addition at 2° C. experiments recovered insufficient solids from all solvents for XRPD analysis except acetone, acetonitrile, 1-butanol, 2-butanone, 1,2-dimethoxyethane, 1,4-dioxane, ethanol, ethyl acetate, MIBK, 1-propanol and THF. From the solids that were analyzed by XRPD, all returned Compound 1 (Form I) with varying degrees of crystallinity.

Evaporation

Saturated solutions of Compound 1 were transferred to 2 mL vials, these vials were then uncapped and allowed to evaporate at ambient temperature to recover material. Any material recovered was analyzed by XRPD. The evaporation experiments recovered insufficient solids from all solvents for XRPD analysis except acetone, acetonitrile, 2-butanone, cyclopropylmethyl ether, 1,2-dimethoxyethane, 1,4-dioxane, ethanol, ethyl acetate, 2-ethoxy ethanol, isobutyl acetate, isopropyl acetate, methanol, MIBK, 2-propanol, 1-propanol and THF. From the solids that were analyzed by XRPD, all returned Compound 1 (Form I) with varying degrees of crystallinity.

### Example 3—Characterization of Crystalline Compound 1 (Form I)

X-Ray Powder Diffraction (XRPD)

Form I of crystalline (6R,15R)-9-fluoro-15-methyl-2,11,16,20,21,24-hexaazapentacyclo[16.5.2.0^{2,6}.0^{7,12}.0^{21,25}]pentacosa-1(24),7,9,11,18(25),19,22-heptaen-17-one (Compound 1 free base) was characterized by XRPD. The XRPD pattern is shown in FIG. 1 and XRPD data is provided in Table 21.

TABLE 21

| 2-Theta (°) | Height | H % |
|---|---|---|
| 7.9 | 692 | 4.0 |
| 9.1 | 10133 | 58.1 |
| 11.2 | 6232 | 35.7 |
| 12.8 | 695 | 4.0 |
| 13.4 | 4471 | 25.6 |
| 14.8 | 2667 | 15.3 |
| 15.2 | 479 | 2.8 |
| 15.5 | 144 | 0.8 |
| 16.8 | 929 | 5.3 |
| 18.3 | 2049 | 11.8 |
| 18.6 | 2818 | 16.2 |
| 19.5 | 792 | 4.5 |
| 20.2 | 17437 | 100.0 |
| 21.4 | 1327 | 7.6 |
| 22.7 | 1668 | 9.6 |
| 23.2 | 210 | 1.2 |
| 23.6 | 1908 | 10.9 |
| 24.9 | 6322 | 36.3 |
| 25.8 | 783 | 4.5 |
| 26.1 | 447 | 2.6 |
| 26.5 | 537 | 3.1 |
| 27.0 | 1478 | 8.5 |
| 27.7 | 220 | 1.3 |
| 28.4 | 259 | 1.5 |
| 28.8 | 228 | 1.3 |
| 29.4 | 1795 | 10.3 |
| 30.0 | 142 | 0.8 |
| 30.3 | 358 | 2.1 |
| 31.2 | 197 | 1.1 |
| 32.1 | 359 | 2.1 |
| 32.3 | 357 | 2.1 |
| 33.3 | 248 | 1.4 |
| 34.4 | 70 | 0.4 |

As shown in FIG. 1, according to the XRPD analysis, the material is crystalline. PLM analysis showed birefringence with irregular morphology.

Thermogravimetric/Differential Thermal Analysis (TG/DTA)

Form I of crystalline (6R,15R)-9-fluoro-15-methyl-2,11,16,20,21,24-hexaazapentacyclo[16.5.2.0^{2,6}.0^{7,12}.0^{21,25}]pentacosa-1 (24),7,9,11,18(25), 19,22-heptaen-17-one (Compound 1 free base) was characterized by TGA and DTA. TGA showed a weight loss of approximately 1.1% from outset up to 200° C., while DTA showed an endothermal “melting” event at onset about 315° C. (peak at 317° C.). The TG/DTA thermogram is shown in FIG. 2.

Differential Scanning Calorimetry (DSC)

Form I of crystalline (6R,15R)-9-fluoro-15-methyl-2,11,16,20,21,24-hexaazapentacyclo[16.5.2.0^{2,6}.0^{7,12}.0^{21,25}]pentacosa-1 (24),7,9,11,18(25), 19,22-heptaen-17-one (Compound 1 free base) was characterized by DSC. DSC analysis in the first heat showed a sharp endothermal event at onset 315° C. (peak at 317° C.) which is consistent with TG/DTA. No thermal events were seen in the cooling cycle. The second heating cycle showed a small endothermal event at onset around 118° C. (peak at 124° C.) which is highly likely to be a glass transition (T_{g}). The DSC thermograms are shown in FIG. 3

In sum, Compound 1 exists as one crystalline form (Form I) with favorable thermal properties with a melting point of 315° C. and low hygroscopicity with a mass uptake of 0.3% at 90% RH and no changes to form or crystallinity after exposure to GVS humidity conditions.

### Example 4—Recrystallization of Compound 1 and Characterization of the Recrystallized Material

Compound 1 was recrystallized from 1-propanol as follows. 500 mg of Compound 1 was weighed into a 20 mL vial. To this vial, 20 mL of 1-propanol was added gradually over 3 hours. The sample was placed in a 95° C. heated block to aid dissolution. The sample was slow to dissolve but a clear solution was achieved. The sample was cooled to 10° C. at 5° C./min. Once the cooling cycle had reached 10° C. the sample remained at 10° C. for a further 24 hours to recover material. The solids were then recovered and dried using a vacuum oven at ambient temperature.

XRPD analysis of the recrystallized solid showed no changes in crystalline form, and PLM analysis showed the material to be birefringent with irregular morphology.

TGA showed a weight loss of approximately 0.7% from the outset up to 250° C., whilst DTA showed an endothermal ‘melting’ event at onset approximately at 314° C. (peak at about 318° C.).

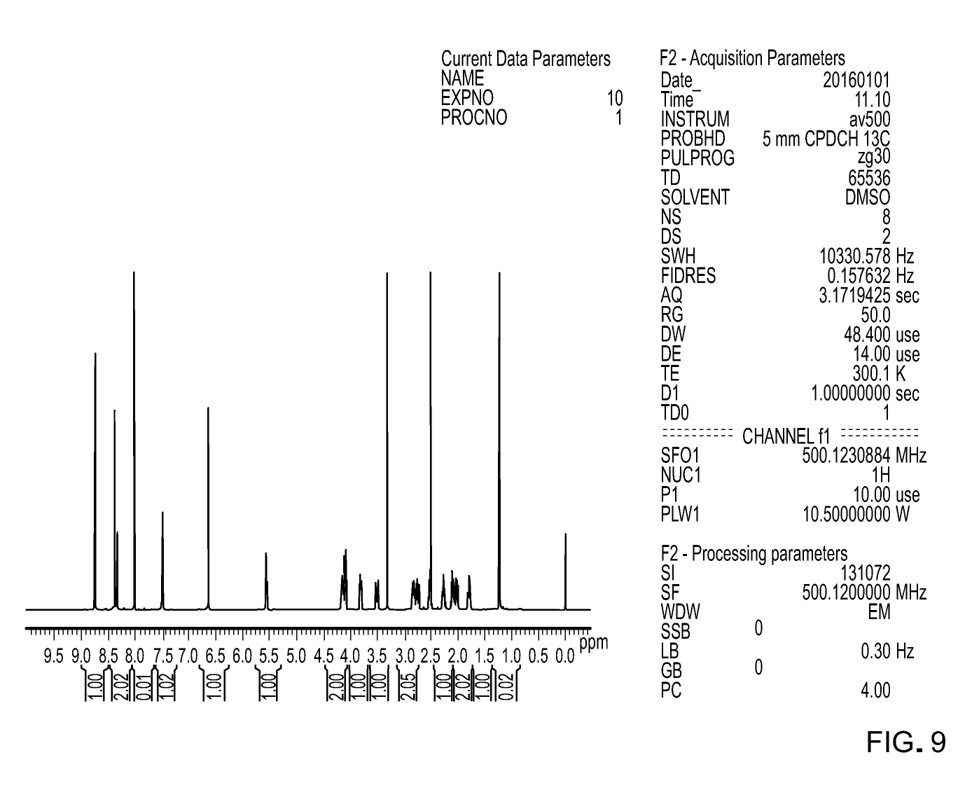

Purity of recrystallized solid is 99.2% as determined by HPLC-UV ¹H-NMR analysis shows that the spectrum is consistent with the structure and shows little if any obvious residual process solvents. ¹H NMR spectrum is shown in FIG. 9.

Differential Scanning Calorimetry (DSC)

DSC analysis in the first heating cycle showed a sharp endothermal event at onset approximately 316° C. (peak at 317° C.). This endothermal event is consistent with TG/DTA. In the first cooling cycle of the DSC analysis, a slow broad recrystallization is observed with a peak at about 284° C. shows the thermogram of the first cooling cycle. DSC analysis in the second heating cycle showed a series of exothermic events which could be potential recrystallizations which were followed by a sharp endothermal event at onset about 313° C. (peak at about 316° C.) shows the thermogram of the second heating cycle of the recrystallized Compound 1 free base.

Infra-Red Analysis (IR)

Recrystallized Form I of crystalline Compound 1 was characterized by IR. FIG. 8 shows IR spectrum and the peaks are listed in Table 22.

TABLE 22

| Wave Number | Abs. Intensity | Rel. Intensity | Width |
|---|---|---|---|
| 3344.4 | 0.8 | 0.1 | 27.8 |
| 3066.3 | 0.9 | 0.0 | 31.6 |
| 3019.9 | 0.9 | 0.0 | 15.3 |
| 2962.0 | 0.8 | 0.0 | 18.7 |
| 2929.8 | 0.8 | 0.0 | 25.5 |
| 2870.4 | 0.8 | 0.1 | 143.0 |
| 1649.6 | 0.6 | 0.1 | 2538.9 |
| 1625.8 | 0.5 | 0.4 | 43.1 |
| 1599.0 | 0.8 | 0.0 | 61.5 |
| 1566.9 | 0.6 | 0.3 | 17.6 |
| 1537.4 | 0.6 | 0.2 | 13.5 |
| 1492.1 | 0.5 | 0.2 | 2106.0 |
| 1450.6 | 0.4 | 0.5 | 63.7 |
| 1365.4 | 0.6 | 0.1 | 1743.6 |
| 1346.7 | 0.6 | 0.2 | 44.5 |
| 1281.8 | 0.7 | 0.1 | 8.5 |
| 1257.5 | 0.7 | 0.1 | 8.0 |
| 1234.4 | 0.6 | 0.2 | 40.6 |
| 1219.2 | 0.6 | 0.0 | 5.5 |
| 1167.6 | 0.7 | 0.1 | 35.4 |
| 1156.0 | 0.7 | 0.0 | 5.6 |
| 1114.8 | 0.8 | 0.0 | 9.4 |
| 1093.2 | 0.8 | 0.0 | 9.7 |
| 1070.8 | 0.7 | 0.1 | 10.7 |
| 992.3 | 0.8 | 0.0 | 318.0 |
| 964.0 | 0.7 | 0.1 | 13.0 |
| 945.2 | 0.8 | 0.0 | 132.0 |
| 923.0 | 0.8 | 0.1 | 6.9 |
| 903.6 | 0.7 | 0.1 | 7.3 |
| 890.8 | 0.7 | 0.2 | 35.4 |
| 859.2 | 0.8 | 0.0 | 175.9 |
| 796.2 | 0.6 | 0.2 | 11.5 |
| 777.7 | 0.6 | 0.0 | 0.1 |
| 770.5 | 0.6 | 0.3 | 19.5 |
| 740.5 | 0.8 | 0.0 | 147.4 |
| 719.7 | 0.8 | 0.0 | 694.4 |
| 709.6 | 0.7 | 0.1 | 25.8 |
| 686.7 | 0.8 | 0.0 | 170.0 |
| 633.0 | 0.8 | 0.1 | 7.9 |
| 616.0 | 0.7 | 0.1 | 11.2 |
| 552.4 | 0.6 | 0.2 | 43.9 |
| 509.5 | 0.7 | 0.0 | 9.3 |
| 468.8 | 0.8 | 0.1 | 13.6 |
| 442.2 | 0.7 | 0.1 | 16.7 |
| 432.1 | 0.8 | 0.0 | 91.7 |
| 405.0 | 0.7 | 0.1 | 10.1 |

In sum, Compound 1 recrystallized from 1-propanol exhibited the same properties as the compound prior to recrystallization, with an increased purity of >99%. As shown in Example 5, the material showed no change to form or purity after exposure to stability stress conditions and no change to form after an aqueous solubility assessment.

### Example 5—Stability of Compound 1 (Form I)

Compound 1 (Form I) was subjected to various different environmental conditions to assess stability.

Vapor Sorption—Before Recrystallization

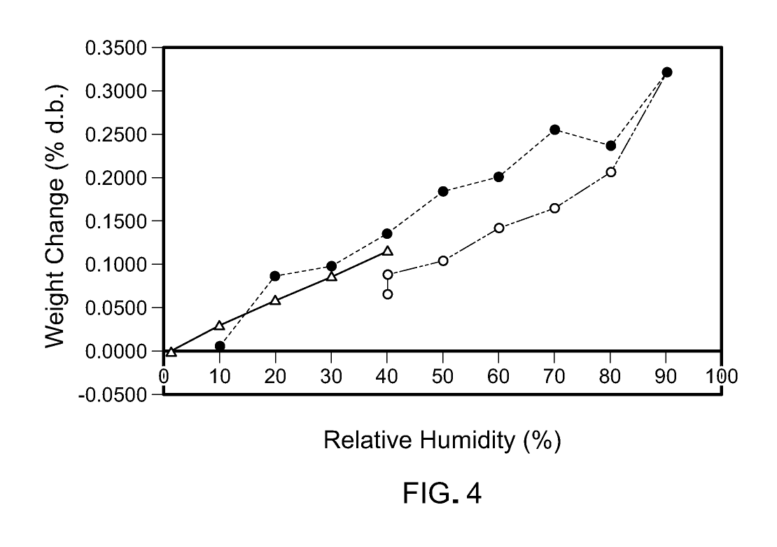

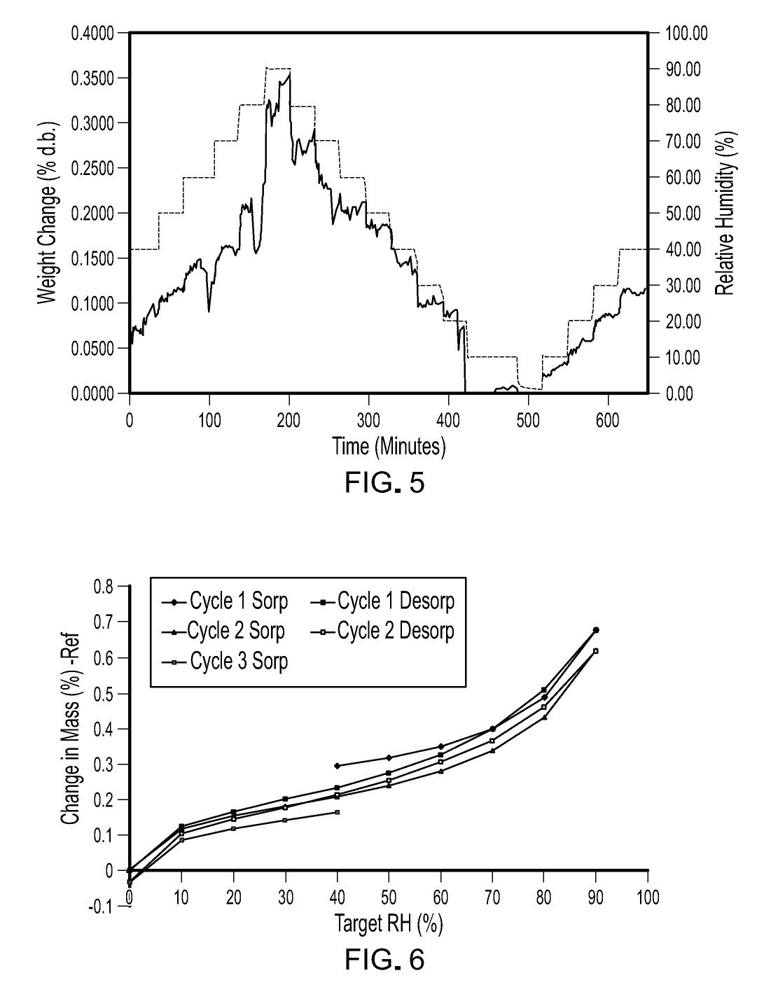

Gravimetric vapor sorption (GVS) showed that Compound 1 exhibits slight hygroscopicity with a mass uptake of approximately 0.3% at 90% RH. FIG. 4 shows GVS isotherm plot and FIG. 5 shows GVS kinetic plot. Post-XRPD analysis showed no changes in crystalline form upon exposure to GVS conditions.

Vapor Sorption—Recrystallized Solid

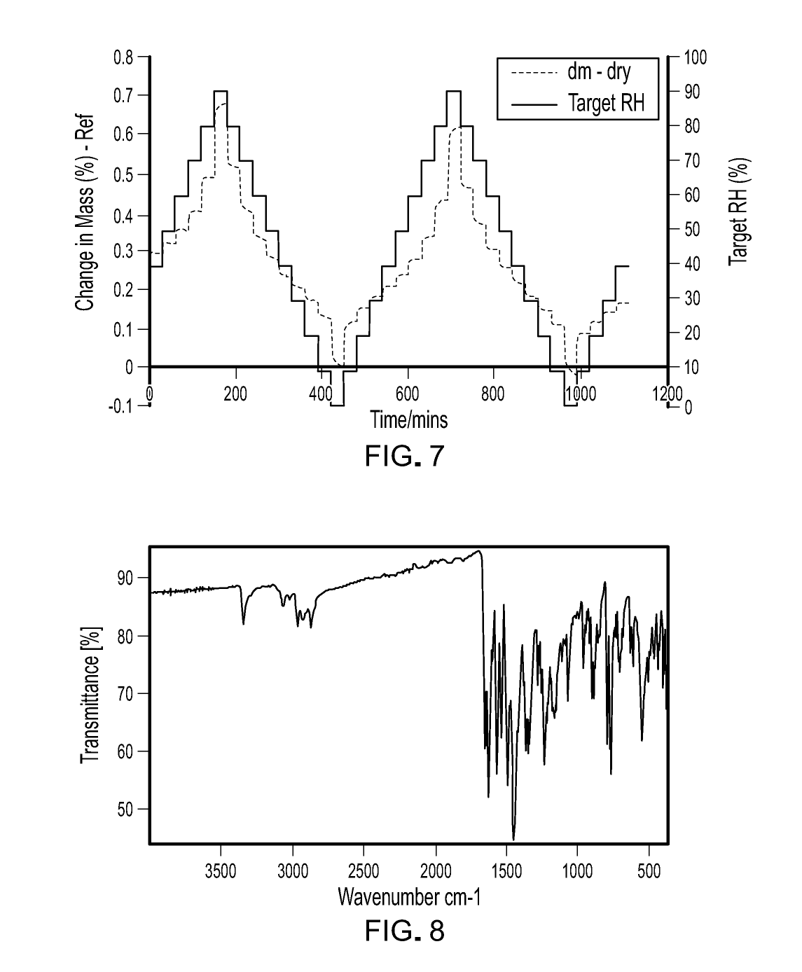

Dynamic vapor sorption (DVS) analysis of the recrystallized compound shows the material to exhibit slight hygroscopicity with a mass uptake of about 0.7% at 90% RH. FIG. 6 shows DVS analysis of the recrystallized compound. FIG. 7 shows DVS kinetic plot of the recrystallized solid. Post-DVS XRPD analysis shows no change in crystalline form upon exposure to DVS humidity conditions.

Humidity, Temperature, Ambient Light Recrystallized Solid

1-week stability tests on recrystallized solid showed no change to form after exposure to 40° C./75% RH, 80° C. and under ambient light. UPLC analysis showed no change in purity of the samples after exposure to stability stress conditions (average purity 99.2 for relative humidity and ambient light tests, and 99.3% for 80° C. test).

### Example 6—Single Crystal X-Ray Analysis of Compound 1 (Form I)

Crystals of Compound 1 (Form 1) were prepared as follows. Compound 1 (2 mg) was dissolved in methanol (500 μL) in a 1.75 clear glass vial then capped with a pierced lid. The solution was left to stand at ambient for several days without agitation to allow for large rod-like crystals to grow that were suitable for interrogation by single crystal X-ray diffraction.

The highest residual Fourier peak was found to be 0.16 e·Å⁻³ approx 0.72 Å from C(4), and the deepest Fourier hole was found to be −0.22 e·Å⁻³ approx 0.75 Å from C(10).

Crystal Data for C₂₀H₂₁FN₆O (M=380.43 g/mol): orthorhombic, space group P2₁2₁2₁ (no. 19), a=6.91792(3) Å, b=13.74742(3) Å, c=19.22580(5) Å, V=1828.442(10) Å³, Z=4, T=207(120) K, μ(CuKα)=0.799 mm-1, Dcalc=1.382 g/cm³, 169333 reflections measured (7.9°≤2Θ≤152.76°), 3833 unique (Rint=0.0639, Rsigma=0.0180) which were used in all calculations. The final R¹ was 0.0338 (>2sigma(I)) and wR2 was 0.0908 (all data). Crystallographic parameters and refinement indicators of Compound 1 (Form I) are shown in Table 23.

TABLE 23

| Empirical formula | C₂₀H₂₁FN₆O |
|---|---|
| Formula weight | 380.43 |
| Temperature/K | 120(1) |
| Crystal system | Orthorhombic |
| Space group | P2₁2₁2₁ |
| a/Å | 6.91792(3) |
| b/Å | 13.74742(3) |
| c/Å | 19.22580(5) |
| α/° | 90.00 |
| β/° | 90.00 |
| γ/° | 90.00 |
| Volume/Å³ | 1828.442(10) |
| Z, Z\grave{ } | 4 |
| ρcalc g/cm³ | 1.382 |
| μ/mm⁻¹ | 0.799 |
| F(000) | 800.0 |
| Crystal size/mm³ | 0.47 × 0.117 × 0.105 |
| Radiation | CuKα (λ = 1.54178) |
| 2Θ range for data collection/° | 7.9 to 152.76 |
| Index ranges | −8 ≤ h ≤ 7, −17 ≤ k ≤ 17, |
| | −24 ≤ 1 ≤ 24 |
| Reflections collected | 169333 |
| Independent reflections | 3833 [Rᵢₙₜ = 0.0639, R_{sigma} = 0.0180] |
| Data/restraints/parameters | 3833/0/258 |
| S | 1.060 |
| Final R indexes [F² > 2σ (F²)] | R₁ = 0.0338, wR₂ = 0.0907 |
| Final R indexes [all data] | R₁ = 0.0340, wR₂ = 0.0908 |
| Δρmax, Δρmin/e Å⁻³ | 0.16/−0.22 |
| Flack parameter | −0.01(15) |

FIG. 10 shows 3-D view of Compound 1 (Form I) with atom labels. FIG. 11 shows ORTEP view of Compound 1 (Form I) with atom labels. All non-hydrogen atoms are shown with thermal ellipsoids set at the 50% probability level.

### Example 7—Single Crystal X-Ray Analysis of Compound 1, Acetonitrile Solvate

Crystals of Compound 1, acetonitrile solvate were prepared vas follows. Compound 1 (2 mg) was dissolved in acetonitrile (500 μl) in a 1.75 clear glass vial then capped with a pierced lid. This solution was left to stand at ambient for several days without agitation to allow for large rod-like crystals to grow that were suitable for interrogation by single crystal X-ray diffraction.

The highest residual Fourier peak was found to be 0.19 e·Å⁻³ approx 0.67 Å from C(11), and the deepest Fourier hole was found to be −0.21 e·Å⁻³ approx 0.81 Å from N(4).

Crystal Data for C₂₄H₂₇FN₈O (M=462.54 g/mol): orthorhombic, space group P2₁2₁2₁ (no. 19), a=6.03307(4) Å, b=16.10794(9) Å, c=23.72624(13) Å, V=2305.73(2) Å³, Z=4, T=294.01(10) K, μ(CuKα)=0.757 mm-1, Dcalc=1.332 g/cm³, 110019 reflections measured (6.64°≤2θ≤152.4°), 4840 unique (Rint=0.0983, Rsigma=0.0211) which were used in all calculations. The final R¹ was 0.0339 (>2sigma(I)) and wR2 was 0.0891 (all data). Crystallographic parameters and refinement indicators of Compound 1 (Form I) are shown in Table 24.

TABLE 24

| Empirical formula | C₂₄H₂₇FN₈O |
|---|---|
| Formula weight | 462.54 |
| Temperature/K | 120(1) |
| Crystal system | orthorhombic |
| Space group | P2₁2₁2₁ |
| a/Å | 6.03307(4) |
| b/Å | 16.10794(9) |
| c/Å | 23.72624(13) |
| α/° | 90.00 |
| β/° | 90.00 |
| γ/° | 90.00 |
| Volume/Å³ | 2305.73(2) |
| Z, Z\grave{ } | 4 |
| ρcalc g/cm³ | 1.332 |
| μ/mm⁻¹ | 0.757 |
| F(000) | 976.0 |
| Crystal size/mm³ | 0.564 × 0.082 × 0.033 |
| Radiation | CuKα (λ = 1.54184) |
| 2Θ range for data collection/° | 6.64 to 152.4 |
| Index ranges | 7 ≤ h ≤ 6, −20 ≤ k ≤ 20, −29 ≤ 1 ≤ 29 |
| Reflections collected | 110019 |
| Independent reflections | 4840 [Rᵢₙₜ = 0.0983, R_{sigma} = 0.0211] |
| Data/restraints/parameters | 4840/0/310 |
| S | 1.096 |
| Final R indexes [F² > 2σ (F²)] | R₁ = 0.0339, wR₂ = 0.0887 |
| Final R indexes [all data] | R₁ = 0.0345, wR₂ = 0.0891 |
| Δρmax, Δρmin/e Å⁻³ | 0.19/−0.21 |
| Flack parameter | −0.02(14) |

FIG. 12 shows 3-D view of Compound 1 bis-acetonitrile solvate with atom labels. FIG. 13 shows ORTEP view of Compound 1 bis-acetonitrile solvate asymmetric unit with atom labels. All non-hydrogen atoms are shown with thermal ellipsoids set at the 50% probability level.

### Example 8—Preparation and Characterization of Compound 1 Benzenesulfonic Acid Salt

250 μL of the appropriate solvent was added to the vials containing 20 mg of compound 1. In a separate vial, 250 μL of the appropriate solvent was added to the vial containing benzenesulfonic acid (8.92 mg). The solutions/slurries were then added to the solvent/compound 1 solution (1.05 eq. of acid to free base). The samples were then temperature cycled between ambient and 40° C. in 4 hour cycles over 24 hrs.

Scale-Up Preparation from Ethanol

About 300 mg of Compound 1 was weighed into a vial and 133 mg of benzenesulfonic acid was weighed into a separate vial. To both vials, 3.75 mL of ethanol was added and the two mixtures combined. The resulting slurry was then temperature cycled for 24 hours (ambient to 40° C. in 4 hours cycles) (1.05 eq. of acid to free base). The resulting slurry was then allowed to evaporate at ambient temperatures to remove excess ethanol.

Observations from the treatment of Compound 1 with benzenesulfonic acid are shown in Table 25 below:

TABLE 25

| Time- | Solvent | | | | | |
|---|---|---|---|---|---|---|
| point | Acetone | Ethanol | Methanol | 2-propanol | TBME | THF |
| Pre- | Slurry | Slurry | Slurry | Slurry | Slurry | Slurry |
| Cycling | | | | | | |
| Post- | Solid | Clear | Gum | Slurry | Slurry | Solid |
| Cycling | | Solution | | | | |

To the samples which were recovered as clear solutions, 2-3 mg of Compound 1 was added to produce a mobile slurry and the sample temperature cycled for a further 2-3 hours. XRPD analysis of benzenesulfonic acid experiments recovered 5 crystalline hits, free base (Form I) recovered from acetone and 2-propanol, pattern 1 was recovered from THF and t-BME (FIG. 17) and pattern 2 recovered from ethanol (FIG. 18). Insufficient solids were recovered from ethanol to determine form.

XRPD data for Compound 1 besylate is provided in Table 26.

TABLE 26

| 2-Theta (°) | Height | H % |
|---|---|---|
| 8.1 | 15179 | 100.0 |
| 9.2 | 864 | 5.7 |
| 10.0 | 85 | 0.6 |
| 11.7 | 591 | 3.9 |
| 12.0 | 1879 | 12.4 |
| 12.4 | 394 | 2.6 |
| 13.4 | 3923 | 25.9 |
| 15.1 | 548 | 3.6 |
| 16.0 | 196 | 1.3 |
| 16.7 | 156 | 1.0 |
| 18.4 | 302 | 2.0 |
| 19.0 | 2184 | 14.4 |
| 19.4 | 1644 | 10.8 |
| 19.9 | 1220 | 8.0 |
| 20.1 | 959 | 6.3 |
| 20.6 | 226 | 1.5 |
| 21.2 | 3809 | 25.1 |
| 21.7 | 587 | 3.9 |
| 21.9 | 362 | 2.4 |
| 22.5 | 749 | 4.9 |
| 23.3 | 165 | 1.1 |
| 23.7 | 114 | 0.8 |
| 24.1 | 80 | 0.5 |
| 25.5 | 1263 | 8.3 |
| 25.8 | 545 | 3.6 |
| 26.0 | 183 | 1.2 |
| 26.4 | 159 | 1.1 |
| 26.7 | 420 | 2.8 |
| 27.0 | 768 | 5.1 |
| 27.8 | 126 | 0.8 |
| 28.1 | 66 | 0.4 |
| 28.5 | 153 | 1.0 |
| 28.9 | 39 | 0.3 |
| 29.3 | 478 | 3.2 |
| 30.3 | 127 | 0.8 |
| 30.8 | 50 | 0.3 |
| 32.0 | 806 | 5.3 |
| 32.7 | 1080 | 7.1 |
| 33.2 | 155 | 1.0 |
| 33.4 | 177 | 1.2 |
| 33.8 | 153 | 1.0 |
| 34.7 | 260 | 1.7 |

TG/DT Analysis

TGA of besylate pattern 1 from tBME showed a total weight loss of approximately 13% from the outset to about 150° C. DTA showed an endothermal event at onset about 241° C. (peak at about 247° C.). TGA of besylate pattern 1 from ethanol showed a total weight loss of approximately 0.4% from the outset to about 250° C. DTA showed an endothermal event at onset about 244° C. (peak at about 248° C.).

Result of Stability Studies

XRPD analysis of post-stability besylate pattern 1 recovered from THF showed an increase to crystallinity but no changes to form after exposure to stability conditions. XRPD analysis of post-stability besylate pattern 1 recovered from TBME showed preferred orientation but no changes to form after exposure to stability conditions. XRPD analysis of post-stability besylate pattern 1 recovered from ethanol showed a decrease in crystallinity after exposure to stability conditions.

Secondary Salt Scale Up

XRPD analysis of besylate scale up showed successful formation of besylate pattern 2 from ethanol seen in the salt screen, a large amount of preferred orientation is seen in the sample.

TGA (FIG. 37) showed a weight loss of approximately 0.7% from the outset up to around 250° C. whilst DTA showed an endothermal “melting” event at onset around 244° C. (peak at around 248° C.).

DSC analysis (FIG. 38) in the first heating cycle showed a sharp endothermal event at onset around 246° C. (peak at 249° C.). This endothermal event is consistent with TG/DTA and no thermal events were seen in the cooling or second heating cycle.

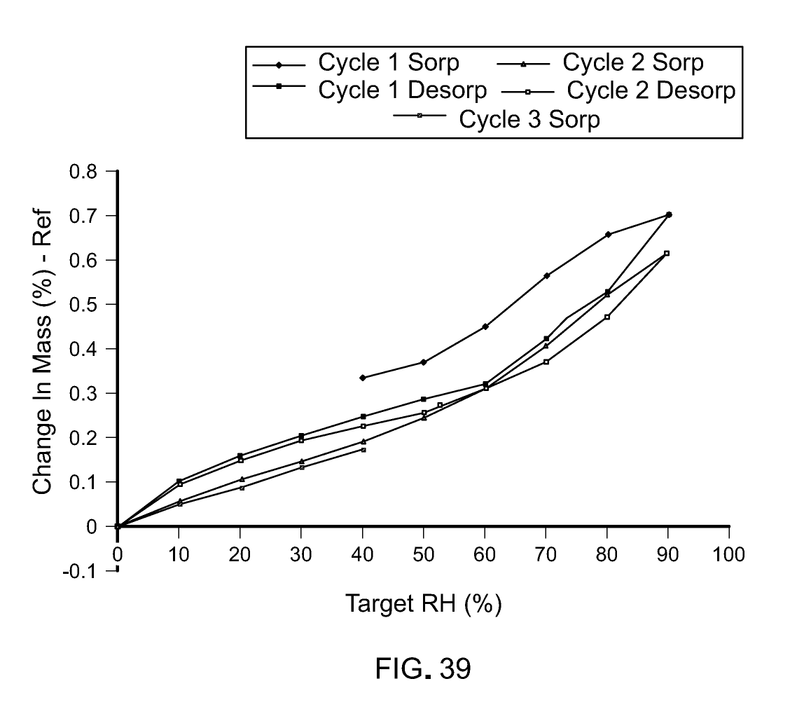

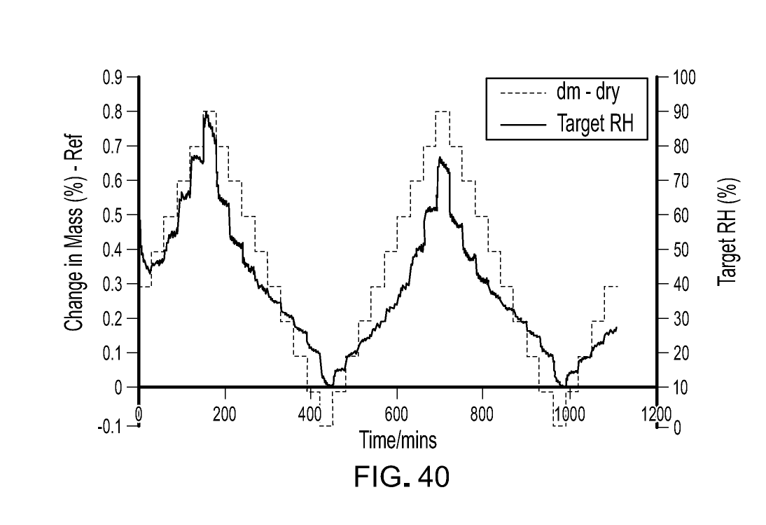

Compound 1 besylate exhibits low hygroscopicity when exposed by DVS conditions with a mass uptake of about 0.7% at 90% RH (FIGS. 39 and 40). Post-DVS XRPD analysis shows no changes in crystalline form after exposure, a large amount of preferred orientation is seen in the sample. The hysteresis observed is most likely caused by a small amount of amorphous content which appears to crystallize at 90% RH.

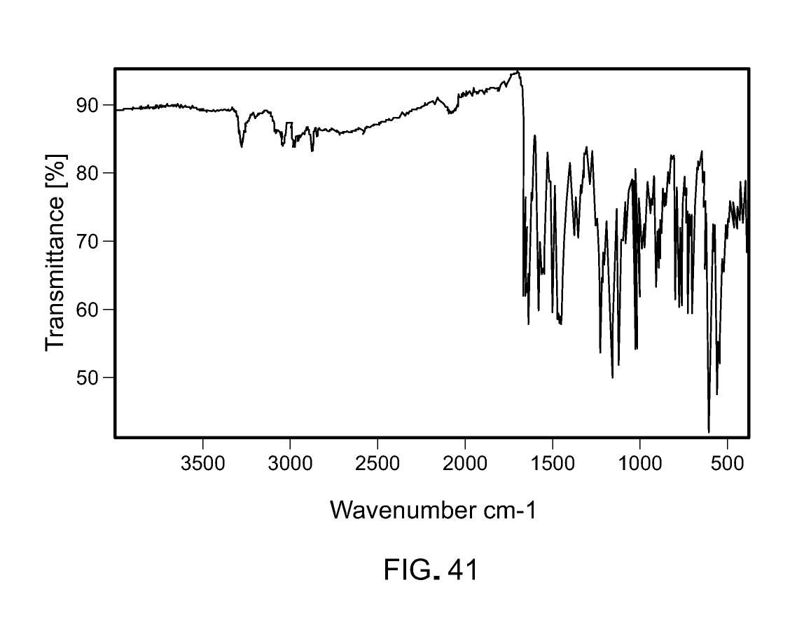

An IR spectrum of Compound 1 besylate was taken for reference which can be found in FIG. 41 with peak lists in Table 27.

TABLE 27

| Wave | Wave | Wave | Wave |
|---|---|---|---|
| number | number | number | number |
| 3271 | 1343 | 902 | 549 |
| 3033 | 1279 | 846 | 526 |
| 2974 | 1248 | 828 | 503 |
| 2864 | 1222 | 791 | 476 |
| 2069 | 1197 | 772 | 459 |
| 1657 | 1157 | 757 | 445 |
| 1634 | 1119 | 734 | 415 |
| 1573 | 1077 | 723 | |
| 1544 | 1032 | 708 | |
| 1496 | 1018 | 692 | |
| 1464 | 994 | 640 | |
| 1456 | 968 | 628 | |
| 1446 | 933 | 609 | |
| 1371 | 923 | 561 | |

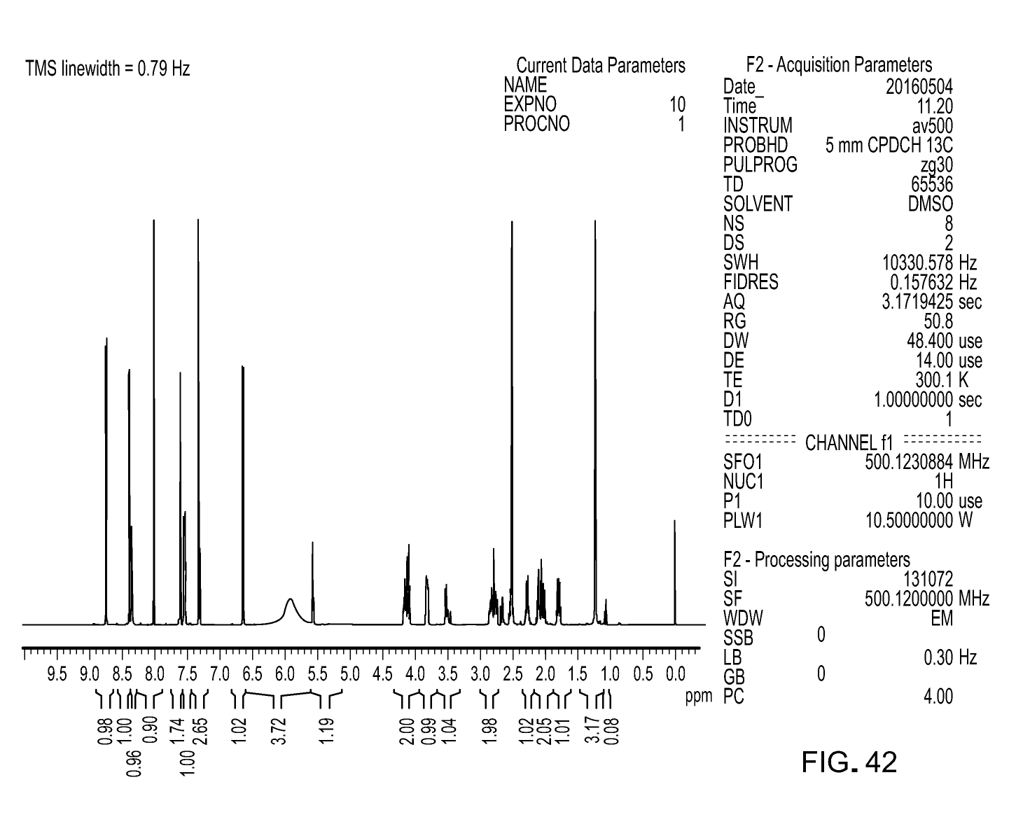

¹H-NMR spectrum shown in FIG. 42 shows 0.88 eq. benzenesulfonic acid, and 0.028 eq. EtOH. UPLC analysis of Compound 1 besylate gave an average purity of 99.4%.

1 week stability tests at 80° C. and under ambient light showed no change to form after exposure and no change to purity. However, by XRPD analysis, the sample held at 40° C./75% RH appears to be a mixture of besylate salt and something else.

Thermodynamic solubility studies of Compound 1 besylate show the salt is highly soluble in pH 1, moderately soluble in 4.5 and unbuffered water. The sample shows low solubility in pH 6.8. pH and concentration values can be found in Table 28.

TABLE 28

| Sample ID | Concentration (mg/mL) |
|---|---|
| pH 1 | 30.8 |
| pH 4.5 | 12.7 |
| pH 6.8 | 1.9 |
| Un-buffered Water | 17.9 |

XRPD analysis showed insufficient solids were recovered from pH 1, an unknown form was recovered from pH 4.5 and poorly crystalline free base was recovered from pH 6.8 and unbuffered water. Salt disproportionation studies of Compound 1 besylate showed the recovered material to be poorly crystalline free base by XRPD analysis.

Hydration studies of Compound 1 besylate found insufficient solids were recovered from medium water activity and poorly crystalline besylate salt recovered from low and high water activities, by the poor crystallinity of the recovered material and a peak at around 21 degrees indicate the potential of a hydrate formation.

### Example 9—Preparation and Characterization of Compound 1 Citric Acid Salt

250 μL of the appropriate solvent was added to the vials containing 20 mg of compound 1. In a separate vial, 250 μL of the appropriate solvent was added to the vial containing citric acid (10.67 mg). The solutions/slurries were then added to the solvent/compound 1 solution (1.05 eq. of acid to free base). The samples were then temperature cycled between ambient and 40° C. in 4 hour cycles over 24 hrs.

Scale-Up Preparation from Acetone

About 300 mg of compound 1 was weighed into a vial and 160 mg of citric acid was weighed into a separate vial. To both vials, 3.75 mL of acetone was added and the two mixtures combined. The resulting slurry was then temperature cycled for 24 hours (ambient to 40° C. in 4 hours cycles). The resulting slurry was then allowed to evaporate at ambient temperature to remove excess acetone (1.05 eq. of acid to free base).

Observations from the treatment of Compound 1 with citric acid are shown in Table 29 below:

TABLE 29

| Time- | Solvent | | | | | |
|---|---|---|---|---|---|---|
| point | Acetone | Ethanol | Methanol | 2-propanol | TBME | THF |
| Pre- | Slurry | Slurry | Slurry | Slurry | Slurry | Slurry |
| Cycling | | | | | | |
| Post- | Slurry | Slurry | Slurry | Slurry | Slurry | Slurry |
| Cycling | | | | | | |

XRPD analysis of citric acid experiments recovered 6 crystalline hits, free base (Form I) recovered from ethanol, methanol, 2-propanol, and THF and Form I recovered from acetone and TBME (FIG. 21).

XRPD data for Form I is provided in Table 30.

TABLE 30

| 2-Theta (°) | Height | H % |
|---|---|---|
| 6.5 | 1116 | 17.6 |
| 8.9 | 4365 | 68.8 |
| 9.2 | 1294 | 20.4 |
| 11.1 | 2946 | 46.5 |
| 13.9 | 1576 | 24.9 |
| 14.4 | 2604 | 41.1 |
| 15.4 | 2495 | 39.3 |
| 15.9 | 1182 | 18.6 |
| 18.0 | 755 | 11.9 |
| 19.2 | 2335 | 36.8 |
| 19.6 | 1370 | 21.6 |
| 20.7 | 6342 | 100.0 |
| 21.6 | 4090 | 64.5 |
| 22.3 | 274 | 4.3 |
| 22.7 | 348 | 5.5 |
| 23.3 | 1387 | 21.9 |
| 23.7 | 962 | 15.2 |
| 24.2 | 737 | 11.6 |
| 24.8 | 4022 | 63.4 |
| 25.6 | 2421 | 38.2 |
| 26.3 | 533 | 8.4 |
| 26.5 | 788 | 12.4 |
| 26.8 | 581 | 9.2 |
| 27.9 | 927 | 14.6 |
| 28.9 | 378 | 6.0 |
| 29.1 | 350 | 5.5 |
| 30.2 | 533 | 8.4 |
| 30.6 | 180 | 2.9 |
| 31.8 | 205 | 3.2 |
| 32.5 | 365 | 5.8 |
| 33.1 | 137 | 2.2 |
| 33.7 | 347 | 5.5 |
| 34.3 | 151 | 2.4 |
| 34.5 | 138 | 2.2 |

TG/DT Analysis

TGA of citrate Form A showed a total weight loss of approximately 1% from the outset up to about 175° C. DTA showed several endothermal events; first event at onset about 187° C. (peak at about 194° C.) and the second event at onset about 316° C. (peak at about 318° C.).

Result of Stability Studies

XRPD analysis of post-stability citrate Form A recovered from acetone showed a decrease to crystallinity but no change to form after exposure to stability conditions. XRPD analysis of post-stability citrate Form A recovered from TBME showed a decrease to crystallinity but no change to form after exposure to stability conditions.

Secondary Salt Scale Up

XRPD analysis of the scaled up citrate salt shows successful formation of citrate Form A from acetone seen in the salt screen.

TGA (FIG. 43) showed a total weight loss of approximately 3% from the outset up to 175° C. DTA showed several endothermal events, the first event at onset around 188° C. (peak at around 194° C.) and the second event at onset around 316° C. (peak at around 318° C.).

DSC analysis in the first heating cycle (FIG. 44) showed a potential overlap of two endothermal events (peaks at 194 and 205° C.). No thermal events were seen in the cooling or second heating cycle.

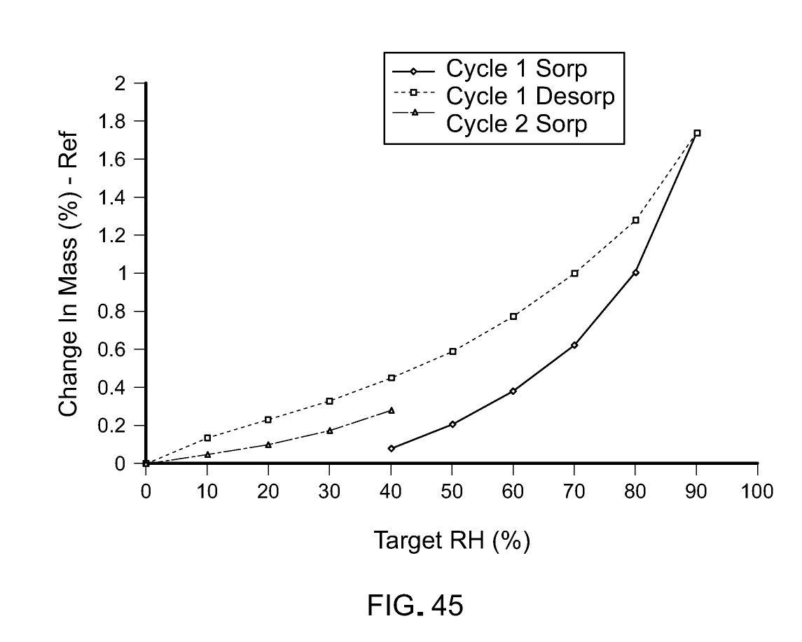

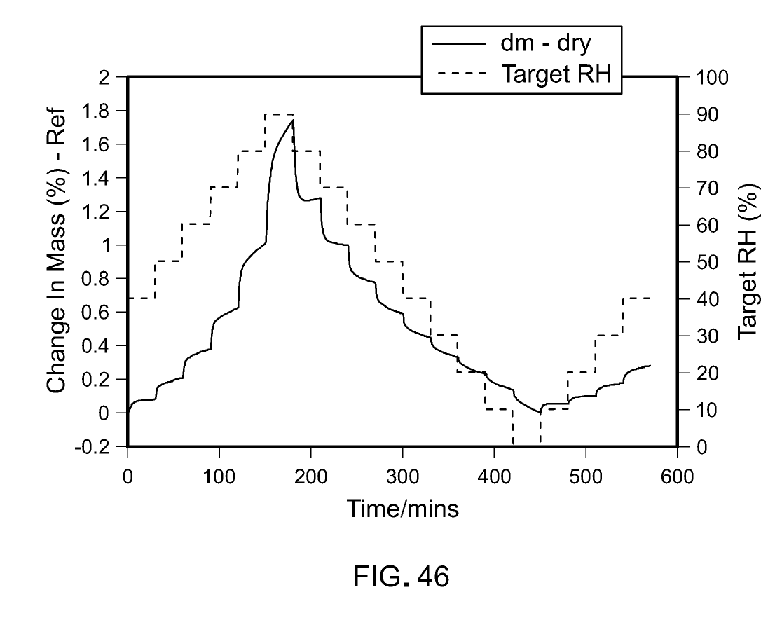

Compound 1 citrate exhibits low hygroscopicity by DVS analysis (FIG. 45) with a mass uptake of around 1.8% at 90% RH. Post-DVS XRPD analysis of the material showed no changes in crystalline form upon exposure to DVS conditions.

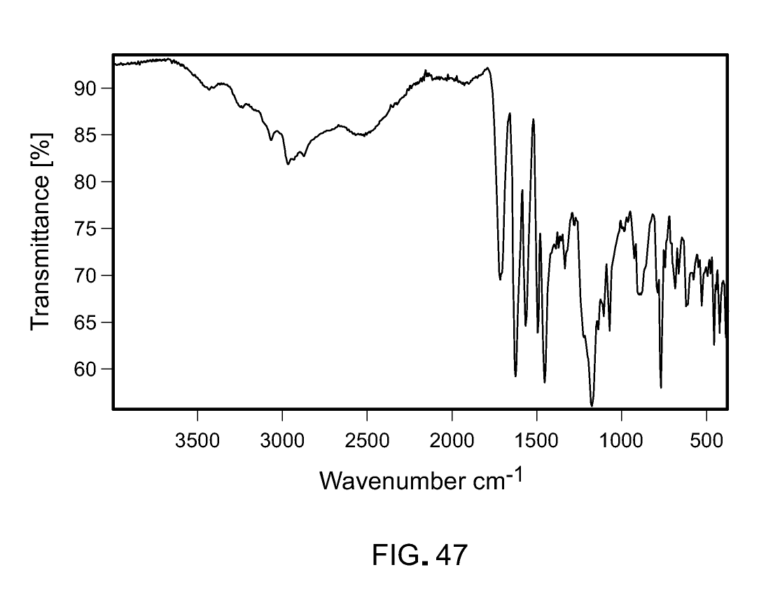

An IR spectrum of Compound 1 citrate was taken for reference which can be found in FIG. 47 with peak lists in Table 31.

TABLE 31

| Wave | Wave | Wave | Wave |
|---|---|---|---|
| Number | Number | Number | Number |
| 3430 | 1497 | 987 | 578 |
| 3066 | 1456 | 929 | 550 |
| 2967 | 1373 | 899 | 532 |
| 2518 | 1338 | 790 | 497 |
| 2033 | 1281 | 771 | 478 |
| 1929 | 1178 | 747 | 459 |
| 1718 | 1141 | 687 | 446 |
| 1626 | 1109 | 667 | 425 |
| 1568 | 1074 | 623 | |

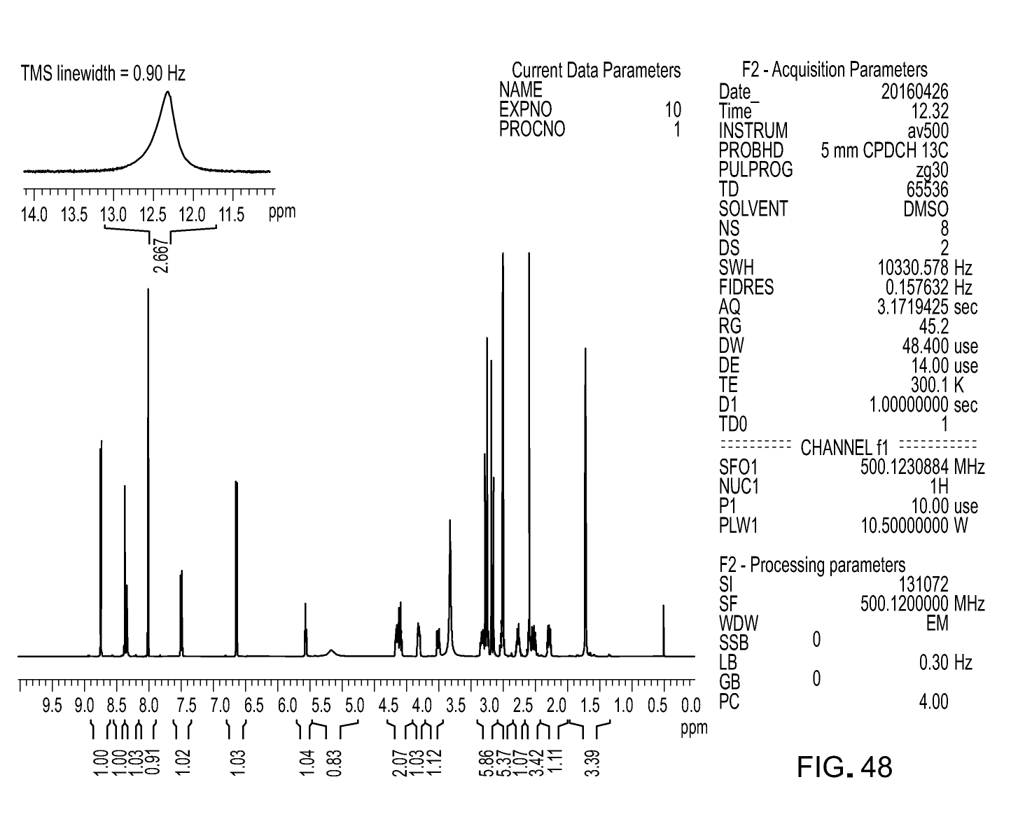

¹H-NMR spectrum shown in FIG. 48 shows 0.97 eq. citric acid and 0.24 eq. acetone. UPLC analysis of Compound 1 citrate gave an average purity of 99.4%. 1 week stability tests at 40° C./75% RH, 80° C. and under ambient light showed no change to form after exposure and no change to purity. Thermodynamic solubility studies of Compound 1 citrate show the salt is highly soluble in un-buffered water and has high solubility at pH 1 with a lower solubility at 4.5 and 6.8. pH and concentration values can be found in Table 32.

TABLE 32

| Sample ID | Concentration (mg/mL) |
|---|---|
| pH 1 | 18.6 |
| pH 4.5 | 0.3 |
| pH 6.8 | 0.9 |
| Un-Buffered Water | 21.0 |

XRPD analysis showed poorly crystalline solids were recovered from pH 1, Compound 1 citrate was recovered from pH 4.5 and un-buffered water and poorly crystalline free base was recovered from pH 6.8. Salt disproportionation studies of Compound 1 citrate showed the recovered material to be poorly crystalline citrate salt by XRPD analysis.

Hydration studies of Compound 1 citrate found poorly crystalline citrate salt recovered from high and low water activities and unknown form, referred to here as Form B, recovered from medium water activity. XRPD diffractogram of the Compound I citrate Form B is shown in FIG. 49.

### Example 10—Preparation and Characterization of Compound 1 Methanesulfonic Acid Salt

A stock solution of methanesulfonic acid was prepared in water (36 μL of methane sulfuric acid in 964 μL H₂O). 400 μL of the appropriate solvent was added to the vial containing the weighed compound 1, 100 μL of the methanesulfonic acid stock solution was then added to the solvent/compound 1 slurry (1.05 eq. of acid to free base). The samples were then temperature cycled between ambient and 40° C. in 4 hour cycles over 24 hrs.

Scale-Up Preparation from Acetone

About 300 mg of Compound 1 was weighed into a vial and a stock solution of methanesulfonic acid was prepared in water (538 μL of acid in 10 mL of water). To the weighed compound 1, 6 mL of acetone was added which was then followed by 1.5 mL of the acid stock solution, this slurry was then temperature cycled for 24 hours (ambient to 40° C. in 4 hour cycles) (1.05 eq. of acid to free base). The resulting clear solution was allowed to evaporate to recover solids; to which a crystal/oil mixture was recovered. To this mixture, acetone was added and the vial sonicated to produce solids. These solids were then filtered and dried for 72 hours under vacuum at ambient temperature.

Observations from the treatment of Compound 1 with methanesulfonic acid are shown in a Table 33 below:

TABLE 33

| Time- | Solvent | | | | | |
|---|---|---|---|---|---|---|
| point | Acetone | Ethanol | Methanol | 2-propanol | TBME | THF |
| Pre- | Slurry | Slurry | Slurry | Slurry | Slurry | Slurry |
| Cycling | | | | | | |
| Post- | Slurry | Clear | Slurry | Clear | Slurry | Clear |
| Cycling | | Solution | | Solution | | Solution |

To the samples which were recovered as clear solutions, 2-3 mg of Compound 1 was added to produce a mobile slurry and the sample temperature cycled for a further 2-3 hours. XRPD analysis of methanesulfonic acid experiments recovered 4 crystalline hits, free base (Form I) recovered from THF and pattern 1 from acetone, methanol and 2-propanol (FIG. 16). Insufficient solids were recovered from ethanol and TBME.

TG/DT Analysis

TGA of crystalline mesylate (FIG. 25) showed a total weight loss of approximately 3% from the outset up to about 200° C. DTA showed an endothermal event at onset about 229° C. (peak at about 232° C.).

Result of Stability Studies

XRPD analysis of post-stability crystalline mesylate recovered from acetone showed no changes to crystallinity or form after exposure to stability conditions. XRPD analysis of post-stability crystalline mesylate recovered from methanol showed a decrease in crystallinity but no changes to form after exposure to stability conditions. XRPD analysis of post-stability crystalline mesylate recovered from isopropanol showed a slight increase in crystallinity but no changes to form after exposure to stability conditions.

Secondary Salt Scale Up

XRPD analysis of the scaled up mesylate from acetone (shown in FIG. 30) showed a different form than seen previously.

TGA showed a series of weight losses with a total of around 9% up to 228° C. (FIG. 31). The weight loss seen at around 120° C. indicates the material to be an acetone solvate. DTA (FIG. 31) showed a small endothermal event at onset around 120° C. (peak at around 125° C.). This event is likely associated with the 6.74% weight loss, which would equate to about 0.59 equivalents of acetone. A larger endothermal “melting” event at onset about 228° C. (peak at about 232° C.). This event is consistent with the earlier collected mesylate TG/DTA.

DSC Analysis in the first heating cycle (FIG. 32) showed a sharp endothermal event at onset around 230° C. (peak at 233° C.). This endothermal event shown is consistent with TG/DTA. At this point, the material had already believed to have been desolvated otherwise there should have been an endothermal event relating to the weight loss.

A broad recrystallization event can be seen in the first cooling cycle with an onset of around 193° C. (peak at around 181° C.) and in the second heating cycle showed an endothermal event at onset around 223° C. (peak at 229° C.).

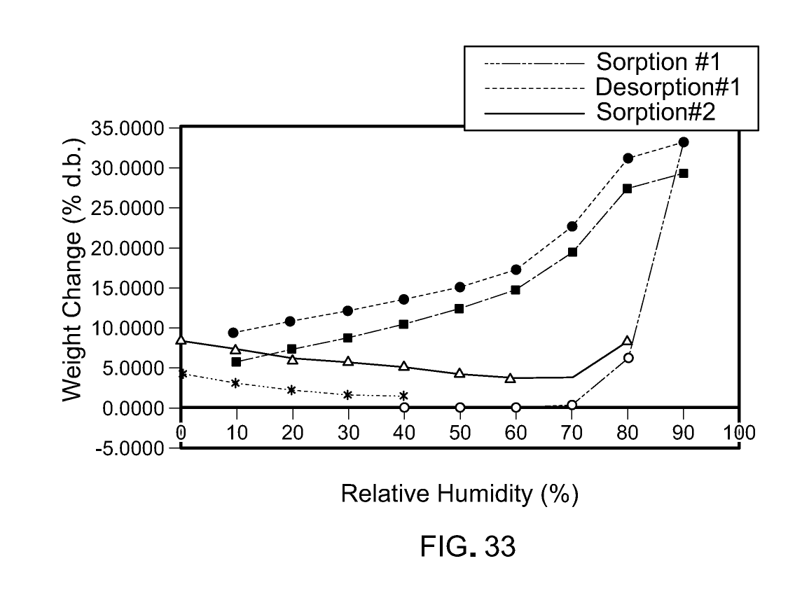

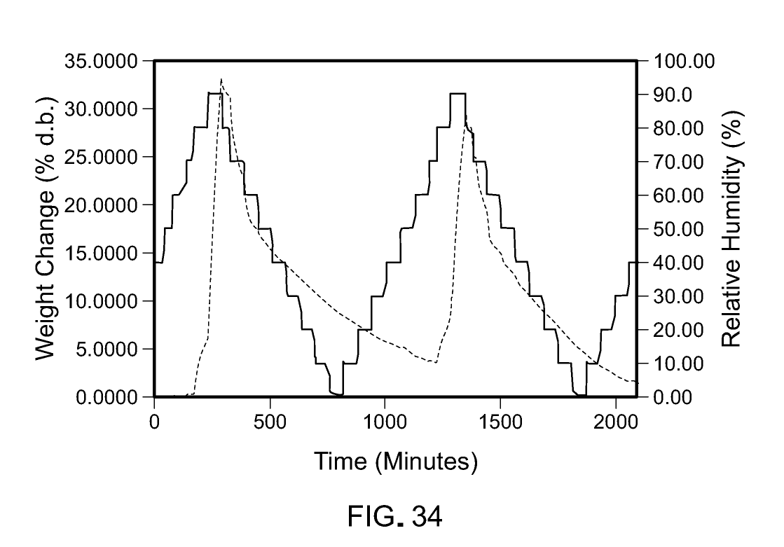

Compound 1 mesylate salt exhibits high hygroscopicity by upon exposure to GVS humidity conditions (FIGS. 33 and 34); mass uptake of about 32% at 90% RH. Post-GVS XRPD analysis of the mesylate salt shows the material to desolvate and become the mesylate form seen in the salt screen. At 30% RH the material deliquesced and upon drying crystallized to the same form seen in the primary salt screen.

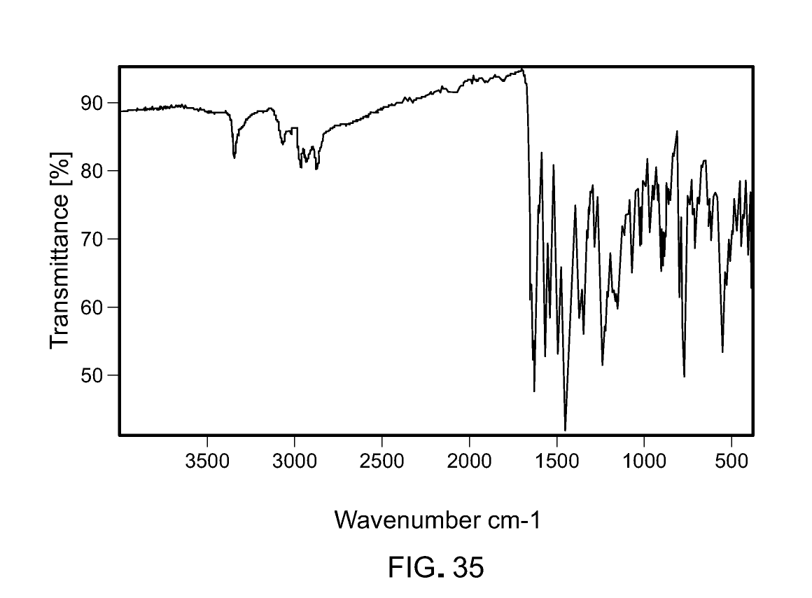

An IR spectrum of Compound 1 mesylate salt was taken for reference which can be found in FIG. 35 and peak listings in Table 34.

TABLE 34

| Wave | Wave | Wave | Wave |
|---|---|---|---|
| Number | Number | Number | Number |
| 3344 | 1538 | 1020 | 720 |
| 3068 | 1492 | 991 | 708 |
| 3020 | 1451 | 964 | 687 |
| 2963 | 1367 | 944 | 633 |
| 2930 | 1347 | 923 | 616 |
| 2870 | 1282 | 904 | 552 |
| 1805 | 1235 | 891 | 528 |
| 1649 | 1167 | 859 | 509 |
| 1626 | 1153 | 796 | 469 |
| 1600 | 1115 | 770 | 442 |
| 1566 | 1071 | 740 | 405 |

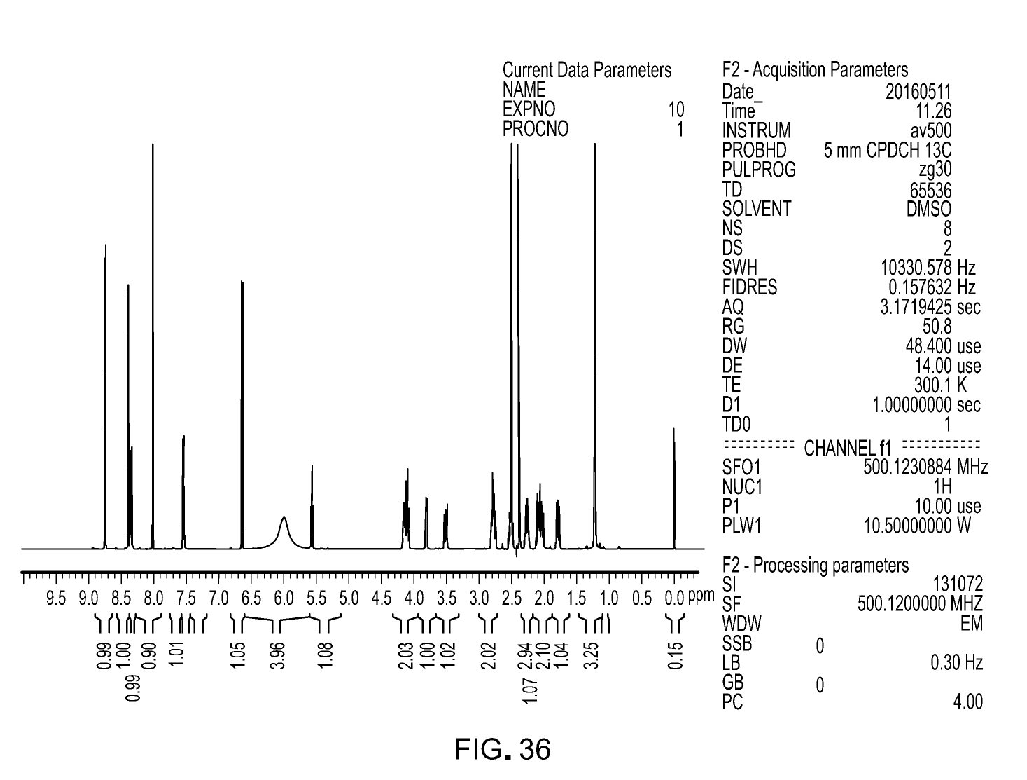

¹H NMR Spectrum shown in FIG. 36 shows about 1 eq. of sulfonic acid. It is not possible to accurately quantify any residual acetone from this data due to spectral overlap but the levels, if there are any, are considered low.

UPLC analysis of Compound 1 mesylate gave an average purity of 99.4%.

1 Week stability tests at 40° C./75% RH, 80° C. and under ambient light showed change to form after exposure by XRPD. However, changes to the mesylate form seen previously in the salt screen and no change to purity.

Thermodynamic solubility studies of Compound 1 mesylate show the salt is moderately soluble in pH 1, 4.5 and unbuffered water. The sample shows low solubility in pH 6.8. pH and concentration values can be found in Table 35.

TABLE 35

| Sample ID | Concentration (mg/mL) |
|---|---|
| pH 1 | 14.3 |
| pH 4.5 | 9.3 |
| pH 6.8 | 1.5 |
| Unbuffered Water | 9.6 |

XRPD analysis showed insufficient solids were recovered from pH 1, mesylate salt was recovered from pH 4.5 and free base was recovered from pH 6.8 and un-buffered water. Salt disproportionation studies of Compound 1 mesylate showed no change to form by XRPD analysis but crystallinity reduced. Hydration studies of Compound 1 mesylate showed mesylate salt recovered from medium water activities, a mixture of free base and salt recovered from low water activities and free base recovered from high water activities.

### Example 11—Preparation and Characterization of Compound 1 1,2-Ethane Disulfonic Acid Salt

250 μL of the appropriate solvent was added to the vials containing 20 mg of compound 1. In a separate vial, 250 μL of the appropriate solvent was added to the vial containing 1,2-ethane disulfonic acid (12.94 mg). The solutions/slurries were then added to the solvent/API solution (1.05 eq. of acid to free base). The samples were then temperature cycled between ambient and 40° C. in 4 hour cycles over 24 hrs.

Observations from the treatment of Compound 1 with 1,2-ethane disulfonic acid are shown in Table 36 below:

TABLE 36

| Time- | Solvent | | | | | |
|---|---|---|---|---|---|---|
| point | Acetone | Ethanol | Methanol | 2-propanol | TBME | THF |
| Pre- | Slurry | Slurry | Slurry | Slurry | Slurry | Slurry |
| Cycling | | | | | | |
| Post- | Yellow | Clear | Slurry | Slurry | Slurry | Slurry |
| Cycling | solution/ | solution | | | | |
| | dark | | | | | |
| | solids | | | | | |

To the samples which were recovered as clear solutions, 2-3 mg of Compound 1 was added to produce a mobile slurry and the sample temperature cycled for a further 2-3 hours. XRPD analysis of 1,2-ethane disulfonic acid experiments recovered 4 crystalline hits, free base (Form I) recovered from acetone, THF and TBME and pattern 1 from 2-propanol (FIG. 14). Insufficient solids were recovered from ethanol and methanol.

Result of Stability Studies

XRPD analysis of post-stability edisylate recovered from isopropanol showed the material to become amorphous after exposure to stability conditions.

### Example 12—Preparation and Characterization of Compound 1 p-Toluene Sulfonic Acid Salt

250 μL of the appropriate solvent was added to the vials containing 20 mg of compound 1. In a separate vial, 250 μL of the appropriate solvent was added to the vial containing p-toluene sulfonic acid (10.84 mg). The solutions/slurries were then added to the solvent/compound 1 solution (1.05 eq. of acid to free base). The samples were then temperature cycled between ambient and 40° C. in 4 hour cycles over 24 hrs.

Observations from the treatment of compound 1 with p-toluene sulfonic acid are shown in Table 37 below:

TABLE 37

| Time- | Solvent | | | | | |
|---|---|---|---|---|---|---|
| point | Acetone | Ethanol | Methanol | 2-propanol | TBME | THF |
| Pre- | Slurry | Slurry | Slurry | Slurry | Slurry | Slurry |
| Cycling | | | | | | |
| Post- | Solid | Clear | Slurry | Slurry | Slurry | Solid |
| Cycling | | Solution | | | | |

To the samples which were recovered as clear solutions, 2-3 mg of Compound 1 was added to produce a mobile slurry and the sample temperature cycled for a further 2-3 hours. XRPD analysis of p-toluene sulfonic acid experiments recovered 4 crystalline hits, free base (Form I) recovered from 2-propanol and TBME and pattern 1 from acetone and THF (FIG. 15). Insufficient solids were recovered from ethanol and methanol.

TG/DT Analysis

TGA of p-toluene sulfonate (FIG. 24) showed a total weight loss of approximately 14% from the outset up to about 250° C. DTA showed an endothermal event at onset about 84° C. (peak at about 90° C.).

Result of Stability Studies

XRPD analysis of post-stability p-toluene sulfonate recovered from acetone showed the material to become amorphous after exposure to stability conditions.

### Example 13—Preparation and Characterization of Compound 1 Oxalic Acid Salt

250 μL of the appropriate solvent was added to the vials containing 20 mg of compound 1. In a separate vial, 250 μL of the appropriate solvent was added to the vial containing oxalic acid (5.08 mg). The solutions/slurries were then added to the solvent/compound 1 solution (1.05 eq. of acid to free base). The samples were then temperature cycled between ambient and 40° C. in 4 hour cycles over 24 hrs.

Observations from the treatment of Compound 1 with oxalic acid are shown in Table 38 below:

TABLE 38

| Time- | Solvent | | | | | |
|---|---|---|---|---|---|---|
| point | Acetone | Ethanol | Methanol | 2-propanol | TBME | THF |
| Pre- | Slurry | Slurry | Slurry | Slurry | Slurry | Slurry |
| Cycling | | | | | | |
| Post- | Slurry | Slurry | Solid | Slurry | Slurry | Slurry |
| Cycling | | | | | | |

XRPD analysis of oxalic acid experiments recovered 6 crystalline hits, free base (Form I) recovered from acetone (which was mostly amorphous), 2-propanol, THF and TBME and pattern 1 recovered from ethanol and methanol (FIG. 19).

TG/DT Analysis

TGA of oxalate (FIG. 26) showed a total weight loss of approximately 17% from the outset up to about 300° C. DTA showed a small endothermal event at onset about 314° C. (peak at about 317° C.).

Result of Stability Studies

XRPD analysis of post-stability oxalate recovered from ethanol showed a change in crystallinity and form after exposure to stability conditions. XRPD analysis of post-stability oxalate recovered from methanol showed no change to crystallinity however, changes to form were seen after exposure to stability conditions.

### Example 14—Preparation and Characterization of Compound 1 Fumaric Acid Salt

250 μL of the appropriate solvent was added to the vials containing 20 mg of compound 1. In a separate vial, 250 μL of the appropriate solvent was added to the vial containing fumaric acid (6.48 mg). The solutions/slurries were then added to the solvent/compound 1 solution (1.05 eq. of acid to free base). The samples were then temperature cycled between ambient and 40° C. in 4 hour cycles over 24 hrs.

Observations from the treatment of Compound 1 with fumaric acid are shown in Table 39 below

TABLE 39

| Time- | Solvent | | | | | |
|---|---|---|---|---|---|---|
| point | Acetone | Ethanol | Methanol | 2-propanol | TBME | THF |
| Pre- | Slurry | Slurry | Slurry | Slurry | Slurry | Slurry |
| Cycling | | | | | | |
| Post- | Slurry | Slurry | Slurry | Slurry | Slurry | Slurry |
| Cycling | | | | | | |

XRPD analysis of fumaric acid experiments recovered 6 crystalline hits, free base (Form I) recovered from ethanol, methanol, 2-propanol, THF and TBME and pattern 1 recovered from acetone (FIG. 20).

TG/DT Analysis

TGA of crystalline fumarate (FIG. 27) showed a total weight loss of approximately 22% from the outset up to about 250° C. DTA showed several endothermal events; first event at onset about 164° C. (peak at about 166° C.), second event at onset about 189° C. (peak at about 191° C.), third event at onset of about 198° C. (peak at about 201° C.) and forth event at onset about 310° C. (peak at about 312° C.).

Result of Stability Studies

XRPD analysis of post-stability crystalline fumarate recovered from acetone showed a slight decrease to crystallinity however, no change to form after exposure to stability conditions.

### Example 15—Preparation and Characterization of Compound 1 L-Malic Acid Salt

250 μL of the appropriate solvent was added to the vials containing 20 mg of compound 1. In a separate vial, 250 μL of the appropriate solvent was added to the vial containing L-malic acid (7.49 mg). The solutions/slurries were then added to the solvent/compound 1 solution (1.05 eq. of acid to free base). The samples were then temperature cycled between ambient and 40° C. in 4 hour cycles over 24 hrs.

Observations from the treatment of Compound 1 with L-malic acid are shown in Table 40 below:

TABLE 40

| Time- | Solvent | | | | | |
|---|---|---|---|---|---|---|
| point | Acetone | Ethanol | Methanol | 2-propanol | TBME | THF |
| Pre- | Slurry | Slurry | Slurry | Slurry | Slurry | Slurry |
| Cycling | | | | | | |
| Post- | Slurry | Slurry | Slurry | Slurry | Slurry | Solid |
| Cycling | | | | | | |

XRPD analysis of L-malic acid experiments recovered 6 crystalline hits, free base (Form I) recovered from acetone (with a large amount of preferred orientation), ethanol, methanol, 2-propanol, and THF and pattern 1 recovered from TBME (FIG. 22).

TG/DT Analysis

TGA of crystalline L-malate (FIG. 28) showed a total weight loss of approximately 26% from the outset up to about 250° C. DTA showed several endothermal events; first event at onset about 158° C. (peak at about 162° C.) and the second event at onset about 310° C. (peak at about 313° C.).

Result of Stability Studies

XRPD analysis of post-stability crystalline L-malate prepared from TBME showed no change to crystallinity and form after exposure to stability conditions.

### Example 16—Preparation and Characterization of Compound 1 Succinic Acid Salt

250 μL of the appropriate solvent was added to the vials containing 20 mg of compound 1. In a separate vial, 250 μL of the appropriate solvent was added to the vial containing succinic acid (6.59 mg). The solutions/slurries were then added to the solvent/compound 1 solution (1.05 eq. of acid to free base). The samples were then temperature cycled between ambient and 40° C. in 4 hour cycles over 24 hrs.

Observations from the treatment of Compound 1 with succinic acid are shown in Table 41 below:

TABLE 41

| Time- | Solvent | | | | | |
|---|---|---|---|---|---|---|
| point | Acetone | Ethanol | Methanol | 2-propanol | TBME | THF |
| Pre- | Slurry | Slurry | Slurry | Slurry | Slurry | Slurry |
| Cycling | | | | | | |
| Post- | Slurry | Slurry | Slurry | Slurry | Slurry | Slurry |
| Cycling | | | | | | |

XRPD analysis of succinic acid experiments recovered 6 crystalline hits, free base (Form I) recovered from ethanol, methanol, 2-propanol, THF and TBME and pattern 1 recovered from acetone (FIG. 23).

TG/DT Analysis

TGA of succinate FIG. 29) showed a total weight loss of approximately 22% from the outset about 210° C. DTA showed several endothermal events; first event at onset about 147° C. (peak at about 151° C.) and the second event at onset about 315° C. (peak at about 315° C.).

Result of Stability Studies

XRPD analysis of post-stability crystalline succinate recovered from acetone showed a decrease in crystallinity but no change to form after exposure to stability conditions.

### Example 17—Preparation and Characterization of Compound 1 Hydrochloric Acid Salt

A stock solution of HCl was prepared in water (46 μL of HCl in 954 μL H₂O). 400 μL of the appropriate solvent was added to the vial containing the weighed compound 1, 100 μL of the HCl stock solution was then added to the solvent/compound 1 slurry (1.05 eq. of acid to free base). The samples were then temperature cycled between ambient and 40° C. in 4 hour cycles over 24 hrs.

Observations from the treatment of Compound 1 with HCl are shown in Table 42 below:

TABLE 42

| Time- | Solvent | | | | | |
|---|---|---|---|---|---|---|
| point | Acetone | Ethanol | Methanol | 2-Propanol | TBME | THF |
| Pre- | Slurry | Slurry | Slurry | Slurry | Slurry | Slurry |
| Cycling | | | | | | |
| Post- | Slurry | Clear | Clear | Clear | Clear | Slurry |
| Cycling | | Solution | Solution | Solution | Solution | |

To the samples which were recovered as clear solutions, 2-3 mg of compound 1 was added to produce a mobile slurry and the sample temperature cycled for a further 2-3 hours. Further solids were recovered from ethanol, methanol, 2-propanol, TBME and THF through anti-solvent additions described in Materials and methods section. XRPD analysis of HCl experiments recovered 6 crystalline hits. Freebase (Form I) was recovered from all solvent systems analyzed.

### Example 18—Preparation and Characterization of Compound 1 Sulfuric Acid Salt

A stock solution of sulfuric acid was prepared in water (31 μL of sulfuric acid in 969 μL H₂O). 400 μL of the appropriate solvent was added to the vial containing the weighed compound 1, 100 μL of the sulfuric acid stock solution was then added to the solvent/compound 1 slurry (1.05 eq. of acid to free base). The samples were then temperature cycled between ambient and 40° C. in 4 hour cycles over 24 hrs.

Observations from the treatment of Compound 1 with sulfuric acid are shown in Table 43 below:

TABLE 43

| Time- | Solvent | | | | | |
|---|---|---|---|---|---|---|
| point | Acetone | Ethanol | Methanol | 2-propanol | TBME | THF |
| Pre- | Slurry | Slurry | Slurry | Slurry | Slurry | Slurry |
| Cycling | | | | | | |
| Post- | Clear | Clear | Clear | Clear | Clear | Clear |
| Cycling | solution | solution | solution | solution | solution | solution |

To the samples which were recovered as clear solutions, 2-3 mg of Compound 1 was added to produce a mobile slurry and the sample temperature cycled for a further 2-3 hours. Further solids were recovered from ethanol, methanol, 2-propanol, TBME and THF through anti-solvent additions described. XRPD analysis of sulfuric acid experiments recovered 6 amorphous hits from all solvent systems analyzed.

### Example 19—Preparation and Characterization of Compound 1 Naphthalene-2-Sulfonic Acid Salt

250 μL of the appropriate solvent was added to the vials containing 20 mg of compound 1. In a separate vial, 250 μL of the appropriate solvent was added to the vial containing naphthalene-2-sulfonic acid (14.14 mg). The solutions/slurries were then added to the solvent/compound 1 solution (1.05 eq. of acid to free base). The samples were then temperature cycled between ambient and 40° C. in 4 hour cycles over 24 hrs.

Observations from the treatment of Compound 1 with naphthalene-2-sulfonic acid are shown in Table 44 below.

TABLE 44

| Time- | Solvent | | | | | |
|---|---|---|---|---|---|---|
| point | Acetone | Ethanol | Methanol | 2-propanol | TBME | THF |
| Pre- | Slurry | Slurry | Slurry | Slurry | Slurry | Slurry |
| Cycling | | | | | | |
| Post- | Clear | Clear | Clear | Clear | Slurry | Solid |
| Cycling | Solution | Solution | Solution | Solution | | |

To the samples which were recovered as clear solutions, 2-3 mg of Compound 1 was added to produce a mobile slurry and the sample temperature cycled for a further 2-3 hours. XRPD analysis of naphthalene-2-sulfonic acid experiments recovered 3 crystalline hits, free base (Form I) recovered from ethanol, THF and TBME. Insufficient solids were recovered from acetone, methanol and 2-propanol.

### Example 20—Preparation and Characterization of Compound 1 2-Hydroxy Ethanesulfonic Acid Salt

250 μL of the appropriate solvent was added to the vials containing 20 mg of compound 1. In a separate vial, 250 μL of the appropriate solvent was added to the vial containing 2-hydroxy ethanesulfonic acid (8.19 mg). The solutions/slurries were then added to the solvent/compound 1 solution. The samples were then temperature cycled between ambient and 40° C. in 4 hour cycles over 24 hrs.

Observations from the treatment of Compound 1 with 2-hydroxy-ethanesulfonic acid are shown in Table 45 below:

TABLE 45

| Time- | Solvent | | | | | |
|---|---|---|---|---|---|---|
| point | Acetone | Ethanol | Methanol | 2-propanol | TBME | THF |
| Pre- | Slurry | Slurry | Slurry | Slurry | Slurry | Slurry |
| Cycling | | | | | | |
| Post- | Slurry | Slurry | Solid | Slurry | Slurry | Slurry |
| Cycling | | | | | | |

XRPD analysis of 2-hydroxy ethanesulfonic acid experiments recovered 6 crystalline hits, free base (Form I) recovered from all solvent systems analyzed.

### Example 21—Preparation and Characterization of Compound 1 L-Aspartic Acid Salt

250 μL of the appropriate solvent was added to the vials containing 20 mg of compound 1. In a separate vial, 250 μL of the appropriate solvent was added to the vial containing L-aspartic acid (7.36 mg). The solutions/slurries were then added to the solvent/compound 1 solution (1.05 eq. of acid to free base). The samples were then temperature cycled between ambient and 40° C. in 4 hour cycles over 24 hrs.

Observations from the treatment of Compound 1 with L-aspartic acid are shown in Table 46 below:

TABLE 46

| Time- | Solvent | | | | | |
|---|---|---|---|---|---|---|
| point | Acetone | Ethanol | Methanol | 2-propanol | TBME | THF |
| Pre- | Slurry | Slurry | Slurry | Slurry | Slurry | Slurry |
| Cycling | | | | | | |
| Post- | Slurry | Slurry | Slurry | Slurry | Slurry | Slurry |
| Cycling | | | | | | |

XRPD analysis of L-aspartic acid experiments recovered 6 crystalline hits, free base (Form I) recovered from all solvent systems analyzed.

### Example 22—Preparation and Characterization of Compound 1 Maleic Acid Salt

250 μL of the appropriate solvent was added to the vials containing 20 mg of compound 1. In a separate vial, 250 μL of the appropriate solvent was added to the vial containing maleic acid (6.48 mg). The solutions/slurries were then added to the solvent/compound 1 solution (1.05 eq. of acid to free base). The samples were then temperature cycled between ambient and 40° C. in 4 hour cycles over 24 hrs.

Observations from the treatment of Compound 1 with maleic acid are shown in Table 47 below:

TABLE 47

| Time- | Solvent | | | | | |
|---|---|---|---|---|---|---|
| point | Acetone | Ethanol | Methanol | 2-propanol | TBME | THF |
| Pre- | Slurry | Slurry | Slurry | Slurry | Slurry | Slurry |
| Cycling | | | | | | |
| Post- | Solid | Slurry | Gum | Slurry | Slurry | Slurry |
| Cycling | | | | | | |

XRPD analysis of maleic acid experiments recovered 6 crystalline hits, free base (Form I) recovered from all solvent systems analyzed.

### Example 23—Preparation and Characterization of Compound 1 Phosphoric Acid Salt

250 μL of the appropriate solvent was added to the vials containing 20 mg of compound 1. In a separate vial, 250 μL of the appropriate solvent was added to the vial containing phosphoric acid (5.42 mg). The solutions/slurries were then added to the solvent/compound 1 solution (1.05 eq. of acid to free base). The samples were then temperature cycled between ambient and 40° C. in 4 hour cycles over 24 hrs.

Observations from the treatment of Compound 1 with phosphoric acid are shown in Table 48 below:

TABLE 48

| Time- | Solvent | | | | | |
|---|---|---|---|---|---|---|
| point | Acetone | Ethanol | Methanol | 2-propanol | TBME | THF |
| Pre- | Slurry | Slurry | Slurry | Slurry | Slurry | Slurry |
| Cycling | | | | | | |
| Post- | Solid | Clear | Clear | Slurry | Slurry | Slurry |
| Cycling | | Solution | Solution | | | |

To the samples which were recovered as clear solutions, 2-3 mg of Compound 1 was added to produce a mobile slurry and the sample temperature cycled for a further 2-3 hours. XRPD analysis of phosphoric acid experiments recovered 3 crystalline hits, free base (Form I) recovered from all solvent systems analyzed.

### Example 24—Preparation and Characterization of Compound 1 Ethanesulfonic Acid Salt

A stock solution of ethane sulfonic acid was prepared in water (47 μL of sulfuric acid in 953 μL H₂O). 400 μL of the appropriate solvent was added to the vial containing the weighed compound 1, 100 μL of the ethane sulfonic acid stock solution was then added to the solvent/compound 1 slurry (1.05 eq. of acid to free base). The samples were then temperature cycled between ambient and 40° C. in 4 hour cycles over 24 hrs.

Observations from the treatment of Compound 1 with ethanesulfonic acid are shown Table 49 below:

TABLE 49

| Time- | Solvent | | | | | |
|---|---|---|---|---|---|---|
| point | Acetone | Ethanol | Methanol | 2-propanol | TBME | THF |
| Pre- | Slurry | Slurry | Slurry | Slurry | Slurry | Slurry |
| Cycling | | | | | | |
| Post- | Clear | Clear | Clear | Clear | Clear | Slurry |
| Cycling | Solution | Solution | Solution | Solution | Solution | |

To the samples which were recovered as clear solutions, 2-3 mg of Compound 1 was added to produce a mobile slurry and the sample temperature cycled for a further 2-3 hours. XRPD analysis of ethanesulfonic acid experiments recovered 4 crystalline hits, free base (Form I) recovered from acetone, THF and TBME. Insufficient solids were recovered from methanol, ethanol and 2-propanol.

### Example 25—Preparation and Characterization of Compound 1 L-Glutamic Acid Salt

250 μL of the appropriate solvent was added to the vials containing 20 mg of compound 1. In a separate vial, 250 μL of the appropriate solvent was added to the vial containing L-glutamic acid (8.13 mg). The solutions/slurries were then added to the solvent/compound 1 solution (1.05 eq. of acid to free base). The samples were then temperature cycled between ambient and 40° C. in 4 hour cycles over 24 hrs.

Observations from the treatment of Compound 1 with L-glutamic acid are shown in Table 50 below:

TABLE 50

| Time- | Solvent | | | | | |
|---|---|---|---|---|---|---|
| point | Acetone | Ethanol | Methanol | 2-propanol | TBME | THF |
| Pre- | Slurry | Slurry | Slurry | Slurry | Slurry | Slurry |
| Cycling | | | | | | |
| Post- | Slurry | Slurry | Slurry | Slurry | Slurry | Slurry |
| Cycling | | | | | | |

XRPD analysis of L-glutamic acid experiments recovered 6 crystalline hits, free base (Form I) recovered from all solvent systems analyzed.

### Example 26—Preparation and Characterization of Compound 1 L-Tartaric Acid Salt

250 μL of the appropriate solvent was added to the vials containing 20 mg of compound 1. In a separate vial, 250 μL of the appropriate solvent was added to the vial containing L-tartaric acid (8.34 mg). The solutions/slurries were then added to the solvent/compound 1 solution. The samples were then temperature cycled between ambient and 40° C. in 4 hour cycles over 24 hrs.

Observations from the treatment of Compound 1 with L-tartaric acid are shown in Table 51 below:

TABLE 51

| Time- | Solvent | | | | | |
|---|---|---|---|---|---|---|
| point | Acetone | Ethanol | Methanol | 2-propanol | TBME | THF |
| Pre- | Slurry | Slurry | Slurry | Slurry | Slurry | Slurry |
| Cycling | | | | | | |
| Post- | Slurry | Slurry | Slurry | Slurry | Slurry | Slurry |
| Cycling | | | | | | |

XRPD analysis of L-tartaric acid experiments recovered 6 crystalline hits, free base (Form I) recovered from all solvent systems analyzed.

### Example 27—Preparation and Characterization of Compound 1 D-Glucuronic Acid Salt

250 μL of the appropriate solvent was added to the vials containing 20 mg of compound 1. In a separate vial, 250 μL of the appropriate solvent was added to the vial containing D-glucuronic acid (10.73 mg). The solutions/slurries were then added to the solvent/compound 1 solution (1.05 eq. of acid to free base). The samples were then temperature cycled between ambient and 40° C. in 4 hour cycles over 24 hrs.

Observations from the treatment of Compound 1 with D-glucuronic acid are shown in Table 52 below:

TABLE 52

| Time- | Solvent | | | | | |
|---|---|---|---|---|---|---|
| point | Acetone | Ethanol | Methanol | 2-propanol | TBME | THF |
| Pre- | Slurry | Slurry | Slurry | Slurry | Slurry | Slurry |
| Cycling | | | | | | |
| Post- | Slurry | Slurry | Slurry | Slurry | Slurry | Slurry |
| Cycling | | | | | | |

XRPD analysis of D-glucuronic acid experiments recovered 6 crystalline hits, free base (Form I) recovered from all solvent systems analyzed.

### Example 28—Preparation and Characterization of Compound 1 Hippuric Acid Salt

250 μL of the appropriate solvent was added to the vials containing 20 mg of compound 1. In a separate vial, 250 μL of the appropriate solvent was added to the vial containing hippuric acid (10.1 mg). The solutions/slurries were then added to the solvent/compound 1 solution (1.05 eq. of acid to free base). The samples were then temperature cycled between ambient and 40° C. in 4 hour cycles over 24 hrs.

Observations from the treatment of Compound 1 with hippuric acid are shown in Table 53 below:

TABLE 53

| Time- | Solvent | | | | | |
|---|---|---|---|---|---|---|
| point | Acetone | Ethanol | Methanol | 2-propanol | TBME | THF |
| Pre- | Slurry | Slurry | Slurry | Slurry | Slurry | Slurry |
| Cycling | | | | | | |
| Post- | Slurry | Slurry | Slurry | Slurry | Slurry | Slurry |
| Cycling | | | | | | |

XRPD analysis of hippuric acid experiments recovered 6 crystalline hits, free base (Form I) recovered from all solvent systems analyzed.

### Example 29—Preparation and Characterization of Compound 1 D-Gluconic Acid Salt

A stock solution of D-gluconic acid was prepared in water (176 μL of D-gluconic acid in 824 μL H₂O). 400 μL of the appropriate solvent was added to the vial containing the weighed compound 1, 100 μL of the D-gluconic stock solution was then added to the solvent/compound 1 slurry (1.05 eq. of acid to free base). The samples were then temperature cycled between ambient and 40° C. in 4 hour cycles over 24 hrs.

Observations from the treatment of Compound 1 with D-gluconic acid are shown in Table 54 below:

TABLE 54

| Time- | Solvent | | | | | |
|---|---|---|---|---|---|---|
| point | Acetone | Ethanol | Methanol | 2-propanol | TBME | THF |
| Pre- | Slurry | Slurry | Slurry | Slurry | Slurry | Slurry |
| Cycling | | | | | | |
| Post- | Clear | Clear | Clear | Clear | Clear | Slurry |
| Cycling | Solution | Solution | Solution | Solution | Solution | |

To the samples which were recovered as clear solutions, 2-3 mg of Compound 1 was added to produce a mobile slurry and the sample temperature cycled for a further 2-3 hours. XRPD analysis of D-gluconic acid experiments recovered 1 crystalline hit, free base (Form I) recovered from TBME and insufficient solids recovered from acetone, ethanol, methanol, 2-propanol and THF.

### Example 30—Preparation and Characterization of Compound 1 DL-Lactic Acid Salt

A stock solution of DL-lactic acid was prepared in water (48 μL of DL-lactic acid in 952 μL H₂O). 400 μL of the appropriate solvent was added to the vial containing the weighed compound 1, 100 μL of the DL-lactic acid stock solution was then added to the solvent/compound 1 slurry (1.05 eq. of acid to free base). The samples were then temperature cycled between ambient and 40° C. in 4 hour cycles over 24 hrs.

Observations from the treatment of Compound 1 with DL-lactic acid are shown in Table 55 below:

TABLE 55

| Time- | Solvent | | | | | |
|---|---|---|---|---|---|---|
| point | Acetone | Ethanol | Methanol | 2-propanol | TBME | THF |
| Pre- | Slurry | Slurry | Slurry | Slurry | Slurry | Slurry |
| Cycling | | | | | | |
| Post- | Clear | Clear | Clear | Clear | Gum | Slurry |
| Cycling | Solution | Solution | Solution | Solution | | |

To the samples which were recovered as clear solutions, 2-3 mg of Compound 1 was added to produce a mobile slurry and the sample temperature cycled for a further 2-3 hours. XRPD analysis of DL-lactic acid experiments recovered 5 crystalline hits, free base (Form I) recovered from acetone, ethanol, methanol, THF and TBME. Insufficient solids recovered from 2-propanol.

### Example 31—Preparation and Characterization of Compound 1 L-Ascorbic Acid Salt

250 μL of the appropriate solvent was added to the vials containing 20 mg of Compound 1. In a separate vial, 250 μL of the appropriate solvent was added to the vial containing L-ascorbic acid (9.73 mg). The solutions/slurries were then added to the solvent/compound 1 solution (1.05 eq. of acid to free base). The samples were then temperature cycled between ambient and 40° C. in 4 hour cycles over 24 hrs.

Observations from the treatment of Compound 1 with L-ascorbic acid are shown in Table 56 below:

TABLE 56

| Time- | Solvent | | | | | |
|---|---|---|---|---|---|---|
| point | Acetone | Ethanol | Methanol | 2-propanol | TBME | THF |
| Pre- | Slurry | Slurry | Slurry | Slurry | Slurry | Slurry |
| Cycling | | | | | | |
| Post- | Slurry | Slurry | Slurry | Slurry | Slurry | Slurry |
| Cycling | | | | | | |

XRPD analysis of L-ascorbic acid experiments recovered 6 crystalline hits, free base (Form I) recovered from all solvent systems analyzed.

### Example 32—Preparation and Characterization of Compound 1 Benzoic Acid Salt

250 μL of the appropriate solvent was added to the vials containing 20 mg of compound 1. In a separate vial, 250 μL of the appropriate solvent was added to the vial containing benzoic acid (6.82 mg). The solutions/slurries were then added to the solvent/compound 1 solution (1.05 eq. of acid to free base). The samples were then temperature cycled between ambient and 40° C. in 4 hour cycles over 24 hrs.

Observations from the treatment of Compound 1 with benzoic acid are shown in Table 57 below:

TABLE 57

| Time- | Solvent | | | | | |
|---|---|---|---|---|---|---|
| point | Acetone | Ethanol | Methanol | 2-propanol | TBME | THF |
| Pre- | Slurry | Slurry | Slurry | Slurry | Slurry | Slurry |
| Cycling | | | | | | |
| Post- | Slurry | Slurry | Slurry | Slurry | Slurry | Slurry |
| Cycling | | | | | | |

XRPD analysis of benzoic acid experiments recovered 6 crystalline hits, free base (Form I) recovered from all solvent systems analyzed.

### Example 33. Preparation of Pharmaceutical Composition Comprising Compound 1 and a Compounding Agent

Suspension Components:

Water—98.15%

Colloidal MCC—1.0%

Xanthan gum—0.4%

Carrageenan—0.1%

Calcium sulfate—0.1%

Sucralose—0.25%

490.75 g of water were heated to ˜70 C. While stirring, 2.0 g of xanthan gum, 0.5 g of carrageenan (iota grade), 0.5 g of calcium sulfate, and 1.25 g of sucralose were added. Stirring was continued until dissolved. 5 g of colloidal microcrystalline cellulose were weighed. While stirring vigorously, colloidal microcrystalline cellulose was slowly added, with vigorous stirring to reduce clumping. The heat was turned off. Stirring was continued until colloidal microcrystalline cellulose was fully dispersed. The suspension was observed to be opaque and did not settle over time.
To prepare a 20 mg/mL suspension of Compound 1, 10.0 g of the compound were weighed. The 10 compound was added to the suspension (adding the suspension to the compound also worked). The mixture was shaken vigorously until well suspended and no visible clumps of Compound 1 remained.

It is to be understood that while the present application has been described in conjunction with the detailed description thereof, the foregoing description is intended to illustrate and not limit the scope of the present application, which is defined by the scope of the appended claims. Other aspects, advantages, and modifications are within the scope of the following claims.

1. A pharmaceutical composition comprising 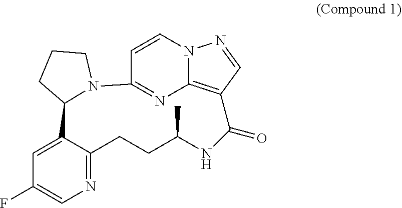 [SMILES: C[C@@H]1CCc2ncc(F)cc2[C@H]2CCCN2c2ccn3ncc(c3n2)C(=O)N1]
and an aqueous compounding agent comprising microcrystalline cellulose, carboxymethylcellulose sodium, xanthan gum, carrageenan, or a combination thereof.

2. The pharmaceutical composition of claim 1, further comprising at least one of citric acid, a citrate, a lactate, a phosphate, a maleate, a tartrate, a succinate, a sulfate, or an acetate.

3. The pharmaceutical composition of claim 2, wherein the composition comprises trisodium phosphate, sodium phosphate, citric acid, calcium sulfate, or a combination thereof.

4. The pharmaceutical composition of claim 1, wherein the composition has a pH of about 3 to about 8.

5. The pharmaceutical composition of claim 1, further comprising a sweetener.

6. The pharmaceutical composition of claim 5, wherein the sweetener comprises sucrose, saccharin, mannitol, sorbitol, dextrose, acesulfame, aspartame, fructose, maltitol, sucralose, or a combination thereof, wherein the sweetener or at least one sweetener in a combination of sweeteners is optionally in a salt form.

7. The pharmaceutical composition of claim 6, wherein the sweetener comprises sucralose.

8. A pharmaceutical composition comprising:
(a) 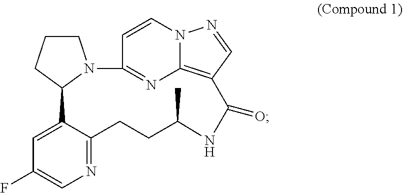 [SMILES: C[C@@H]1CCc2ncc(F)cc2[C@H]2CCCN2c2ccn3ncc(c3n2)C(=O)N1]
(b) a compounding agent comprising microcrystalline cellulose, carboxymethylcellulose sodium, xanthan gum, carrageenan, or a combination thereof;
(c) at least one of citric acid, a citrate, a lactate, a phosphate, a maleate, a tartrate, a succinate, a sulfate, or an acetate;
and optionally
(d) a sweetener;
wherein the composition has a pH of about 3 to about 8.

9. A pharmaceutical composition comprising:
Compound 1;
about 0.1 wt. % to about 2.0 wt. % of microcrystalline cellulose;
about 0.1 wt. % to about 1.0 wt. % of xanthan gum;
about 0.01 wt. % to about 1.0 wt. % of carrageenan;
and
about 0.01 wt. % to about 1.0 wt. % of CaSO₄.

10. The pharmaceutical composition of claim 1, wherein Compound 1 is present in a diastereomeric excess (d.e.) of at least 80% relative to the diastereomeric compound of formula I′:

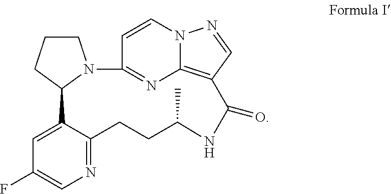 [SMILES: C[C@H]1CCc2ncc(F)cc2[C@H]2CCCN2c2ccn3ncc(c3n2)C(=O)N1]

11.-13. (canceled)

14. The pharmaceutical composition of claim 10, wherein Compound 1 is present in a d.e. of at least 96% relative to the compound of formula I′.

15. (canceled)

16. The pharmaceutical composition of claim 1, wherein Compound 1 is present as crystalline Form.

17. The pharmaceutical composition of claim 1, wherein the pharmaceutical composition is a suspension.

18. A method of treating a Trk-associated cancer in a subject, the method comprising administering to the subject in need thereof a therapeutically effective amount of the pharmaceutical composition of claim 1.

19. The method of claim 18, wherein the cancer is selected from the group consisting of adenocarcinoma, adrenal gland cortical carcinoma, adrenal gland neuroblastoma, anus squamous cell carcinoma, appendix adenocarcinoma, bladder urothelial carcinoma, bile duct adenocarcinoma, bladder carcinoma, bladder urothelial carcinoma, bone chordoma, bone marrow leukemia lymphocytic chronic, bone marrow leukemia non-lymphocytic acute myelocytic, bone marrow lymph proliferative disease, bone marrow multiple myeloma, bone sarcoma, brain astrocytoma, brain glioblastoma, brain medulloblastoma, brain meningioma, brain oligodendroglioma, breast adenoid cystic carcinoma, breast carcinoma, breast ductal carcinoma in situ, breast invasive ductal carcinoma, breast invasive lobular carcinoma, breast metaplastic carcinoma, cervix neuroendocrine carcinoma, cervix squamous cell carcinoma, colon adenocarcinoma, colon carcinoid tumor, duodenum adenocarcinoma, endometrioid tumor, esophagus adenocarcinoma, eye intraocular melanoma, eye intraocular squamous cell carcinoma, eye lacrimal duct carcinoma, fallopian tube serous carcinoma, gallbladder adenocarcinoma, gallbladder glomus tumor, gastroesophageal junction adenocarcinoma, head and neck adenoid cystic carcinoma, head and neck carcinoma, head and neck neuroblastoma, head and neck squamous cell carcinoma, kidney chromophore carcinoma, kidney medullary carcinoma, kidney renal cell carcinoma, kidney renal papillary carcinoma, kidney sarcomatoid carcinoma, kidney urothelial carcinoma, leukemia lymphocytic, liver cholangiocarcinoma, liver hepatocellular carcinoma, lung adenocarcinoma, lung adenosquamous carcinoma, lung atypical carcinoid, lung carcinosarcoma, lung large cell neuroendocrine carcinoma, lung non-small cell lung carcinoma, lung sarcoma, lung sarcomatoid carcinoma, lung small cell carcinoma, lung small cell undifferentiated carcinoma, lung squamous cell carcinoma, lymph node lymphoma diffuse large B cell, lymph node lymphoma follicular lymphoma, lymph node lymphoma mediastinal B-cell, lymph node lymphoma plasmablastic lung adenocarcinoma, lymphoma follicular lymphoma, lymphoma, non-Hodgkin's lymphoma, nasopharynx and paranasal sinuses undifferentiated carcinoma, ovary carcinoma, ovary carcinosarcoma, ovary clear cell carcinoma, ovary epithelial carcinoma, ovary granulosa cell tumor, ovary serous carcinoma, pancreas carcinoma, pancreas ductal adenocarcinoma, pancreas neuroendocrine carcinoma, peritoneum mesothelioma, peritoneum serous carcinoma, placenta choriocarcinoma, pleura mesothelioma, prostate acinar adenocarcinoma, prostate carcinoma, rectum adenocarcinoma, rectum squamous cell carcinoma, skin adnexal carcinoma, skin basal cell carcinoma, skin melanoma, skin Merkel cell carcinoma, skin squamous cell carcinoma, small intestine adenocarcinoma, small intestine gastrointestinal stromal tumors (GISTs), soft tissue angiosarcoma, soft tissue Ewing sarcoma, soft tissue hemangioendothelioma, soft tissue inflammatory myofibroblastic tumor, soft tissue leiomyosarcoma, soft tissue liposarcoma, soft tissue neuroblastoma, soft tissue paraganglioma, soft tissue perivascular epitheliod cell tumor, soft tissue sarcoma, soft tissue synovial sarcoma, stomach adenocarcinoma, stomach adenocarcinoma diffuse-type, stomach adenocarcinoma intestinal type, stomach adenocarcinoma intestinal type, stomach leiomyosarcoma, thymus carcinoma, thymus thymoma lymphocytic, thyroid papillary carcinoma, unknown primary adenocarcinoma, unknown primary carcinoma, unknown primary malignant neoplasm, unknown primary melanoma, unknown primary sarcomatoid carcinoma, unknown primary squamous cell carcinoma, unknown undifferentiated neuroendocrine carcinoma, unknown primary undifferentiated small cell carcinoma, uterus carcinosarcoma, uterus endometrial adenocarcinoma, uterus endometrial adenocarcinoma endometrioid, uterus endometrial adenocarcinoma papillary serous, and uterus leiomyosarcoma.

20. A method of treating a subject having a cancer, the method comprising:
(a) detecting a dysregulation of a NTRK gene, a Trk kinase, or the expression or activity or level of any of the same;
(b) administering one or more doses of a first Trk inhibitor to the subject for a period of time;
(c) after (a) and (b), determining whether (i) the cancer in the subject has relapsed during therapy with the first Trk inhibitor; and/or (ii) the cancer in the subject is not responding to therapy with the first Trk inhibitor; and/or (iii) the subject is intolerant to the first Trk inhibitor; and
(d) administering a treatment including one or more doses of a second Trk inhibitor or a pharmaceutically acceptable salt thereof, to a subject in which (i) the cancer in the subject has relapsed during therapy with the first Trk inhibitor; and/or (ii) the cancer in the subject is not responding to therapy with the first Trk inhibitor; and/or (iii) the subject is intolerant to the first Trk inhibitor; or
(e) administering additional doses of the first Trk inhibitor to a subject in which (i) the cancer has not relapsed during therapy with the first Trk inhibitor; and/or (ii) the cancer in the subject is responding to therapy with the first Trk inhibitor; and/or (iii) the subject is not intolerant to the first Trk inhibitor.

21. The method of claim 20, wherein the second Trk inhibitor is Compound 1, or a pharmaceutically acceptable salt, amorphous, or polymorph form thereof.

22. (canceled)

23. (canceled)

24. The method of claim 20, wherein step (d) further comprises administration of another anticancer agent or anticancer therapy.

25. (canceled)

26. The method of claim 20, wherein the dysregulation of a NTRK gene, a Trk kinase, or the expression or activity or level of any of the same is at least one NTRK1, NTRK2, and/or NTRK3 fusion.

27.-84. (canceled)

85. The method of claim 20, wherein the cancer is selected from the group consisting of: adenocarcinoma, adrenal gland cortical carcinoma, adrenal gland neuroblastoma, anus squamous cell carcinoma, appendix adenocarcinoma, bladder urothelial carcinoma, bile duct adenocarcinoma, bladder carcinoma, bladder urothelial carcinoma, bone chordoma, bone marrow leukemia lymphocytic chronic, bone marrow leukemia non-lymphocytic acute myelocytic, bone marrow lymph proliferative disease, bone marrow multiple myeloma, bone sarcoma, brain astrocytoma, brain glioblastoma, brain medulloblastoma, brain meningioma, brain oligodendroglioma, breast adenoid cystic carcinoma, breast carcinoma, breast ductal carcinoma in situ, breast invasive ductal carcinoma, breast invasive lobular carcinoma, breast metaplastic carcinoma, cervix neuroendocrine carcinoma, cervix squamous cell carcinoma, colon adenocarcinoma, colon carcinoid tumor, duodenum adenocarcinoma, endometrioid tumor, esophagus adenocarcinoma, eye intraocular melanoma, eye intraocular squamous cell carcinoma, eye lacrimal duct carcinoma, fallopian tube serous carcinoma, gallbladder adenocarcinoma, gallbladder glomus tumor, gastroesophageal junction adenocarcinoma, head and neck adenoid cystic carcinoma, head and neck carcinoma, head and neck neuroblastoma, head and neck squamous cell carcinoma, kidney chromophore carcinoma, kidney medullary carcinoma, kidney renal cell carcinoma, kidney renal papillary carcinoma, kidney sarcomatoid carcinoma, kidney urothelial carcinoma, leukemia lymphocytic, liver cholangiocarcinoma, liver hepatocellular carcinoma, lung adenocarcinoma, lung adenosquamous carcinoma, lung atypical carcinoid, lung carcinosarcoma, lung large cell neuroendocrine carcinoma, lung non-small cell lung carcinoma, lung sarcoma, lung sarcomatoid carcinoma, lung small cell carcinoma, lung small cell undifferentiated carcinoma, lung squamous cell carcinoma, lymph node lymphoma diffuse large B cell, lymph node lymphoma follicular lymphoma, lymph node lymphoma mediastinal B-cell, lymph node lymphoma plasmablastic lung adenocarcinoma, lymphoma follicular lymphoma, non-Hodgkin's lymphoma, nasopharynx and paranasal sinuses undifferentiated carcinoma, ovary carcinoma, ovary carcinosarcoma, ovary clear cell carcinoma, ovary epithelial carcinoma, ovary granulosa cell tumor, ovary serous carcinoma, pancreas carcinoma, pancreas ductal adenocarcinoma, pancreas neuroendocrine carcinoma, peritoneum mesothelioma, peritoneum serous carcinoma, placenta choriocarcinoma, pleura mesothelioma, prostate acinar adenocarcinoma, prostate carcinoma, rectum adenocarcinoma, rectum squamous cell carcinoma, skin adnexal carcinoma, skin basal cell carcinoma, skin melanoma, skin Merkel cell carcinoma, skin squamous cell carcinoma, small intestine adenocarcinoma, small intestine gastrointestinal stromal tumors (GISTs), soft tissue angiosarcoma, soft tissue Ewing sarcoma, soft tissue hemangioendothelioma, soft tissue inflammatory myofibroblastic tumor, soft tissue leiomyosarcoma, soft tissue liposarcoma, soft tissue neuroblastoma, soft tissue paraganglioma, soft tissue perivascular epitheliod cell tumor, soft tissue sarcoma, soft tissue synovial sarcoma, stomach adenocarcinoma, stomach adenocarcinoma diffuse-type, stomach adenocarcinoma intestinal type, stomach adenocarcinoma intestinal type, stomach leiomyosarcoma, thymus carcinoma, thymus thymoma lymphocytic, thyroid papillary carcinoma, unknown primary adenocarcinoma, unknown primary carcinoma, unknown primary malignant neoplasm, unknown primary melanoma, unknown primary sarcomatoid carcinoma, unknown primary squamous cell carcinoma, unknown undifferentiated neuroendocrine carcinoma, unknown primary undifferentiated small cell carcinoma, uterus carcinosarcoma, uterus endometrial adenocarcinoma, uterus endometrial adenocarcinoma endometrioid, uterus endometrial adenocarcinoma papillary serous, and uterus leiomyosarcoma.

86.-147. (canceled)
